(19) United States
(12) Patent Application Publication
(10) Pub. No.: US 2022/0332765 A1
(43) Pub. Date: Oct. 20, 2022

(54) CORONAVIRUS VACCINE FORMULATIONS

(71) Applicant: Novavax, Inc., Gaithersburg, MD (US)

(72) Inventors: Gale SMITH, Germantown, MD (US); Michael J. MASSARE, Mt. Airy, MD (US); Jing-Hui TIAN, Germantown, MD (US)

(21) Appl. No.: 17/700,945

(22) Filed: Mar. 22, 2022

Related U.S. Application Data
(60) Provisional application No. 63/164,487, filed on Mar. 22, 2021.
(60) Provisional application No. 63/176,825, filed on Apr. 19, 2021.
(60) Provisional application No. 63/177,059, filed on Apr. 20, 2021.
(60) Provisional application No. 63/195,986, filed on Jun. 2, 2021.
(60) Provisional application No. 63/280,395, filed on Nov. 17, 2021.
(60) Provisional application No. 63/290,439, filed on Dec. 16, 2021.
(60) Provisional application No. 63/292,196, filed on Dec. 21, 2021.
(60) Provisional application No. 63/293,468, filed on Dec. 23, 2021.

(51) Int. Cl. C07K 14/005; A61K 39/39; A61K 39/215; A61P 31/14

(52) U.S. Cl. CPC C07K 14/005; A61K 39/39; A61K 39/215; A61P 31/14; A61K 2039/55577

(57) ABSTRACT

Disclosed herein are coronavirus Spike (S) proteins and nanoparticles comprising the same, which are suitable for use in vaccines. The nanoparticles present antigens from pathogens surrounded to and associated with a detergent core resulting in enhanced stability and good immunogenicity. Dosages, formulations, and methods for preparing the vaccines and nanoparticles are also disclosed.

### CROSS-REFERENCE TO RELATED APPLICATIONS

This application claims priority to U.S. Provisional Application No. 63/164,487, filed Mar. 22, 2021; U.S. Provisional Application No. 63/176,825, filed Apr. 19, 2021; U.S. Provisional Application No. 63/177,059, filed Apr. 20, 2021; U.S. Provisional Application No. 63/195,986 filed Jun. 2, 2021; U.S. Provisional Application No. 63/280,395 filed Nov. 17, 2021; U.S. Provisional Application No. 63/290,439 filed Dec. 16, 2021; U.S. Provisional Application No. 63/292,196 filed Dec. 21, 2021; and U.S. Provisional Application No. 63/293,468 filed Dec. 23, 2021. The contents of each of the aforementioned applications are incorporated by reference herein in their entireties. This application also incorporates by reference herein International Publication No. 2021/0154812 and U.S. Pat. No. 10,953,089 in their entireties.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: NOVV_092_01WO_SeqList_ST25.txt, date recorded: Mar. 9, 2022; file size: 974 kilobytes).

### FIELD

The present disclosure is generally related to non-naturally occurring coronavirus (CoV) Spike (S) polypeptides and nanoparticles and vaccines comprising the same, which are useful for stimulating immune responses. The nanoparticles provide antigens, for example, glycoprotein antigens, optionally associated with a detergent core and are typically produced using recombinant approaches. The nanoparticles have improved stability and enhanced epitope presentation. The disclosure also provides compositions containing the nanoparticles, methods for producing them, and methods of stimulating immune responses.

### BACKGROUND OF THE INVENTION

Infectious diseases remain a problem throughout the world. While progress has been made on developing vaccines against some pathogens, many remain a threat to human health. The outbreak of sudden acute respiratory syndrome coronavirus 2 (SARS-CoV-2) has infected more 79 million people in the United States alone, with at least 960,000 deaths. Worldwide, the death toll has surpassed 6 million. The SARS-CoV-2 coronavirus belongs to the same family of viruses as severe acute respiratory syndrome coronavirus (SARS-CoV) and Middle East respiratory syndrome coronavirus (MERS-CoV), which have killed hundreds of people in the past 17 years. SARS-CoV-2 causes the disease COVID-19. The World Health Organization classified COVID-19 as a global pandemic in March 2020. The pandemic is still ongoing. Efforts to control it have been hindered by the emergence of variants of SARS-CoV-2.

The development of vaccines that prevent or reduce the severity of life-threatening infectious diseases caused by SARS-CoV-2 and variants thereof is desirable. However, human vaccine development remains challenging because of the highly sophisticated evasion mechanisms of pathogens and difficulties stabilizing vaccines. Optimally, a vaccine must both induce antibodies that block or neutralize infectious agents and remain stable in various environments, including environments that do not enable refrigeration.

### SUMMARY OF THE INVENTION

The present disclosure provides non-naturally occurring CoV S polypeptides suitable for inducing immune responses against SARS-CoV-2 and SARS-CoV-2 variants. The disclosure also provides nanoparticles containing the glycoproteins as well as methods of stimulating immune responses.

The present disclosure also provides CoV S polypeptides suitable for inducing immune responses against multiple coronaviruses, including SARS-CoV-2 and variants thereof, Middle East Respiratory Syndrome (MERS), and Severe Acute Respiratory Syndrome (SARS).

Provided herein are CoV S polypeptides comprising:

(i) an S1 subunit with an inactivated furin cleavage site, wherein the S1 subunit comprises an N-terminal domain (NTD), a receptor binding domain (RBD), subdomains 1 and 2 (SD1/2), wherein the inactivated furin cleavage site has an amino acid sequence of QQAQ;
wherein the NTD optionally comprises one or more modifications selected from the group consisting of:
(a) deletion of one or more amino acids selected from the group consisting of amino acid 56, 57, 131, 132, 144, 145, 228, 229, 230, 231, 234, 235, 236, 237, 238, 239, 240 and combinations thereof;
(b) insertion of 1, 2, 3, or 4 amino acids after amino acid 132; and
(c) mutation of one or more amino acids selected from the group consisting of amino acid 5, 6, 7, 13, 39, 51, 53, 54, 56, 57, 62, 63, 67, 82, 125, 129, 131, 132, 133, 139, 143, 144, 145, 177, 200, 201, 202, 209, 229. 233, 240, 245, and combinations thereof;
wherein the RBD optionally comprises mutation of one or more amino acids selected from the group consisting of amino acid 333, 404, 419, 426, 439, 440, 464, 465, 471, 477, 481, 488, and combinations thereof;
wherein the SD1/2 domain optionally comprises mutation of one or more amino acids selected from the group consisting of 557, 600, 601, 642, 664, 668, and combinations thereof; and

(ii) an S2 subunit, wherein amino acids 973 and 974 are proline, wherein the S2 subunit optionally comprises one or more modifications selected from the group consisting of:
(a) deletion of one or more amino acids from 676-685, 676-702, 702-711, 775-793, 806-815 and combinations thereof;
(b) mutation of one or more amino acids selected from the group consisting of 688, 703, 846, 875, 937, 969, 973, 974, 1014, 1058, 1105, and 1163 and combinations thereof; and
(c) deletion of one or more amino acids from the TMCT; wherein the amino acids of the CoV S glycoprotein are numbered with respect to a polypeptide having the sequence of SEQ ID NO: 2.

In embodiments, the coronavirus S glycoprotein comprises deletion of amino acids 676-685. In embodiments, the coronavirus S glycoprotein comprises deletion of amino acids 702-711. In embodiments, the coronavirus S glycoprotein comprises deletion of amino acids 806-815. In embodiments, the coronavirus S glycoprotein comprises deletion of amino acids 775-793. In embodiments, the coronavirus S glycoprotein comprises deletion of amino acids 1-292 of the NTD. In embodiments, the coronavirus S glycoprotein comprises deletion of amino acids 1201-1260 of the TMCT. In embodiments, the coronavirus S glycoprotein comprises or consists of an amino acid sequence selected from the group consisting of SEQ ID NOS: 85-89, 105, 106, and 112-115, 164-168 or an amino acid sequence with at least 90%, at least 91%, at least 92%, at least 93%, at least 94%, at least 95%, at least 96%, at least 97%, at least 98%, at least 99%, or 100% identity to any one of SEQ ID NOS: 85-89, 105, 106, and 112-115, 164-168. In embodiments, the coronavirus S glycoprotein comprises a signal peptide, optionally wherein the signal peptide comprises an amino acid sequence of SEQ ID NO: 5 or SEQ ID NO: 117. In embodiments, the coronavirus S glycoprotein comprises a C-terminal fusion protein. In embodiments, the C-terminal fusion protein is a hexahistidine tag. In embodiments, the C-terminal fusion protein is a foldon. In embodiments, the foldon has an amino acid sequence corresponding to SEQ ID NO: 68. In embodiments, the coronavirus S glycoprotein has a ΔHcal that is at least 2-fold greater than the ΔHcal of the wild-type CoV S glycoprotein (SEQ ID NO: 2). In embodiments, provided herein is a coronavirus S glycoprotein having an S2 subunit, NTD, RBD, and SD1/2 that is at least 90%, at least 91%, at least 92%, at least 93%, at least 94%, at least 95%, at least 96%, at least 97%, at least 98%, at least 99%, or at least 99.5% identical to the corresponding subunit or domain of the CoV S glycoprotein having an amino acid sequence of SEQ ID NO: 2.

Provided herein is an isolated nucleic acid encoding a CoV S glycoprotein described herein.

Provided herein is vector comprising an isolated nucleic acid encoding a CoV S glycoprotein described herein.

Provided herein is a nanoparticle comprising a CoV S glycoprotein described herein. In embodiments, the nanoparticle has a Zavg diameter of between about 20 nm and about 35 nm. In embodiments, the nanoparticle has a polydispersity index from about 0.2 to about 0.45. Provided herein is a cell expressing a CoV S glycoprotein described herein.

Provided herein is a vaccine composition comprising a nanoparticle comprising a CoV S glycoprotein described herein. In embodiments, the vaccine composition comprises an adjuvant. In embodiments, the adjuvant comprises at least two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. In embodiments, fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant. In embodiments, fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant. In embodiments, the vaccine composition comprises about 50 μg adjuvant.

Provided herein is a method of stimulating an immune response against SARS-CoV-2 in a subject comprising administering a vaccine composition described herein. In embodiments, the subject is administered a first dose at day 0 and a boost dose at day 21. In embodiments, the subject is administered from about 3 μg to about 25 μg of coronavirus S glycoprotein. In embodiments, the subject is administered about 5 μg of coronavirus S glycoprotein. In embodiments, the vaccine composition is administered intramuscularly. In embodiments, a single dose of the vaccine composition is administered. In embodiments, multiple doses of the vaccine composition are administered. In embodiments, the vaccine composition is coadministered with an influenza glycoprotein.

Provided herein is an immunogenic composition comprising: (i) a nanoparticle comprising a CoV S glycoprotein described herein, and a non-ionic detergent core; (ii) a pharmaceutically acceptable buffer; and (iii) a saponin adjuvant. In embodiments, the immunogenic composition comprises from about 3 μg to about 25 μg of CoV S glycoprotein. In embodiments, the immunogenic composition comprises about 5 μg of CoV S glycoprotein. In embodiments, the saponin adjuvant comprises at least two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. In embodiments, fraction A of Quillaja Saponaria Molina accounts for 50-96% by weight and fraction C of Quillaja Saponaria Molina accounts for the remainder, respectively, of the sum of the weights of fraction A of Quillaja Saponaria Molina and fraction C of Quillaja Saponaria Molina in the adjuvant. In embodiments, fraction A of Quillaja Saponaria Molina and fraction C of Quillaja Saponaria Molina account for about 85% by weight and about 15% by weight, respectively, of the sum of the weights of fraction A of Quillaja Saponaria Molina and fraction C of Quillaja Saponaria Molina in the adjuvant. In embodiments, the immunogenic composition comprises about 50 μg of saponin adjuvant. In embodiments, the non-ionic detergent is selected from the group consisting of polysorbate-20 (PS20), polysorbate-40 (PS40), polysorbate-60 (PS60), polysorbate-65 (PS65), and polysorbate-80 (PS80).

In embodiments, provided herein is a method of stimulating an immune response against SARS-CoV-2 or a heterogeneous SARS-CoV-2 strain in a subject comprising administering the immunogenic composition or vaccine composition provided herein. In embodiments, the method comprises administering from about 3 μg to about 25 μg of CoV S glycoprotein. In embodiments, the method comprises administering about 5 μg of CoV S glycoprotein. In embodiments, the saponin adjuvant comprises at least two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. In embodiments, fraction A of Quillaja Saponaria Molina accounts for 50-96% by weight and fraction C of Quillaja Saponaria Molina accounts for the remainder, respectively, of the sum of the weights of fraction A of Quillaja Saponaria Molina and fraction C of Quillaja Saponaria Molina in the adjuvant. In embodiments, fraction A of Quillaja Saponaria Molina and fraction C of Quillaja Saponaria Molina account for about 85% by weight and about 15% by weight, respectively, of the sum of the weights of fraction A of Quillaja Saponaria Molina and fraction C of Quillaja Saponaria. In embodiments, the method comprises administering about 50 μg of the saponin adjuvant. In embodiments, the non-ionic detergent is selected from the group consisting of polysorbate-20 (PS20), polysorbate-40 (PS40), polysorbate-60 (PS60), polysorbate-65 (PS65), and polysorbate-80 (PS80). In embodiments, the subject is administered a first dose at day 0 and a boost dose at day 21. In embodiments, a single dose of the immunogenic composition is administered. In embodiments, the method comprises administering a second immunogenic composition. In embodiments, the second immunogenic composition comprises an mRNA encoding a SARS-Cov-2 Spike glycoprotein, a plasmid DNA encoding a SARS-Cov-2 Spike glycoprotein, a viral vector encoding a SARS-Cov-2 Spike glycoprotein, or an inactivated SARS-CoV-2 virus. In embodiments, the heterogenous SARS-CoV-2 strain is selected from the group consisting of a B.1.1.7 SARS-CoV-2 strain, B.1.351 SARS-CoV-2 strain, P.1 SARS-CoV-2 strain, B.1.617.2 SARS-CoV-2 strain, B.1.525 SARS-CoV-2 strain, B.1.526 SARS-CoV-2 strain, B.1.617.1 SARS-CoV-2 strain, a C.37 SARS-CoV-2 strain, B.1.621 SARS-CoV-2 strain, and a Ca1.20C SARS-CoV-2 strain. In embodiments, the efficacy of the immunogenic composition for preventing coronavirus disease-19 (COVID-19) is at least about 50%, at least about 55%, at least about 60%, at least about 65%, at least about 70%, at least about 75%, at least about 80%, at least about 85%, about least about 90%, at least about 91%, at least about 92%, at least about 93%, at least about 94%, at least about 95%, at least about 96%, at least about 97%, at least about 98%, at least about 99%, or about 100% for up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, or up to about 12 months after administration of the immunogenic composition. In embodiments, the efficacy of the immunogenic composition for preventing coronavirus disease-19 (COVID-19) is from about 50% to about 99%, from about 50% to about 95%, from about 50% to about 90%, from about 50% to about 85%, from about 50% to about 80%, from about 60% to about 99%, from about 60% to about 95%, from about 60% to about 90%, from about 60% to about 85%, from about 60% to about 80%, from about 40% to about 99%, from about 40% to about 95%, from about 40% to about 90%, from about 40% to about 85%, from about 40% to about 80%, from about 40% to about 75%, from about 40% to about 70%, from about 40% to about 65%, from about 40% to about 55%, or from about 40% to about 50% for up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, or up to about 12 months after administration of the immunogenic composition. In embodiments, the COVID-19 is mild COVID-19. In embodiments, the COVID-19 is moderate COVID-19. In embodiments, the COVID-19 is severe COVID-19. In embodiments, provided herein is a method of inducing a protective immune response against a heterogenous SARS-CoV-2 strain, comprising administering to a subject a nanoparticle comprising a coronavirus S (CoV S) glycoprotein having the amino acid sequence of SEQ ID NO: 87, and a non-ionic detergent core, a pharmaceutically acceptable buffer, and (iii) a saponin adjuvant, wherein the heterogenous SARS-CoV-2 strain has a SARS-CoV-2 S glycoprotein having from about 1 to about 60 modifications compared to a SARS-CoV-2 glycoprotein of SEQ ID NO: 1. In embodiments, the heterogeneous SARS-CoV-2 strain has a SARS-CoV-2 S glycoprotein having from about 1 to about 20 modifications, from about 1 to about 10 modifications, from about 10 to about 20 modifications, from 10 to about 30 modifications, from about 10 to about 40 modifications, from 10 to about 50 modifications, from 10 to about 60 modifications, from 20 to about 60 modifications, from 20 to about 50 modifications, from about 20 to about 40 modifications, from about 5 to about 15 modifications, or from about 5 to about 10 modifications compared to a SARS-CoV-2 glycoprotein of SEQ ID NO: 1.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic of the wild-type amino acid sequence of the SARS-CoV-2 Spike (S) protein (SEQ ID NO: 1). The furin cleavage site RRAR (SEQ ID NO: 6) is highlighted in bold, and the signal peptide is underlined.

FIG. 2 shows the primary structure of a SARS-CoV-2 S polypeptide, which has an inactive furin cleavage site, a fusion peptide deletion, and K986P and V987P mutations. The domain positions are numbered with respect to the amino acid sequence of the wild-type CoV S polypeptide from SARS-CoV-2 containing a signal peptide (SEQ ID NO: 1).

FIG. 3 shows the primary structure of the BV2378 CoV S polypeptide, which has an inactive furin cleavage site, a fusion peptide deletion of amino acids 819-828, and K986P and V987P mutations. The domain positions are numbered with respect to the amino acid sequence of the wild-type CoV S polypeptide from SARS-CoV-2 containing a signal peptide (SEQ ID NO: 1).

FIG. 4 shows purification of the CoV S polypeptides BV2364, BV2365, BV2366, BV2367, BV2368, BV2369, BV2373, BV2374, and B V2375. The data reveal that BV2365 (SEQ ID NO: 4) and BV2373 (SEQ ID NO: 87) which has an inactive furin cleavage site having an amino acid sequence of QQAQ (SEQ ID NO: 7) is expressed as a single chain (S0). In contrast, CoV S polypeptides containing an intact furin cleavage site (e.g. BV2364, BV2366, and BV2374) are cleaved, as evident by the presence of the cleavage product S2.

FIG. 5 shows that the CoV S polypeptides BV2361, BV2365, BV2369, BV2365, BV2373, and BV2374 bind to human angiotensin-converting enzyme 2 precursor (hACE2) by bio-layer interferometry.

FIG. 6 shows that BV2361 from SARS-CoV-2 does not bind the MERS-CoV receptor, dipeptidyl peptidase IV (DPP4) and the MERS S protein does not bind to human angiotensin-converting enzyme 2 precursor (hACE2) by bio-layer interferometry.

FIG. 7 shows that BV2361 binds to hACE2 by enzyme-linked immunosorbent assay (ELISA).

FIG. 8 shows the primary structure of the BV2373 CoV S polypeptide and modifications to the furin cleavage site, K986P, and V987P.

FIG. 9 shows purification of the wild type CoV S polypeptide and the CoV S polypeptides BV2365 and BV2373.

FIG. 10 shows a cryo-electron microscopy (cryoEM) structure of the BV2373 CoV S polypeptide overlaid on the cryoEM structure of the SARS-CoV-2 spike protein (EMB ID: 21374).

FIGS. 11A-F show that the CoV S Spike polypeptides BV2365 and BV2373 bind to hACE2. Bio-layer interferometry reveals that BV2365 (FIG. 11B) and BV2373 (FIG. 11C) bind to hACE2 with similar dissociation kinetics to the wild-type CoV S polypeptide (FIG. 11A) ELISA shows that the wild-type CoV S polypeptide (FIG. 11D) and BV2365 (FIG. 11E) bind to hACE2 with similar affinity while BV2373 binds to hACE2 at a higher affinity (FIG. 11F).

FIGS. 12A-B show the effect of stress conditions, such as temperature, two freeze/thaw cycles, oxidation, agitation, and pH extremes on binding of the CoV S polypeptides BV2373 (FIG. 12A) and BV2365 (FIG. 12B) to hACE2.

FIGS. 13A-B show anti-CoV S polypeptide IgG titers 13 days, 21 days, and 28 days after immunization of mice with two doses (FIG. 13A) and one dose of 0.1 μg to 10 μg of BV2373 with or without Fraction A and Fraction C iscom matrix (i.e., MATRIX-M™) (FIG. 13B).

FIG. 14 shows the induction of antibodies that block interaction of hACE2 in mice immunized with one dose or two doses of 0.1 μg to 10 μg of BV2373 with or without MATRIX-M™.

FIG. 15 shows virus neutralizing antibodies detected in mice immunized with one dose or two doses of 0.1 μg to 10 μg of BV2373 with or without MATRIX-M™.

FIG. 16 shows the virus load (SARS-CoV-2) in the lungs of Ad/CMV/hACE2 mice immunized with either a single dose of BV2373 or two doses of BV2373 spaced 14 days apart with or without MATRIX-M™.

FIGS. 17A-C shows weight loss exhibited by mice after immunization with BV2373. FIG. 17A shows the effect of immunization on weight loss with a single 0.01 μg, 0.1 μg, 1 μg, or 10 μg of BV2373 plus MATRIX-M™. FIG. 17B shows the effect of immunization on weight loss with two doses of BV2373 (0.01 μg, 0.1 μg, 1 μg) plus MATRIX-M™. FIG. 17C shows the effect of immunization on weight loss with two doses of BV2373 (10 μg) in the presence or absence of MATRIX-M™.

FIGS. 18A-B shows the effect of BV2373 on lung histopathology of mice four days (FIG. 18A) or seven days (FIG. 18B) after infection with SARS-CoV-2.

FIG. 19 shows the number of secreting cells after ex vivo stimulation in the spleens of mice immunized with BV2373 in the absence of adjuvant compared to mice immunized with BV2373 in the presence of MATRIX-M™.

FIGS. 20A-E shows the frequency of cytokine secreting CD4+ T cells in the spleens of mice immunized with BV2373 in the presence or absence of MATRIX-M™. FIG. 20A shows the frequency of IFN-γ secreting CD4+ T cells. FIG. 20B shows the frequency of TNF-α secreting CD4+ T cells. FIG. 20C shows the frequency of IL-2 secreting CD4+ T cells. FIG. 20D shows the frequency of CD4+ T cells that secrete two cytokines selected from IFN-γ, TNF-α, and IL-2. FIG. 20E shows the frequency of CD4+ T cells that express IFN-γ, TNF-α, and IL-2.

FIGS. 21A-E shows the frequency of cytokine secreting CD8⁺ T cells in the spleens of mice immunized with BV2373 in the presence or absence of MATRIX-M™. FIG. 21A shows the frequency of IFN-γ secreting CD8⁺ T cells. FIG. 21B shows the frequency of TNF-α secreting CD8⁺ cells. FIG. 21C shows the frequency of IL-2 secreting CD8⁺ T cells. FIG. 20D shows the frequency of CD8⁺ T cells that secrete two cytokines selected from IFN-γ, TNF-α, and IL-2. FIG. 21E shows the frequency of CD8⁺ T cells that express IFNγ, TNF-α, and IL-2.

FIG. 22 illustrates the frequency of CD4⁺ or CD8⁺ cells that express one (single), two (double), or three (triple) cytokines selected from IFN-γ, TNF-α, and IL-2 in the spleens of mice immunized with BV2373 in the presence or absence of MATRIX-M™.

FIGS. 23A-C illustrate the effect of immunization with BV21373 in the presence or absence of MATRIX-M™ on type 2 cytokine secretion from CD4⁺ T cells. FIG. 23A shows the frequency of IL-4 secreting cells. FIG. 23B shows the frequency of IL-5 CD4⁺ secreting cells. FIG. 23C shows the ratio of IFN-γ secreting to IL-4 secreting CD4⁺ T cells.

FIGS. 24A-B illustrate the effect of mouse immunization with BV2373 in the presence or absence of MATRIX-M™ on germinal center formation by assessing the presence of CD4⁺ T follicular helper cells (TFH). FIG. 24A shows the frequency of CD4⁺ T follicular helper cells in spleens, and FIG. 24B shows the phenotype (e.g. CD4⁺ CXCR5⁺ PD-1⁺) of the CD4⁺ T follicular helper cells.

FIGS. 25A-B illustrate the effect of mouse immunization with BV2373 in the presence or absence of MATRIX-M™ on germinal center formation by assessing the presence of germinal center (GC) B cells. FIG. 25A shows the frequency of GC B cells in spleens, and FIG. 25B reveals the phenotype (e.g. CD19⁺ GL7⁺ CD-95⁺) of the CD4⁺ T follicular helper cells.

FIGS. 26A-C show the effect of immunization with BV2373 in the presence or absence of MATRIX-M™ on antibody response in olive baboons. FIG. 26A shows the anti-SARS-CoV-2 S polypeptide IgG titer in baboons after immunization with BV2373. FIG. 26B shows the presence of hACE2 receptor blocking antibodies in baboons following a single immunization with 5 μg or 25 μg of BV2373 in the presence of MATRIX-M™. FIG. 26C shows the titer of virus neutralizing antibodies following a single immunization with BV2373 and MATRIX-M™.

FIG. 27 shows the significant correlation between anti-SARS-CoV-2 S polypeptide IgG and neutralizing antibody titers in olive baboons after immunization with BV2373.

FIG. 28 shows the frequency of IFN-γ secreting cells in peripheral blood mononuclear cells (PBMC) of olive baboons immunized with BV2373 in the presence or absence of MATRIX-M™.

FIGS. 29A-E shows the frequency of cytokine secreting CD4+ T cells in the PBMC of olive baboons immunized with BV2373 in the presence or absence of MATRIX-M™. FIG. 29A shows the frequency of IFN-γ secreting CD4+ T cells. FIG. 29B shows the frequency of IL-2 secreting CD4+ T cells. FIG. 29C shows the frequency of TNF-α secreting CD4+ cells. FIG. 29D shows the frequency of CD4+ T cells that secrete two cytokines selected from IFN-γ, TNF-α, and IL-2. FIG. 29E shows the frequency of CD4+ T cells that express IFN-γ, TNF-α, and IL-2.

FIG. 30 shows a schematic of the coronavirus Spike (S) protein (SEQ ID NO: 109) (BV2384). The furin cleavage site GSAS (SEQ ID NO: 97) is underlined once, and the K986P and V987P mutations are underlined twice.

FIG. 31 shows a schematic of the coronavirus Spike (S) protein (SEQ ID NO: 86) (BV2373). The furin cleavage site QQAQ (SEQ ID NO: 7) is underlined once, and the K986P and V987P mutations are underlined twice.

FIG. 32 shows purification of the CoV S polypeptides BV2373 (SEQ ID NO: 87) and BV2384 (SEQ ID NO: 109).

FIG. 33 shows a scanning densitometry plot of BV2384 (SEQ ID NO: 109) purity after purification.

FIG. 34 shows a scanning densitometry plot of BV2373 (SEQ ID NO: 87) purity after purification

FIGS. 35A-B illustrates induction of anti-S antibodies (FIG. 35A) and neutralizing antibodies (FIG. 35B) in response to administration of BV2373 and MATRIX-M™. Cynomolgus macaques were administered one or two doses (Day 0 and Day 21) of 2.5 μg, 5 μg, or 25 μg of BV2373 with 25 μg or 50 μg MATRIX-M™ adjuvant. Controls received neither BV2373 or MATRIX-M™. Antibodies were measured at Days 21 and 33.

FIGS. 36A-B illustrates a decrease of SARS-CoV-2 viral replication by vaccine formulations disclosed herein as assessed in broncheoalveol lavage (BAL) in Cynomolgus macaques. Cynomolgus macaques were administered BV2373 and MATRIX-M™ as shown. Subjects were immunized Day 0 and in the groups with two doses Day 0 and Day 21. Subject animals were challenged Day 37 with 1×10⁴ pfu SARS-CoV-2 virus. Viral RNA (FIG. 36A, corresponding to total RNA present) and viral sub-genomic RNA (FIG. 36B, corresponding to replicating virus) levels were assessed in bronchiolar lavage (BAL) at 2 days and 4 days post-challenge with infectious virus (d2pi and d4pi). Most subjects showed no viral RNA. At Day 2 small amounts of RNA were measured in some subjects. By Day 4, no RNA was measured except for two subjects at the lowest dose of 2.5 μg. Sub-genomic RNA was not detected at either 2 Days or 4 days except for 1 subject, again at the lowest dose.

FIGS. 37A-B illustrates a decrease of SARS-CoV-2 viral replication by vaccine formulations disclosed herein as assessed in nasal swab in Cynomolgus macaques. Cynomolgus macaques were administered BV2373 with MATRIX-M™ as shown. Subjects were immunized Day 0 and in the groups with two doses Day 0 and Day 21. Subject animals were challenged Day 37 with 1×10⁴ SARS-CoV-2 virus. Viral RNA (FIG. 37A) and viral sub-genomic (sg) RNA (FIG. 37B) were assessed by nasal swab at 2 days and 4 days post-infection (d2pi and d4pi). Most subjects showed no viral RNA. At Day 2 and Day 4 small amounts of RNA were measured in some subjects. Sub-genomic RNA was not detected at either 2 Days or 4 days. Subjects were immunized Day 0 and in the groups with two doses Day 0 and Day 21. These data show that the vaccine decreases nose total virus RNA by 100-1000 fold and sgRNA to undetectable levels, and confirm that immune response to the vaccine will block viral replication and prevent viral spread.

FIGS. 38A-B show anti-CoV S polypeptide IgG titers 21 days and 35 days after immunization of Cynomolgus macaques with one dose (FIG. 38A) or two doses of BV2373 and 25 μg or 50 μg of MATRIX-M™ (FIG. 38B).

FIGS. 38C-38D shows the hACE2 inhibition titer of Cynomolgus macaques 21 days and 35 days after immunization of Cynomolgus macaques with one dose (FIG. 38C) or two doses of BV2373 (5 μg) and MATRIX-M™ (25 μg or 50 μg) (FIG. 38D).

FIG. 38E shows the significant correlation between anti-CoV S polypeptide IgG titer and hACE2 inhibition titer in Cynomolgus macaques after administration of BV2373 and MATRIX-M™. Data is shown for Groups 2-6 of Table 4.

FIG. 39 shows the anti-CoV S polypeptide titers and hACE2 inhibition titer of Cynomolgus macaques 35 days after immunization with two doses of BV2373 and MATRIX-M™ or after immunization with convalescent human serum (Groups 2, 4, and 6) of Table 4. These data show that the anti-CoV S polypeptide and hACE2 inhibition titers of Cynomologus macaques immunized with BV2373 and MATRIX-M™ is superior to Cynomolgus macaques immunized with convalescent serum.

FIGS. 40A-B shows the SARS-CoV-2 neutralizing titers of Cynomolgus macaques immunized with BV2373 and MATRIX-M™ determined by cytopathic effect (CPE) (FIG. 40A) and plaque reduction neutralization test (PRNT) (FIG. 40B).

FIG. 41 shows administration timings of a clinical trial that evaluated the safety and efficacy of a vaccine comprising BV2373 and optionally MATRIX-M™. AESI denotes an adverse event of special interest. MAEE denotes a medically attended adverse event, and SAE denotes a serious adverse event.

FIGS. 42A-B show the local (FIG. 42A) and systemic adverse events (FIG. 42B) experienced by patients in a clinical trial which evaluated a vaccine comprising BV2373 and MATRIX-M™. Groups A-E are identified in Table 5. The data shows that the vaccine was well tolerated and safe.

FIGS. 43A-B show the anti-CoV S polypeptide IgG (FIG. 43A) and neutralization titers (FIG. 43B) 21 days and 35 days after immunization of participants in a clinical trial which evaluated a vaccine comprising BV2373 and MATRIX-M™. Horizontal bars represent interquartile range (IRQ) and median area under the curve, respectively. Whisker endpoints are equal to the maximum and minimum values below or above the median±1.5 times the IQR. The convalescent serum panel includes specimens from PCR-confirmed COVID-19 participants from Baylor College of Medicine (29 specimens for ELISA and 32 specimens for microneutralization (MN IC_{>99}). Severity of COVID-19 is denoted as a red mark for hospitalized patients (including intensive care setting), a blue mark for outpatient-treated patients (sample collected in emergency department), and a green mark for asymptomatic (exposed) patients (sample collected from contact/exposure assessment).

FIGS. 44A-C shows the correlation between anti-CoV S polypeptide IgG and neutralizing antibody titers in patients administered convalescent sera (FIG. 44A), two 25 μg doses of BV2373 (FIG. 44B), and two doses (5 μg and 25 μg) of BV2373 with MATRIX-M™ (FIG. 44C). A strong correlation was observed between neutralizing antibody titers and anti-COV-S IgG titers in patients treated with convalescent sera or with adjuvanted BV2373, but not in patients treated with BV2373 in the absence of adjuvant.

FIGS. 45A-D show the frequencies of antigen-specific CD4⁺ T cells producing T helper 1 (Th1) cytokines interferon-gamma (IFN-γ), tumor necrosis factor-alpha (TNF-α), and interleukin (IL)-2 and T helper 2 (Th2) cytokines IL-5 and IL-13 indicated cytokines from participants in Groups A (placebo, FIG. 45A), B (25 μg BV2373, FIG. 45B), C (5 μg BV2373 and 50 μg MATRIX-M™, FIG. 45C), and D (25 μg BV2373 and 50 μg MATRIX-M™, FIG. 45D) following stimulation with BV2373. “Any 2” in Th1 cytokine panel means CD4⁺ T cells that can produce two types of Th1 cytokines at the same time. “All 3” indicates CD4⁺ T cells that produce IFN-γ, TNF-α, and IL-2 simultaneously. “Both” in Th2 panel means CD4⁺ T cells that can produce Th2 cytokines IL-5 and IL-13 at the same time.

FIG. 46A shows the primary structure of a wild-type SARS-CoV-2 S polypeptide, containing a signal peptide, numbered with respect to SEQ ID NO: 1. FIG. 46B shows the primary structure of a wild-type SARS-CoV-2 S polypeptide, without a signal peptide, numbered with respect to SEQ ID NO: 2.

FIG. 47 shows the randomization of subjects in a Phase 3 clinical trial that evaluated the efficacy, immunogenicity, and safety of BV2373 in combination with Fraction A and Fraction C iscom matrix (MATRIX-M™) adjuvant.

FIG. 48 is a Kaplan-Meyer plot showing the incidence of symptomatic COVID-19 (Cumulative Event Rate (%) experienced by subjects after vaccination with BV2373 in combination with Fraction A and Fraction C iscom matrix (MATRIX-M™) or placebo.

FIG. 49 shows vaccine efficacy of BV2373 in combination with Fraction A and Fraction C iscom matrix (MATRIX-M™) against SARS-CoV-2 comprising a CoV S polypeptide having the amino acid sequence of SEQ ID NO: 1 or the heterogeneous B.1.1.7 SARS-CoV-2 strain which comprises a CoV S polypeptide having deletions of amino acids 69, 70, and 144 and mutations of N501Y, A570D, D614G, P681H, T716I, S982A, and D1118H.

FIG. 50 is a graph showing adverse events experienced by subjects after a first vaccination dose (labeled “Vaccination 1”) and a second vaccination dose (labeled “Vaccination 2”) with BV2373 in combination with Fraction A and Fraction C iscom matrix (MATRIX-M™) (labeled “A”) or placebo (labeled “B”).

FIG. 51 shows a diagram of the BV2438 CoV S polypeptide. Structural elements include the cleavable signal peptide (SP), N-terminal domain (NTD), receptor binding domain (RBD), subdomains 1 and 2 (SD1 and SD2), S2 cleavage site (S2′), fusion peptide (FP), heptad repeat 1 (HR1), central helix (CH), heptad repeat 2 (HR2), transmembrane domain (TM), and cytoplasmic tail (CT). Amino acid changes from the CoV S polypeptide having an amino acid sequence of SEQ ID NO: 1 are shown in black text underneath the linear diagram.

FIG. 52A shows a reduced SDS-PAGE gel with Coomassie blue staining of purified full-length BV2438 showing the main protein product at the expected molecular weight of ˜170 kD. FIG. 52B shows a graph of the scanning densitometry. FIG. 52C shows negative stain transmission electron micrographs of BV2438. BV2438 forms a well-defined lightbulb-shaped particle with a length of 15 nm and a width of 11 nm (left panel). Trimers exhibited an 8 nm flexible linker connected to PS-80 micelles (left panel). Class average images showed a good fit of the rS-B.1.351 trimer with a cryo-EM solved structure of the prefusion SARS-CoV-2 trimeric spike protein ectodomain (PDB ID 6VXX) overlaid on the 2D image (middle panel). The right panel shows two BV2438 trimers anchored into a PS-80 micelle.

FIG. 53 shows the mouse study design of Example 10. Groups of mice (n=20/group) were immunized in a prime/boost regimen on study days 0 and 14 with various combinations of recombinant S (rS) BV2438 (SA) or BV2373 (WU) protein. Mice were either primed and boosted with BV2438, primed and boosted with BV2373, primed with BV2373 and boosted with BV2438, or primed and boosted with bivalent BV2373+BV2438. Antigen doses were 1 μg rS for each monovalent immunization, or 1 μg rS for each construct upon bivalent immunization (2 μg rS total). All antigen doses were administered with 5 μg saponin adjuvant. A control group received formulation buffer (Placebo). Sera and tissues were collected at the timepoints listed in the diagram.

FIGS. 54A-B shows Anti-SARS-CoV-2 S IgG serum titers in sera collected on Day 21 of the mouse study of Example 10. ELISA was used to measure antibody titers against the Wuhan-Hu-1 spike protein (FIG. 54A) or B.1.351 spike protein (FIG. 54B). Bars indicate the geometric mean titer (GMT) and error bars represent 95% confidence interval (CI) for each group. Individual animal titers are indicated with colored symbols. FIGS. 54C-D show functional antibody titers (as measured by ELISA) in sera collected on Day 21 capable of disrupting binding between the SARS-CoV-2 receptor hACE2 and Wuhan-Hu-1 spike protein (FIG. 54C) or B.1.351 spike protein (FIG. 54D). Bars indicate the geometric mean titer (GMT) and error bars represent 95% confidence interval (CI) for each group. Individual animal titers are indicated with colored symbols. FIG. 54E shows SARS-CoV-2 neutralizing antibody titers in sera collected on Day 32 from n=5 animals/group were determined using a PRNT assay. Sera were evaluated for their ability to neutralize SARS-CoV-2 USA-WA1, B.1.351 variant, or B.1.1.7 variant. Bars indicate the geometric mean titer (GMT) and error bars represent 95% confidence interval (CI) for each group. Individual animal titers are indicated with symbols. Statistical significance was calculated by performing one-way ANOVA with Tukey's post hoc test on log to-transformed data.

FIGS. 55A-F show the protective efficacy of immunization with SARS-CoV-2 rS based on Wuhan-Hu-1 or B.1.351 against challenge with live SARS-CoV-2 B.1.351 or B.1.1.7 virus. The study design was described in FIG. 53. Immunized mice (n=10/group) were challenged with live SARS-CoV-2 B.1.351 (left panels) or B.1.1.7 (right panels). For four days after challenge, mice were weighed daily and their percentage weight loss was calculated relative to their body weight on challenge day FIG. 55A and FIG. 55B show the mean percentage body weight loss with symbols. Error bars represent standard error of the mean. Half of the mice were sacrificed at 2 days post-challenge and lung tissue was subjected to a plaque formation assay to determine lung viral titers (FIG. 55C, FIG. 55D). The remaining mice were sacrificed at 4 days post-challenge. FIG. 55E and FIG. 55F show levels of SARS-CoV-2 subgenomic RNA in lung tissue and expressed as fold-change in RNA relative to the mean in the respective Placebo group on Day 2 post-challenge. Horizontal bars represent group mean fold-change from n=5 mice at each timepoint and error bars represent standard deviation.

FIGS. 56A-H show cell-mediated immunity induced upon immunization with BV2373 or BV2438 regimens in mice. FIG. 56A shows the mouse study design. Groups of mice (n=8/group) were immunized in a prime/boost regimen on Days 0 and 21 with various combinations of SARS-CoV-2 rS based on BV2373 or BV2438. Mice were either primed and boosted with BV2438, primed and boosted with BV2373, primed with BV2373 and boosted with BV2438, or primed and boosted with bivalent BV2373 and BV2438. Antigen doses were 1 μg rS for each monovalent immunization, or 1 μg rS for each construct upon bivalent immunization (2 μg rS total). All immunizations were administered with 5 μg Matrix-M1 adjuvant. A control group received formulation buffer (Placebo, n=5). Spleens were harvested on Day 28 for cell collection. Splenocytes were stimulated with BV2373 or BV2438, then subjected to ELISA to determine IFN-γ-positive cells as a representative Th1 cytokine (FIG. 56B) and IL-5-positive cells as a representative Th2 cytokine (FIG. 56C). Data from FIG. 56B and FIG. 56C were used to calculate the Th1/Th2 balance of responses to immunization (FIG. 56D). FIG. 56E shows the numbers of multifunctional CD4+ T cells that stained positively for three Th1 cytokines (IFN-γ, IL-2, and TNF-α) using intracellular cytokine staining were quantified and expressed as the number of triple cytokine positive cells per 10⁶ CD4+ T cells. FIG. 56F shows quantification of T follicular helper cells. T follicular helper cells were quantified by determining the percentage of PD-1+CXCR5+ cells among all CD4+ T cells. FIG. 56G shows germinal center formation Germinal center formation was evaluated by determining the percentage of GL7+CD95+ cells among CD19+ B cells using flow cytometry. Gray bars represent means and error bars represent standard deviation. Individual animal data are shown with colored symbols. An example of the gating strategy is shown in FIG. 56H. Differences among experimental groups were evaluated by one-way ANOVA with Tukey's post-hoc test (data in FIG. 56B were log₁₀-transformed before analysis). P values<0.05 were considered statistically significant; ****=p<0.0001.

FIGS. 57A-E show the CD4+ and CD8+ T cell response from immunization with BV2373 or BV2438. Groups of mice (n=8/group) were immunized in a prime/boost regimen on Days 0 and 21 with various combinations of BV2373 or BV2438. Mice were either primed and boosted with BV2438, primed and boosted with BV2373, primed with BV2373 and boosted with BV2438, or primed and boosted with bivalent BV2373 and BV2438. Antigen doses were 1 μg rS for each monovalent immunization, or 1 μg rS for each construct upon bivalent immunization (2 μg rS total). All antigen doses were administered with 5 μg saponin adjuvant. A control group received formulation buffer (Placebo, n=5). Spleens were harvested on Day 28 for cell collection. Isolated splenocytes were stimulated with either rS-WU1 or rS-B.1.351, then subjected to intracellular cytokine staining to determine whether CD4+ T cells were positive for IFN-γ (FIG. 57A), IL-2 (FIG. 57B), TNF-α (FIG. 57C), or IL-4 (FIG. 57D). To examine CD8+ T cell responses, cells were stimulated with a peptide pool corresponding to the entire Wuhan-Hu-1 spike protein sequence, then subjected to ICS for IFN-γ, IL-2, and TNF-α (FIG. 57E).

FIGS. 58A-G show the immunogenicity of one or two booster BV2438 doses approximately one year after immunization with BV2373 in baboons. FIG. 58A shows the study design. A small cohort of baboons (n=2-3/group) were originally immunized with 1 μg, 5 μg, or 25 μg BV2373 with saponin adjuvant or unadjuvanted 25 μg BV2373 on Day 0 and 21 (Week 0 and 3, respectively). Approximately 1 year later, all animals were boosted with one or two doses of 3 μg BV2438 with 50 μg saponin adjuvant on Day 318 and 339 (Week 45 and 48, respectively). FIG. 58B show the anti-CoV S IgG titer over the course of the study. Individual animals' titers are shown over time, different colored symbols and lines represent different dose groups for the initial rS-WU1 immunization series. Sera collected before BV2438 boost (Day 303) as well as 7, 21, 35, and 81 days after the boost were analyzed to determine anti-rS-WUI1 (FIG. 58C) and rS-B.1.351 (FIG. 58D) IgG titers by ELISA (horizontal lines represent means), antibody titers capable of disrupting the interaction between rS-WU1 or rS-B.1.351 and the hACE2 receptor by ELISA (FIG. 58E, horizontal lines represent means), and antibody titers capable of neutralizing SARS-CoV-2 strains USA-WA1, B.1.351, and B.1.1.7 with a PRNT assay (FIG. 58F, gray bars represent geometric means and error bars represent 95% confidence intervals). The presence of multifunctional CD4+ T cells positive for 3 Th1 cytokines (IFN-γ, IL-2, and TNF-α) was evaluated with intracellular cytokine staining after stimulation with BV2373 or BV2438 (FIG. 58G). Gray bars represent means and colored symbols represent individual animal data.

FIGS. 59A-G show individual Cytokine Responses to BV2438 boost in baboons. A small cohort of baboons (n=2-3/group) was immunized with 1 μg, 5 μg, or 25 μg BV2373 with 50 μg saponin adjuvant or unadjuvanted 25 μg BV2373 on Day 0 and 21 (Week 0 and 3, respectively). Approximately 1 year later, all animals were boosted with one or two doses of 3 μg BV2438 with 50 μg saponin adjuvant on Day 318 and 339 (Weeks 45 and 48, respectively). PBMCs collected pre-boost (Day 303; Week 43), 7 days after the first rS-B.1.351 boost (Day 325; Week 46), and 35 days after the first rS-B.1.351 boost (Day 353; Week 50). PBMCs were stimulated with BV2373 or BV2438 and subjected to ELISA to measure (FIG. 59A) IFN-γ producing cells as a Th1 cytokine and (FIG. 59B) IL-4 producing cells as a Th2 cytokine. CD4+ T cells were also stimulated with BV2373 or BV2438, then subjected to ICS to measure cells producing IFN-γ (FIG. 59C), IL-2 (FIG. 59D), TNF-α (FIG. 59E), IL-5 (FIG. 59F), and IL-13 (FIG. 59G).

FIGS. 60A-B show SARS-CoV-2 variant neutralizing titers from human subjects immunized with BV2373. Serum samples from clinical study participants (n=30) were subjected to a PRNT assay to determine the presence of neutralizing antibodies against USA-WA1 compared to B.1.1.7 (FIG. 60A) and B.1.351 (FIG. 60B). Individual subjects' titers are shown with black circles, lines connect individuals' titers against USA-WA1 to their titer against the respective variant.

FIGS. 61A-B shows anti-S protein IgG titers before and after boost with BV2373 and a saponin adjuvant for the following SARS-CoV-2 variants: (i) SARS-CoV-2 virus having a CoV S polypeptide with a D614G mutation compared to the protein having an amino acid sequence of SEQ ID NO: 1; (ii) a SARS-CoV-2 alpha strain, a SARS-CoV-2 beta strain, and a SARS-CoV-2 delta strain. FIG. 61A shows the fold increase from day 35 to day 217. FIG. 61B shows the fold increase from day 189 to day 217.

FIGS. 62A-B shows the functional hACE2 inhibition before and after boost with BV2373 and a saponin adjuvant for the following SARS-CoV-2 variants: (i) SARS-CoV-2 virus having a CoV S polypeptide with a D614G mutation compared to the protein having an amino acid sequence of SEQ ID NO: 1; (ii) a SARS-CoV-2 alpha strain, a SARS-CoV-2 beta strain, and a SARS-CoV-2 delta strain. FIG. 62A shows the fold increase from day 35 to day 217. FIG. 62B shows the fold increase from day 189 to day 217.

FIG. 63 shows a diagram of booster dosing for participants of the trial described in Example 11.

FIGS. 64A-B show local (FIG. 64A) and systemic (FIG. 64B) reactogenicity of patients in Group B2 of the trial described in Example 11.

FIG. 65 shows serum IgG titers to the ancestral SARS-CoV-2 strain by study day of the patients described in Example 11.

FIG. 66 shows neutralizing antibody activity for the ancestral SARS-CoV-2 strain by study day of the patients described in Example 11.

FIG. 67 shows the neutralizing antibody 99 (neut99) values for the immunogenic composition comprising BV2373 and saponin adjuvant of Example 11 against the SARS-CoV-2 strain containing a D614G mutation and the B.1.617.2 (delta variant).

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, the term “about” or “approximately” when preceding a numerical value indicates the value plus or minus a range of 10%. For example, “about 100” encompasses 90 and 110.

As used herein, the terms “immunogen,” “antigen,” and “epitope” refer to substances such as proteins, including glycoproteins, and peptides that are capable of eliciting an immune response.

As used herein, an “immunogenic composition” is a composition that comprises an antigen where administration of the composition to a subject results in the development in the subject of a humoral and/or a cellular immune response to the antigen.

As used herein, a “subunit” composition, for example a vaccine, that includes one or more selected antigens but not all antigens from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen. The immune response may include formation of antibodies and/or a cell-mediated response. Depending on context, the term “vaccine” may also refer to a suspension or solution of an immunogen that is administered to a subject to produce an immune response. Preferably, vaccines induces an immune response that is effective at preventing infection from SARS-CoV-2 or a variant thereof.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In aspects, the adults are seniors about 65 years or older, or about 60 years or older. In aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

In aspects, the subject is immunocompromised. In embodiments, the immunocompromised subject is administered a medication that causes immunosuppression. Non-limiting examples of medications that cause immunosuppression include corticosteroids (e.g., prednisone), alkylating agents (e.g., cyclophosphamide), antimetabolites (e.g., azathioprine or 6-mercaptopurine), transplant-related immunosuppressive drugs (e.g., cyclosporine, tacrolimus, sirolimus, or mycophenolate mofetil), mitoxantrone, chemotherapeutic agents, methotrexate, tumor necrosis factor (TNF)-blocking agents (e.g., etanercept, adalimumab, infliximab). In embodiments, the immunocompromised subject is infected with a virus (e.g., human immunodeficiency virus or Epstein-Barr virus). In embodiments, the virus is a respiratory virus, such as respiratory syncytial virus, influenza, parainfluenza, adenovirus, or a picornavirus. In embodiments, the immunocompromised subject has acquired immunodeficiency syndrome (AIDS). In embodiments, the immunocompromised subject is a person living with human immunodeficiency virus (HIV). In embodiments, the immunocompromised subject is immunocompromised due to a treatment regiment designed to prevent inflammation or prevent rejection of a transplant. In embodiments, the immunocompromised subject is a subject who has received a transplant. In embodiments, the immunocompromised subject has undergone radiation therapy or a splenectomy. In embodiments, the immunocompromised subject has been diagnosed with cancer, an autoimmune disease, tuberculosis, a substance use disorder (e.g., an alcohol, opioid, or cocaine use disorder), stroke or cerebrovascular disease, a solid organ or blood stem cell transplant, sickle cell disease, thalassemia, autoimmune lymphoproliferative syndrome (ALPS), autoimmune polyglandular syndrome type 1 (APS-1), B-cell expansion with NF-kB and T-cell anergy (BENTA) disease, Caspase-8 deficiency state (CEDS), chronic granulomatous disease (CGD), common variable immunodeficiency (CVID), congenital neutropenia syndromes, a deficiency in the cytotoxic T-lymphocyte-associated antigen 4 (CTLA-4), a DOCK8 deficiency, a GATA2 deficiency, a glycosylation disorder with immunodeficiency, a hyper-immunoglobulin E syndrome (HIES), hyper-immunoglobulin M syndrome, diabetes, type 1 diabetes, type 2 diabetes, interferon gamma deficiency, interleukin 12 deficiency, interleukin 23 deficiency, leukocyte adhesion deficiency, lipopolysaccharide-responsive beige-like anchor (LRBA) deficiency, PI3 kinase disease, PLCG2-associated antibody deficiency and immune dysregulation (PLAID), severe combined immunodeficiency (SCID), STAT3 dominant-negative disease, STAT3 gain-of-function disease, warts, hypogammaglobulinemia, infections, and myelokathexis (WHIM) syndrome, Wisckott-Aldrich syndrome (WAS), X-linked agammaglobulinemia (XLA), X-linked lymphoproliferative disease (XLP), uremia, malnutrition, or XMEN disease. In embodiments, the immunocompromised subject is a current or former cigarette smoker. In embodiments, the immunocompromised subject has a B-cell defect, T-cell defect, macrophage defect, cytokine defect, phagocyte deficiency, phagocyte dysfunction, complement deficiency or a combination thereof.

In embodiments, the subject is overweight or obese. In embodiments, an overweight subject has a body mass index (BMI)≥25 kg/m² and <30 kg/m². In embodiments, an obese subject has a BMI that is ≥30 kg/m². In embodiments, the subject has a mental health condition. In embodiments, the mental health condition is depression, schizophrenia, or anxiety.

As used herein, the term “pharmaceutically acceptable” means being approved by a regulatory agency of a U.S. Federal or a state government or listed in the U.S. Pharmacopeia, European Pharmacopeia or other generally recognized pharmacopeia, for use in mammals, and more particularly in humans. These compositions can be useful as a vaccine and/or antigenic compositions for inducing a protective immune response in a vertebrate.

As used herein, the term “about” means plus or minus 10% of the indicated numerical value.

As used herein, the term “NVX-CoV2373” refers to a vaccine composition comprising the BV2373 Spike glycoprotein (SEQ ID NO: 87) and Fraction A and Fraction C iscom matrix (e.g., MATRIX-M™).

As used herein, the term “modification” as it refers to a CoV S polypeptide refers to mutation, deletion, or addition of one or more amino acids of the CoV S polypeptide. The location of a modification within a CoV S polypeptide can be determined based on aligning the sequence of the polypeptide to SEQ ID NO: 1 (CoV S polypeptide containing signal peptide) or SEQ NO: 2 (mature CoV S polypeptide lacking a signal peptide).

The term variant of SARS-CoV-2 used interchangeably herein with a “heterogeneous SARS-CoV-2 strain” is a SARS-CoV-2 virus comprising a CoV S polypeptide having at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, or at least about 35 modifications, between about 2 and about 35 modifications, between about 5 and about 10 modifications, between about 5 and about 20 modifications, between about 10 and about 20 modifications, between about 15 and about 25 modifications, between about 20 and 30 modifications, between about 20 and about 40 modifications, between about 25 about 45 modifications, as compared to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with at least about 70%, at least about 75%, at least about 80%, at least about 85%, at least about 90%, at least about 95%, at least about 96%, at least about 97%, at least about 98%, or at least about 99% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 70% and about 99.9% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 70% and about 99.5% identity to a CoV S polypeptide having the ammo acid sequence of SEQ ID NO: 1 or SEQ ID NO: 2. In embodiments, the heterogeneous SARS-CoV-2 strain is a SARS-CoV-2 virus comprising a CoV S polypeptide with between about 90% and about 99.9% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 90% and about 99.8% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 95% and about 99.9% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 95% and about 99.8% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 95% and about 99% identity to a CoV S polypeptide having the amino acid sequence of SEQ ID NO: 1 or SEQ ID NO: 2.

The term “B.1.1.7 SARS-CoV-2 strain” (also referred to as an “alpha” strain) refers to a heterogenous SARS-CoV-2 strain having a CoV S polypeptide comprising deletions of amino acids 69, 70, and 144 and mutations of N501Y, A570D, D614G, P681H or P681R, T716I, S982A, and D1118H, wherein the CoV S polypeptide is numbered with respect to the wild-type SARS-CoV-2 S polypeptide having the amino acid sequence of SEQ ID NO: 1. The CoV S polypeptide of a B.1.1.7 SARS-CoV-2 strain may optionally contain a deletion of amino acid 145, mutation of E484K, L432R, or S494P, or a combination thereof.

The term “B.1.351 SARS-CoV-2 strain” (also referred to as a “beta” strain) refers to a heterogenous SARS-CoV-2 strain having a CoV S polypeptide comprising mutations of D80A, K417N, E484K, N501Y, D614G, and A701V, wherein the CoV S polypeptide is numbered with respect to the wild-type SARS-CoV-2 S polypeptide having the amino acid sequence of SEQ ID NO: 1. The CoV S polypeptide of a B.1.617.2 SARS-CoV-2 strain may optionally contain one or more of the following mutations: D215G; L242H; R246I; or deletion of 1, 2, or 3 amino acids of 241-243, wherein the CoV S polypeptide is numbered with respect to the wild-type SARS-CoV-2 S polypeptide having the amino acid sequence of SEQ ID NO: 1. In embodiments, the beta strain's CoV S polypeptide comprises mutations of D80A, D215G, L242H, K417N, E484K, N501Y, D614G, and A701V, wherein the CoV S polypeptide is numbered with respect to the wild-type SARS-CoV-2 S polypeptide having the amino acid sequence of SEQ ID NO: 1. In embodiments, the beta strain's CoV S polypeptide comprises mutations of D80A, D215G, deletion of 1, 2, or 3 amino acids of amino acids 241-243, K417N, E484K, N501Y, D614G, and A701V, wherein the CoV S polypeptide is numbered with respect to the wild-type SARS-CoV-2 S polypeptide having the amino acid sequence of SEQ ID NO: 1. In embodiments, the beta strain comprises mutations of D80A, L242H, R246I, N501Y, K417N, E484K, D614G, and A701V, wherein the CoV S polypeptide is numbered with respect to the wild-type SARS-CoV-2 S polypeptide having the amino acid sequence of SEQ ED NO: 1.

The term “P.1 SARS-CoV-2 strain” (also referred to as a “gamma” strain) refers to a heterogenous SARS-CoV-2 strain having a CoV S polypeptide containing the mutations L18F, T20N, P26S, D138Y, R190S, K417T, E484K, N501Y, D614G, H655Y, T1027I, and V1176F, wherein the CoV S polypeptide is numbered with respect to the wild-type SARS-CoV-2 S polypeptide having the amino acid sequence of SEQ ID NO: 1.

The term “Cal.20C SARS-CoV-2 strain” refers to a heterogeneous SARS-CoV-2 strain having a CoV S polypeptide containing the mutations S131, W152C, and L452R, wherein the CoV S polypeptide is numbered with respect to the wild-type SARS-CoV-2 S polypeptide having the amino acid sequence of SEQ ID NO: 1.

The term “B.1.617.2 strain” (also referred to as “delta” strain) refers to a heterogeneous SARS-CoV-2 strain having a CoV S polypeptide comprising deletions of amino acids 157 and 158 and mutations of T19R, E156G, L452R, T478K, D614G, P681R, and D950N, wherein the CoV S polypeptide is numbered with respect to the wild-type SARS-CoV-2 S polypeptide having the amino acid sequence of SEQ ID NO: 1. The CoV S polypeptide of a B.1.617.2 SARS-CoV-2 strain may optionally contain one or more of the following mutations: G142D; W64H; H66W; V70F; T95I; Y145H; D213V; L214R; A222V; W258I or W258L; K417N; N439K; E484K or E484Q; N501 Y; and Q613H, wherein the CoV S polypeptide is numbered with respect to the wild-type SARS-CoV-2 S polypeptide having the amino acid sequence of SEQ ID NO: 1. In embodiments, the delta strain comprises a CoV S polypeptide comprising deletions of amino acids 157 and 158 and mutations of T19R, G142D, E156G, L452R, T478K, D614G, P681R, and D950N. In embodiments, the delta strain comprises a CoV S polypeptide comprising deletions of amino acids 157 and 158 and mutations of T19R, T95I, G142D, Y145H, E156G, A222V, K417N L452R, T478K, D614G, P681R, and D950N, wherein the CoV S polypeptide is numbered with respect to the wild-type SARS-CoV-2 S polypeptide having the amino acid sequence of SEQ ID NO: 1. In embodiments, the delta strain comprises a CoV S polypeptide comprising deletions of amino acids 157 and 158 and mutations of T19R, G142D, E156G, W258I, K417N, N439K, L452R, T478K, E484K, N501Y, D614G, P681R, and D950N, wherein the CoV S polypeptide is numbered with respect to the wild-type SARS-CoV-2 S polypeptide having the amino acid sequence of SEQ ID NO: 1. In embodiments, the delta strain comprises a CoV S polypeptide comprising deletions of amino acids 157 and 158 and mutations of T19R, W64H, H66W, G142D, E156G, D213V, L214R, W258I, K417N, N439K, L452R, T478K, E484K, N501Y, D614G, P681R, and D950N, wherein the CoV S polypeptide is numbered with respect to the wild-type SARS-CoV-2 S polypeptide having the amino acid sequence of SEQ ID NO: 1. In embodiments, the delta strain comprises a CoV S polypeptide comprising deletions of amino acids 157 and 158 and mutations of T19R, G142D, K417N, L452R, T478K, E484Q, D614G, P681R, and D950N, wherein the CoV S polypeptide is numbered with respect to the wild-type SARS-CoV-2 S polypeptide having the amino acid sequence of SEQ ID NO: 1.

The term “B.1.525 strain” (also referred to as “eta” strain) refers to a heterogeneous SARS-CoV-2 strain having a CoV S polypeptide containing the mutations Q52R; A67V; E484K; D614G; Q677H; F888L; and deletion of 1, 2, 3, or 4 of amino acids 69, 70, 144, 145, wherein the CoV S polypeptide is numbered with respect to the wild-type SARS-CoV-2 S polypeptide having the amino acid sequence of SEQ ID NO: 1.

The term “B.1.526 strain” (also referred to as “iota” strain) refers to a heterogeneous SARS-CoV-2 strain having a CoV S polypeptide containing the mutations L5F; T95I; D253G; E484K; D614G; and A701V, wherein the CoV S polypeptide is numbered with respect to the wild-type SARS-CoV-2 S polypeptide having the amino acid sequence of SEQ ID NO: 1.

The term “B.1.617.1 strain” (also referred to as “kappa” strain) refers to a heterogeneous SARS-CoV-2 strain having a CoV S polypeptide containing the mutations L452R; E484Q; D614G; P681R; and Q1071H, wherein the CoV S polypeptide is numbered with respect to the wild-type SARS-CoV-2 S polypeptide having the amino acid sequence of SEQ ID NO: 1.

The term “C.37 strain” (also referred to as “lambda” strain) refers to a heterogeneous SARS-CoV-2 strain having a CoV S polypeptide containing the mutations G75V; T76I; R246N; L452Q; F490S; D614G; T859N; and deletion of 1, 2, 3, 4, 5, or 6 of amino acids 247-253, wherein the CoV S polypeptide is numbered with respect to the wild-type SARS-CoV-2 S polypeptide having the amino acid sequence of SEQ ID NO: 1.

The term “B.1.621 strain” (also referred to as “mu” strain) refers to a heterogeneous SARS-CoV-2 strain having a CoV S polypeptide containing the mutations T95I; Y144S; Y145N; R346K; E484K; N501Y; D614G; P681H; and D950N, wherein the CoV S polypeptide is numbered with respect to the wild-type SARS-CoV-2 S polypeptide having the amino acid sequence of SEQ ID NO: 1.

The term “efficacy” of an immunogenic composition or vaccine composition described herein refers to the percentage reduction of disease (e.g., COVID-19) in a group administered an immunogenic composition as compared to a group that is not administered the immunogenic composition. In embodiments, efficacy (E) is calculated using the following equation: E (%)=(1−RR)×100, where RR=relative risk of incidence rates between the group administered the immunogenic composition and the group that is not administered the immunogenic composition. In embodiments, immunogenic compositions described herein have an efficacy against a SARS-CoV-2 virus or heterogeneous SARS-CoV-2 strain that is at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, between about 50% and about 99%, between about 50% and about 98%, between about 60% and about 99%, between about 60% and about 98%, between about 70% and about 98%, between about 70% and about 95%, between about 70% and about 99%, between about 80% and about 99%, between about 80% and about 98%, between about 80% and about 95%, between about 85% and about 99%, between about 85% and about 98%, between about 85% and about 95%, between about 90% and about 95%, between about 90% and 98%, or between about 90% and about 99%

A subject that is “positive” for SARS-CoV-2 or a variant thereof has a positive PCR or serological test for SARS-CoV-2 or a variant thereof. A positive PCR test detects genetic material from SARS-CoV-2 or a variant thereof. A positive serological test shows the presence of antibodies against a SARS-CoV-2 protein, typically the nucleocapsid protein from SARS-CoV-2 or a variant thereof.

The term “asymptomatic” refers to a subject that is positive for SARS-CoV-2 or a SARS-CoV-2 variant thereof, but does not experience any symptoms of COVID-19.

The term “mild” as it refers to COVID-19 refers to a subject that has a positive or serological test for SARS-CoV-2 or a variant thereof and has one or more of the following symptoms: (i) fever; (ii) new onset cough; (iii) or two additional COVID-19 symptoms selected from new onset or worsening of shortness of breath or difficulty breathing; fatigue; generalized muscle or body aches; headache; loss of taste or smell; sore throat, congestion, or runny nose; or nausea, vomiting, or diarrhea.

The term “moderate” as it refers to COVID-19 refers to a subject that has a positive PCR or serological test for SARS-CoV-2 or a variant thereof and one or more of the following symptoms: (i) a high fever of ≥38.4° C. for three or more days; (ii) any evidence of significant lower respiratory tract infection (LRTI), wherein the evidence is selected from: (a) shortness of breath with or without exertion; (b) tachypnea (24 to 29 breaths per minute at rest); (c) SpO2 of 94% to 95%; (d) an abnormal chest x-ray or computerized tomography (CT) consistent with pneumonia or LRTI; or (e) adventitious sounds on lung auscultation (e.g., crackles/rales, wheeze, rhonchi, pleural rub, stridor).

The term “severe” as it refers to COVID-19 refers to a subject that has a positive PCR or serological test for SARS-CoV-2 or a variant thereof and one or more of the following symptoms: (i) tachypnea of ≥30 breaths per minute at rest; (ii) resting heart rate of ≥125 beats per minute; (iii) SpO2 of ≤93% or PaO2/FiO2<300 mmHg; (iv) requirement for high flow oxygen therapy or non-invasive ventilation, non-invasive positive pressure ventilation (e.g., continuous positive airway pressure (CPAP) or bilevel positive airway pressure (BiPAP)); (v) requirement for mechanical ventilation or extracorporeal membrane oxygenation (ECMO); (vi) one or more major organ system dysfunctions or failure selected from (a) acute respiratory failure, including acute respiratory distress syndrome (ARDS); (b) acute renal failure; (c) acute hepatic failure; (d) acute right or left heart failure; (e) septic or cardiogenic shock (with shock defined as systolic blood pressure (SBP) of <90 mmHg or diastolic blood pressure (DBP) or <60 mmHg); (f) acute stroke (ischemic or hemorrhagic); (g) acute thrombotic event, such as acute myocardial infarction (AMI), deep vein thrombosis (DVT), or pulmonary embolism (PE); (h) a requirement for vasopressors, systemic corticosteroids, or hemodialysis; (vii) admission to an intensive care unit; or (viii) death.

### Vaccine Compositions Containing Coronavirus (CoV) Spike (S) Proteins

The disclosure provides non-naturally occurring coronavirus (CoV) Spike (S) polypeptides, nanoparticles containing CoV S polypeptides, and immunogenic compositions and vaccine compositions containing either non-naturally occurring CoV S polypeptides or nanoparticles containing CoV S polypeptides. In embodiments, provided herein are methods of using CoV S polypeptides, nanoparticles, immunogenic compositions, and vaccine compositions to stimulate an immune response.

Also provided herein are methods of manufacturing the nanoparticles and vaccine compositions. Advantageously, the methods provide nanoparticles that are substantially free from contamination by other proteins, such as proteins associated with recombinant expression of proteins in insect cells. In embodiments, expression occurs in baculovirus/Sf9 systems.

### CoV S Polypeptide Antigens

The vaccine compositions of the disclosure contain non-naturally occurring CoV S polypeptides. CoV S polypeptides may be derived from coronaviruses, including but not limited to SARS-CoV-2, for example from SARS-CoV-2, from MERS CoV, and from SARS CoV. In embodiments, the CoV S polypeptide is derived from a variant of SARS-CoV-2. In embodiments, the variant of SARS-CoV-2 is SARS-CoV-2 VUI 202012/01, B.1.1.7 (also called “501Y.V1” and “alpha”), B.1.351 (also called “501Y.V2” and “beta”), B.1.617.2 (also called “delta”), Cal.20C (also called “epsilon”), or P.1 (also called “gamma”). The variant of SARS-CoV-2 is designated by a World Health Organization (WHO) label (e.g., alpha, beta, gamma, delta, etc.), by its Phylogenetic Assignment of Named Global Outbreak (PANGO) lineage, by its GISAID clade, or by its Nextstrain clade.

The table below provides a list of variant SARS-CoV-2 strains:

| | | | | | Optional |
|---|---|---|---|---|---|
| | | | | Modifications | Modifications |
| WHO | Pango | GISAID | Nextstrain | compared to | compared to |
| label | lineage* | clade | clade | SEQ ID NO: 1 | SEQ ID NO: 1 |
| Alpha | B.1.1.7 | GRY | 20I (V1) | Deletion of | Deletion of |
| | | | | amino acids 69 | amino acid |
| | | | | and 70; | 145; E484K; |
| | | | | deletion of | L432R, S494P |
| | | | | amino acids 144; | |
| | | | | N501Y; A570D; | |
| | | | | D614G; P681H or | |
| | | | | P681R, T716I; | |
| | | | | S982A; and D1118H | |
| Beta | B.1.351 | GH/501Y.V2 | 20H (V2) | D80A; K417N; | D215G; L242H; |
| | | | | E484K; N501Y; | R246I; |
| | | | | D614G; A701V; | Deletion of 1, |
| | | | | | 2, or 3 amino |
| | | | | | acids of 241-243 |
| Gamma | P.1 | GR/501Y.V3 | 20J (V3) | L18F; T20N; | |
| | | | | P26S, D138Y; | |
| | | | | R190S; K417T; | |
| | | | | E484K; N501Y; | |
| | | | | D614G; H655Y; | |
| | | | | T1027I; and | |
| | | | | V1176F | |
| Delta | B.1.617.2 | G/478K.V1 | 21A, 21I, | T19R; E156G; | G142D; |
| | | | 21J | deletion of | W64H; H66W; |
| | | | | amino acids | V70F; T95I; |
| | | | | 157 and 158; | Y145H; D213V; |
| | | | | L452R; T478K; | L214R; A222V; |
| | | | | D614G; P681R; | W258I or W258L; |
| | | | | D950N | K417N; N439K; |
| | | | | | E484K or E484Q; |
| | | | | | N501Y; Q613H |
| Eta | B.1.525 | | | Q52R; A67V; | |
| | | | | deletions of | |
| | | | | amino acids | |
| | | | | 69-70; | |
| | | | | deletions of | |
| | | | | amino acids | |
| | | | | 144-145; | |
| | | | | E484K; D614G; | |
| | | | | Q677H; F888L | |
| Iota | B.1.526 | | | L5F, T95I, | |
| | | | | D253G; E484K; | |
| | | | | D614G; A701V | |
| Kappa | B.1.617.1 | | | L452R; E484Q; | |
| | | | | D614G; P681R; | |
| | | | | Q1071H | |
| Lambda | C.37 | | | G75V, T76I, | |
| | | | | R246N, | |
| | | | | deletion of 1, | |
| | | | | 2, 3, 4, 5, or 6 | |
| | | | | amino acids of | |
| | | | | 247-253; | |
| | | | | L452Q; F490S; | |
| | | | | D614G; T859N | |
| Mu | B.1.621 | | | T95I; Y144S; | |
| | | | | Y145N; R346K; | |
| | | | | E484K; N501Y; | |
| | | | | D614G; P681H; | |
| | | | | D950N | |

In embodiments, the SARS-CoV-2 virus has a CoV S polypeptide having the amino acid sequence of SEQ ID NO: 1, and the variant of SARS-CoV-2 comprises a CoV S polypeptide having at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35 modifications, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, or at least about 60 modifications compared to SEQ ID NO: 1.

In contrast, to the SARS-CoV S protein, the SARS-CoV-2 S protein has a four amino acid insertion in the S1/S2 cleavage site resulting in a polybasic RRAR furin-like cleavage motif. The SARS-CoV-2 S protein is synthesized as an inactive precursor (S0) that is proteolytically cleaved at the furin cleavage site into S1 and S2 subunits which remain non-covalently linked to form prefusion trimers. The S2 domain of the SARS-CoV-2 S protein comprises a fusion peptide (FP), two heptad repeats (HR1 and HR2), a transmembrane (TM) domain, and a cytoplasmic tail (CT). The S1 domain of the SARS-CoV-2 S protein folds into four distinct domains: the N-terminal domain (NTD) and the C-terminal domain, which contains the receptor binding domain (RBD) and two subdomains SD1 and SD2. The prefusion SARS-CoV-2 S protein trimers undergo a structural rearrangement from a prefusion to a postfusion conformation upon S-protein receptor binding and cleavage.

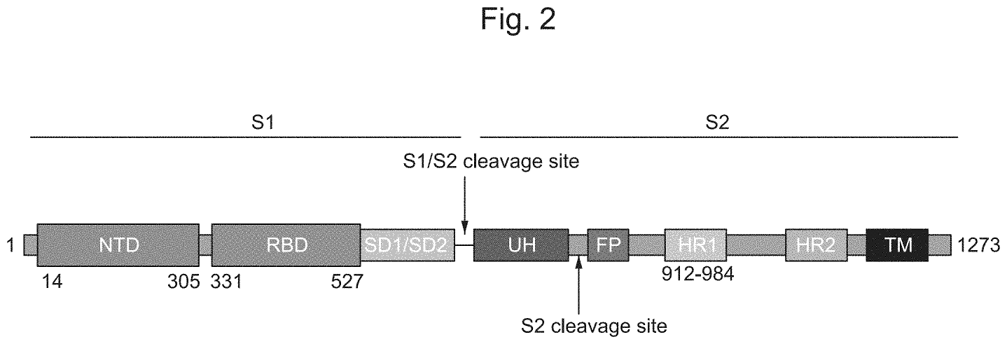

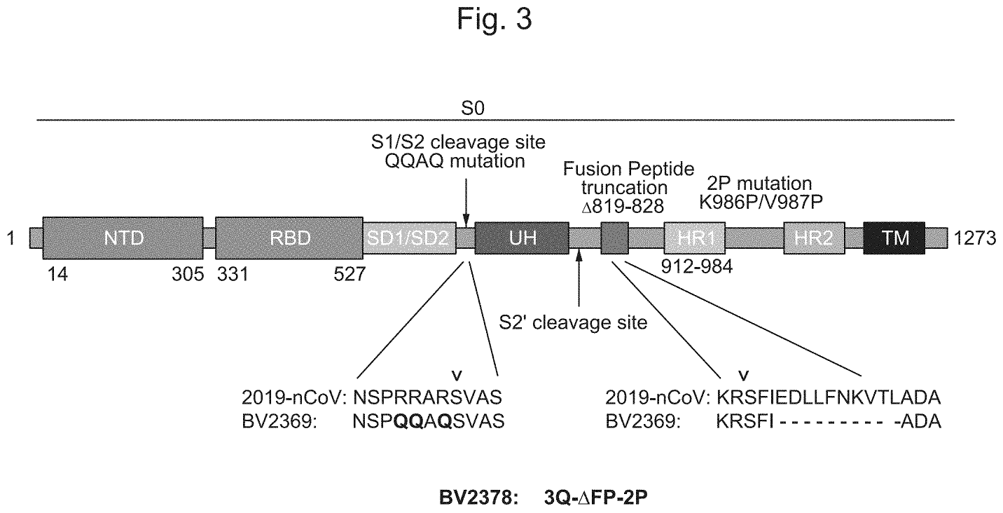

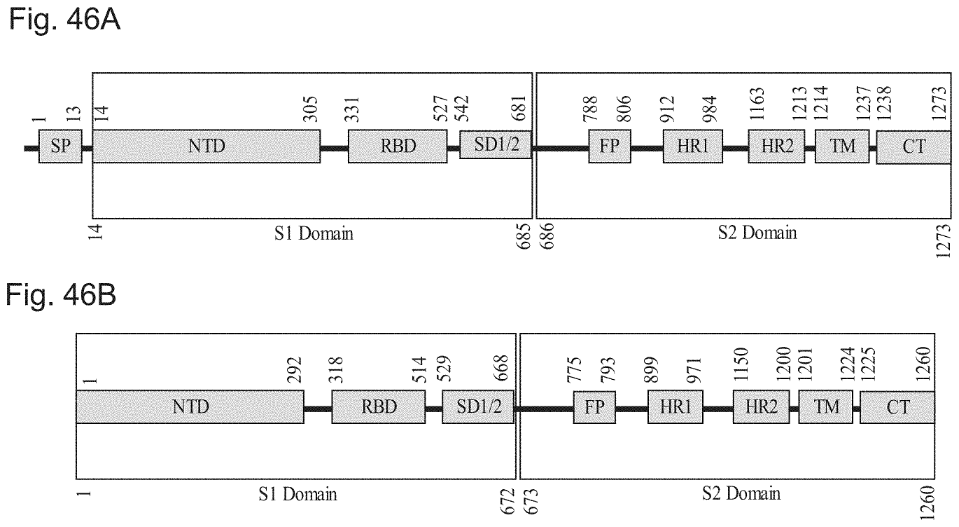

In embodiments, the CoV S polypeptides are glycoproteins, due to post-translational glycosylation. The glycoproteins comprise one or more of a signal peptide, an S1 subunit, an S2 subunit, a NTD, a, RBD, two subdomains (SD1 and SD2, labeled SD1/2 in FIGS. 46A-B and referred to as “SD1/2” herein), an intact or modified fusion peptide, an HR1 domain, an HR2 domain, a TM, and a CD. In embodiments, the amino acids for each domain are given in FIG. 2 and FIG. 46A (shown according to SEQ ID NO: 1), FIG. 46B (shown according to SEQ ID NO: 2), and FIG. 3 (shown corresponding to SEQ ID NO: 1). In embodiments, each domain may have at least 90%, at least 91%, at least 92%, at least 93%, at least 94%, at least 95%, at least 96%, at least 97%, at least 98%, at least 99%, or at least 99.5% identity to the sequences for each domain as in SEQ ID NO: 1 or SEQ ID NO: 2. Each domain may have a deletion, an insertion, or mutation of up to about 1, up to about 2, up to about 3, up to about 4, up to about 5, up to about 10, up to about 20, up to about 30, up to about 35, up to about 40, up to about 45, up to about 50, up to about 55, up to about 60, up to about 65, or up to about 70 amino acids compared to those shown in SEQ ID NO: 1 or SEQ ID NO: 2. Each domain may have a deletion, an insertion, or mutation of between about 1 and about 5 amino acids, between about 3 and about 10 amino acids, between about 5 and about 10 amino acids, between about 8 and about 12 amino acids, between about 10 and about 15 amino acids, between about 12 and about 17 amino acids, between about 15 and about 20 amino acids, between about 18 and about 23 amino acids, between about 20 and about 25 amino acids, between about 22 and about 27 amino acids, between about 25 and about 30 amino acids, between about 30 and about 35 amino acids, between about 35 and about 40 amino acids, between about 40 and about 45 amino acids, between about 45 and about 50 amino acids, between about 50 and about 55 amino acids, or between about 55 and about 60 amino acids as compared to those shown in SEQ ID NO: 1 or SEQ ID NO: 2. Note that FIGS. 2 and 3 illustrate the 13-amino acid N-terminal signal peptide that is absent from the mature peptide. The CoV S polypeptides may be used to stimulate immune responses against the native CoV Spike (S) polypeptide.

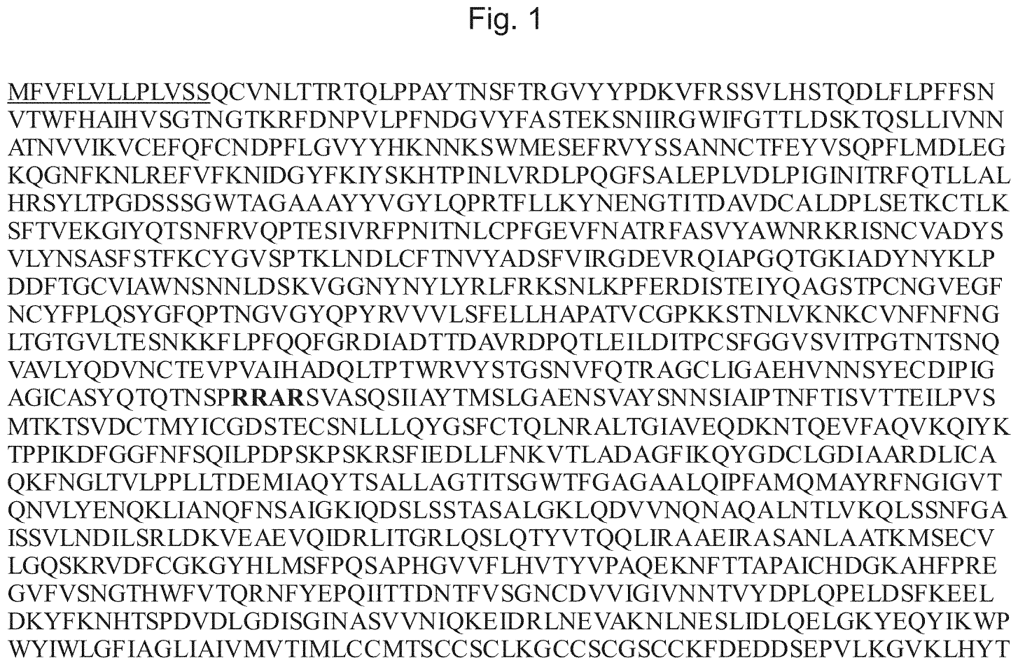

In embodiments, the native CoV Spike (S) polypeptide (SEQ ID NO: 2) is modified resulting in non-naturally occurring CoV Spike (S) polypeptides (FIG. 1). In embodiments, the CoV Spike (S) glycoproteins comprise a S1 subunit and a S2 subunit, wherein the S1 subunit comprises an NTD, an RBD, a SD1/2, and an inactive furin cleavage site (amino acids 669-672), and wherein the S2 subunit comprises mutations of amino acids 973 and 974;

wherein the NTD (amino acids 1-318) optionally comprises one or more modifications selected from the group consisting of:
(a) deletion of one or more amino acids selected from the group consisting of amino acid 56, 57, 131, 132, 144, 145, 228, 229, 230, 231, 234, 235, 236, 237, 238, 239, 240 and combinations thereof;
(b) insertion of 1, 2, 3, or 4 amino acids after amino acid 132; and

(c) mutation of one or more amino acids selected from the group consisting of amino acid 5, 6, 7, 13, 39, 51, 53, 54, 56, 57, 62, 63, 67, 82, 125, 129, 131, 132, 133, 139, 143, 144, 145, 177, 200, 201, 202, 209, 229, 233, 240, 245, and combinations thereof;

wherein the RBD optionally comprises mutation of one or more amino acids selected from the group consisting of amino acid 333. 404, 419, 426, 439, 440, 464, 465. 471, 477, 481, 488, and combinations thereof;

wherein the SD1/2 domain optionally comprises mutation of one or more amino acids selected from the group consisting of 557, 600, 601, 642, 664, 668, and combinations thereof; and

wherein the S2 subunit optionally comprises one or more modifications selected from the group consisting of:
(a) deletion of one or more amino acids from 676-685, 676-702, 702-711, 775-793, 806-815 and combinations thereof;
(b) mutation of one or more amino acids selected from the group consisting of 688, 703, 846, 875, 937, 969, 973, 974, 1014, 1058, 1105, and 1163 and combinations thereof; and

(c) deletion of one or more amino acids from the transmembrane and cytoplasmic domain (TMCT) (amino acids 1201-1260),

wherein the amino acids of the CoV S glycoprotein are numbered with respect to SEQ ID NO: 2.

FIG. 3 shows a CoV S polypeptide called BV2378, which has an inactive furin cleavage site, deleted fusion peptide (e.g., deletion of amino acids 819-828), a K986P, and a V987 mutation, wherein the amino acids are numbered with respect to SEQ ID NO: 1. The mature BV2378 polypeptide lacks one or more amino acids of the signal peptide, which are amino acids 1-13 of SEQ ID NO: 1.

In embodiments, the CoV S polypeptides described herein exist in a prefusion conformation. In embodiments, the CoV S polypeptides described herein comprise a flexible HR2 domain. Unless otherwise mentioned, the flexibility of a domain is determined by transition electron microscopy (TEM) and 2D class averaging. A reduction in electron density corresponds to a flexible domain.

### CoV S Polypeptide Antigens—Modifications to S1 Subunit

In embodiments, the CoV S polypeptides contain one or more modifications to the S1 subunit having an amino acid sequence of SEQ ID NO: 121.

The amino acid sequence of the S1 subunit (SEQ ID NO: 121) is shown below.

QCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTP
INLVRDLPQGFSALEPLVDLPIGINITRFQTLLALHRSYLTPGDSSSGW
TAGAAAYYVGYLQPRTFLLKYNENGTITDAVDCALDPLSETKCTLKSFT
VEKGIYQTSNFRVQPTESIVRFPNITNLCPFGEVFNATRFASVYAWNRK
RISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYR
LFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCL
IGAEHVNNSYECDIPIGAGICASYQTQTNSPRRAR

Underlined regions of SEQ ID NO: 121 represent amino acids within the S1 subunit that may be modified.

In embodiments, the CoV S polypeptides described herein comprise an S1 subunit with at least 95%, at least 96%, at least 97%, at least 98%, at least 99%, or at least 99.5%, identity to the S1 subunit of SEQ ID NO: 1 or SEQ ID NO: 2. The S1 subunit may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the S1 subunit of SEQ ID NO: 1 or SEQ ID NO: 2. The S1 subunit may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the S1 subunit of SEQ ID NO: 1 or SEQ ID NO: 2.

In embodiments, the S1 subunit may contain any combination of modifications shown in Table 1A.

TABLE 1A
Modifications to S1 (SEQ ID NO: 121)

| Position | Position | Position | |
|---|---|---|---|
| within | within | within | |
| SEQ ID | SEQ ID | SEQ ID | |
| NO: 1 | NO: 2 | NO: 121 | Potential Modifications |
| 14-305 | 1-292 | 1-292 | deletion of up to about 1, 2, 3, 4, 5, 10, |
| | | | 20, 30, 40, 50, 60, 70, 80, 90, 100, 110, |
| | | | 120, 130, 140, 150, 160, 170, 180, 190, |
| | | | 200, 210, 220, 230, 240, 250, 260, 270, |
| | | | 280, 290, or 292 amino acids |
| 18 | 5 | 5 | mutation to phenylalanine |
| | | | mutation to tyrosine |
| | | | mutation to tryptophan |
| 19 | 6 | 6 | mutation to arginine |
| | | | mutation to lysine |
| | | | mutation to histidine |
| 20 | 7 | 7 | mutation to asparagine |
| | | | mutation to glutamine |
| | | | mutation to isoleucine |
| | | | mutation to valine |
| 26 | 13 | 13 | mutation to serine |
| | | | mutation to threonine |
| 52 | 39 | 39 | mutation to arginine |
| | | | mutation to lysine |
| | | | mutation to histidine |
| 64 | 51 | 51 | mutation to histidine |
| | | | mutation to lysine |
| | | | mutation to arginine |
| 66 | 53 | 53 | mutation to tryptophan |
| | | | mutation to tyrosine |
| | | | mutation to phenylalanine |
| 67 | 54 | 54 | mutation to valine |
| | | | mutation to isoleucine |
| | | | mutation to leucine |
| 69 | 56 | 56 | Deletion of amino acid |
| 70 | 57 | 57 | Deletion of amino acid |
| | | | Mutation to phenylalanine |
| | | | Mutation to tyrosine |
| | | | Mutation to tryptophan |
| 75 | 62 | 62 | Mutation to valine |
| | | | Mutation to leucine |
| | | | Mutation to isoleucine |
| 76 | 63 | 63 | Mutation to isoleucine |
| | | | Mutation to valine |
| | | | Mutation to leucine |
| 80 | 67 | 67 | mutation to alanine |
| | | | mutation to glycine |
| 95 | 82 | 82 | mutation to beta branched amino acid |
| | | | mutation to isoleucine |
| | | | mutation to valine |
| 138 | 125 | 125 | mutation to tyrosine |
| | | | mutation to phenylalanine |
| | | | mutation to tryptophan |
| 142 | 129 | 129 | mutation to aspartic acid |
| | | | mutation to glutamic acid |
| 144 | 131 | 131 | Deletion of amino acid |
| | | | Mutation to serine |
| 145 | 132 | 132 | Deletion of amino acid |
| | | | Mutation to histidine |
| | | | Mutation to asparagine |
| | | | Mutation to glutamine |
| | | | insertion of 1, 2, 3, or 4 amino acids |
| | | | after amino acid 132 |
| | | | (e.g., asparagine) |
| 146 | 133 | 133 | mutation to aromatic amino acid |
| | | | mutation to tyrosine |
| | | | mutation to phenylalanine |
| | | | mutation to tryptophan |
| 152 | 139 | 139 | mutation to cysteine |
| | | | mutation to methionine |
| | | | mutation to serine |
| | | | mutation to threonine |
| 156 | 143 | 143 | mutation to glycine |
| | | | mutation to alanine |
| 157 | 144 | 144 | deletion of amino acid |
| 158 | 145 | 145 | deletion of amino acid |
| 190 | 177 | 177 | mutation to serine |
| | | | mutation to threonine |
| | | | mutation to cysteine |
| 213 | 200 | 200 | mutation to valine |
| | | | mutation to leucine |
| | | | mutation to isoleucine |
| | | | mutation to beta branched amino acid |
| 214 | 201 | 201 | mutation to arginine |
| | | | mutation to lysine |
| | | | mutation to histidine |
| 215 | 202 | 202 | mutation to glycine |
| | | | mutation to alanine |
| 222 | 209 | 209 | mutation to valine |
| | | | mutation to leucine |
| | | | mutation to isoleucine |
| 241-244 | 228-231 | 228-231 | deletion of 1, 2, 3, or 4 amino acids |
| 242 | 229 | 229 | mutation to histidine |
| | | | mutation to lysine |
| | | | mutation to arginine |
| 246 | 233 | 233 | mutation to beta-branched amino acid |
| | | | mutation to isoleucine |
| | | | mutation to valine |
| | | | mutation to threonine |
| | | | mutation to asparagine |
| 247 | 234 | 234 | deletion of amino acid |
| 248 | 235 | 235 | deletion of amino acid |
| 249 | 236 | 236 | deletion of amino acid |
| 250 | 237 | 237 | deletion of amino acid |
| 251 | 238 | 238 | deletion of amino acid |
| 252 | 239 | 239 | deletion of amino acid |
| 253 | 240 | 240 | mutation to glycine |
| | | | deletion of amino acid |
| 258 | 245 | 245 | mutation to isoleucine |
| | | | mutation to valine |
| | | | mutation to leucine |
| | | | mutation to beta branched amino acid |
| 346 | 333 | 333 | mutation to lysine |
| | | | mutation to arginine |
| | | | mutation to histidine |
| 417 | 404 | 404 | mutation to asparagine |
| | | | mutation to threonine |
| | | | mutation to isoleucine |
| | | | mutation to valine |
| | | | mutation to serine |
| | | | mutation to glutamine |
| | | | mutation to beta-branched amino acid |
| 432 | 419 | 419 | mutation to lysine |
| | | | mutation to arginine |
| | | | mutation to histidine |
| 439 | 426 | 426 | mutation to lysine |
| | | | mutation to arginine |
| | | | mutation to histidine |
| 452 | 439 | 439 | mutation to arginine |
| | | | mutation to lysine |
| | | | mutation to histidine |
| | | | mutation to glutamine |
| | | | mutation to asparagine |
| 453 | 440 | 440 | mutation to phenylalanine |
| | | | mutation to tryptophan |
| 477 | 464 | 464 | mutation to asparagine |
| | | | mutation to glutamine |
| 478 | 465 | 465 | mutation to lysine |
| | | | mutation to arginine |
| | | | mutation to histidine |
| 484 | 471 | 471 | mutation to lysine |
| | | | mutation to arginine |
| | | | mutation to histidine |
| | | | mutation to glutamine |
| | | | mutation to asparagine |
| 490 | 477 | 477 | mutation to serine |
| | | | mutation to threonine |
| 494 | 481 | 481 | mutation to proline |
| 501 | 488 | 488 | mutation to tyrosine |
| | | | mutation to phenylalanine |
| | | | mutation to tryptophan |
| 570 | 557 | 557 | Mutation to aspartic acid |
| | | | Mutation to glutamic acid |
| 613 | 600 | 600 | Mutation to histidine |
| | | | Mutation to lysine |
| | | | Mutation to arginine |
| 614 | 601 | 601 | Mutation to glycine |
| | | | Mutation to alanine |
| 655 | 642 | 642 | Mutation to tyrosine |
| | | | Mutation to phenylalanine |
| | | | Mutation to tryptophan |
| 677 | 664 | 664 | Mutation to histidine |
| 681 | 668 | 668 | Mutation to histidine |
| | | | Mutation to lysine |
| | | | Mutation to arginine |
| 682-685 | 669-672 | 669-672 | inactive furin cleavage site (See Table 1E) |

* amino acids 14-685 of SEQ ID NO: 1 and amino acids 1-672 of SEQ ID NO: 2

### CoV S Polypeptide Antigens—Modifications to S1 Subunit—NTD

In embodiments, the CoV S polypeptides contain one or more modifications to the NTD. In embodiments, the NTD has an amino acid sequence of SEQ ID NO: 118, which corresponds to amino acids 14-305 of SEQ ID NO: 1 or amino acids 1-292 of SEQ ID NO: 2.

The amino acid sequence of an NTD (SEQ ID NO: 118) is shown below.

QCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTP
INLVRDLPQGFSALEPLVDLPIGINITRFQTLLALHRSYLTPGDSSSGW
TAGAAAYYVGYLQPRTFLLKYNENGTITDAVDCALDPLSETKCTLKS

In embodiments, the NTD has an amino acid sequence of SEQ ID NO: 45, which corresponds to amino acids 14 to 331 of SEQ ID NO: 1 or amino acids 1-318 of SEQ ID NO: 2. The amino acid sequence of an NTD (SEQ ID NO: 45) is shown below.

QCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTP
INLVRDLPQGFSALEPLVDLPIGINITRFQTLLALHRSYLTPGDSSSGW
TAGAAAYYVGYLQPRTFLLKYNENGTITDAVDCALDPLSETKCTLKSFT
VEKGIYQTSNFRVQPTESIVRFPN

In embodiments, the NTD and RBD overlap by up to about 1 amino acid, up to about 5 amino acids, up to about 10 amino acids, or up to about 20 amino acids.

In embodiments, an NTD as provided herein may be extended at the C-terminus by up to 5, up to 10, up to 15, up to 20, up to 25, or up to 30 amino acids.

In embodiments, the CoV S polypeptides described herein comprise a NTD with at least 95%, at least 96%, at least 97%, at least 98%, at least 99%, or at least 99.5%, identity to the NTD of SEQ ID NO: 1 or SEQ ID NO: 2. The NTD may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the NTD of SEQ ID NO: 1 or SEQ ID NO: 2. The NTD may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the NTD of SEQ ID NO: 1 or SEQ ID NO: 2.

In embodiments, the CoV S polypeptides contain a deletion of one or more amino acids from the N-terminal domain (NTD) (corresponding to amino acids 1-292 of SEQ ID NO: 2. In embodiments, the CoV S polypeptides contain a deletion of up to about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 292 amino acids of the NTD.

In embodiments, the CoV S polypeptides contain a deletion of one or more amino acids from the NTD (corresponding to amino acids 1-318 of SEQ ID NO: 2). In embodiments, the CoV S polypeptides contain a deletion of amino acids 1-318 of the NTD of SEQ ID NO: 2. In embodiments, deletion of the NTD enhances protein expression of the COV Spike (S) polypeptide. In embodiments, the CoV S polypeptides which have an NTD deletion have amino acid sequences represented by SEQ ID NOS: 46, 48, 49, 51, 52, and 54. In embodiments, the CoV S polypeptides which have an NTD deletion are encoded by an isolated nucleic acid sequence selected from the group consisting of SEQ ID NO: 47, SEQ ID NO: 50, and SEQ ID NO: 53.

In embodiments, the NTD may contain any combination of modifications shown in Table 1B. The modifications are shown with respect to SEQ ID NO:2, the mature S polypeptide sequence for reference.

TABLE 1B
Modifications to NTD (SEQ ID NO: 118)

| | | SEQ ID | |
|---|---|---|---|
| Position | | NO: 121 | |
| within | SEQ ID | or SEQ ID | |
| SEQ ID | NO: 2 | NO: 45 | |
| NO: 1 | residue | residue | Modifications |
| 18 | 5 | 5 | mutation to phenylalanine |
| | | | mutation to tyrosine |
| | | | mutation to tryptophan |
| 19 | 6 | 6 | mutation to arginine |
| | | | mutation to lysine |
| | | | mutation to histidine |
| 20 | 7 | 7 | mutation to asparagine |
| | | | mutation to glutamine |
| | | | mutation to isoleucine |
| | | | mutation to valine |
| 26 | 13 | 13 | mutation to serine |
| | | | mutation to threonine |
| 64 | 51 | 51 | mutation to histidine |
| | | | mutation to lysine |
| | | | mutation to arginine |
| 66 | 53 | 53 | mutation to tryptophan |
| | | | mutation to tyrosine |
| | | | mutation to phenylalanine |
| 69 | 56 | 56 | Deletion of amino acid |
| 70 | 57 | 57 | Deletion of amino acid |
| | | | Mutation to phenylalanine |
| | | | Mutation to tyrosine |
| | | | Mutation to tryptophan |
| 75 | 62 | 62 | Mutation to valine |
| | | | Mutation to leucine |
| | | | Mutation to isoleucine |
| 76 | 63 | 63 | Mutation to isoleucine |
| | | | Mutation to valine |
| | | | Mutation to leucine |
| 80 | 67 | 67 | mutation to alanine |
| | | | mutation to glycine |
| 95 | 82 | 82 | mutation to beta branched amino acid |
| | | | mutation to isoleucine |
| | | | mutation to valine |
| 138 | 125 | 125 | mutation to tyrosine |
| | | | mutation to phenylalanine |
| | | | mutation to tryptophan |
| 142 | 129 | 129 | mutation to aspartic acid |
| | | | mutation to glutamic acid |
| 144 | 131 | 131 | Deletion of amino acid |
| | | | Mutation to serine |
| 145 | 132 | 132 | Deletion of amino acid |
| | | | Mutation to histidine |
| | | | Mutation to asparagine |
| | | | Mutation to glutamine |
| | | | insertion of 1, 2, 3, or 4 amino |
| | | | acids after this position |
| 146 | 133 | 133 | Mutation to aromatic amino acid |
| | | | Mutation to tyrosine |
| | | | Mutation to phenylalanine |
| | | | Mutation to tryptophan |
| 152 | 139 | 139 | mutation to cysteine |
| | | | mutation to methionine |
| | | | mutation to serine |
| | | | mutation to threonine |
| | | | mutation to arginine |
| 156 | 143 | 143 | mutation to glycine |
| | | | mutation to alanine |
| 157 | 144 | 144 | deletion of amino acid |
| 158 | 145 | 145 | deletion of amino acid |
| 190 | 177 | 177 | mutation to serine |
| | | | mutation to threonine |
| | | | mutation to cysteine |
| 213 | 200 | 200 | mutation to valine |
| | | | mutation to leucine |
| | | | mutation to isoleucine |
| | | | mutation to beta branched amino acid |
| 214 | 201 | 201 | mutation to arginine |
| | | | mutation to lysine |
| | | | mutation to histidine |
| 215 | 202 | 202 | mutation to glycine |
| | | | mutation to alanine |
| 222 | 209 | 209 | mutation to valine |
| | | | mutation to leucine |
| | | | mutation to isoleucine |
| 242 | 229 | 229 | mutation to histidine |
| | | | mutation to lysine |
| | | | mutation to arginine |
| 241-244 | 228-231 | 228-231 | deletion of 1, 2, 3, or 4 amino acids |
| 246 | 233 | 233 | mutation to beta-branched amino acid |
| | | | mutation to isoleucine |
| | | | mutation to valine |
| | | | mutation to threonine |
| | | | mutation to asparagine |
| 247 | 234 | 234 | deletion of amino acid |
| 248 | 235 | 235 | deletion of amino acid |
| 249 | 236 | 236 | deletion of amino acid |
| 250 | 237 | 237 | deletion of amino acid |
| 251 | 238 | 238 | deletion of amino acid |
| 252 | 239 | 239 | deletion of amino acid |
| 253 | 240 | 240 | mutation to glycine |
| | | | deletion of amino acid |
| 258 | 245 | 245 | mutation to isoleucine |
| | | | mutation to valine |
| | | | mutation to leucine |
| | | | mutation to beta branched amino acid |

* amino acids 14-305 of SEQ ID NO: 1 and amino acids 1-292 of SEQ ID NO: 2

### CoV S Polypeptide Antigens—Modifications to S1 Subunit—RBD

In embodiments, the CoV S polypeptides contain one or more modifications to the RBD.

In embodiments, the RBD has an amino acid sequence of SEQ ID NO: 126, which corresponds to amino acids 331-527 of SEQ ID NO: 1 or amino acids 318-514 of SEQ ID NO: 2.

The amino acid sequence of the RBD is shown below:
(SEQ ID NO: 126)
NITNLCPFGEVFNATRFASVYAWNRKRISNCVADYSVLYNSASFSTFKC
YGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCG
P

In embodiments, the RBD has an amino acid sequence of SEQ ID NO: 116, which corresponds to amino acids 335-530 of SEQ ID NO: 1 or amino acids 322-517 of SEQ NO: 2.

The amino acid sequence of the RBD (SEQ ID NO: 116) is shown below.

LCPFGEVFNATRFASVYAWNRKRISNCVADYSVLYNSASFSTFKCYGVS
PTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCN
GVEGFNCYFPLQSYGFQPTNGVGYQPYRVVVLSFELLHAPATVCGPKKS

In embodiments, an RBD as provided herein may be extended at the N-terminus or C-terminus by up to 1 amino acid, up to 5 amino acids, up to 10 amino acids, up to 15 amino acids, up to 20 amino acids, up to 25 amino acids, or up to 30 amino acids.

In embodiments, the CoV S polypeptides described herein comprise a RBD with at least 95%, at least 96%, at least 97%, at least 98%, at least 99%, or at least 99.5%, identity to the RBD of SEQ ID NO: 1 or SEQ ID NO: 2. The RBD may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the RBD of SEQ ID NO: 1 or SEQ ID NO: 2. The RBD may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the RBD of SEQ ID NO: 1 or SEQ ID NO: 2.

In embodiments, the CoV S polypeptide has at least one, at least two, at least three, at least four, at least four, at least 5, at least 6, at least 7, at least 8, at least 9, at least 10, at least 11, at least 12, at least 13, at least 14, at least 15, at least 16, at least 17, at least 18, at least 19, or at least 20 mutations in the RBD. In embodiments, the RBD may contain any combination of modifications as shown in Table 1C.

TABLE 1C
Modifications to RBD (SEQ ID NO: 126)

| Position | Position | Position | |
|---|---|---|---|
| within | within | within | |
| SEQ ID | SEQ ID | SEQ ID | |
| NO: 1 | NO: 2 | NO: 126 | Potential Modifications |
| 346 | 333 | 16 | mutation to lysine |
| | | | mutation to arginine |
| | | | mutation to histidine |
| 417 | 404 | 87 | mutation to asparagine |
| | | | mutation to threonine |
| | | | mutation to isoleucine |
| | | | mutation to valine |
| | | | mutation to serine |
| | | | mutation to glutamine |
| | | | mutation to beta-branched amino acid |
| 432 | 419 | 102 | mutation to lysine |
| | | | mutation to arginine |
| | | | mutation to histidine |
| 439 | 426 | 109 | mutation to lysine |
| | | | mutation to arginine |
| | | | mutation to histidine |
| 452 | 439 | 122 | mutation to arginine |
| | | | mutation to lysine |
| | | | mutation to histidine |
| | | | mutation to glutamine |
| | | | mutation to asparagine |
| 453 | 440 | 123 | mutation to phenylalanine |
| | | | mutation to tryptophan |
| 477 | 464 | 147 | mutation to asparagine |
| | | | mutation to glutamine |
| 478 | 465 | 148 | mutation to lysine |
| | | | mutation to arginine |
| | | | mutation to histidine |
| 484 | 471 | 154 | mutation to lysine |
| | | | mutation to arginine |
| | | | mutation to histidine |
| | | | mutation to glutamine |
| | | | mutation to asparagine |
| 490 | 477 | 160 | mutation to serine |
| | | | mutation to threonine |
| 494 | 481 | 164 | mutation to proline |
| 501 | 488 | 171 | mutation to tyrosine |
| | | | mutation to phenylalanine |
| | | | mutation to tryptophan |

* amino acids 331-527 of SEQ ID NO: 1 and amnio acids 318-514 of SEQ ID NO: 2

### CoV S Polypeptide Antigens—Modifications to SD1/2

In embodiments, the CoV S polypeptides contain one or more modifications to the SD1/2 domain having an amino acid sequence of SEQ ID NO: 122, which corresponds to amino acids 542-681 of SEQ ID NO: 1 or amino acids 529-668 of SEQ ID NO: 2.

The amino acid sequence of the SD1/2 (SEQ ID NO: 122) domain is shown below.

NFNGLTGTGVLTESNKKFLPFQQFGRDIADTTDAVRDPQTLEILDITPC
SFGGVSVITPGTNTSNQVAVLYQDVNCTEVPVAIHADQLTPTWRVYSTG
SNVFQTRAGCLIGAEHVNNSYECDIPIGAGICASYQTQTNSP

In embodiments, the CoV S polypeptides described herein comprise a SD1/2 domain with at least 95%, at least 96%, at least 97%, at least 98%, at least 99%, or at least 99.5%, identity to the SD1/2 of SEQ ID NO: 1 or SEQ ID NO: 2. The SD1/2 domain may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the SD1/2 of SEQ ID NO: 1 or SEQ ID NO: 2. The SD1/2 domain may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the SD1/2 domain of SEQ ID NO: 1 or SEQ ID NO: 2.

In embodiments, the CoV S polypeptide has at least one, at least two, at least three, at least four, at least four, at least 5, at least 6, at least 7, at least 8, at least 9, at least 10, at least 11, at least 12, at least 13, at least 14, at least 15, at least 16, at least 17, at least 18, at least 19, or at least 20 mutations in the SD1/2 domain. In embodiments, the SD1/2 domain may contain any combination of modifications as shown in Table 1D.

TABLE 1D
Modifications to SD1/2 (SEQ ID NO: 122)

| Position | Position | Position | |
|---|---|---|---|
| within | within | within | |
| SEQ ID | SEQ ID | SEQ ID | |
| NO: 1 | NO: 2 | NO: 122 | Potential Modifications |
| 570 | 557 | 29 | Mutation to aspartic acid |
| | | | Mutation to glutamic acid |
| 613 | 600 | 600 | Mutation to histidine |
| | | | Mutation to lysine |
| | | | Mutation to arginine |
| 614 | 601 | 73 | Mutation to glycine |
| | | | Mutation to alanine |
| 655 | 642 | 114 | Mutation to tyrosine |
| | | | Mutation to phenylalanine |
| | | | Mutation to tryptophan |
| 677 | 664 | 664 | Mutation to histidine |
| 681 | 668 | 140 | Mutation to histidine |
| | | | Mutation to lysine |
| | | | Mutation to arginine |

* amino acids 542-681 of SEQ ID NO: 1 or amino acids 529-668 of SEQ ID NO: 2

### CoV S Polypeptide Antigens—Modifications to Furin Cleavage Site

In embodiments, the CoV S polypeptides contain a furin site (RRAR), which corresponds to amino acids 682-685 of SEQ ID NO: 1 or amino acids 669-672 of SEQ ID NO: 2, that is inactivated by one or more mutations. Inactivation of the furin cleavage site prevents furin from cleaving the CoV S polypeptide. In embodiments, the CoV S polypeptides described herein which contain an inactivated furin cleavage site are expressed as a single chain.

In embodiments, one or more of the amino acids comprising the native furin cleavage site is mutated to any natural amino acid. In embodiments, the amino acids are L-amino acids. Non-limiting examples of amino acids include alanine, arginine, glycine, asparagine, aspartic acid, cysteine, glutamine, glutamic acid, serine, threonine, histidine, lysine, methionine, proline, valine, isoleucine, leucine, tyrosine, tryptophan, and phenylalanine.

In embodiments, one or more of the amino acids comprising the native furin cleavage site is mutated to glutamine. In embodiments, 1, 2, 3, or 4 amino acids may be mutated to glutamine. In embodiments, one of the arginines comprising the native furin cleavage site is mutated to glutamine. In embodiments, two of the arginines comprising the native furin cleavage site are mutated to glutamine. In embodiments, three of the arginines comprising the native furin cleavage site are mutated to glutamine.

In embodiments, one or more of the amino acids comprising the native furin cleavage site, is mutated to alanine. In embodiments, 1, 2, 3, or 4 amino acids may be mutated to alanine. In embodiments, one of the arginines comprising the native furin cleavage site is mutated to alanine. In embodiments, two of the arginines comprising the native furin cleavage site are mutated to alanine. In embodiments, three of the arginines comprising the native furin cleavage site are mutated to alanine.

In embodiments, one or more of the amino acids in the native furin cleavage site is mutated to glycine. In embodiments, 1, 2, 3, or 4 amino acids may be mutated to glycine. In embodiments, one of the arginines of the native furin cleavage site is mutated to glycine. In embodiments, two of the arginines in the native furin cleavage site are mutated to glycine. In embodiments, three of the arginines comprising the native furin cleavage site are mutated to glycine.

In embodiments, one or more of the amino acids in the native furin cleavage site, is mutated to asparagine. For example 1, 2, 3, or 4 amino acids may be mutated to asparagine. In embodiments, one of the arginines in the native furin cleavage site is mutated to asparagine. In embodiments, two of the arginines in the native furin cleavage site are mutated to asparagine. In embodiments, three of the arginines in the native furin cleavage site are mutated to asparagine.

Non-limiting examples of the amino acid sequences of the inactivated furin sites contained within the CoV S polypeptides are found in Table 1E.

TABLE 1E

| Amino Acid Sequence of | Active or Inactive |
|---|---|
| Furin Cleavage Site | Furin Cleavage Site |
| RRAR (SEQ ID NO: 6) | Active |
| QQAQ (SEQ ID NO: 7) | Inactive |
| QRAR (SEQ ID NO: 8) | Inactive |
| RQAR (SEQ ID NO: 9) | Inactive |
| RRAQ (SEQ ID NO: 10) | Inactive |
| QQAR (SEQ ID NO: 11) | Inactive |
| RQAQ (SEQ ID NO: 12) | Inactive |
| QRAQ (SEQ ID NO: 13) | Inactive |
| NNAN (SEQ ID NO: 14) | Inactive |
| NRAR (SEQ ID NO: 15) | Inactive |
| RNAR (SEQ ID NO: 16) | Inactive |
| RRAN (SEQ ID NO: 17) | Inactive |
| NNAR (SEQ ID NO: 18) | Inactive |
| RNAN (SEQ ID NO: 19) | Inactive |
| NRAN (SEQ ID NO: 20) | Inactive |
| AAAA (SEQ ID NO: 21) | Inactive |
| ARAR (SEQ ID NO: 22) | Inactive |
| RAAR (SEQ ID NO: 23) | Inactive |
| RRAA (SEQ ID NO: 24) | Inactive |
| AAAR (SEQ ID NO: 25) | Inactive |
| RAAA (SEQ ID NO: 26) | Inactive |
| ARAA (SEQ ID NO: 27) | Inactive |
| GGAG (SEQ ID NO: 28) | Inactive |
| GRAR (SEQ ID NO: 29) | Inactive |
| RGAR (SEQ ID NO: 30) | Inactive |
| RRAG (SEQ ID NO: 31) | Inactive |
| GGAR (SEQ ID NO: 32) | Inactive |
| RGAG (SEQ ID NO: 33) | Inactive |
| GRAG (SEQ ID NO: 34) | Inactive |
| GSAS (SEQ ID NO: 97) | Inactive |
| GSGA (SEQ ID NO: 111) | Inactive |

In embodiments, in lieu of an active furin cleavage site (SEQ ID NO: 6) the CoV S polypeptides described herein contain an inactivated furin cleavage site. In embodiments, the amino acid sequence of the inactivated furin cleavage site is represented by any one of SEQ ID NO: 7-34 or SEQ ID NO: 97. In embodiments, the amino acid sequence of the inactivated furin cleavage site is QQAQ (SEQ ID NO: 7). In embodiments, the amino acid sequence of the inactivated furin cleavage site is GSAS (SEQ ID NO: 97). In embodiments, the amino acid sequence of the inactivated furin cleavage site is GSGA (SEQ ID NO: 111). In embodiments, the amino acid sequence of the inactivated furin cleavage site is GG, GGG (SEQ ID NO: 127), GGGG (SEQ ID NO: 128), or GGGGG (SEQ ID NO: 129).

### CoV S Polypeptide Antigens—Modifications to S2 Subunit

In embodiments, the CoV S polypeptides contain one or more modifications to the S2 subunit having an amino acid sequence of SEQ ID NO: 120, which corresponds to amino acids 686-1273 of SEQ ID NO: 1 or amino acids 673-1260 of SEQ ID NO: 2.

The amino acid sequence of the S2 subunit (SEQ ID NO: 120) is shown below.

SVASQSIIAYTMSLGAENSVAYSNNSIAIPTNFTISVTTEILPVSMTKT
SVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGT
ITSGWTFGAGAALQIPFAMQMAYRFNGIGVTQNVLYENQKLIANQFNSA
IGKIQDSLSSTASALGKLQDVVNQNAQALNTLVKQLSSNFGAISSVLND
ILSRLDKVEAEVQIDRLITGRLQSLQTYVTQQLIRAAEIRASANLAATK
MSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNC
DVVIGIVNNTVYDPLQPELDSFKEELDKYFKNHTSPDVDLGDISGINAS
VVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT

In embodiments, the CoV S polypeptides described herein comprise an S2 subunit with at least 95%, at least 96%, at least 97%, at least 98%, at least 99%, or at least 99.5%, identity to the S2 subunit of SEQ ID NO: 1 or SEQ ID NO: 2. The S2 subunit may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the S2 subunit of SEQ ID NO: 1 or SEQ ID NO: 2. The S2 subunit may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the S2 subunit of SEQ ID NO: 1 or SEQ ID NO: 2.

In embodiments, the S2 subunit may contain any combination of modifications as shown in Table 1F.

TABLE 1F
Modifications to S2 (SEQ ID NO: 120)

| Position | Position | Position | |
|---|---|---|---|
| within | within | within | |
| SEQ ID | SEQ ID | SEQ ID | |
| NO: 1 | NO: 2 | NO: 120 | Possible Modifications |
| 689-698 | 676-685 | 4-13 | Deletion of up to about 1, up to |
| | | | about 2, up to about 3, up to about |
| | | | 4, up to about 5, up to about 6, |
| | | | up to about 7, up to about 8, up to |
| | | | about 9, or up to about 10 amino |
| | | | acids |
| 701 | 688 | 16 | Mutation to beta-branched amino acid |
| | | | Mutation to valine |
| | | | Mutation to isoleucine |
| | | | Mutation to threonine |
| 715-724 | 702-711 | 30-39 | Deletion of up to about 1, up to |
| | | | about 2, up to about 3, up to about |
| | | | 4, up to about 5, up to about 6, |
| | | | up to about 7, up to about 8, up to |
| | | | about 9, or up to about 10 amino |
| | | | acids |
| 716 | 703 | 31 | Mutation to beta-branched amino acid |
| | | | Mutation to valine |
| | | | Mutation to isoleucine |
| 788-806 | 775-793 | 103-121 | Deletion of up to about 1, up to |
| | | | about 2, up to about 3, up to about |
| | | | 4, up to about 5, up to about 6, |
| | | | up to about 7, up to about 8, up to |
| | | | about 9, up to about 10, up to about |
| | | | 11, up to about 12, up to about 13, |
| | | | up to about 14, up to about 15, up to |
| | | | about 16, up to about 17, up to about |
| | | | 18, or up to about 19 amino acids |
| 819-828 | 806-815 | 134-143 | Deletion of up to about 1, up to |
| | | | about 2, up to about 3, up to about |
| | | | 4, up to about 5, up to about 6, |
| | | | up to about 7, up to about 8, up to |
| | | | about 9, or up to about 10 amino |
| | | | acids |
| 859 | 846 | 174 | Mutation to asparagine |
| | | | Mutation to glutamine |
| 888 | 875 | 203 | Mutation to leucine |
| | | | Mutation to isoleucine |
| | | | Mutation to valine |
| 950 | 937 | 265 | Mutation to asparagine |
| | | | Mutation to glutamine |
| 982 | 969 | 297 | Mutation to alanine |
| | | | Mutation to glycine |
| | | | Mutation to threonine |
| 986 | 973 | 301 | Mutation to proline |
| | | | Mutation to glycine |
| 987 | 974 | 302 | Mutation to proline |
| | | | Mutation to glycine |
| 1027 | 1014 | 342 | Mutation to isoleucine |
| | | | Mutation to valine |
| | | | Mutation to serine |
| 1071 | 1058 | 386 | Mutation to histidine |
| | | | Mutation to arginine |
| | | | Mutation to lysine |
| 1118 | 1105 | 433 | Mutation to histidine |
| | | | Mutation to lysine |
| | | | Mutation to arginine |
| | | | Mutation to asparagine |
| | | | Mutation to glutamine |
| 1176 | 1163 | 491 | Mutation to phenylalanine |
| | | | Mutation to tyrosine |
| | | | Mutation to tryptophan |
| 1214-1237 | 1201-1224 | 1-24 | Deletion of one or more amino |
| | | | acids of TM |
| 1238-1273 | 1225-1260 | 1-36 | Deletion of one or more amino |
| | | | acids of CD |

* ammo acids 686-1273 of SEQ ID NO: 1 and amino acids 673-1260 of SEQ ID NO: 2

In embodiments, the CoV S polypeptides contain a deletion, corresponding to one or more deletions within amino acids 676-685 of the native CoV Spike (S) polypeptide (SEQ ID NO: 2). In embodiments, 1, 2, 3, 4, 5, 6, 7, 8, 9, or 10 amino acids of amino acids 676-685 of the native CoV Spike (S) polypeptide (SEQ ID NO:2) are deleted. In embodiments, the deletions of amino acids within amino acids 676-685 are consecutive e.g. amino acids 676 and 677 are deleted or amino acids 680 and 681 are deleted. In embodiments, the deletions of amino acids within amino acids 676-685 are non-consecutive e.g. amino acids 676 and 680 are deleted or amino acids 677 and 682 are deleted. In embodiments, CoV S polypeptides containing a deletion, corresponding to one or more deletions within amino acids 676-685, have an amino acid sequence selected from the group consisting of SEQ ID NO: 62 and SEQ ID NO: 63.

In embodiments, the CoV S polypeptides contain a deletion, corresponding to one or more deletions within amino acids 702-711 of the native CoV Spike (S) polypeptide (SEQ ID NO: 2). In embodiments, 1, 2, 3, 4, 5, 6, 7, 8, 9, or 10 amino acids of amino acids 702-711 of the native SARS-CoV-2 Spike (S) polypeptide (SEQ ID NO:2) are deleted. In embodiments, the one or more deletions of amino acids within amino acids 702-711 are consecutive e.g. amino acids 702 and 703 are deleted or amino acids 708 and 709 are deleted. In embodiments, the deletions of amino acids within amino acids 702-711 are non-consecutive e.g. amino acids 702 and 704 are deleted or amino acids 707 and 710 are deleted. In embodiments, the CoV S polypeptides containing a deletion, corresponding to one or more deletions within amino acids 702-711, have an amino acid sequence selected from the group consisting of SEQ ID NO: 64 and SEQ ID NO: 65.

In embodiments, the CoV S polypeptides contain a deletion, corresponding to one or more deletions within amino acids 775-793 of the native CoV S polypeptide (SEQ ID NO: 2). In embodiments, up to about 1, 2, 3, 4, 5, 6, 7, 8, 9, 10, 11, 12, 13, 14, 15, 16, 17, 18, or 19 amino acids of amino acids 775-793 of the native SARS-CoV-2 Spike (S) polypeptide (SEQ ID NO:2) are deleted. In embodiments, the one or more deletions of amino acids within amino acids 775-793 are consecutive e.g. amino acids 776 and 777 are deleted or amino acids 780 and 781 are deleted. In embodiments, the deletions of amino acids within amino acids 775-793 are non-consecutive e.g. amino acids 775 and 790 are deleted or amino acids 777 and 781 are deleted.

In embodiments, the CoV S polypeptides contain a deletion of the fusion peptide (SEQ ID NO: 104), which corresponds to amino acids 806-815 of SEQ ID NO: 2. In embodiments, 1, 2, 3, 4, 5, 6, 7, 8, 9, or 10 amino acids of the fusion peptide of the CoV Spike (S) polypeptide (SEQ ID NO:2) are deleted. In embodiments, the deletions of amino acids within the fusion peptide are consecutive e.g. amino acids 806 and 807 are deleted or amino acids 809 and 810 are deleted. In embodiments, the deletions of amino acids within the fusion peptide are non-consecutive e.g. amino acids 806 and 808 are deleted or amino acids 810 and 813 are deleted. In embodiments, the CoV S polypeptides containing a deletion, corresponding to one or more amino acids of the fusion peptide, have an amino acid sequence selected from SEQ ID NOS: 66, 77, and 105-108.

In embodiments, the CoV S polypeptides contain a mutation at Lys-973 of the native CoV Spike (S) polypeptide (SEQ ID NO: 2). In embodiments, Lys-973 is mutated to any natural amino acid. In embodiments, Lys-973 is mutated to proline. In embodiments, Lys-973 is mutated to glycine. In embodiments, the CoV S polypeptides containing a mutation at amino acid 973 are selected from the group consisting of SEQ ID NO: 84-89, 105-106, and 109-110.

In embodiments, the CoV S polypeptides contain a mutation at Val-974 of the native CoV Spike (S) polypeptide (SEQ ID NO: 2). In embodiments, Val-974 is mutated to any natural amino acid. In embodiments, Val-974 is mutated to proline. In embodiments, Val-974 is mutated to glycine. In embodiments, the CoV S polypeptides containing a mutation at amino acid 974 are selected from the group consisting of SEQ ID NO: 84-89, 105-106, and 109-110.

In embodiments, the CoV S polypeptides contain a mutation at Lys-973 and Val-974 of the native CoV Spike (S) polypeptide (SEQ ID NO: 2). In embodiments, Lys-973 and Val-974 are mutated to any natural amino acid. In embodiments, Lys-973 and Val-974 are mutated to proline. In embodiments, the CoV S polypeptides containing a mutation at amino acids 973 and 974 are selected from SEQ ID NOS: 84-89, 105-106, and 109-110.

### CoV S Polypeptide Antigens—Modifications to S2 Subunit—HR1 Domain

In embodiments, the CoV S polypeptides contain one or more modifications to the HR1 domain having an amino acid sequence of SEQ ID NO: 119, which corresponds to amino acids 912-984 of SEQ ID NO: 1 or amino acids 889-971 of SEQ ID NO: 2.

The amino acid sequence of the HR1 domain (SEQ ID NO: 119) is shown below.

MAYRFNGIGVTQNVLYENQKLIANQFNSAIGKIQDSLSSTASALGKLQD
VVNQNAQALNTLVKQLSSNFGAISSVLNDILSRL

In embodiments, the CoV S polypeptides described herein comprise an HR1 domain with at least 95%, at least 96%, at least 97%, at least 98%, at least 99%, or at least 99.5%, identity to the HR1 domain of SEQ ID NO: 1 or SEQ ID NO: 2. The HR1 domain may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the HR1 domain of SEQ ID NO: 1 or SEQ ID NO: 2. The HR1 domain may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the HR1 domain of SEQ ID NO: 1 or SEQ ID NO: 2.

In embodiments, the HR1 domain may contain any combination of modifications as shown in Table 1G.

TABLE 1G
Modifications to HR1 (SEQ ID NO: 119)

| Position | Position | Position | |
|---|---|---|---|
| within | within | within | |
| SEQ ID | SEQ ID | SEQ ID | |
| NO: 1 | NO: 2 | NO: 119 | Possible Modifications |
| 982 | 969 | 81 | Mutation to alanine |
| | | | Mutation to glycine |
| | | | Mutation to threonine |

* amino acids 912-984 of SEQ ID NO: 1 and amino acids 889-971 of SEQ ID NO: 2)

### CoV S Polypeptide Antigens—Modifications to S2 Subunit—HR2 Domain

In embodiments, the CoV S polypeptides contain one or more modifications to the HR2 domain having an amino acid sequence of SEQ ID NO: 125, which corresponds to amino acids 1163-1213 of SEQ ID NO: 1 or amino acids 1150-1200 of SEQ ID NO: 2.

The amino acid sequence of the HR2 domain (SEQ ID NO: 125) is shown below.

DVDLGDISGINASVVNIQKEIDRLNEVAKNLNESLIDLQELGKYEQYIK
WP

In embodiments, the CoV S polypeptides described herein comprise an HR2 domain with at least 95%, at least 96%, at least 97%, at least 98%, at least 99%, or at least 99.5%, identity to the HR2 domain of SEQ ID NO: 1 or SEQ ID NO: 2. The HR2 domain may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the HR2 domain of SEQ ID NO: 1 or SEQ ID NO: 2. The HR2 domain may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 2.7 amino acids, or between about 25 and 30 amino acids as compared to the HR2 domain of SEQ ID NO: 1 or SEQ ID NO: 2.

### CoV S Polypeptide Antigens—Modifications to the TM Domain

In embodiments, the CoV S polypeptides contain one or more modifications to the TM domain having an amino acid sequence of SEQ ID NO: 123, which corresponds to amino acids 1214-1237 of SEQ ID NO: 1 or amino acids 1201-1224 of SEQ ID NO: 2.

The amino acid sequence of the TM domain (SEQ ID NO: 123) is shown below.

WYIWLGFIAGLIAIVMVTIMLCCM

In embodiments, the CoV S polypeptides described herein comprise a TM domain with at least 95%, at least 96%, at least 97%, at least 98%, at least 99%, or at least 99.5%, identity to the TM domain of SEQ ID NO: 1 or SEQ ID NO: 2. The TM domain may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the TM domain of SEQ ID NO: 1 or SEQ ID NO: 2. The TM domain may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the TM domain of SEQ ID NO: 1 or SEQ ID NO: 2.

In embodiments, the CoV S polypeptides described herein lack the entire TM domain. In embodiments, the CoV S polypeptides comprise the TM domain.

### CoV S Polypeptide Antigens—Modifications to the CT

In embodiments, the CoV S polypeptides contain one or more modifications to the CT having an amino acid sequence of SEQ ID NO: 124, which corresponds to amino acids 1238-1273 of SEQ ID NO: 1 or amino acids 1225-1260 of SEQ ID NO: 2.

The amino acid sequence of the CT (SEQ ID NO: 124) is shown below:

TSCCSCLKGCCSCGSCCKFDEDDSEPVLKGVKLHYT

In embodiments, the CoV S polypeptides described herein comprise a CT with at least 95%, at least 96%, at least 97%, at least 98%, at least 99%, or at least 99.5%, identity to the CT of SEQ ID NO: 1 or SEQ ID NO: 2. The CT may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the CT of SEQ ID NO: 1 or SEQ ID NO: 2. The CT may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the CT of SEQ ID NO: 1 or SEQ ID NO: 2.

In embodiments, the CoV S polypeptides described herein lack a CT. In embodiments, the CoV S polypeptides comprise the CT.

In embodiments, the CoV S polypeptides comprise a TM and a CT. In embodiments, the CoV Spike (S) polypeptides contain a deletion of one or more amino acids from the transmembrane and cytoplasmic tail (TMCT) (corresponding to amino acids 1201-1260). The amino acid sequence of the TMCT is represented by SEQ ID NO: 39. In embodiments, the CoV S polypeptides which have a deletion of one or more residues of the TMCT have enhanced protein expression. In embodiments, the CoV Spike (S) polypeptides which have one or more deletions from the TMCT have an amino acid sequence selected from the group consisting of SEQ ID NO: 40, 41, 42, 52, 54, 59, 61, 88, and 89. In embodiments, the CoV S polypeptides which have one or more deletions from the TM-CD are encoded by an isolated nucleic acid sequence selected from the group consisting of SEQ ID NO: 39, 43, 53, and 60.

### CoV S Polypeptide Antigens—Non-Limiting Combinations of Mutations

In embodiments, the CoV S polypeptides contain a deletion of amino acids 56 and 57 of the native CoV Spike (S) polypeptide (SEQ ID NO: 2).

In embodiments, the CoV S polypeptides contain deletions of amino acids 131 and 132 of the native CoV Spike (S) polypeptide (SEQ ID NO: 2).

In embodiments, the CoV S polypeptides contain a deletion of amino acids 56 and 131 of the native CoV Spike (S) polypeptide (SEQ ID NO: 2). In embodiments, the CoV S polypeptides contain a deletion of amino acids 57 and 131 of the native CoV Spike (S) polypeptide (SEQ ID NO: 2).

In embodiments, the CoV S polypeptides contain a deletion of amino acids 56, 57, and 131 of the native CoV Spike (S) polypeptide (SEQ ID NO: 2).

In embodiments, the CoV S polypeptides contain a deletion of amino acids 56 and 132 of the native CoV Spike (S) polypeptide (SEQ ID NO: 2).

In embodiments, the CoV S polypeptides contain a deletion of amino acids 57 and 132 of the native CoV Spike (S) polypeptide (SEQ ID NO: 2).

In embodiments, the CoV S polypeptides contain a deletion of amino acids 56, 57, and 132 of the native CoV Spike (S) polypeptide (SEQ ID NO: 2).

In embodiments, the CoV S polypeptides contain a deletion of amino acids 56, 57, 131, and 132 of the native CoV Spike (S) polypeptide (SEQ ID NO: 2).

In embodiments, the CoV S polypeptides contain mutations that stabilize the prefusion conformation of the CoV S polypeptide. In embodiments, the CoV S polypeptides contain proline or glycine substitutions which stabilize the prefusion conformation. This strategy has been utilized for to develop a prefusion stabilized MERS-CoV S protein as described in the following documents which are each incorporated by reference herein in their entirety: Proc Natl Acad Sci USA. 2017 Aug. 29; 114(35):E7348-E7357; Sci Rep. 2018 Oct. 24; 8(1):15701; U.S. Publication No. 2020/0061185; and PCT Application No. PCT/US2017/058370.

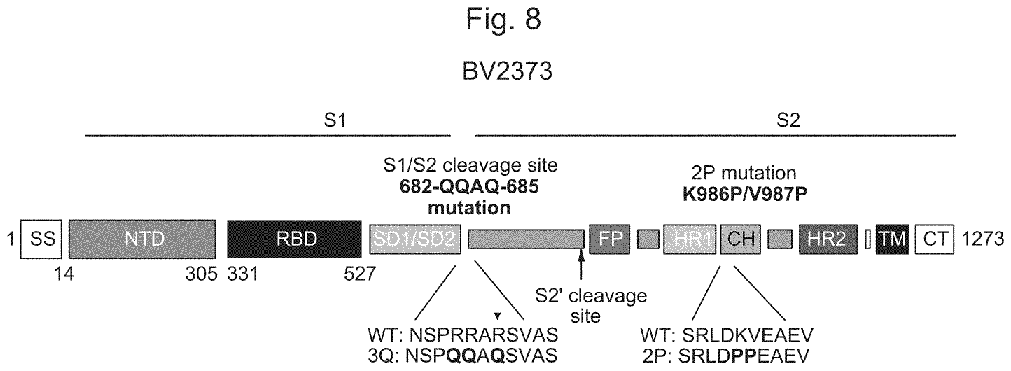

In embodiments, the CoV S polypeptides contain a mutation at Lys-973 and Val-974 and an inactivated furin cleavage site. In embodiments, the CoV S polypeptides contain mutations of Lys-973 and Val-974 to proline and an inactivated furin cleavage site, having the amino acid sequence of QQAQ (SEQ ID NO: 7) or GSAS (SEQ ID NO: 96). An exemplary CoV S polypeptide containing a mutation at Lys-973 and Val-974 and an inactivated thrill cleavage site is depicted in FIG. 8. In embodiments, the CoV S polypeptides containing mutations of Lys-973 and Val-974 to proline and an inactivated furin cleavage site have an amino acid sequences of SEQ ID NOS: 86 or 87 and a nucleic acid sequence of SEQ ID NO: 96.

In embodiments, the CoV S polypeptides contain a mutation at Lys-973 and Val-974, an inactivated furin cleavage site, and a deletion of one or more amino acids of the fusion peptide. In embodiments, the CoV S polypeptides contain mutations of Lys-973 and Val-974 to proline, an inactivated furin cleavage site having the amino acid sequence of QQAQ (SEQ ID NO: 7) or GSAS (SEQ ID NO: 96), and deletion of one or more amino acids of the fusion peptide. In embodiments, the CoV S polypeptides containing mutations of Lys-973 and Val-974 to proline, an inactivated furin cleavage site, and deletion of one or more amino acids of the fusion peptide having an amino acid sequence of SEQ ID NO: 105 or 106. In embodiments, the CoV S polypeptide contains a mutation of Leu-5 to phenylalanine, mutation of Thr-7 to asparagine, mutation of Pro-13 to serine, mutation of Asp-125 to tyrosine, mutation of Arg-177 to serine, mutation of Lys-404 to threonine, mutation of Glu-471 to lysine, mutation of Asn-488 to tyrosine, mutation of His-642 to tyrosine, mutation of Thr-1014 to isoleucine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike (S) polypeptide (SEQ ID NO: 2).

In embodiments, the CoV S polypeptide contains a mutation of Trp-139 to cysteine, mutation of Leu-439 to arginine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike (S) polypeptide (SEQ ID NO: 2). In embodiments, the CoV S polypeptide contains a mutation of Trp-152 to cysteine, mutation of Leu-452 to arginine, mutation of Ser-13 to isoleucine, mutations of Lys-986 and Val-987 to proline, and an inactivated furin cleavage site having the amino acid sequence of QQAQ (SEQ ID NO: 7) or GSAS (SEQ ID NO: 96) relative to the native CoV Spike (S) polypeptide (SEQ ID NO: 1).

In embodiments, the CoV S polypeptide contains a mutation of Lys-404 to threonine or asparagine, mutation of Glu-471 to lysine, mutation of Asn-488 to tyrosine, mutation of Leu-5 to phenylalanine, mutation of Asp-67 to alanine, mutation of Asp-202 to glycine, deletion of one or more of amino acids 229-231, mutation of Arg-233 to isoleucine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike (S) polypeptide (SEQ ID NO: 2).

In embodiments, the CoV S polypeptide contains a mutation of Asn-488 to tyrosine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike (S) polypeptide (SEQ ID NO: 2). In embodiments, the CoV S polypeptide having a mutation of Asn-488 to tyrosine, mutations of Lys-973 and Val-974 to proline, and an inactivated furin cleavage site having the amino acid sequence of QQAQ (SEQ ID NO: 7) or DSAS (SEQ ID NO: 96) comprises an amino acid sequence of SEQ ID NO: 112.

In embodiments, the CoV S polypeptide contains a mutation of Asp-601 to glycine, a mutation of Asn-488 to tyrosine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike (S) polypeptide (SEQ ID NO: 2). In embodiments, the CoV S polypeptide having a mutation of Asn-488 to tyrosine, mutations of Lys-973 and Val-974 to proline, and an inactivated furin cleavage site having the amino acid sequence of QQAQ (SEQ ID NO: 7) or GSAS (SEQ ID NO: 96) comprises an amino acid sequence of SEQ ID NO: 113.

In embodiments, the CoV S polypeptide contains deletion of amino acids 56, 57, and 131, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of QQAQ (SEQ ID NO: 7), GSAS (SEQ ID NO: 96), or GG relative to the native CoV Spike (S) polypeptide (SEQ ID NO: 2). In embodiments, the CoV S polypeptide having deletion of amino acids 56, 57, and 131, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of QQAQ (SEQ ID NO: 7) or GSAS (SEQ ID NO: 96) comprises an amino acid sequence of SEQ ID NO: 114. In embodiments, the CoV S polypeptide having deletion of amino acids 56, 57, and 131, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of QQAQ (SEQ ID NO: 7) or DSAS (SEQ ID NO: 96) or GG comprises an amino acid sequence of SEQ ID NO: 136. In embodiments, the CoV S polypeptide having deletion of amino acids 56, 57, and 131, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of GG comprises an amino acid sequence of SEQ ID NO: 137 or SEQ ID NO: 138. In some embodiments, the CoV S polypeptide having an amino acid sequence of SEQ ID NO: 114 or SEQ ID NO: 136 is encoded by a nucleic acid having a nucleic acid sequence of SEQ ID NO: 135. In some embodiments, the CoV S polypeptide having an amino acid sequence of SEQ ID NO: 137 or SEQ ID NO: 138 is encoded by a nucleic acid having a sequence of SEQ ID NO: 139.

In embodiments, the CoV S polypeptide contains deletion of amino acids 56, 57, and 132, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of QQAQ (SEQ ID NO: 7) or GSAS (SEQ ID NO: 96 relative to the native CoV Spike (S) polypeptide (SEQ ID NO: 2). In embodiments, the CoV S polypeptide having a deletion of amino acids 56, 57, and 132, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of QQAQ (SEQ ID NO: 7) or GSAS (SEQ ID NO: 96) comprises an amino acid sequence of SEQ ID NO: 114.

In embodiments, the CoV S polypeptide contains mutation of Asn-488 to tyrosine, mutation of Asp-67 to alanine, mutation of Leu-229 to histidine, mutation of Asp-202 to glycine, mutation of Lys-404 to asparagine, mutation of Glu-471 to lysine, mutation of Ala-688 to valine, mutation of Asp-601 to glycine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike (S) polypeptide (SEQ ID NO: 2). In embodiments, the CoV S polypeptide having a mutation of Asn-488 to tyrosine, mutation of Asp-67 to alanine, mutation of Leu-229 to histidine, mutation of Asp-202 to glycine, mutation of Lys-404 to asparagine, mutation of Glu-471 to lysine, mutation of Ala-688 to valine, mutation of Asp-601 to glycine, mutations of Lys-973 and Val-974 to proline, and an inactivated furin cleavage site having the amino acid sequence of QQAQ (SEQ ID NO: 7) or GSAS (SEQ ID NO: 96) comprises an amino acid sequence of SEQ ID NO: 115.

In embodiments, the CoV S polypeptide contains one or more modifications selected from: K973P, V974P, an inactivated furin cleavage site, deletions of amino acid 56, deletion of amino acid 57, deletion of amino acid 131, N488Y, A557D, D601G, P668H, T703I, S969A, and D1105H, wherein the amino acids are numbered with respect to a CoV S polypeptide having an amino acid sequence of SEQ ID NO: 2. In embodiments, the inactivated furin cleavage site has the amino acid sequence of QQAQ (SEQ ID NO: 7). In embodiments, the inactivated furin cleavage site has the amino acid sequence of GG.

In embodiments, the CoV S polypeptide contains one or more modifications selected from: K973P, V974P, an inactivated furin cleavage site, D67A, D202G, L229H, K404N, E471K, N488Y, D601G, and A688V, wherein the amino acids are numbered with respect to a CoV S polypeptide having an amino acid sequence of SEQ ID NO: 2. In embodiments, the inactivated furin cleavage site has the amino acid sequence of QQAQ (SEQ ID NO: 7). In embodiments, the inactivated furin cleavage site has the amino acid sequence of GG.

In embodiments, the CoV S polypeptide contains one or more modifications selected from: K973P, V974P, an inactivated furin cleavage site, deletion of amino acids 229-231, D67A, D202G, K404N, E471K, N488Y, D601G, and A688V, wherein the amino acids are numbered with respect to a CoV S polypeptide having an amino acid sequence of SEQ ID NO: 2.

In embodiments, the CoV S polypeptide contains one or more modifications selected from: K973P, V974P, an inactivated furin cleavage site having the amino acid sequence of QQAQ (SEQ ID NO: 7), deletion of amino acids 229-231, L5F, D67A, D202G, K404N, E471K, N488Y, D601G, and A688V, wherein the amino acids are numbered with respect to a CoV S polypeptide having an amino acid sequence of SEQ ID NO: 2. In embodiments, the CoV S polypeptide having one or more modifications selected from K973P, V974P, an inactivated furin cleavage site having the amino acid sequence of QQAQ (SEQ II) NO: 7), deletion of amino acids 229-231, L5F, D67A, D202G, K404N, E471K, N488Y, D601G, and A688V, wherein the amino acids are numbered with respect to a CoV S polypeptide having an amino acid sequence of SEQ ID NO: 2 comprises the amino acid sequence of SEQ ID NO: 144. In embodiments, the CoV S polypeptide having the amino acid sequence of SEQ ID NO: 144 is encoded by a nucleic acid having a sequence of SEQ ID NO: 145.

In embodiments, the CoV S polypeptide contains one or more modifications selected from: K973P, V974P, an inactivated furin cleavage site having the amino acid sequence of GG, deletion of amino acids 229-231, L5F, D67A, D202G, K404N, E471K, N488Y, D601G, and A688V, wherein the amino acids are numbered with respect to a CoV S polypeptide having an amino acid sequence of SEQ ID NO: 2. In embodiments, the CoV S polypeptide having one or more modifications selected from K973P, V974P, an inactivated furin cleavage site having the amino acid sequence of GG, deletion of amino acids 229-231, L5F, D67A, D202G, K404N, E471K, N488Y, D601G, and A688V, wherein the amino acids are numbered with respect to a CoV S polypeptide having an amino acid sequence of SEQ ID NO: 2 comprises the amino acid sequence of SEQ ID NO: 144. In embodiments, the CoV S polypeptide having the amino acid sequence of SEQ ID NO: 144 is encoded by a nucleic acid having a sequence of SEQ ID NO: 145.

In embodiments, the CoV S polypeptide contains one or more modifications selected from: K973P, V974P, an inactivated furin cleavage site, L5F, T7N, P13S, D125Y, R177S, K404T, E471K, N488Y, D601G, H642Y, T1014I, and V1163F, wherein the amino acids are numbered with respect to a CoV S polypeptide having an amino acid sequence of SEQ ID NO: 2. In embodiments, the CoV S polypeptide containing one or more modifications selected from: K973P, V974P, an inactivated furin cleavage site, L5F, T7N, P13S, D125Y, R177S, K404T, E471K, N488Y, D601G, H642Y, T1014I, and V1163F, wherein the amino acids are numbered with respect to a CoV S polypeptide having an amino acid sequence of SEQ ID NO: 2, has an amino acid sequence of SEQ ID NO: 151. In embodiments, the CoV S polypeptide having an amino acid sequence of SEQ ID NO: 151 is encoded by a nucleic acid having a sequence of SEQ ID NO: 150.

In embodiments, the CoV S polypeptide contains one or more modifications selected from: K973P, V974P, an inactivated furin cleavage site, deletion of amino acids 229-231, L5F, D67A, D202G, L229H, K404N, E471K, N488Y, D601G, and A688V, wherein the amino acids are numbered with respect to a CoV S polypeptide having an amino acid sequence of SEQ ID NO: 2.

In embodiments, the CoV S polypeptide contains one or more modifications selected from: K973P, V974P, an inactivated furin cleavage site, K404N, E471K, N488Y, L5F, D67A, D202G, L229H, D601G, A688V, and deletion of amino acids 229-231, wherein the amino acids are numbered with respect to a CoV S polypeptide having an amino acid sequence of SEQ ID NO: 2. In embodiments, the inactivated furin cleavage site has the amino acid sequence of QQAQ (SEQ ID NO: 7). In embodiments, the inactivated furin cleavage site has the amino acid sequence of GG

In embodiments, the CoV S polypeptide contains one or more modifications selected from: K973P, V974P, an inactivated furin cleavage site. K404N, E471K, and N488K wherein the amino acids are numbered with respect to a CoV S polypeptide having an amino acid sequence of SEQ ID NO: 2. In embodiments, the CoV S polypeptide contains one or more modifications selected from: K973P, V974P, an inactivated furin cleavage site, K404N, E471K, and N488Y. In embodiments, the CoV S polypeptide is the RBD of the CoV S polypeptide having one or more modifications selected from K973P, V974P, an inactivated furin cleavage site, K404N, E471K, and N488K wherein the amino acids are numbered with respect to a CoV S polypeptide having an amino acid sequence of SEQ ID NO: 2. In embodiments, the CoV S polypeptide is the RBD of the CoV S polypeptide having one or more modifications selected from K973P, V974P, an inactivated furin cleavage site, K404N, E471K, and N488Y wherein the amino acids are numbered with respect to a CoV S polypeptide having an amino acid sequence of SEQ ID NO: 2.

In embodiments, the CoV S polypeptide contains one or more modifications selected from: K973P, V974P, an inactivated furin cleavage site having the amino acid sequence of GG, D601G, E404N, E471K, and N488Y. In embodiments, the CoV S polypeptide contains one or more modifications selected from: K973P, V974P, an inactivated furin cleavage site having the amino acid sequence of GG, and a D601G mutation, wherein the amino acids are numbered with respect to a CoV S polypeptide having an amino acid sequence of SEQ ID NO: 2. In embodiments, the CoV S polypeptide containing modifications selected from: K973P, V974P, an inactivated furin cleavage site having the amino acid sequence of GG, and a D601G mutation has an amino acid sequence of SEQ ID NO: 133.

In embodiments, the CoV S polypeptide contains one or more modifications selected from: K973P, V974P, an inactivated furin cleavage site, optionally wherein the inactivated furin cleavage site is QQAQ (SEQ ID NO: 7) or GG, K404N, E471K, N488K, D67A, D202G, L229H, D601G, and A688V, wherein the amino acids are numbered with respect to a CoV S polypeptide having an amino acid sequence of SEQ ID NO: 2. In embodiments, the CoV S polypeptide containing one or more modifications selected from: K973P, V974P, an inactivated furin cleavage site, optionally wherein the inactivated furin cleavage site is QQAQ (SEQ ID NO: 7) or GG, K404N, E471K, N488K, D67A, D202G, L229H, D601G, and A688V has an amino acid sequence of SEQ ID NO: 132 or SEQ ID NO: 141. In embodiments, the CoV S polypeptide having an amino acid sequence of SEQ ID NO: 132 is encoded by a nucleic acid having a nucleic acid sequence of SEQ ID NO: 131. In embodiments, the CoV S polypeptide having an amino acid sequence of SEQ ID NO: 132 is encoded by a nucleic acid having a nucleic acid sequence of SEQ ID NO: 142.

In embodiments, the CoV S polypeptide contains one or more modifications selected from: K973P, V974P, an inactivated furin cleavage site, W139C and L439R, wherein the amino acids are numbered with respect to a CoV S polypeptide having an amino acid sequence of SEQ ID NO: 2. In embodiments, the CoV S polypeptide comprising K973P, V974P, an inactivated furin cleavage site, W139C and L439R modifications is expressed with a signal peptide having an amino acid sequence of SEQ ID NO: 117 or SEQ ID NO: 5. In embodiments, the CoV S polypeptide comprises one or more modifications selected from: K973P, V974P, an inactivated furin cleavage site, D601G, W139C, and L439R, wherein the amino acids are numbered with respect to a CoV S polypeptide having an amino acid sequence of SEQ ID NO: 2. In embodiments, the CoV S polypeptide comprises K973P, V974P, an inactivated furin cleavage site, D601G, W139C, and L439R modifications and is expressed with a signal peptide having an amino acid sequence of SEQ ID NO: 117 or SEQ ID NO: 5.

In embodiments, the CoV S polypeptide comprises one or more modifications selected from: K973P, V974P, an inactivated furin cleavage site, D601G, L5F, D67A, D202G, deletions of amino acids 229-231, R233I, K404N, E471K, N488Y, and A688V, wherein the amino acids are numbered with respect to a CoV S polypeptide having an amino acid sequence of SEQ ID NO: 2.

In embodiments, the CoV S polypeptide contains one or more modifications selected from: K973P, V974P, an inactivated furin cleavage site, optionally wherein the inactivated furin cleavage site is QQAQ (SEQ ID NO: 7), W139C, S481P, D601G, and L439R, wherein the amino acids are numbered with respect to a CoV S polypeptide having an ammo acid sequence of SEQ ID NO: 2. In embodiments, the CoV S polypeptide contains one or more modifications selected from: K973P, V974P, an inactivated furin cleavage site, optionally wherein the inactivated furin cleavage site is QQAQ (SEQ ID NO: 7), W139C, D601G, and L439R, wherein the amino acids are numbered with respect to a CoV S polypeptide having an amino acid sequence of SEQ ID NO: 2. In embodiments, the CoV S polypeptide contains one or more modifications selected from: K973P, V974P, an inactivated furin cleavage site, optionally wherein the inactivated furin cleavage site is QQAQ (SEQ ID NO: 7), W139C, S481P, and D601G wherein the amino acids are numbered with respect to a CoV S polypeptide having an amino acid sequence of SEQ ID NO: 2. In embodiments, the CoV S polypeptide containing one or more modifications selected from: K973P, V974P, an inactivated furin cleavage site, optionally wherein the inactivated furin cleavage site is QQAQ (SEQ ID NO: 7), W139C, S481P, D601G, and L439R has the amino acid sequence of SEQ ID NO: 153. In embodiments, the CoV S polypeptide having the amino acid sequence of SEQ ID NO: 153 comprises a signal peptide having an amino acid sequence of SEQ ID NO: 117. In embodiments, the CoV S polypeptide having the amino acid sequence of SEQ ID NO: 153 comprises a signal peptide having an amino acid sequence of SEQ ID NO: 5.

In embodiments, the CoV S polypeptide contains one or more modifications selected from: K973P, V974P, an inactivated furin cleavage site, optionally wherein the inactivated furin cleavage site is QQAQ (SEQ ID NO: 7), T82I, D240G, E471K, D601G, and A688V, wherein the amino acids are numbered with respect to a CoV S polypeptide having an amino acid sequence of SEQ ID NO: 2. In embodiments, the CoV S polypeptide containing one or more modifications selected from: K973P, V974P, an inactivated furin cleavage site, optionally wherein the inactivated furin cleavage site is QQAQ (SEQ ID NO: 7), T82I, D240G, E471K, D601G, and A688V, wherein the amino acids are numbered with respect to a CoV S polypeptide having an amino acid sequence of SEQ ID NO: 2, has an amino acid sequence of SEQ ID NO: 156. In embodiments, the CoV S polypeptide containing one or more modifications selected from: K973P, V974P, an inactivated furin cleavage site, optionally wherein the inactivated furin cleavage site is QQAQ (SEQ ID NO: 7), T82I, D240G, E471K, D601G, and A688V, wherein the amino acids are numbered with respect to a CoV S polypeptide having an amino acid sequence of SEQ ID NO: 2, comprises a signal peptide having an amino acid sequence of SEQ ID NO: 154 or SEQ ID NO: 5.

In embodiments, the CoV S polypeptide contains one or more modifications selected from: K973P, V974P, an inactivated furin cleavage site, optionally wherein the inactivated furin cleavage site is QQAQ (SEQ ID NO: 7), T82I, D240G, S464N, D601G, and A688V, wherein the amino acids are numbered with respect to a CoV S polypeptide having an amino acid sequence of SEQ ID NO: 2. In embodiments, the CoV S polypeptide containing one or more modifications selected from: K973P, V974P, an inactivated furin cleavage site, optionally wherein the inactivated furin cleavage site is QQAQ (SEQ ID NO: 7), T82I, D240G, S464N, D601G, and A688V, wherein the amino acids are numbered with respect to a CoV S polypeptide having an amino acid sequence of SEQ ID NO: 2, has an amino acid sequence of SEQ ID NO: 158. In embodiments, the CoV S polypeptide containing one or more modifications selected from: K973P, V974P, an inactivated furin cleavage site, optionally wherein the inactivated furin cleavage site is QQAQ (SEQ ID NO: 7), T82I, D240G, S464N, D601G, and A688V, wherein the amino acids are numbered with respect to a CoV S polypeptide having an amino acid sequence of SEQ ID NO: 2, comprises a signal peptide of SEQ ID NO: 154.

In embodiments, the CoV S polypeptide contains one or more modifications selected from: K973P, V974P, an inactivated furin cleavage site, optionally wherein the inactivated furin cleavage site is QQAQ (SEQ ID NO: 7), deletion of amino acid 56, deletion of amino acid 57, deletion of amino acid 131, a N488Y mutation, an A557D mutation, a D601G mutation, a P668H mutation, a T703I mutation, a S969A mutation, and a D1105H mutation, wherein the CoV polypeptide is numbered with respect to the wild-type SARS-CoV-2 S polypeptide having the amino acid sequence of SEQ ID NO: 2. In embodiments, the CoV S polypeptide contains one or more modifications selected from: K973P, V974P, an inactivated furin cleavage site, optionally wherein the inactivated furin cleavage site is QQAQ (SEQ ID NO: 7), deletion of amino acid 56, deletion of amino acid 57, deletion of amino acid 132, a N488Y mutation, an A557D mutation, a D601G mutation, a P668H mutation, a T703I mutation, a S969A mutation, and a D1105H mutation, wherein the CoV S polypeptide is numbered with respect to the wild-type SARS-CoV-2 S polypeptide having the amino acid sequence of SEQ ID NO: 2.

In embodiments, the CoV S polypeptide contains one or more modifications selected from: K973P, V974P, an inactivated furin cleavage site, optionally wherein the inactivated furin cleavage site is QQAQ (SEQ ID NO: 7), a D67A mutation, a L229H mutation, a R233I mutation, an A688V mutation, an N488Y mutation, a K404N mutation, a E471K mutation, and a D601G mutation, wherein the CoV S polypeptide is numbered with respect to the wild-type SARS-CoV-2 S polypeptide having the amino acid sequence of SEQ ID NO: 2.

In embodiments, the CoV S polypeptide contains one or more modifications selected from: K973P, V974P, an inactivated furin cleavage site, optionally wherein the inactivated furin cleavage site is QQAQ (SEQ ID NO: 7), a L5F mutation, a T7N mutation, a P13S mutation, a D125Y mutation, a R177S mutation, a K404T mutation, a E471K mutation, a N488Y mutation, a D601G mutation, a H642Y mutation, a T1014I mutation, and a T1163F mutation, wherein the CoV S polypeptide is numbered with respect to the wild-type SARS-CoV-2 S polypeptide having the amino acid sequence of SEQ ID NO: 2.

In embodiments, the CoV S polypeptide contains one or more modifications selected from: K986P, V987P, an inactivated furin cleavage site, optionally wherein the inactivated furin cleavage site is QQAQ (SEQ II) NO: 7), a S13I mutation, a W152C mutation, and a L452R mutation, wherein the CoV S polypeptide is numbered with respect to the wild-type SARS-CoV-2 S polypeptide having the amino acid sequence of SEQ ID NO: 1. In embodiments, the CoV S polypeptide contains one or more modifications selected from: K986P, V987P, an inactivated furin cleavage site, optionally wherein the inactivated furin cleavage site is QQAQ (SEQ ID NO: 7), a S13I mutation, a W152C mutation, and a L452R mutation, wherein the CoV S polypeptide is numbered with respect to the wild-type SARS-CoV-2 S polypeptide having the amino acid sequence of SEQ ID NO: 1 lacks an N-terminal signal peptide.

In embodiments, the CoV Spike (S) polypeptides comprise a polypeptide linker. In embodiments, the polypeptide linker contains glycine and serine. In embodiments, the linker has about 50%, about 55%, about 60%, about 65%, about 70%, about 75%, about 80%, about 85%, about 90%, about 95%, or about 100% glycine.

In embodiments, the polypeptide linker has a repeat of (SGGG)ₙ (SEQ ID NO: 91), wherein n is an integer from 1 to 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50). In embodiments, the polypeptide linker has an amino acid sequence corresponding to SEQ ID NO: 90.

In embodiments, the polypeptide linker has a repeat of (GGGGS)ₙ (SEQ ID NO: 93), wherein n is an integer from 1 to 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50).

In embodiments, the polypeptide linker has a repeat of (GGGS)ₙ (SEQ ID NO: 92), wherein n is an integer from 1 to 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50).

In aspects, the polypeptide linker is a poly-(Gly)n linker, wherein n is 1, 2, 3, 4, 5, 6, 7, 8, 9, 10, 15, 16, 17, 18, 19, or 20. In other embodiments, the linker is selected from the group consisting of: dipeptides, tripeptides, and quadripeptides. In embodiments, the linker is a dipeptide selected from the group consisting of alanine-serine (AS), leucine-glutamic acid (LE), and serine-arginine (SR).

In embodiments, the polypeptide linker comprises between 1 to 100 contiguous amino acids of a naturally occurring CoV S polypeptide or of a CoV S polypeptide disclosed herein. In embodiments, the polypeptide linker has an amino acid sequence corresponding to SEQ ID NO: 94.

In embodiments, the CoV Spike (S) polypeptides comprise a foldon. In embodiments, the TMCT is replaced with a foldon. In embodiments, a foldon causes trimerization of the CoV Spike (S) polypeptide. In embodiments, the foldon is an amino acid sequence known in the art. In embodiments, the foldon has an amino acid sequence of SEQ ID NO: 68. In embodiments, the foldon is a T4 fibritin trimerization motif. In embodiments, the T4 fibritin trimerization domain has an amino acid sequence of SEQ ID NO: 103. In embodiments, the foldon is separated in amino acid sequence from the CoV Spike (S) polypeptide by a polypeptide linker. Non-limiting examples of polypeptide linkers are found throughout this disclosure.

In embodiments, the disclosure provides CoV S polypeptides comprising a fragment of a coronavirus S protein and nanoparticles and vaccines comprising the same. In embodiments, the fragment of the coronavirus S protein is between 10 and 1500 amino acids in length (e.g. about 10, about 20, about 30, about 40, about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, or about 1500 amino acids in length). In embodiments, the fragment of the coronavirus S protein is selected from the group consisting of the receptor binding domain (RBD), subdomain 1, subdomain 2, upper helix, fusion peptide, connecting region, heptad repeat 1, central helix, heptad repeat 2, NTD, and TMCT.

In embodiments, the CoV S polypeptide comprises an RBD and a subdomain 1. In embodiments, the CoV S polypeptide comprising an RBD and a subdomain 1 is amino acids 319 to 591 of SEQ ID NO: 1.

In embodiments, the CoV S polypeptide contains a fragment of a coronavirus S protein, wherein the fragment of the coronavirus S protein is the RBD. Non-limiting examples of RBDs include the RBD of SARS-CoV-2 (amino acid sequence=SEQ ID NO: 69), the RBD of SARS (amino acid sequence=SEQ ID NO: 70), and the RBD of MERS, (amino acid sequence=SEQ ID NO: 71).

In embodiments, the CoV S polypeptide contains two or more RBDs, which are connected by a polypeptide linker. In embodiments, the polypeptide linker has an amino acid sequence of SEQ ID NO: 90 or SEQ ID NO: 94.

In embodiments, the CoV S polypeptide contains 1, 2, 3, 4, 5, 6, 7, 8, 9, 10, 11, 14, 15, 16, 17, 18, 19, or 20 RBDs.

In some embodiments, the CoV S polypeptide contains two or more SARS-CoV-2 RBDs, which are connected by a polypeptide linker. In embodiments, the antigen containing two or more SARS-CoV-2 RBDs has an amino acid sequence corresponding to one of SEQ ID NOS: 72-75.

In embodiments, the CoV S polypeptide contains a SARS-CoV-2 RBD and a SARS RBD. In embodiments, the CoV S polypeptide comprises a SARS-CoV-2 RBD and a SARS RBD, wherein each RBD is separated by a polypeptide linker. In embodiments, the CoV S polypeptide comprising a SARS-CoV-2 RBD and a SARS RBD has an amino acid sequence selected from the group consisting of SEQ ID NOS: 76-79.

In embodiments, the CoV S polypeptide contains a SARS-CoV-2 RBD and a MERS RBD. In embodiments, the CoV S polypeptide comprises a SARS-CoV-2 RBD and a MERS RBD, wherein each RBD is separated by a polypeptide linker.

In embodiments, the CoV S polypeptide comprises a SARS RBD and a MERS RBD. In embodiments, the CoV S polypeptide comprises a SARS RBD and a MERS RBD, wherein each RBD is separated by a polypeptide linker.

In embodiments, the CoV S polypeptide contains a SARS-CoV-2 RBD, a SARS RBD, and a MERS RBD. In embodiments, the CoV S polypeptide contains a SARS-CoV-2 RBD, a SARS RBD, and a MERS RBD, wherein each RBD is separated by a polypeptide linker. In embodiments, the CoV S polypeptide comprising a SARS-CoV-2 RBD, a SARS RBD, and a MERS RBD has an amino acid sequence selected from the group consisting of SEQ ID NOS: 80-83.

In embodiments, the CoV S polypeptides described herein are expressed with an N-terminal signal peptide. In embodiments, the N-terminal signal peptide has an amino acid sequence of SEQ ID NO: 5 (MFVFLVLLPLVSS). In embodiments, the N-terminal signal peptide has an amino acid sequence of SEQ ID NO: 117 (MFVFLVLLPLVSI). In embodiments, the N-terminal signal peptide has an amino acid sequence of SEQ ID NO: 154 (MFVFFVLLPLVSS). In embodiments, the signal peptide may be replaced with any signal peptide that enables expression of the CoV S protein. In embodiments, one or more of the CoV S protein signal peptide amino acids may be deleted or mutated. An initiating methionine residue is maintained to initiate expression. In embodiments, the CoV S polypeptides are encoded by a nucleic acid sequence selected from the group consisting of SEQ ID NO: 35, SEQ ID NO: 37, SEQ ID NO: 95, SEQ ID NO: 43, SEQ ID NO: 47, SEQ ID NO: 50, SEQ ID NO: 53, SEQ ID NO: 55, SEQ ID NO: 57, SEQ ID NO: 96, SEQ ID NO: 60, SEQ ID NO: 131, SEQ ID NO: 135, SEQ ID NO: 142, SEQ ID NO: 145, SEQ ID NO: 148, and SEQ ID NO: 150. In embodiments, the N-terminal signal peptide of the CoV S polypeptide contains a mutation at Ser-13 relative to the native CoV Spike (S) signal polypeptide (SEQ ID NO: 5). In embodiments, Ser-13 is mutated to any natural amino acid. In embodiments, Ser-13 is mutated to alanine, methionine, isoleucine, leucine, threonine, or valine. In embodiments, Ser-13 is mutated to isoleucine.

Following expression of the CoV S protein in a host cell, the N-terminal signal peptide is cleaved to provide the mature CoV protein sequence (SEQ ID NOS: 2, 4, 38, 41, 44, 48, 51, 54, 58, 61, 63, 65, 67, 73, 75, 78, 79, 82, 83, 85, 87, 89, 106, 110. 132, 133, 114, 138, 141, 144, 147, 151, 153, 156, and 158, 164-168). In embodiments, the signal peptide is cleaved by host cell proteases. In aspects, the full-length protein may be isolated from the host cell and the signal peptide cleaved subsequently.

Following cleavage of the signal peptide from the CoV Spike (S) polypeptide with an amino acid sequence corresponding to SEQ ID NOS: 1, :3, 36, 40, 42, 46, 49, 52, 56, 59, 62, 64, 66, 72, 74, 76, 77, 80, 81, 84, 86, 7 105, 107, 88, 109, 130, 134, 136, 137, 140, 143, 146, 149, 152, 155, 157, 159-163 during expression and purification, a mature polypeptide having an amino acid sequence selected from the group consisting of SEQ ID NOS: 2, 4, 38, 41, 44, 48, 51, 54, 58, 61, 63, 65, 67, 73, 75, 78, 79, 82, 83, 85, 106, 108, 89, and 110, 112-115, 132, 133, 114, 138, 141, 144 147, 151, 153, 156, and 158, 164-168 is obtained and used to produce a CoV S nanoparticle vaccine or CoV S nanoparticles.

Advantageously, the disclosed CoV S polypeptides may have enhanced protein expression and stability relative to the native CoV Spike (S) protein.

In embodiments, the CoV S polypeptides described herein contain further modifications from the native coronavirus S protein (SEQ ID NO: 2). In embodiments, the coronavirus S proteins described herein exhibit at least 80%, or at least 90%, or at least 95%, or at least 97%, or at least 99% identity to the native coronavirus S protein. A person of skill in the art would use known techniques to calculate the percent identity of the recombinant coronavirus S protein to the native protein or to any of the CoV S polypeptides described herein. For example, percentage identity can be calculated using the tools CLUSTALW2 or Basic Local Alignment Search Tool (BLAST), which are available online. The following default parameters may be used for CLUSTALW2 Pairwise alignment: Protein Weight Matrix=Gonnet; Gap Open=10; Gap Extension=0.1.

In embodiments, the CoV S polypeptides described herein are at least 95%, at least 96%, at least 97%, at least 98%, at least 99%, or at least 99.5% identical to the CoV S polypeptide having an amino acid sequence of SEQ ID NO: 87. A CoV S polypeptide may have a deletion, an insertion, or mutation of up to about 1, up to about 2, up to about 3, up to about 4, up to about 5, up to about 10, up to about 15, up to about 20, up to about 25, up to about 30, up to about 35, up to about 40, up to about 45, or up to about 50 amino acids compared to the amino acid sequence of the CoV S polypeptide having an amino acid sequence of SEQ ID NO: 87. A CoV S polypeptide may have may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, between about 25 and 30 amino acids, between about 30 and 35 amino acids, between about 35 and 40 amino acids, between about 40 and 45 amino acids, or between about 45 and 50 amino acids, as compared to the CoV S polypeptide having an amino acid sequence of SEQ ID NO: 87. In embodiments, the CoV S polypeptides described herein comprise about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 substitutions compared to the coronavirus S protein (SEQ ID NO: 87).

In embodiments, the CoV S polypeptides described herein are at least 95%, at least 96%, at least 97%, at least 98%, at least 99%, or at least 99.5% identical to the CoV S polypeptide having an amino acid sequence selected from any one of SEQ ID NOS: 2, 4, 38, 41, 44, 48, 51, 54, 58, 61, 63, 65, 67, 73, 75, 78, 79, 82, 83, 85, 106, 108, 89, and 110, 112-115, 132, 133, 114, 138, 141, 144, 147, 151, 153, 156, and 158, 164-168. A CoV S polypeptide may have a deletion, an insertion, or mutation of up to about 1, up to about 2, up to about 3, up to about 4, up to about 5, up to about 10, up to about 15, up to about 20, up to about 25, up to about 30, up to about 35, up to about 40, up to about 45, or up to about 50 amino acids compared to the amino acid sequence of the CoV S polypeptide having an amino acid sequence selected from any one of SEQ ID NOS: 2, 4, 38, 41, 44, 48, 51, 54, 58, 61, 63, 65, 67, 73, 75, 78, 79, 82, 83, 85, 106, 108, 89, and 110, 112-115, 132, 133, 114, 138, 141, 144, 147, 151, 153, 156, and 158, 164-168. A CoV S polypeptide may have may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 2.7 amino acids, between about 25 and 30 amino acids, between about 30 and 35 amino acids, between about 35 and 40 amino acids, between about 40 and 45 amino acids, or between about 45 and 50 amino acids, as compared to the CoV S polypeptide having an amino acid sequence selected from any one of SEQ ID NOS: 2, 4, 38, 41, 44, 48, 51, 54, 58, 61, 63, 65, 67, 73, 75, 78, 79, 82, 83, 85, 106, 108, 89, and 110, 112-115, 132, 133, 114, 138, 141, 144, 147, 151, 153, 156, and 158, 164-168. In embodiments, the CoV S polypeptides described herein comprise about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 substitutions compared to the coronavirus S protein (SEQ ID NO: 87).

In embodiments, the coronavirus S polypeptide is extended at the N-terminus, the C-terminus, or both the N-terminus and the C-terminus. In aspects, the extension is a tag useful for a function, such as purification or detection. In aspects the tag contains an epitope. For example, the tag may be a polyglutamate tag, a FLAG-tag, a HA-tag, a polyHis-tag (having about 5-10 histidines) (SEQ ID NO: 101), a hexahistidine tag (SEQ ID NO: 100), an 8×-His-tag (having eight histidines) (SEQ ID NO: 102), a Myc-tag, a Glutathione-S-transferase-tag, a Green fluorescent protein-tag, Maltose binding protein-tag, a Thioredoxin-tag, or an Fc-tag. In other aspects, the extension may be an N-terminal signal peptide fused to the protein to enhance expression. While such signal peptides are often cleaved during expression in the cell, some nanoparticles may contain the antigen with an intact signal peptide. Thus, when a nanoparticle comprises an antigen, the antigen may contain an extension and thus may be a fusion protein when incorporated into nanoparticles. For the purposes of calculating identity to the sequence, extensions are not included. In embodiments, the tag is a protease cleavage site. Non-limiting examples of protease cleavage sites include the HRV3C protease cleavage site, chymotrypsin, trypsin, elastase, endopeptidase, caspase-1, caspase-2, caspase-3, caspase-4, caspase-5, caspase-6, caspase-7, caspase-8, caspase-9, caspase-10, enterokinase, factor Xa, Granzyme B, TEV protease, and thrombin. In embodiments, the protease cleavage site is an HRV3C protease cleavage site. In embodiments, the protease cleavage site comprises an amino acid sequence of SEQ ID NO: 98.

In embodiments, the CoV S glycoprotein comprises a fusion protein. In embodiments, the CoV S glycoprotein comprises an N-terminal fusion protein. In embodiments, the Cov S glycoprotein comprises a C-terminal fusion protein. In embodiments, the fusion protein encompasses a tag useful for protein expression, purification, or detection. In embodiments, the tag is a polyHis-tag (having about 5-10 histidines), a Myc-tag, a Glutathione-S-transferase-tag, a Green fluorescent protein-tag, Maltose binding protein-tag, a Thioredoxin-tag, a Strep-tag, a Twin-Strep-tag, or an Fc-tag. In embodiments, the tag is an Fc-tag. In embodiments, the Fc-tag is monomeric, dimeric, or trimeric. In embodiments, the tag is a hexahistidine tag, e.g. a polyHis-tag which contains six histidines (SEQ ID NO: 100). In embodiments, the tag is a Twin-Strep-tag with an amino acid sequence of SEQ ID NO: 99.

In embodiments, the CoV S polypeptide is a fusion protein comprising another coronavirus protein. In embodiments, the other coronavirus protein is from the same coronavirus. In embodiments, the other coronavirus protein is from a different coronavirus.

In aspects, the CoV S protein may be truncated. For example, the N-terminus may be truncated by about 10 amino acids, about 30 amino acids, about 50 amino acids, about 75 amino acids, about 100 amino acids, or about 200 amino acids. The C-terminus may be truncated instead of or in addition to the N-terminus. For example, the C-terminus may be truncated by about 10 amino acids, about 30 amino acids, about 50 amino acids, about 75 amino acids, about 100 amino acids, or about 200 amino acids. For purposes of calculating identity to the protein having truncations, identity is measured over the remaining portion of the protein.

### Nanoparticles Containing CoV Spike (S) Polypeptides

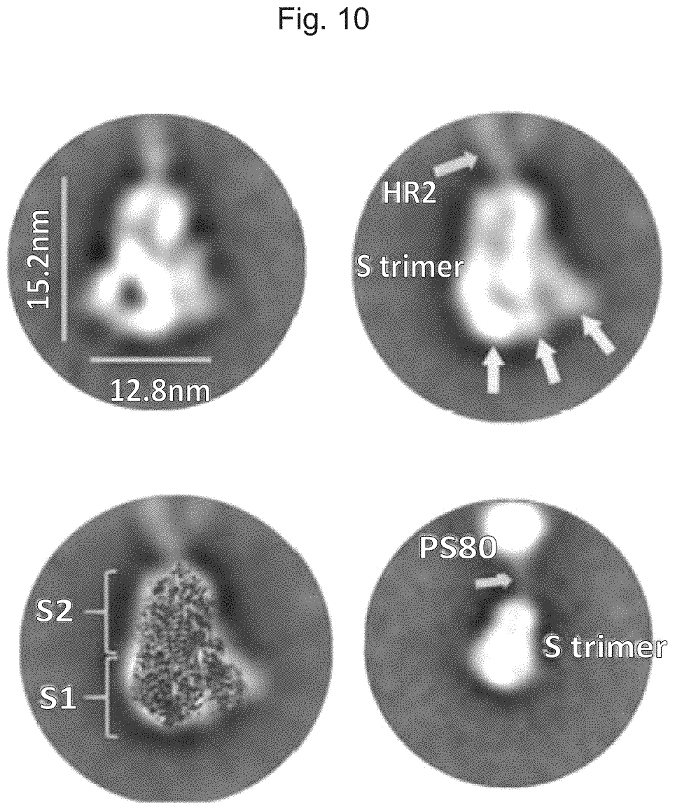

In embodiments, the mature CoV S polypeptide antigens are used to produce a vaccine comprising coronavirus S nanoparticles. In embodiments, nanoparticles of the present disclosure comprise the CoV S polypeptides described herein. in embodiments, the nanoparticles of the present disclosure comprise CoV S polypeptides associated with a detergent core. The presence of the detergent facilitates formation of the nanoparticles by forming a core that organizes and presents the antigens. In embodiments, the nanoparticles may contain the CoV S polypeptides assembled into multi-oligomeric glycoprotein-detergent (e.g. PS80) nanoparticles with the head regions projecting outward and hydrophobic regions and PS80 detergent forming a central core surrounded by the glycoprotein. In embodiments, the CoV S polypeptide inherently contains or is adapted to contain a transmembrane domain to promote association of the protein into a detergent core. In embodiments, the CoV S polypeptide contains a head domain. FIG. 10 shows an exemplary structure of a CoV S polypeptide of the disclosure. Primarily the transmembrane domains of a CoV S polypeptide trimer associate with detergent; however, other portions of the polypeptide may also interact. Advantageously, the nanoparticles have improved resistance to environmental stresses such that they provide enhanced stability and/or improved presentation to the immune system due to organization of multiple copies of the protein around the detergent.

In embodiments, the detergent core is a non-ionic detergent core. In embodiments, the CoV S polypeptide is associated with the non-ionic detergent core. In embodiments, the detergent is selected from the group consisting of polysorbate-20 (PS20), polysorbate-40 (PS40), polysorbate-60 (PS60), polysorbate-65 (PS65) and polysorbate-80 (PS80).

In embodiments, the detergent is PS80.

In embodiments, the CoV S polypeptide forms a trimer. In embodiments, the CoV S polypeptide nanoparticles are composed of multiple polypeptide trimers surrounding a non-ionic detergent core. In embodiments, the nanoparticles contain at least about 1 trimer or more. In embodiments, the nanoparticles contain at least about 5 trimers to about 30 trimers of the Spike protein. In embodiments, each nanoparticle may contain 1, 2, 3, 4, 5, 6, 7, 8, 9, 10. 11, 12, or 15, 20, 25, or 30 trimers, including all values and ranges in between. Compositions disclosed herein may contain nanoparticles having different numbers of trimers. For example, a composition may contain nanoparticles where the number of trimers ranges from 2-9; in embodiments, the nanoparticles in a composition may contain from 2-6 trimers. In embodiments, the compositions contain a heterogeneous population of nanoparticles having 2 to 6 trimers per nanoparticle, or 2 to 9 trimers per nanoparticle. In embodiments, the compositions may contain a substantially homogenous population of nanoparticles. For example, the population may contain about 95% nanoparticles having 5 trimers.

The nanoparticles disclosed herein range in particle size. In embodiments, the nanoparticles disclosed herein range in particle size from a Z-ave size from about 20 nm to about 60 nm, about 20 nm to about 50 nm, about 20 nm to about 45 nm, about 20 nm to about 35 nm, about 20 nm to about 30 nm, about 25 nm to about 35 nm, or about 25 nm to about 45 nm. Particle size (Z-ave) is measured by dynamic light scattering (DLS) using a Zetasizer NanoZS (Malvern, UK), unless otherwise specified.

In embodiments, the nanoparticles comprising the CoV S polypeptides disclosed herein have a reduced particle size compared to nanoparticles comprising a wild-type CoV S polypeptide. In embodiments, the CoV S polypeptides are at least about 40% smaller in particle size, for example, at least about 40%, at least about 45%, at least about 50%, at least about 55%, at least about 60%, at least about 65%, at least about 70%, at least about 75% , at least about 80%, or at least about 85% smaller in particle size.

The nanoparticles comprising CoV S polypeptides disclosed herein are more homogenous in size, shape, and mass than nanoparticles comprising a wild-type CoV S polypeptide. The polydispersity index (PDI), which is a measure of heterogeneity, is measured by dynamic light scattering using a Malvern Setasizer unless otherwise specified. In embodiments, the particles measured herein have a PDI from about 0.2 to about 0.45, for example, about 0.2, about 0.25, about 0.29, about 0.3, about 0.35, about 0.40, or about 0.45. In embodiments, the nanoparticles measured herein have a PDI that is at least about 25% smaller than the PDI of nanoparticles comprising the wild-type CoV S polypeptide, for example, at least about 25%, at least about 30%, at least about 35%, at least about 40%, at least about 45%, at least about 50%, at least about 55%, or at least about 60%, smaller.

The CoV S polypeptides and nanoparticles comprising the same have improved thermal stability as compared to the wild-type CoV S polypeptide or a nanoparticle thereof. The thermal stability of the CoV S polypeptides is measured using differential scanning calorimetry (DSC) unless otherwise specified. The enthalpy of transition (ΔHcal) is the energy required to unfold a CoV S polypeptide. In embodiments, the CoV S polypeptides have an increased ΔHcal as compared to the wild-type CoV S polypeptide. In embodiments, the ΔHcal of a CoV S polypeptide is about 2-fold, about 3-fold, about 4-fold, about 5-fold, about 6-fold, about 7-fold, about 8-fold, about 9-fold, or about 10-fold greater than the ΔHcal of a wild-type CoV S polypeptide.

Several nanoparticle types may be included in vaccine compositions disclosed herein. In aspects, the nanoparticle type is in the form of an anisotropic rod, which may be a dimer or a monomer. In other aspects, the nanoparticle type is a spherical oligomer. In yet other aspects, the nanoparticle may be described as an intermediate nanoparticle, having sedimentation properties intermediate between the first two types. Formation of nanoparticle types may be regulated by controlling detergent and protein concentration during the production process. Nanoparticle type may be determined by measuring sedimentation co-efficient.

### Production of Nanoparticles Containing CoV S Polypeptide Antigens

The nanoparticles of the present disclosure are non-naturally occurring products, the components of which do not occur together in nature. Generally, the methods disclosed herein use a detergent exchange approach wherein a first detergent is used to isolate a protein and then that first detergent is exchanged for a second detergent to form the nanoparticles.

The antigens contained in the nanoparticles are typically produced by recombinant expression in host cells. Standard recombinant techniques may be used. In embodiments, the CoV S polypeptides are expressed in insect host cells using a baculovirus system. In embodiments, the baculovirus is a cathepsin-L knock-out baculovirus, a chitinase knock-out baculovirus. Optionally, the baculovirus is a double knock-out for both cathepsin-L and chitinase. High level expression may be obtained in insect cell expression systems. Non limiting examples of insect cells are, Spodoptera frugiperda (Sf) cells, e.g. Sf9, Sf21, Trichoplusia ni cells, e.g. High Five cells, and Drosophila S2 cells. In embodiments, the CoV S polypeptide described herein are produced in any suitable host cell. In embodiments, the host cell is an insect cell. In embodiments, the insect cell is an Sf9 cell.

Typical transfection and cell growth methods can be used to culture the cells. Vectors, e.g., vectors comprising polynucleotides that encode fusion proteins, can be transfected into host cells according to methods well known in the art. For example, introducing nucleic acids into eukaryotic cells can be achieved by calcium phosphate co-precipitation, electroporation, microinjection, lipofection, and transfection employing polyamine transfection reagents. In one embodiment, the vector is a recombinant baculovirus.

Methods to grow host cells include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In other embodiment, the pre-sterilized plastic bags are about 50 L to 3500 L bags.

### Extraction and Purification of Nanoparticles Containing CoV Spike (S) Protein Antigens

After growth of the host cells, the protein may be harvested from the host cells using detergents and purification protocols. Once the host cells have grown for 48 to 96 hours, the cells are isolated from the media and a detergent-containing solution is added to solubilize the cell membrane, releasing the protein in a detergent extract. Triton X-100 and TERGITOL® nonylphenol ethoxylate, also known as NP-9, are each preferred detergents for extraction. The detergent may be added to a final concentration of about 0.1% to about 1.0%. For example, the concentration may be about 0.1%, about 0.2%, about 0.3%, about 0.5%, about 0.7%, about 0.8%, or about 1.0%. The range may be about 0.1% to about 0.3%. In aspects, the concentration is about 0.5%.

In other aspects, different first detergents may be used to isolate the protein from the host cell. For example, the first detergent may be Bis(polyethylene glycol bis[imidazoylcarbonyl]), nonoxynol-9, Bis(polyethylene glycol bis[imidazoyl carbonyl]), BRIJ® Polyethylene glycol dodecyl ether 35, BRIJ® Polyethylene glycol (3) cetyl ether 56, BRIJ® alcohol ethoxylate 72, BRIJ® Polyoxyl 2 stearyl ether 76, BRIJ® polyethylene glycol monoolelyl ether 92V, BRIJ® Polyoxyethylene (10) oleyl ether 97, BRIJ® Polyethylene glycol hexadecyl ether 58P, CRENTOPHOR® EL Macrogolglycerol ricinoleate, Decaethyleneglycol monododecyl ether, N-Decanoyl-N-methylglucamine, n-Decyl alpha-Dglucopyranoside, Decyl beta-D-maltopyranoside, n-Dodecanoyl-N-methylglucamide, nDodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-0-(N-heptylcarbamoyl)-alpha-D-glucopyranoside, Nonaethylene glycol monododecyl ether, N-Nonanoyl-N-methylglucamine, N-NonanoyN-methylglucamine, Octaethylene glycol monodecyl ether, Octaethylene glycolmonododecyl ether, Octaethylene glycol monohexadecyl ether, Octaethylene glycol monooctadecyl ether, Octaethylene glycol monotetradecyl ether, Octyl-beta-D glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene stearate, Polyoxyethylene 8 stearate, Polyoxyethylene bis(imidazolyl carbonyl), Polyoxyethylene 25 propylene glycol stearate, Saponin from Quillaja bark, SPAN® 20 sorbitan laurate, SPAN® 40 sorbitan monopalmitate, SPAN® 60 sorbitan stearate, SPAN® 65 sorbitan tristearate, SPAN® 80 sorbitane monooleate, SPAN® 85 sorbitane trioleate, TERGITOL® secondary alcohol ethoxylate Type 15-S-12, TERGITOL® secondary alcohol ethoxylate Type 15-S-30, TERGITOL® secondary alcohol ethoxylate Type 15-S-5, TERGITOL® secondary alcohol ethoxylate Type 15-S-7, TERGITOL® secondary alcohol ethoxylate Type 15-S-9, TERGITOL® nonylphenol ethoxylate Type NP-10, TERGITOL® nonylphenol ethoxylate Type NP-4, TERGUOL® nonylphenol ethoxylate Type NP-40, TERGITOL® nonylphenol ethoxylate Type NP-7, TERGITOL® nonylphenol ethoxylate Type NP-9, TERGITOL® branched secondary alcohol ethoxylate Type TMN-10, TERGITOL® branched secondary alcohol ethoxylate Type TMN-6, TRITON® X-100 Polyethylene glycol tert-octylphenyl ether or combinations thereof.

The nanoparticles may then be isolated from cellular debris using centrifugation. In embodiments, gradient centrifugation, such as using cesium chloride, sucrose and iodixanol, may be used. Other techniques may be used as alternatives or in addition, such as standard purification techniques including, e.g., ion exchange, affinity, and gel filtration chromatography.

For example, the first column may be an ion exchange chromatography resin, such as FRACTOGEL® EMD methacrylate based polymeric beads TMAE (EMD Millipore), the second column may be a lentil (Lens culinaris) lectin affinity resin, and the third column may be a cation exchange column such as a FRACTOGEL® EMD methacrylate based polymeric beads SO3 (EMD Millipore) resin. In other aspects, the cation exchange column may be an MMC column or a Nuvia C Prime column (Bio-Rad Laboratories, Inc). Preferably, the methods disclosed herein do not use a detergent extraction column; for example a hydrophobic interaction column. Such a column is often used to remove detergents during purification but may negatively impact the methods disclosed here.

### Detergent Exchange of Nanoparticles Containing CoV S Polypeptide Antigens

To form nanoparticles, the first detergent, used to extract the protein from the host cell is substantially replaced with a second detergent to arrive at the nanoparticle structure. NP-9 is a preferred extraction detergent. Typically, the nanoparticles do not contain detectable NP-9 when measured by HPLC. The second detergent is typically selected from the group consisting of PS20, PS40, PS60, PS65, and PS80. Preferably, the second detergent is PS80.

In particular aspects, detergent exchange is performed using affinity chromatography to bind glycoproteins via their carbohydrate moiety. For example, the affinity chromatography may use a legume lectin column. Legume lectins are proteins originally identified in plants and found to interact specifically and reversibly with carbohydrate residues. See, for example, Sharon and Lis, “Legume lectins—a large family of homologous proteins,” FASEB J. 1990 November; 4(14):3198-208; Liener, “The Lectins: Properties, Functions, and Applications in Biology and Medicine,” Elsevier, 2012. Suitable lectins include concanavalin A (con A), pea lectin, sainfoin lest, and lentil lectin. Lentil lectin is a preferred column for detergent exchange due to its binding properties. Lectin columns are commercially available; for example, Capto Lentil Lectin, is available from GE Healthcare. In certain aspects, the lentil lectin column may use a recombinant lectin. At the molecular level, it is thought that the carbohydrate moieties bind to the lentil lectin, freeing the amino acids of the protein to coalesce around the detergent resulting in the formation of a detergent core providing nanoparticles having multiple copies of the antigen, e.g., glycoprotein oligomers which can be dimers, trimers, or tetramers anchored in the detergent. In embodiments, the CoV S polypeptides form trimers. In embodiments, the CoV S polypeptide trimers are anchored in detergent. In embodiments, each CoV S polypeptide nanoparticle contains at least one trimer associated with a non-ionic core.

The detergent, when incubated with the protein to form the nanoparticles during detergent exchange, may be present at up to about 0.1% (w/v) during early purifications steps and this amount is lowered to achieve the final nanoparticles having optimum stability. For example, the non-ionic detergent (e.g., PS80) may be about 0.005% (v/v) to about 0.1% (v/v), for example, about 0.005% (v/v), about 0.006% (v/v), about 0.007% (v/v), about 0.008% (v/v), about 0.009% (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095 (v/v), or about 0.1% (v/v) PS80. In embodiments, the nanoparticle contains about 0.03% to about 0.05% PS80. In embodiments, the nanoparticle contains about 0.01% (v/v) PS80.

In embodiments, purified CoV S polypeptides are dialyzed. In embodiments, dialysis occurs after purification. In embodiments, the CoV S polypeptides are dialyzed in a solution comprising sodium phosphate, NaCl, and PS80. In embodiments, the dialysis solution comprising sodium phosphate contains between about 5 mM and about 100 mM of sodium phosphate, for example, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 about 90 mM, about 95 mM, or about 100 mM sodium phosphate. In embodiments, the pH of the solution comprising sodium phosphate is about 6.5, about 6.6, about 6.7, about 6.8, about 6.9, about 7.0, about 7.1, about 7.2, about 7.3, about 7.4, or about 7.5. In embodiments, the dialysis solution comprising sodium chloride comprises about 50 mM NaCl to about 500 mM NaCl, for example, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM NaCl. In embodiments, the dialysis solution comprising PS80 comprises about 0.005% (v/v), about 0.006% (v/v), about 0.007 (v/v), about 0.008% (v/v), about 0.009 (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055 (v/v), about 0.06 (v/v), about 0.065 (v/v), about 0.07% (v/v), about 0.075% (v/v), about 0.08% (v/v), about 0.085% (v/v), about 0.09% (v/v), about 0.095% (v/v), or about 0.1% (v/v) PS80. In embodiments, the dialysis solution comprises about 25 mM sodium phosphate (pH 7.2), about 300 mM NaCl, and about 0.01% (v/v) PS80.

Detergent exchange may be performed with proteins purified as discussed above and purified, frozen for storage, and then thawed for detergent exchange.

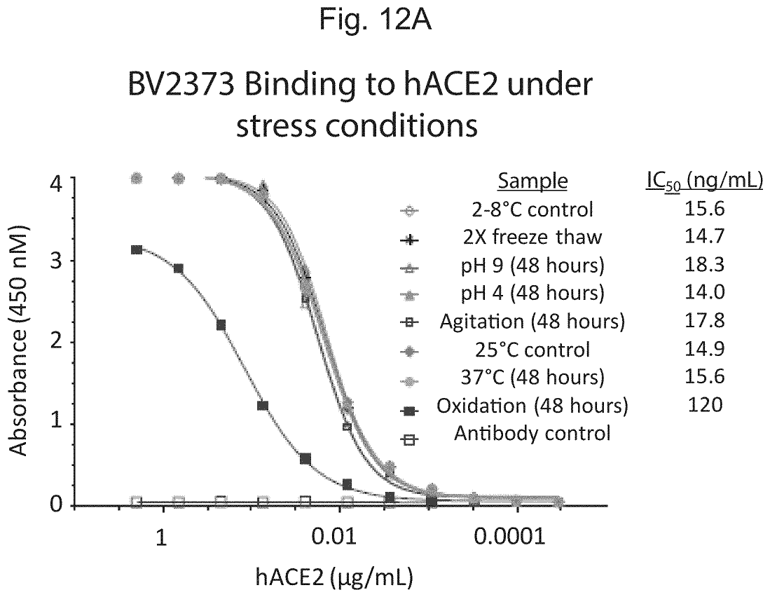

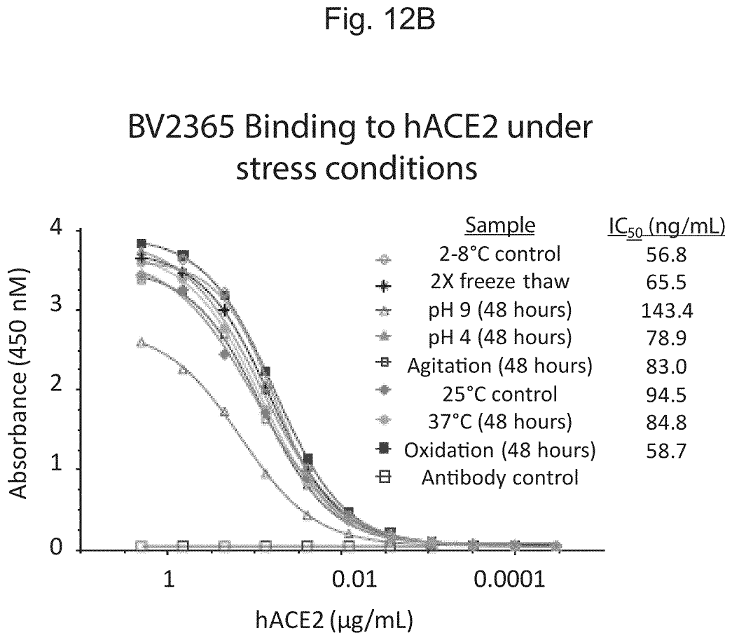

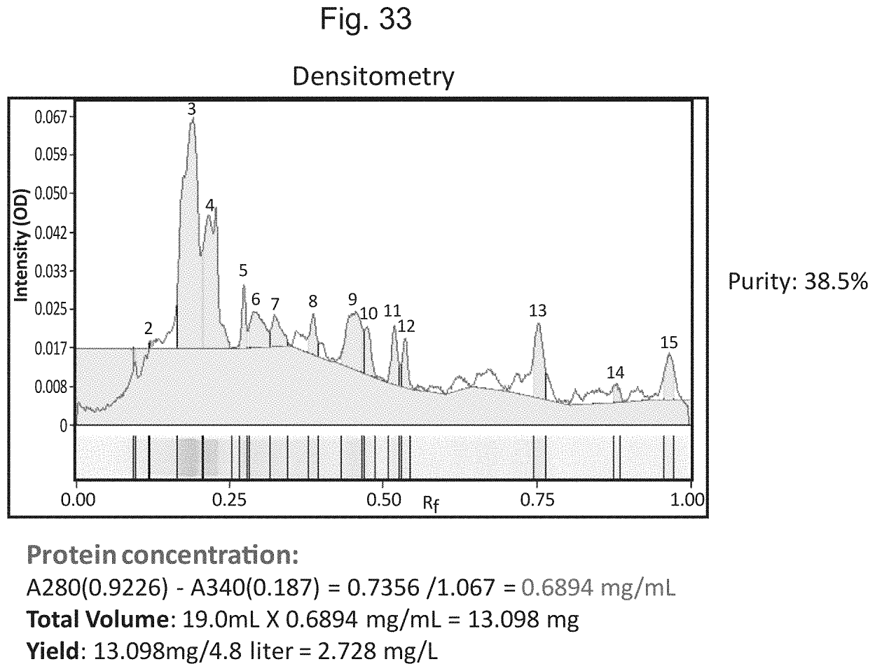

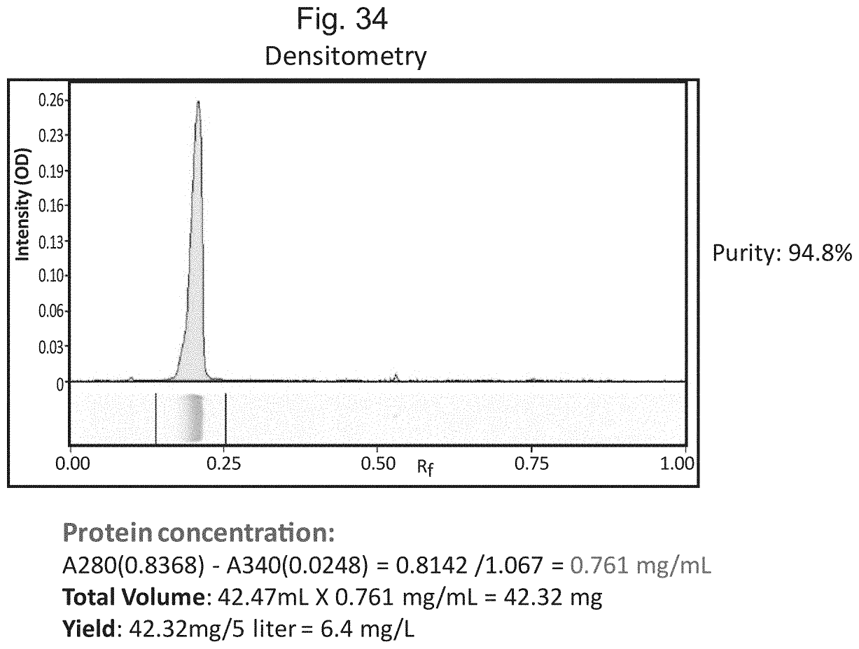

Stability of compositions disclosed herein may be measured in a variety of ways. In one approach, a peptide map may be prepared to determine the integrity of the antigen protein after various treatments designed to stress the nanoparticles by mimicking harsh storage conditions. Thus, a measure of stability is the relative abundance of antigen peptides in a stressed sample compared to a control sample. For example, the stability of nanoparticles containing the CoV S polypeptides may be evaluated by exposing the nanoparticles to various pHs, proteases, salt, oxidizing agents, including but not limited to hydrogen peroxide, various temperatures, freeze/thaw cycles, and agitation. FIGS. 12A-B show that BV2373 (SEQ ID NO: 87) and BV2365 (SEQ ID NO: 4) retain binding to hACE2 under a variety of stress conditions. It is thought that the position of the glycoprotein anchored into the detergent core provides enhanced stability by reducing undesirable interactions. For example, the improved protection against protease-based degradation may be achieved through a shielding effect whereby anchoring the glycoproteins into the core at the molar ratios disclosed herein results in steric hindrance blocking protease access. Stability may also be measured by monitoring intact proteins. FIG. 33 and FIG. 34 compare nanoparticles containing CoV polypeptides having amino acid sequences of SEQ ID NO: 109 and 87, respectively. FIG. 34 indicates that CoV polypeptides having an amino acid sequence of SEQ ID NO: 87 show particularly good stability during purification. The polypeptide of FIG. 34 comprises a furin cleavage site having an amino acid sequence of QQAQ (SEQ ID NO: 7).

### Vaccine Compositions Containing CoV S Polypeptide Antigens

The disclosure provides vaccine compositions comprising CoV S polypeptides, for example, in a nanoparticle. In aspects, the vaccine composition may contain nanoparticles with antigens from more than one viral strain from the same species of virus. In another embodiment, the disclosures provide for a pharmaceutical pack or kit comprising one or more containers filled with one or more of the components of the vaccine compositions.

Compositions disclosed herein may be used either prophylactically or therapeutically, but will typically be prophylactic. Accordingly, the disclosure includes methods for treating or preventing infection. The methods involve administering to the subject a therapeutic or prophylactic amount of the immunogenic compositions of the disclosure. Preferably, the pharmaceutical composition is a vaccine composition that provides a protective effect. In other aspects, the protective effect may include amelioration of a symptom associated with infection in a percentage of the exposed population. For example, the composition may prevent or reduce one or more virus disease symptoms selected from: fever fatigue, muscle pain, headache, sore throat, vomiting, diarrhea, rash, symptoms of impaired kidney and liver function, internal bleeding and external bleeding, compared to an untreated subject.

The nanoparticles may be formulated for administration as vaccines in the presence of various excipients, buffers, and the like. For example, the vaccine compositions may contain sodium phosphate, sodium chloride, and/or histidine. Sodium phosphate may be present at about 10 mM to about 50 mM, about 15 mM to about 25 mM, or about 25 mM; in particular cases, about 22 mM sodium phosphate is present. Histidine may be present about 0.1% (w/v), about 0.5% (w/v), about 0.7% (w/v), about 1% (w/v), about 1.5% (w/v), about 2% (w/v), or about 2.5% (w/v). Sodium chloride, when present, may be about 150 mM. In certain compositions, the sodium chloride may be present in higher concentrations, for example from about 200 mM to about 500 mM. In embodiments, the sodium chloride is present in a high concentration, including but not limited to about 200 mM, about 250 mM, about 300 mM, about 350 mM, about 400 mM, about 450 mM, or about 500 mM.

In embodiments, the nanoparticles described herein have improved stability at certain pH levels. In embodiments, the nanoparticles are stable at slightly acidic pH levels. For example, the nanoparticles that are stable at a slightly acidic pH, for example from pH 5.8 to pH 7.0. In embodiments, the nanoparticles and compositions containing nanoparticles may be stable at pHs ranging from about pH 5.8 to about pH 7.0, including about pH 5.9 to about pH 6.8, about pH 6.0 to about pH 6.5, about pH 6.1 to about pH 6.4, about pH 6.1 to about pH 6.3, or about pH 6.2. In embodiments, the nanoparticles and compositions described herein are stabile at neutral pHs, including from about pH 7.0 to about pH 7.4. In embodiments, the nanoparticles and compositions described herein are stable at slightly alkaline pHs, for example from about pH 7.0 to about pH 8.5, from about pH 7.0 to about pH 8.0, or from about pH 7.0 to about pH 7.5, including all values and ranges in between.

### Adjuvants

In certain embodiments, the compositions disclosed herein may be combined with one or more adjuvants to enhance an immune response. In other embodiments, the compositions are prepared without adjuvants, and are thus available to be administered as adjuvant-free compositions. Advantageously, adjuvant-free compositions disclosed herein may provide protective immune responses when administered as a single dose. Alum-free compositions that induce robust immune responses are especially useful in adults about 60 and older.

### Aluminum-Based Adjuvants

In embodiments, the adjuvant may be alum (e.g. AlPO₄ or Al(OH)₃). Typically, the nanoparticle is substantially bound to the alum. For example, the nanoparticle may be at least 80% bound, at least 85% bound, at least 90% bound or at least 95% bound to the alum. Often, the nanoparticle is 92% to 97% bound to the alum in a composition. The amount of alum is present per dose is typically in a range between about 400 μg to about 1250 μg. For example, the alum may be present in a per dose amount of about 300 μg to about 900 μg, about 400 μg to about 800 μg, about 500 μg to about 700 μg, about 400 μg to about 600 μg, or about 400 μg to about 500 μg. Typically, the alum is present at about 400 μg for a dose of 120 μg of the protein nanoparticle.

### Saponin Adjuvants

Adjuvants containing saponin may also be combined with the immunogens disclosed herein. Saponins are glycosides derived from the bark of the Quillaja saponaria Molina tree. Typically, saponin is prepared using a multi-step purification process resulting in multiple fractions. As used, herein, the term “a saponin fraction from Quillaja saponaria Molina” is used genetically to describe a semi-purified or defined saponin fraction of Quillaja saponaria or a substantially pure fraction thereof.

### Saponin Fractions

Several approaches for producing saponin fractions are suitable. Fractions A, B, and C are described in U.S. Pat. No. 6,352,697 and may be prepared as follows. A lipophilic fraction from Quil A, a crude aqueous Quillaja saponaria Molina extract, is separated by chromatography and eluted with 70% acetonitrile in water to recover the lipophilic fraction. This lipophilic fraction is then separated by semi-preparative HPLC with elution using a gradient of from 25% to 60% acetonitrile in acidic water. The fraction referred to herein as “Fraction A” or “QH-A” is, or corresponds to, the fraction, which is eluted at approximately 39% acetonitrile. The fraction referred to herein as “Fraction B” or “QH-B” is, or corresponds to, the fraction, which is eluted at approximately 47% acetonitrile. The fraction referred to herein as “Fraction C” or “QH-C” is, or corresponds to, the fraction, which is eluted at approximately 49% acetonitrile. Additional information regarding purification of Fractions is found in U.S. Pat. No. 5,057,540. When prepared as described herein, Fractions B and C of Quillaja saponaria Molina each represent groups or families of chemically closely related molecules with definable properties. The chromatographic conditions under which they are obtained are such that the batch-to-batch reproducibility in terms of elution profile and biological activity is highly consistent.

Other saponin fractions have been described. Fractions B3, B4 and B4b are described in EP 0436620. Fractions QA1-QA22 are described EP03632279 B2, Q-VAC (Nor-Feed, AS Denmark), Quillaja saponaria Molina Spikoside (Isconova AB, Ultunaallén 2B, 756 51 Uppsala, Sweden). Fractions QA-1, QA-2, QA-3, QA-4, QA-5, QA-6, QA-7, QA-8, QA-9, QA-10, QA-11, QA-12, QA-13, QA-14, QA-15, QA-16, QA-17, QA-18, QA-19, QA-20, QA-21, and QA-22 of EP 0 3632 279 B2, especially QA-7, QA-17 QA-18, and QA-21 may be used. They are obtained as described in EP 0 3632 279 B2, especially at page 6 and in Example 1 on page 8 and 9.

The saponin fractions described herein and used for forming adjuvants are often substantially pure fractions; that is, the fractions are substantially free of the presence of contamination from other materials. In particular aspects, a substantially pure saponin fraction may contain up to 40% by weight, up to 30% by weight, up to 25% by weight, up to 20% by weight, up to 15% by weight, up to 10% by weight, up to 7% by weight, up to 5% by weight, up to 2% by weight, up to 1% by weight, up to 0.5% by weight, or up to 0.1% by weight of other compounds such as other saponins or other adjuvant materials.

### ISCOM Structures

Saponin fractions may be administered in the form of a cage-like particle referred to as an ISCOM (Immune Stimulating COMplex). ISCOMs may be prepared as described in EP0109942B1, EP0242380B1 and EP0180546 B1. In particular embodiments a transport and/or a passenger antigen may be used, as described in EP 9600647-3 (PCT/SE97/00289).

### Matrix Adjuvants

In embodiments, the ISCOM is an ISCOM matrix complex. An ISCOM matrix complex comprises at least one saponin fraction and a lipid. The lipid is at least a sterol, such as cholesterol. In particular aspects, the ISCOM matrix complex also contains a phospholipid. The ISCOM matrix complexes may also contain one or more other immunomodulatory (adjuvant-active) substances, not necessarily a glycoside, and may be produced as described in EP0436620B1, which is incorporated by reference in its entirety herein.

In other aspects, the ISCOM is an ISCOM complex. An ISCOM complex contains at least one saponin, at least one lipid, and at least one kind of antigen or epitope. The ISCOM complex contains antigen associated by detergent treatment such that that a portion of the antigen integrates into the particle. In contrast, ISCOM matrix is formulated as an admixture with antigen and the association between ISCOM matrix particles and antigen is mediated by electrostatic and/or hydrophobic interactions.

According to one embodiment, the saponin fraction integrated into an ISCOM matrix complex or an ISCOM complex, or at least one additional adjuvant, which also is integrated into the ISCOM or ISCOM matrix complex or mixed therewith, is selected from fraction A, fraction B, or fraction C of Quillaja saponaria, a semipurified preparation of Quillaja saponaria, a purified preparation of Quillaja saponaria, or any purified sub-fraction e.g., QA 1-21.

In particular aspects, each ISCOM particle may contain at least two saponin fractions. Any combinations of weight % of different saponin fractions may be used. Any combination of weight % of any two fractions may be used. For example, the particle may contain any weight % of fraction A and any weight % of another saponin fraction, such as a crude saponin fraction or fraction C, respectively. Accordingly, in particular aspects, each ISCOM matrix particle or each ISCOM complex particle may contain from 0.1 to 99.9 by weight, 5 to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60% by weight, or 50% by weight of one saponin fraction, e.g. fraction A and the rest up to 100% in each case of another saponin e.g. any crude fraction or any other faction e.g. fraction C. The weight is calculated as the total weight of the saponin fractions. Examples of ISCOM matrix complex and ISCOM complex adjuvants are disclosed in U.S Published Application No. 2013/0129770, which is incorporated by reference in its entirety herein.

In particular embodiments, the ISCOM matrix or ISCOM complex comprises from 5-99% by weight of one fraction, e.g. fraction A and the rest up to 100% of weight of another fraction e.g. a crude saponin fraction or fraction C. The weight is calculated as the total weight of the saponin fractions.

In another embodiment, the ISCOM matrix or ISCOM complex comprises from 40% to 99% by weight of one fraction, e.g. fraction A and from 1% to 60% by weight of another fraction, e.g. a crude saponin fraction or fraction C. The weight is calculated as the total weight of the saponin fractions.

In yet another embodiment, the ISCOM matrix or ISCOM complex comprises from 70% to 95% by weight of one fraction e.g., fraction A, and from 30% to 5% by weight of another fraction, e.g., a crude saponin fraction, or fraction C. The weight is calculated as the total weight of the saponin fractions. In other embodiments, the saponin fraction from Quillaja saponaria Molina is selected from any one of QA 1-21.

In addition to particles containing mixtures of saponin fractions, ISCOM matrix particles and ISCOM complex particles may each be formed using only one saponin fraction. Compositions disclosed herein may contain multiple particles wherein each particle contains only one saponin fraction. That is, certain compositions may contain one or more different types of ISCOM-matrix complexes particles and/or one or more different types of ISCOM complexes particles, where each individual particle contains one saponin fraction from Quillaja saponaria Molina, wherein the saponin fraction in one complex is different from the saponin fraction in the other complex particles.

In particular aspects, one type of saponin fraction or a crude saponin fraction may be integrated into one ISCOM matrix complex or particle and another type of substantially pure saponin fraction, or a crude saponin fraction, may be integrated into another ISCOM matrix complex or particle. A composition or vaccine may comprise at least two types of complexes or particles each type having one type of saponins integrated into physically different particles.

In the compositions, mixtures of ISCOM matrix complex particles and/or ISCOM complex particles may be used in which one saponin fraction Quillaja saponaria Molina and another saponin fraction Quillaja saponaria Molina are separately incorporated into different ISCOM matrix complex particles and/or ISCOM complex particles.

The ISCOM matrix or ISCOM complex particles, which each have one saponin fraction, may be present in composition at any combination of weight %. In particular aspects, a composition may contain 0.1% to 99.9% by weight, 5% to 95% by weight, 10% to 90% by weight, 15% to 85% by weight, 20% to 80% by weight, 25% to 75% by weight, 30% to 70% by weight, 35% to 65% by weight, 40% to 60% by weight, 45% to 55% by weight, 40 to 60% by weight, or 50% by weight, of an ISCOM matrix or complex containing a first saponin fraction with the remaining portion made up by an ISCOM matrix or complex containing a different saponin fraction. In aspects, the remaining portion is one or more ISCOM matrix or complexes where each matrix or complex particle contains only one saponin fraction. In other aspects, the ISCOM matrix or complex particles may contain more than one saponin fraction.

In particular compositions, the only saponin fraction in a first ISCOM matrix or ISCOM complex particle is Fraction A and the only saponin fraction in a second ISCOM matrix or ISCOM complex particle is Fraction C.

Preferred compositions comprise a first ISCOM matrix containing Fraction A and a second ISCOM matrix containing Fraction C, wherein the Fraction A ISCOM matrix constitutes about 70% per weight of the total saponin adjuvant, and the Fraction C ISCOM matrix constitutes about 30% per weight of the total saponin adjuvant. In another preferred composition, the Fraction A ISCOM matrix constitutes about 85% per weight of the total saponin adjuvant, and the Fraction C ISCOM matrix constitutes about 15% per weight of the total saponin adjuvant. Thus, in certain compositions, the Fraction A ISCOM matrix is present in a range of about 70% to about 85%, and Fraction C ISCOM matrix is present in a range of about 15% to about 30%, of the total weight amount of saponin adjuvant in the composition. In embodiments, the Fraction A ISCOM matrix accounts for 50-96% by weight and Fraction C ISCOM matrix accounts for the remainder, respectively, of the sums of the weights of Fraction A ISCOM matrix and Fraction C ISCOM in the adjuvant. In a particularly preferred composition, referred to herein as MATRIX-M™, the Fraction A ISCOM matrix is present at about 85% and Fraction C ISCOM matrix is present at about 15% of the total weight amount of saponin adjuvant in the composition. MATRIX-M™ may be referred to interchangeably as Matrix-M1.

Exemplary QS-7 and QS-21 fractions, their production and their use is described in U.S. Pat. Nos. 5,057,540; 6,231,859; 6,352,697; 6,524,584; 6,846,489; 7,776,343, and 8,173,141, which are incorporated by reference herein.

In embodiments, other adjuvants may be used in addition or as an alternative. The inclusion of any adjuvant described in Vogel et al., “A Compendium of Vaccine Adjuvants and Excipients (2nd Edition),” herein incorporated by reference in its entirety for all purposes, is envisioned within the scope of this disclosure. Other adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/TWEEN® polysorbate 80 emulsion. In embodiments, the adjuvant may be a paucilamellar lipid vesicle; for example, NOVASOMES®. NOVASOMES® are paucilamellar nonphospholipid vesicles ranging from about 100 nm to about 500 nm. They comprise BRIJ® alcohol ethoxylate 72, cholesterol, oleic acid and squalene. NOVASOMES® have been shown to be an effective adjuvant (see, U.S. Pat, Nos. 5,629,021, 6,387,373, and 4,911,928.

### Administration and Dosage

In embodiments, the disclosure provides a method for eliciting an immune response against one or more coronaviruses. In embodiments, the response is against one or more of the SARS-CoV-2 virus, MERS, and SARS. In embodiments, the response is against a heterogeneous SARS-CoV-2 strain. Non-limiting examples of heterogeneous SARS-CoV-2 strains include the Cal.20C SARS-CoV-2 strain, P.1 SARS-CoV-2 strain, B.1.351 SARS-CoV-2 strain, and B.1.1.7 SARS-CoV-2 strain. The method involves administering an immunologically effective amount of a composition containing a nanoparticle or containing a recombinant CoV Spike (S) polypeptide to a subject. Advantageously, the proteins disclosed herein induce one or more of particularly useful anti-coronavirus responses.

In embodiments, the nanoparticles or CoV S polypeptides are administered with an adjuvant. In aspects, the nanoparticles or CoV S polypeptides are administered without an adjuvant. In aspects, the adjuvant may be bound to the nanoparticle, such as by a non-covalent interaction. In other aspects, the adjuvant is co-administered with the nanoparticle but the adjuvant and nanoparticle do not interact substantially.

In embodiments, the nanoparticles or CoV S polypeptides may be used for the prevention and/or treatment of one or more of a SARS-CoV-2 infection, a heterogeneous SARS-CoV-2 strain infection, a SARS infection, or a MERS infection. Thus, the disclosure provides a method for eliciting an immune response against one or more of the SARS-CoV-2 virus, heterogeneous SARS-CoV-2 virus, MERS, and SARS. The method involves administering an immunologically effective amount of a composition containing a nanoparticle or a CoV S polypeptide to a subject. Advantageously, the proteins disclosed herein induce particularly useful anti-coronavirus responses.

In embodiments, compositions containing the nanoparticles or CoV S polypeptides described herein induce a protective response against SARS-CoV-2 or a heterogeneous SARS-CoV-2 strain in a subject for up to about 3 months, up to about 4 months, up to about 5 months, up to about 6 months, up to about 7 months, up to about 8 months, up to about 9 months, up to about 10 months, up to about 11 months, up to about 12 months, up to about 13 months, up to about 14 months, up to about 15 months, up to about 16 months, up to about 17 months, up to about 18 months, up to about 19 months, up to about 20 months, up to about 21 months, up to about 22 months, up to about 23 months, up to about 24 months, up to about 2.5 years, up to about 3 years, up to about 3.5 years, up to about 4 years, up to about 4.5 years, up to about 5 years after a last dose of nanoparticle or CoV S polypeptide. In embodiments, the nanoparticles or CoV S polypeptides described herein induce a protective response in a subject for at least 6 months.

In embodiments, the protective response is against an asymptomatic infection caused. by SARS-CoV-2 or a heterogeneous SARS-CoV-2 strain. In embodiments, the protective response is against a symptomatic infection caused by SARS-CoV-2 or a heterogeneous SARS-CoV-2 strain.

In embodiments, compositions containing the nanoparticles or CoV S polypeptides described herein have an efficacy at preventing coronavirus disease-19 (COVID-19) from a SARS-CoV-2 virus or a heterogeneous SARS-CoV-2 strain (e.g., a B.1.1.7 SARS-CoV-2 strain, B.1.351 SARS-CoV-2 strain, P.1 SARS-CoV-2 strain, B.1.617.2 SARS-CoV-2 strain, B.1.525 SARS-CoV-2 strain, B.1.526 SARS-CoV-2 strain, B.1.617.1 SARS-CoV-2 strain, a C.37 SARS-CoV-2 strain, B.1.621 SARS-CoV-2 strain, or a Cal.20C BARS-CoV-2 strain) that is from about 50% to about 99%, from about 80% to about 99%, from about 75% to about 99%, from about 80% to about 95%, from about 90% to about 98%, from about 75% to about 95%, from about 80% to about 90%, from about 85% to about 95%, from about 80% to about 95%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% for up to about 1 month, up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, up to about 12 months, up to about 12.5 months, up to about 13 months, up to about 13.5 months, up to about 14 months, up to about 14.5 months, up to about 15 months, up to about 15.5 months, up to about 16 months, up to about 16.5 months, up to about 17 months, up to about 17.5 months, up to about 18 months, up to about 18.5 months, up to about 19 months, up to about 19.5 months, up to about 20 months, up to about 20.5 months, up to about 21 months, up to about 21.5 months, up to about 22 months, up to about 22.5 months, up to about 23 months, up to about 23.5 months, up to about 24 months, up to about 2.1 years, up to about 2.2 years, up to about 2.3 years, up to about 2.4 years, up to about 2.5 years, up to about 2.6 years, up to about 2.7 years, up to about 2.8 years, up to about 2.9 years, up to about 3 years, or longer after administration of the last dose of nanoparticles or CoV S polypeptides described herein. In embodiments, the COVID-19 is mild COVID-19. In embodiments, the COVID-19 is moderate COVID-19. In embodiments, the COVID-19 is severe COVID-19. In embodiments, the COVID-19 is asymptomatic COVID-19.

In embodiments, compositions containing the nanoparticles or CoV S polypeptides described herein have an efficacy against a SARS-CoV-2 virus or a heterogeneous SARS-CoV-2 strain of at least 82% for up to about 7.5 months after administration of the last dose of nanoparticles or CoV S polypeptides described herein. In embodiments, compositions containing the nanoparticles or CoV S polypeptides described herein have an efficacy against a SARS-CoV-2 virus or a heterogeneous SARS-CoV-2 strain of 80% to about 90% for up to about 7.5 months after administration of the last dose of nanoparticles or CoV S polypeptides described herein.

In embodiments, compositions containing the nanoparticles or CoV S polypeptides have an efficacy of at least 75% against asymptomatic disease. In embodiments, the nanoparticles or CoV S polypeptides have an efficacy of from 80% to 90%, from 80% to 99%, from 82% to 99%, from 82% to 95%, from 85% to 95%, from 85% to 99%, from 85% to 97%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% against symptomatic COVID-19 for up to about 1 month, up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, up to about 12 months, up to about 12.5 months, up to about 13 months, up to about 13.5 months, up to about 14 months, up to about 14.5 months, up to about 15 months, up to about 15.5 months, up to about 16 months, up to about 16.5 months, up to about 17 months, up to about 17.5 months, up to about 18 months, up to about 18.5 months, up to about 19 months, up to about 19.5 months, up to about 20 months, up to about 20.5 months, up to about 21 months, up to about 21.5 months, up to about 22 months, up to about 22.5 months, up to about 23 months, up to about 23.5 months, or up to about 24 months, or more.

In embodiments, compositions containing the nanoparticles or CoV S polypeptides have an efficacy of from 95% to 97%, from 95% to 99%, from 95% to 98%, at least 95%, at least 96%, at least 97%, at least 98%, at least 99%, or 100% against severe COVID-19 for up to about 1 month, up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, up to about 12 months, up to about 12.5 months, up to about 13 months, up to about 13.5 months, up to about 14 months, up to about 14.5 months, up to about 15 months, up to about 15.5 months, up to about 16 months, up to about 16.5 months, up to about 17 months, up to about 17.5 months, up to about 18 months, up to about 18.5 months, up to about 19 months, up to about 19.5 months, up to about 2.0 months, up to about 20.5 months, up to about 21 months, up to about 21.5 months, up to about 22 months, up to about 22.5 months, up to about 23 months, up to about 23.5 months, or up to about 24 months, or more.

In embodiments, compositions containing the nanoparticles or CoV S polypeptides have an efficacy of from 75% to 95%, from 75% to 90%, from 75% to 85%, from 75% to 98%, from 80% to 98%, from 80% to 95%, from 80% to 90%, from 85% to 98%, from 85% to 95%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% against moderate COVID-19 for up to about 1 month, up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8,5 months, up to about 9 months, up to about 9.5 months, up to about 10 months, up to about 10.5 months, up to about 11 months, up to about 11.5 months, up to about 12 months, up to about 12.5 months, up to about 13 months, up to about 13.5 months, up to about 14 months, up to about 14.5 months, up to about 15 months, up to about 15.5 months, up to about 16 months, up to about 16.5 months, up to about 17 months, up to about 17.5 months, up to about 18 months, up to about 18.5 months, up to about 19 months, up to about 19.5 months, up to about 20 months, up to about 20.5 months, up to about 21 months, up to about 21.5 months, up to about 22 months, up to about 22.5 months, up to about 23 months, up to about 23.5 months, or up to about 24 months, or more.

In embodiments, compositions containing the nanoparticles or CoV S polypeptides have an efficacy of from 40% to 95%, from 40% to 90%, from 40% to 85%, from 40% to 80%, from 40% to 75%, from 40% to 70%, from 40% to 65%, from 40% to 60%, from 50% to 95%, from 50% to 90%, from 50% to 85%, from 50% to 80%, from 50% to 75%, from 50% to 70%, from 50% to 65%, from 50% to 60%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57% at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% against mild COVID-19 for up to about 1 month, up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, up to about 12 months, up to about 12.5 months, up to about 13 months, up to about 13.5 months, up to about 14 months, up to about 14.5 months, up to about 15 months, up to about 15.5 months, up to about 16 months, up to about 16.5 months, up to about 17 months, up to about 17.5 months, up to about 18 months, up to about 18.5 months, up to about 19 months, up to about 19.5 months, up to about 20 months, up to about 20.5 months, up to about 21 months, up to about 21.5 months, up to about 22 months, up to about 22.5 months, up to about 23 months, up to about 23.5 months, or up to about 24 months, or more.

Compositions disclosed herein may be administered via a systemic route or a mucosal route or a transdermal route or directly into a specific tissue. As used herein, the term “systemic administration” includes parenteral routes of administration. In particular, parenteral administration includes subcutaneous, intraperitoneal, intravenous, intraarterial, intramuscular, or intrasternal injection, intravenous, or kidney dialytic infusion techniques. Typically, the systemic, parenteral administration is intramuscular injection. As used herein, the term “mucosal administration” includes oral, intranasal, intravaginal, intra-rectal, intra-tracheal, intestinal and ophthalmic administration. Preferably, administration is intramuscular.

Compositions may be administered on a single dose schedule or a multiple dose schedule. Multiple doses may be used in a primary immunization schedule or in a booster immunization schedule. In embodiments, about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 doses are administered. In a multiple dose schedule the various doses may be given by the same or different routes e.g., a parenteral prime and mucosal boost, a mucosal prime and parenteral boost, etc. In aspects, a boost dose is administered about 2 weeks, about 3 weeks, about 4 weeks, about 5 weeks, about 6 weeks, about 2 months, about 3 months, about 4 months, about 5 months, about 6 months, about 7 months, about 8 months, about 9 months, about 10 months, about 11 months, about 12 months (1 year), about 2 years, about 3 years, about 4 years, about 5 years, about 6 years, about 7 years, about 8 y ears, about 9 years, or about 10 years after the first dose. In embodiments, a boost dose is administered every year after administration of the initial dose. In embodiments, the follow-on boost dose is administered 3 weeks or 4 weeks after administration of the prior dose. In embodiments, the first dose is administered at day 0, and the boost dose is administered at day 21. In embodiments, the first dose is administered at day 0, and the boost dose is administered at day 28. In embodiments, the first dose is administered at day 0, a boost dose is administered at day 21, and a second boost dose is administered about six months after administration of the first dose. In embodiments, the first dose is administered at day 0, and the boost dose is administered at day 28, and a second boost dose is administered about six months after administration of the first dose. In embodiments, the first dose is administered at day 0, a boost dose is administered at day 21, and a second boost dose is administered about six months after administration of the second dose. In embodiments, the first dose is administered at day 0, and the boost dose is administered at day 28, and a second boost dose is administered about six months after administration of the second dose.

In embodiments, the boost dose comprises the same immunological composition as the initial dose. In embodiments, the boost dose comprises a different immunological composition than the initial dose. In embodiments, the different immunological composition is a SARS-CoV-2 Spike glycoprotein, an mRNA encoding a SARS-Cov-2 Spike glycoprotein, a plasmid DNA encoding a SARS-Cov-2 Spike glycoprotein, an viral vector encoding a SARS-Cov-2 Spike glycoprotein, or an inactivated SARS-CoV-2 virus. In embodiments, the boost dose comprises the initial composition. In embodiments, the initial dose comprises a SARS-CoV-2 S glycoprotein (e.g., a SARS CoV-2 S glycoprotein having the amino acid sequence of SEQ ID NO: 87), and the boost dose comprises the same SARS-CoV-2 S glycoprotein (e.g., a SARS CoV-2 S glycoprotein having the amino acid sequence of SEQ ID NO: 87). In embodiments, the initial dose comprises a SARS-CoV-2 S glycoprotein (e.g., a SARS CoV-2 S glycoprotein having the amino acid sequence of SEQ ID NO: 87), and the boost dose comprises a different SARS-CoV-2 S glycoprotein (e.g., a SARS CoV-2 S glycoprotein having the amino acid sequence of SEQ ID NO: 132). In embodiments, the initial dose comprises a combination of SARS-CoV-2 S glycoproteins (e.g., a SARS CoV-2 S glycoprotein having the amino acid sequence of SEQ ID NO: 87 and a SARS CoV-2 S glycoprotein having the amino acid sequence of SEQ ID NO: 132). In embodiments, the boost dose comprises a combination of SARS-CoV-2 S glycoproteins (e.g., a SARS CoV-2 S glycoprotein having the amino acid sequence of SEQ ID NO: 87 and a SARS CoV-2 S glycoprotein having the amino acid sequence of SEQ ID NO: 132). In embodiments, the initial dose comprises a SARS-CoV-2 S glycoprotein, a plasmid DNA encoding a SARS-Cov-2 S glycoprotein, an viral vector encoding a SARS-CoV-2 Spike glycoprotein, or an inactivated SARS-CoV-2 virus. In embodiments, the initial dose comprises a SARS-CoV-2 Spike glycoprotein, a plasmid DNA encoding a SARS-CoV-2 Spike glycoprotein, an viral vector encoding a SARS-Cov-2 Spike glycoprotein, or an inactivated SARS-CoV-2 virus, and the boost dose comprises one or more SARS-CoV-2 S glycoproteins.

In embodiments, the dose, as measured in μg, may be the total weight of the dose including the solute, or the weight of the CoV S polypeptide nanoparticles, or the weight of the CoV S polypeptide. Dose is measured using protein concentration assay either A²⁸⁰ or ELISA.

The dose of antigen, including for pediatric administration, may be in the range of about 5 μg to about 25 μg, about 1 μg to about 300 μg, about 90 μg to about 270 μg, about 100 μg to about 160 μg, about 110 μg to about 150 μg, about 120 μg to about 140 μg, or about 140 μg to about 160 μg. In embodiments, the dose is about 120 μg, administered with alum. In aspects, a pediatric dose may be in the range of about 1 μg to about 90 μg. In embodiments, the dose of CoV Spike (S) polypeptide is about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21, about 22, about 23, about 24, about 25 μg, about 26 μg, about 27 μg, about 28 μg, about 29 μg, about 30 μg, about 40 μg, about 50, about 60, about 70, about 80, about 90 about 100 μg, about 110 μg, about 120 μg , about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg , about 260 μg, about 270 μg, about 280 μg, about 290 μg, or about 300 μg, including all values and ranges in between. In embodiments, the dose of CoV S polypeptide is 5 μg. In embodiments, the dose of CoV S polypeptide is 25 μg. In embodiments, the dose of a CoV S polypeptide is the same for the initial dose and for boost doses. In embodiments, the dose of a CoV S polypeptide is the different for the initial dose and for boost doses.

Certain populations may be administered with or without adjuvants. In certain aspects, compositions may be free of added adjuvant. In such circumstances, the dose may be increased by about 10%.

In embodiments, the dose of the adjuvant administered with a non-naturally occurring CoV S polypeptide is from about 1 μg to about 100 μg, for example, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21, about 22, about 23, about 24, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg. about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg of adjuvant. In embodiments, the dose of adjuvant is about 50 μg. In embodiments, the adjuvant is a saponin adjuvant, e.g., MATRIX-M™.

In embodiments, the dose is administered in a volume of about 0.1 mL to about 1.5 mL, for example, about 0.1 mL, about 0.2 mL, about 0.25 mL, about 0.3 mL, about 0.4 mL, about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, or about 1.5 mL. In embodiments, the dose is administered in a volume of 0.25 mL. In embodiments, the dose is administered in a volume of 0.5 mL. In embodiments, the dose is administered in a volume of 0.6 mL.

In particular embodiments for a vaccine against MERS, SARS, or the SARS-CoV-2 coronavirus, the dose may comprise a CoV S polypeptide concentration of about 1 μg/mL to about 50 μg/mL, 10 μg/mL, to about 100 μg/mL, about 10 μg/mL to about 50 μg/mL, about 175 μg/mL to about 325 μg/mL, about 200 μg/mL to about 300 μg/mL, about 220 μg/mL to about 280 μg/mL, or about 240 μg/mL to about 260 μg/mL.

In another embodiment, the disclosure provides a method of formulating a vaccine composition that induces immunity to an infection or at least one disease symptom thereof to a mammal, comprising adding to the composition an effective dose of a nanoparticle or a CoV S polypeptide. The disclosed CoV S polypeptides and nanoparticles are useful for preparing compositions that stimulate an immune response that confers immunity or substantial immunity to infectious agents. Thus, in one embodiment, the disclosure provides a method of inducing immunity to infections or at least one disease symptom thereof in a subject, comprising administering at least one effective dose of a nanoparticle and/or a CoV S polypeptide.

In embodiments, the CoV S polypeptides or nanoparticles comprising the same are administered in combination with an additional immunogenic composition. In embodiments, the additional immunogenic composition induces an immune response against SARS-CoV-2. In embodiments, the additional immunogenic composition is administered within about 1 minute, about 5 minutes, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days of the disclosed CoV S polypeptides or nanoparticles comprising the same. In embodiments, the additional composition is administered with a first dose of a composition comprising a CoV S polypeptide or nanoparticle comprising the same. In embodiments, the additional composition is administered with a boost dose of a composition comprising a CoV S polypeptide or nanoparticle comprising the same.

In embodiments, the additional immunogenic composition comprises an mRNA encoding a SARS-Cov-2 Spike glycoprotein, a plasmid DNA encoding a SARS-Cov-2 Spike glycoprotein, an viral vector encoding a SARS-Cov-2 Spike glycoprotein, or an inactivated SARS-CoV-2 virus.

In embodiments, the additional immunogenic composition comprises mRNA that encodes for a CoV S polypeptide. In embodiments, the mRNA encodes for a CoV S polypeptide comprising proline substitutions at positions 986 and 987 of SEQ ID NO: 1. In embodiments, the mRNA encodes for a CoV S polypeptide comprising an intact furin cleavage site. In embodiments, the mRNA encodes for a CoV S polypeptide comprising proline substitutions at positions 986 and 987 of SEQ NO: 1 and an intact furin cleavage site. In embodiments, the mRNA encodes for a CoV S polypeptide comprising proline substitutions at positions 986 and 987 of SEQ ID NO: 1 and an inactive furin cleavage site. In embodiments, the mRNA encodes for a CoV S polypeptide having an amino acid sequence of SEQ ID NO: 87. In embodiments, the mRNA encoding for a CoV S polypeptide is encapsulated in a lipid nanoparticle. An exemplary immunogenic composition comprising mRNA that encodes for a CoV S polypeptide is described in Jackson et al. N. Eng. J. Med. 2020. An mRNA Vaccine against SARS-CoV-2-preliminary report, which is incorporated by reference in its entirety herein. In embodiments, the composition comprising mRNA that encodes for a CoV S polypeptide is administered at a dose of 25 μg, 100 μg, or 250 μg.

In embodiments, the additional immunogenic composition comprises an adenovirus vector encoding for a CoV S polypeptide. In embodiments, the AAV vector encodes for a wild-type CoV S polypeptide. In embodiments, the AAV vector encodes for a CoV S polypeptide comprising proline substitutions at positions 986 and 987 of SEQ ID NO: 1 and an intact furin cleavage site. In embodiments, the AAV vector encodes for a CoV S polypeptide comprising proline substitutions at positions 986 and 987 of SEQ ID NO: 1 and an inactive furin cleavage site. In embodiments, the AAV vector encodes for a CoV S polypeptide having an amino acid sequence of SEQ ID NO: 87. The following publications describe immunogenic compositions comprising an adenovirus vector encoding for a CoV S polypeptide, each of which is incorporated by, reference in its entirety herein: van Doremalen N. et al. A single dose of ChAdOx1 MERS provides protective immunity in rhesus macaques. Science Advances, 2020; van Doremalen N. et al. ChAdOx1 nCoV-19 vaccination prevents SARS-CoV-2 pneumonia in rhesus macaques. bioRxiv, (2020).

In embodiments, the additional immunogenic composition comprises deoxyribonucleic acid (DNA). In embodiments, the additional immunogenic composition comprises plasmid DNA. In embodiments, the plasmid DNA encodes for a CoV S polypeptide. In embodiments, the DNA encodes for a CoV S polypeptide comprising proline substitutions at positions 986 and 987 of SEQ ID NO: 1 and an intact furin cleavage site. In embodiments, the DNA encodes for a CoV S polypeptide comprising proline substitutions at positions 986 and 987 of SEQ ID NO: 1 and an inactive furin cleavage site. In embodiments, the DNA encodes for a CoV S polypeptide having an amino acid sequence of SEQ ID NO: 87.

In embodiments, the additional immunogenic composition comprises an inactivated virus vaccine.

In embodiments, the CoV S polypeptides or nanoparticles comprising CoV S polypeptides are administered to a patient that has or has previously had a confirmed infection caused by SARS-CoV-2 or a heterogeneous SARS-CoV-2 strain. The infection with SARS-CoV-2 or a heterogeneous SARS-CoV-2 strain may be confirmed by a nucleic acid amplification test (e.g., polymerase chain reaction) or serological testing (e.g., testing for antibodies against a SARS-CoV-2 viral antigen), In embodiments, the CoV S polypeptides or nanoparticles comprising CoV S polypeptides are administered to a patient at least about 3 days, at least about 1 week, at least about 2 weeks, at least about 3 weeks, at least about 4 weeks after a patient has been diagnosed with COVID-19. In embodiments, the CoV S polypeptides or nanoparticles comprising CoV S polypeptides are administered to a patient between 1 week and 1 year after the patient's diagnosis with COVID-19, for example, about 1 week, about 2 weeks, about 3 weeks, about 4 weeks, about 5 weeks, about 6 weeks, about 1 month, about 2 months, about 3 months, about 4 months, about 5 months, about 6 months, about 7 months, about 8 months, about 9 months, about 10 months, about 11 months, or about 1 year. In embodiments, the CoV S polypeptides or nanoparticles comprising CoV S polypeptides are administered to a patient between 1 week and 20 years after the patient's diagnosis with COVID-19, for example, about 1 week, about 2 weeks, about 3 weeks, about 4 weeks, about 5 weeks, about 6 weeks, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, or about 20 years.

In embodiments, the CoV S polypeptides or nanoparticles comprising the same are administered after the patient has been administered a first immunogenic composition. Non-limiting examples of first immunogenic compositions include a SARS-CoV-2 Spike glycoprotein, an mRNA encoding a SARS-Cov-2 Spike glycoprotein, a plasmid DNA encoding a SARS-Cov-2 Spike glycoprotein, an viral vector encoding a SARS-Cov-2 Spike glycoprotein, or an inactivated SARS-CoV-2 virus. In embodiments, the CoV S polypeptides or nanoparticles comprising the same are administered between about 1 week and about 1 year, between about 1 week and 1 month, between about 3 weeks and 4 weeks, between about 1 week and 5 years, between about 1 year and about 5 years, between about 1 year and about 3 years, between about 3 years and about 5 years, between about 5 years and about 10 years, between about 1 year and about 10 years, or between about 1 year and about 2 years after administration of the first immunogenic composition. In embodiments, the CoV S polypeptides or nanoparticles comprising the same are administered between about 1 week and about 1 year after administration of the first immunogenic composition, for example, about 1 week, about 2 weeks, about 3 weeks, about 4 weeks, about 5 weeks, about 6 weeks, about 7 weeks, about 8 weeks, about 9 weeks, about 10 weeks, about 1 month, about 2 months, about 3 months, about 4 months, about 5 months, about 6 months, about 7 months, about 8 months, about 9 months, about 10 months, about 11 months, or about 1 year after administration of the first immunogenic composition.

In embodiments, the CoV S proteins or nanoparticles comprising CoV S proteins are useful for preparing immunogenic compositions to stimulate an immune response that confers immunity or substantial immunity to one or more of MERS, SARS, SARS-CoV-2, and a heterogeneous SARS-CoV-2 strain. Both mucosal and cellular immunity may contribute to immunity to infection and disease. Antibodies secreted locally in the upper respiratory tract are a major factor in resistance to natural infection. Secretory immunoglobulin A (sIgA) is involved in protection of the upper respiratory tract and serum IgG in protection of the lower respiratory tract. The immune response induced by an infection protects against reinfection with the same virus or an antigenically similar viral strain. The antibodies produced in a host after immunization with the nanoparticles disclosed herein can also be administered to others, thereby providing passive administration in the subject.

In embodiments, the CoV S proteins or nanoparticles comprising CoV S proteins induce cross-neutralizing antibodies against SARS-CoV-2 viruses containing S proteins with one or more modifications selected from:

(a) deletion of one or more amino acids of the NTD, wherein the one or more amino acids are selected from the group consisting of amino acid 56, 57, 131, 132, 229, 230, 231, or combinations thereof; and

(b) mutation of one or more amino acids of the NTD, wherein the one or more mutations are selected from the group consisting of amino acid 67, 82, 133, 229, 202, 209, 240, 139, 5, 233, 7, 13, 125, 177, or combinations thereof;

(c) mutation of one or more amino acids of the RBD wherein the one or more mutations is selected from the group consisting of amino acid 488, 404, 471, 464, 439, 481, 426, 440, and combinations thereof;

(d) mutation to one or more amino acids of the SD1/2 , wherein the one or more amino acids is selected from the group consisting of 601, 557, 668, 642, and combinations thereof;

(e) an inactive furin cleavage site (corresponding to one or more mutations in amino acids 669-672);

(f) deletion of one or more amino acids of the S2 subunit, wherein the amino acids are selected from the group consisting of 676-702, 702-711, 775-793, 806-815; and combinations thereof

(g) mutation of one or more amino acids of the S2 subunit, wherein the amino acids are selected from the group consisting of 973, 974, 703, 1105. 688, 969, 1014, and 1163; and combinations thereof

(h) deletion of one or more amino acids from the TMCT (amino acids 1201-1260), wherein the amino acids of the CoV S glycoprotein are numbered with respect to SEQ ID NO: 2.

In embodiments, the CoV S proteins or nanoparticles comprising CoV S proteins induce cross-neutralizing antibodies against SARS-CoV-2 viruses containing S proteins with one or more modifications selected from: deletions of amino acid 56, deletion of amino acid 57, deletion of amino acid 131, N488Y, A557D, D601G, P668H, T703I, S969A, D1105H, N426K, and Y440F, wherein the amino acids are numbered with respect to a CoV S polypeptide having an amino acid sequence of SEQ ID NO: 2.

In embodiments, the CoV S protein or nanoparticle comprising a CoV S protein induces cross-neutralizing antibodies against SARS-CoV-2 viruses containing S proteins with one or more modifications selected from: deletions of amino acid 56, deletion of amino acid 57, deletion of amino acid 131, N488Y, A557D, D601G, P668H, T703I, S969A, and D1105H, wherein the amino acids are numbered with respect to a CoV S polypeptide having an amino acid sequence of SEQ ID NO: 2.

In embodiments, the CoV S protein or nanoparticle comprising a CoV S protein induces cross-neutralizing antibodies against SARS-CoV-2 viruses containing S proteins with one or more modifications selected from: D67A, D202G, L229H, K404N, E471K, N488Y, D601G, and A688V, wherein the amino acids are numbered with respect to a CoV S polypeptide having an amino acid sequence of SEQ ID NO: 2.

In embodiments, the CoV S protein or nanoparticle comprising a CoV S protein induces cross-neutralizing antibodies against SARS-CoV-2 viruses containing S proteins with one or more modifications selected from: deletion of amino acids 229-231, D67A, D202G, K404N, E471K, N488Y, D601G, and A688V, wherein the amino acids are numbered with respect to a CoV S polypeptide having an amino acid sequence of SEQ ID NO: 2.

In embodiments, the CoV S protein or nanoparticle comprising a CoV S protein induces cross-neutralizing antibodies against SARS-CoV-2 viruses containing S proteins with one or more modifications selected from: deletion of amino acids 229-231, L5F, D67A, D202G, K404N, E471K, N488Y, D601G, and A688V wherein the amino acids are numbered with respect to a CoV S polypeptide having an amino acid sequence of SEQ ID NO: 2.

In embodiments, the CoV S protein or nanoparticle comprising a CoV S protein induces cross-neutralizing antibodies against SARS-CoV-2 viruses containing S proteins with one or more modifications selected from: L5F, T7N, P13S, D125Y, R177S, K404T, E471K, N488Y, D601G, H642Y, T1014I, and V1163F, wherein the amino acids are numbered with respect to a CoV S polypeptide having an amino acid sequence of SEQ ID NO: 2.

In embodiments, the CoV S protein or nanoparticle comprising a CoV S protein induces cross-neutralizing antibodies against SARS-CoV-2 viruses with an S protein comprising one or more modifications selected from: W139C and L439R, wherein the amino acids are numbered with respect to a CoV S polypeptide having an amino acid sequence of SEQ ID NO: 2. In embodiments, the CoV S protein comprising W139C and L439R modifications is expressed with a signal peptide having an amino acid sequence of SEQ ID NO: 117 or SEQ ID NO: 5. In embodiments, the CoV S protein or nanoparticle comprising a CoV S protein induces cross-neutralizing antibodies against SARS-CoV-2 viruses with one or more modifications selected from: D601G, W139C, and L439R, wherein the amino acids are numbered with respect to a CoV S polypeptide having an amino acid sequence of SEQ ID NO: 2. In embodiments, the CoV S protein or nanoparticle comprising D601G, W139C, and L439R modifications is expressed with a signal peptide having an amino acid sequence of SEQ ID NO: 117 or SEQ ID NO: 5.

In embodiments, the CoV S protein or nanoparticle comprising a CoV S protein induces cross-neutralizing antibodies against SARS-CoV-2 viruses with one or more modifications selected from: D601G, L5F, D67A, D202G, deletions of amino acids 229-231, R233I, K404N, E471K, N488Y, and A688V, wherein the amino acids are numbered with respect to a CoV S polypeptide having an amino acid sequence of SEQ ID NO: 2. In embodiments, the CoV S protein or nanoparticle comprising a CoV S protein induces cross-neutralizing antibodies against SARS-CoV-2 viruses with one or more modifications selected from: L5F, D67A, D202G, deletions of amino acids 229-231, R233I, K404N, E471K, N488Y, and A688V, wherein the amino acids are numbered with respect to a CoV S polypeptide having an amino acid sequence of SEQ ID NO: 2.

In embodiments, the present disclosure provides a method of producing one or more of high affinity anti-MERS-CoV, anti-SARS-CoV, and anti-SARS-CoV-2 virus antibodies. The high affinity antibodies produced by immunization with the nanoparticles disclosed herein are produced by administering an immunogenic composition comprising an S CoV polypeptide or a nanoparticle comprising an S CoV polypeptide to an animal, collecting the serum and/or plasma from the animal, and purifying the antibody from the serum and/or plasma. In one embodiment, the animal is a human. In embodiments, the animal is a chicken, mouse, guinea pig, rat, rabbit, goat, human, horse, sheep, or cow. In one embodiment, the animal is bovine or equine. In another embodiment, the bovine or equine animal is transgenic. In yet a further embodiment, the transgenic bovine or equine animal produces human antibodies. In embodiments, the animal produces monoclonal antibodies. In embodiments, the animal produces polyclonal antibodies. In one embodiment, the method further comprises administration of an adjuvant or immune stimulating compound. In a further embodiment, the purified high affinity antibody is administered to a human subject. In one embodiment, the human subject is at risk for infection with one or more of MERS, SARS, and SARS-CoV-2.

In embodiments, the CoV S proteins or nanoparticles are co-administered with an influenza glycoprotein or nanoparticle comprising an influenza glycoprotein. Suitable glycoproteins and nanoparticles are described in US Publication No. 2018/0133308 and US Publication No. 2019/0314487, each of which is incorporated by reference herein in its entirety. In embodiments, the CoV S protein or nanoparticle is coadministered with: (a) a detergent-core nanoparticle, wherein the detergent-core nanoparticle comprises a recombinant influenza hemagglutinin (HA) glycoprotein from a Type B influenza strain; and (b) a Hemagglutinin Saponin Matrix Nanoparticle (HaSMaN), wherein the HaSMaN comprises a recombinant influenza HA glycoprotein from a Type A influenza strain and ISCOM matrix adjuvant. In embodiments, the CoV S protein or nanoparticle is coadministered with a nanoparticle comprising a non-ionic detergent core and an influenza HA glycoprotein, wherein the influenza HA glycoprotein contains a head region that projects outward from the non-ionic detergent core and a transmembrane domain that is associated with the non-ionic detergent core, wherein the influenza HA glycoprotein is a HA0 glycoprotein, wherein the amino acid sequence of the influenza HA glycoprotein has 100% identity to the amino acid sequence of the native influenza HA protein. In embodiments, the influenza glycoprotein or nanoparticle is coformulated with the CoV S protein or nanoparticle.

All patents, patent applications, references, and journal articles cited in this disclosure are expressly incorporated herein by reference in their entireties for all purposes.

### EXAMPLES

### Example 1

### Expression and Purification of Coronavirus Spike (S) Polypeptide Nanoparticles

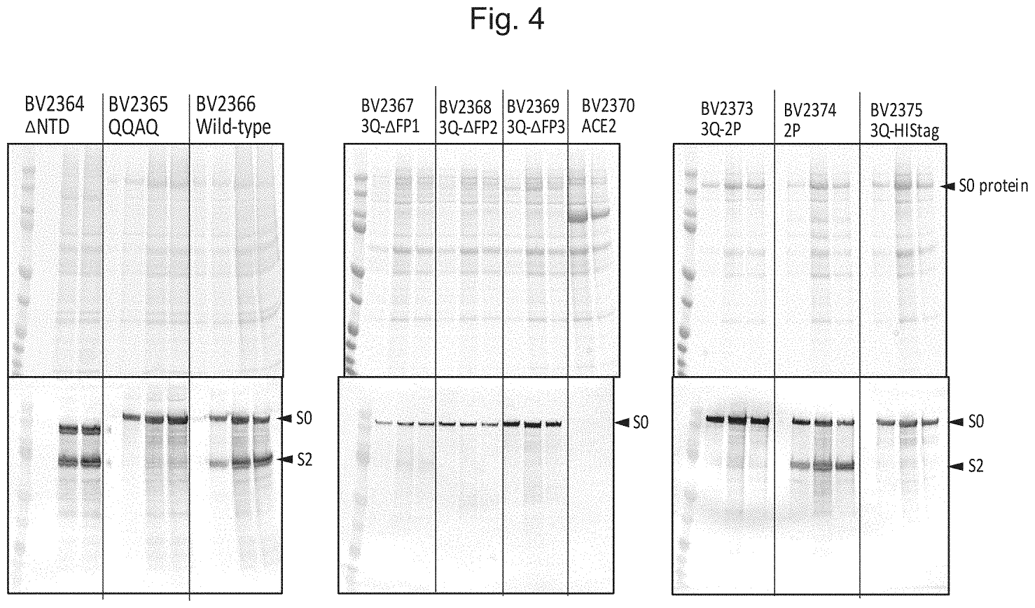

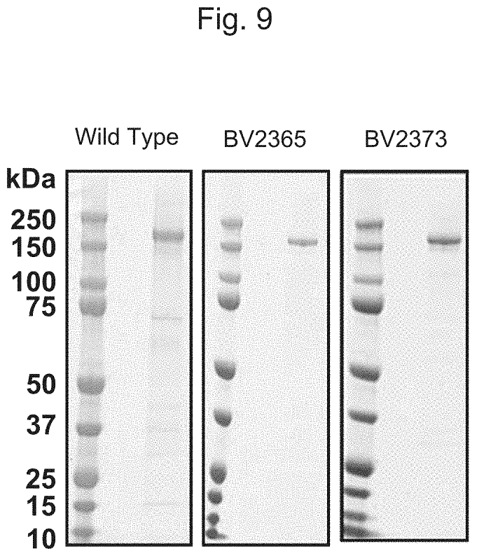

The native coronavirus Spike (S) polypeptide (SEQ ID NO: 1 and SEQ ID NO:2) and CoV Spike polypeptides which have amino acid sequences corresponding to SEQ ID NOS: 3, 4, 38, 41, 44, 48, 51, 54, 58, 61, 63, 65, 67, 73, 75, 78. 79, 82, 83, 85, 87, 106, 108, 89, 112-115, 132, 133, 114, 138, 141, 144, 147, 151, 153, 156, 158, 164-168 have been expressed in a baculovirus expression system and recombinant plaques expressing the coronavirus Spike (S) polypeptides were picked and confirmed. In each case the signal peptide is SEQ ID NO: 5. FIG. 4 and FIG. 9 show successful purification of the CoV Spike polypeptides BV2364, BV2365, BV2366, BV2367, BV2368, BV2369, BV2373, BV2374, and BV2375. Table 2 shows the sequence characteristics of the aforementioned CoV Spike polypeptides.

TABLE 2
Selected CoV Spike Polypeptides

| CoV S polypeptide | Modification | SEQ ID NO. |
|---|---|---|
| BV2364 | Deleted N-Terminal Domain | 48 |
| BV2365 | Inactive furin cleavage site | 4 |
| BV2361/BV2366 | Wild-type | 2 |
| BV2367 | Deletion of amino acids 676-685, | 63 |
| | inactive furin cleavage site | |
| BV2368 | Deletion of amino acids 702-711, | 65 |
| | inactive furin cleavage site | |
| BV2369 | Deletion of amino acids 806-815, | 67 |
| | inactive furin cleavage site | |
| BV2373, formulated | Inactive furin cleavage site, | 87 |
| into a composition | K973P mutation, V974P mutation | |
| referred to herein as | | |
| “NVX-CoV2373” | | |
| BV2374 | K973P mutation, V974P mutation | 85 |
| BV2374 | Inactive furin cleavage site and | 58 |
| | His-tag | |
| BV2384 | Inactive furin cleavage site | 110 |
| | (GSAS), K973P, V974P mutation | |
| BV2425 | Inactive furin cleavage site, | 114 |
| | K973P, V974P mutation, deletions | |
| | of amino acid 56, deletion of | |
| | amino acid 57, deletion of amino | |
| | acid 131, N488Y mutation, | |
| | A557D mutation, D601G | |
| | mutation, P668H mutation, T703I | |
| | mutation, S969A mutation, and | |
| | D1105H mutation | |
| BV2426 | Inactive furin cleavage site, | 115 |
| | K973P mutation, V974P mutation, | |
| | D67A mutation, D202G mutation, | |
| | L229H mutation, K404N | |
| | mutation, E471K mutation, | |
| | N488Y mutation, D601G | |
| | mutation, and A688V mutation | |
| BV2438 | Inactive furin cleavage site | 132 |
| | (QQAQ: SEQ ID NO: 7), K973P | |
| | mutation, V974P mutation, | |
| | K404N mutation, E471K | |
| | mutation, N488Y mutation, D67A | |
| | mutation, D202G mutation, | |
| | L229H mutation, D601G | |
| | mutation, A688V mutation | |
| BV2423 | Inactive furin cleavage site (GG), | 133 |
| | K973P mutation, V974P mutation, | |
| | D601G mutation | |
| BV2425 | Inactive furin cleavage site | 114 |
| | (QQAQ: SEQ ID NO: 7), K973P | |
| | mutation, V974P mutation, | |
| | deletion of amino acids 56, 57, and | |
| | 131, N488Y mutation, A557D | |
| | mutation, D601G mutation, | |
| | P668H mutation, T703I mutation, | |
| | S969A mutation, D1105H | |
| | mutation | |
| BV2425-2 | Inactive furin cleavage site (GG), | 138 |
| | K973P mutation, V974P mutation, | |
| | deletion of amino acids 56, 57, and | |
| | 131, N488Y mutation, A557D | |
| | mutation, D601G mutation, | |
| | P668H mutation, T703I mutation, | |
| | S969A mutation, D1105H | |
| | mutation | |
| BV2439 | Inactive furin cleavage site (GG), | 141 |
| | K973P mutation, V974P mutation, | |
| | K404N mutation, E471K | |
| | mutation, N488K mutation, D67A | |
| | mutation, D202G mutation, | |
| | L229H mutation, D601G | |
| | mutation, A688V mutation | |
| BV2441 | Inactive form cleavage site | 144 |
| | (QQAQ: SEQ ID NO: 7), K973P | |
| | mutation, V974P mutation, | |
| | K404N mutation, E471K | |
| | mutation, N488Y mutation, D67A | |
| | mutation, D202G mutation, | |
| | D601G mutation, A688V | |
| | mutation, deletions of amino acids | |
| | 229-231 | |
| BV2442 | Inactive furin cleavage site (GG), | 147 |
| | K973P mutation, V974P mutation, | |
| | K404N mutation, E471K | |
| | mutation, N488Y mutation, D67A | |
| | mutation, D202G mutation, | |
| | D601G mutation, A688V | |
| | mutation, deletions of amino acids | |
| | 229-231 | |
| BV2443 | Inactive form cleavage site | 151 |
| | (QQAQ: SEQ ID NO: 7), K973P | |
| | mutation, V974P mutation, T7N | |
| | mutation, P13S mutation, D125Y | |
| | mutation, R177S mutation, K404T | |
| | mutation, E471K mutation, | |
| | N488Y mutation, D601G | |
| | mutation, H642Y mutation, | |
| | T1014Y mutation, V1163F | |
| | mutation | |
| BV2448 | Inactive furin cleavage site | 153 |
| | (QQAQ: SEQ ID NO: 7), K973P | |
| | mutation, V974P mutation, | |
| | W139C mutation, S481P | |
| | mutation, D601G mutation, | |
| | L439R mutation | |
| BV1.526NY-1 | Inactive furin cleavage site | 156 |
| | (QQAQ: SEQ ID NO: 7), K973P | |
| | mutation, V974P mutation, T82I | |
| | mutation, D240G mutation, | |
| | E471K mutation, D601G | |
| | mutation, A688V mutation | |
| BV1.526NY-2 | Inactive form cleavage site | 158 |
| | (QQAQ: SEQ ID NO: 7), K973P | |
| | mutation, V974P mutation, T82I | |
| | mutation, D240G mutation, | |
| | E464K mutation, D601G | |
| | mutation, A688V mutation | |
| BV2465 | Mutations: T6R, G129D, R145G, | 164 |
| | L439R, T465K, D601G, P668R, | |
| | D937G, K973P, V974P, Inactive | |
| | Furin Cleavage site (QQAQ: SEQ | |
| | ID NO: 7) | |
| | Deletion: 143, 144 | |
| BV2457 | T82I, G129D, E141K, L439R, | 165 |
| | T471Q, D601G, P668R, Q1058H, | |
| | K973P, V974P, Inactive Furin | |
| | Cleavage site (QQAQ: SEQ ID | |
| | NO: 7) | |
| BV2472 | Mutations: T6R, G129D, R145G, | 166 |
| | K404N, L439R, T465K, D601G, | |
| | P668R, D937G, W245I, K973P, | |
| | V974P, Inactive Furin Cleavage | |
| | site (QQAQ: SEQ ID NO: 7) | |
| | Deletion: 143, 144 | |
| BV2480 | T82I, Y131S, Y132N, R333K, | 167 |
| | E471K, N488Y, D601G, P668H, | |
| | D937N, Inactive Furin Cleavage | |
| | site (QQAQ: SEQ ID NO: 7), | |
| | K973P mutation, V974P mutation | |
| BV2481 | T82I, Y131T, Y132S, R333K, | 168 |
| | E471K, N488Y, D601G, P668H, | |
| | D937N, Inactive Furin Cleavage | |
| | site (QQAQ: SEQ ID NO: 7), | |
| | K973P mutation, V974P mutation | |
| | Insertion of asparagine after amino | |
| | acid 132 | |

The wild-type BV2361 protein (SEQ ID NO: 2) binds to human angiotensin-converting enzyme 2 precursor (hACE2). Bio-layer interferometry and ELISA were performed to assess binding of the CoV S polypeptides.

### Bio-Layer Interferometry (BLI)

The BLI experiments were performed using an Octet QK384 system (Pall Forté Bio, Fremont, Calif.). His-tagged human ACE2 (2 μg mL-1) was immobilized on nickel-charged Ni-NTA biosensor tips. After baseline, SARS-CoV-2 S protein containing samples were 2-fold serially diluted and were allowed to associate for 600 seconds followed by dissociation for an additional 900 sec. Data was analyzed with Octet software HT 101:1 global curve fit.

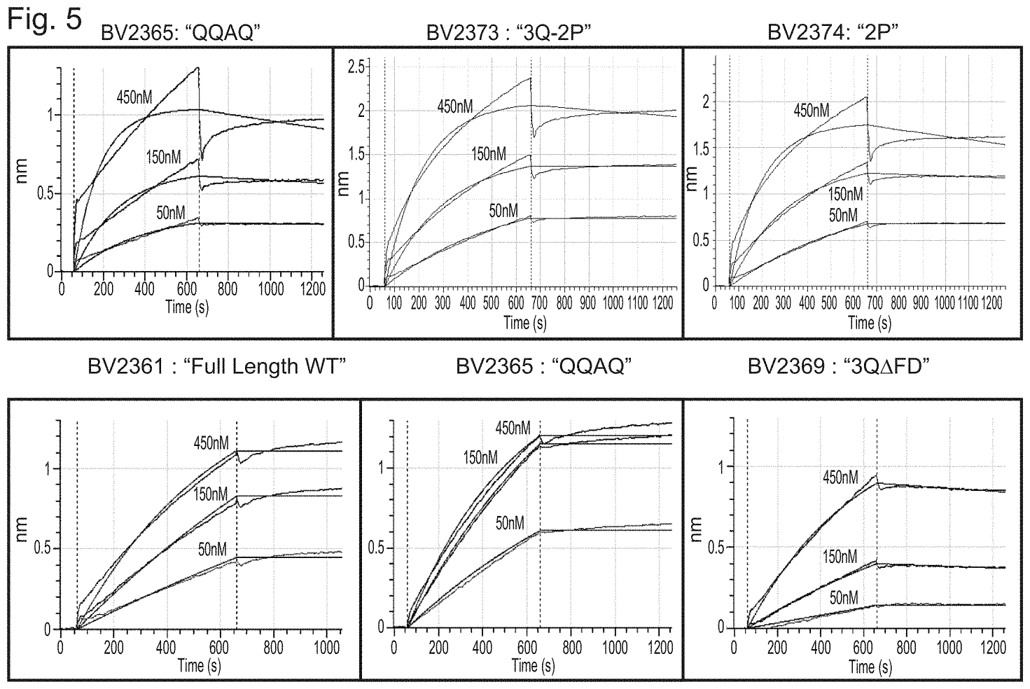

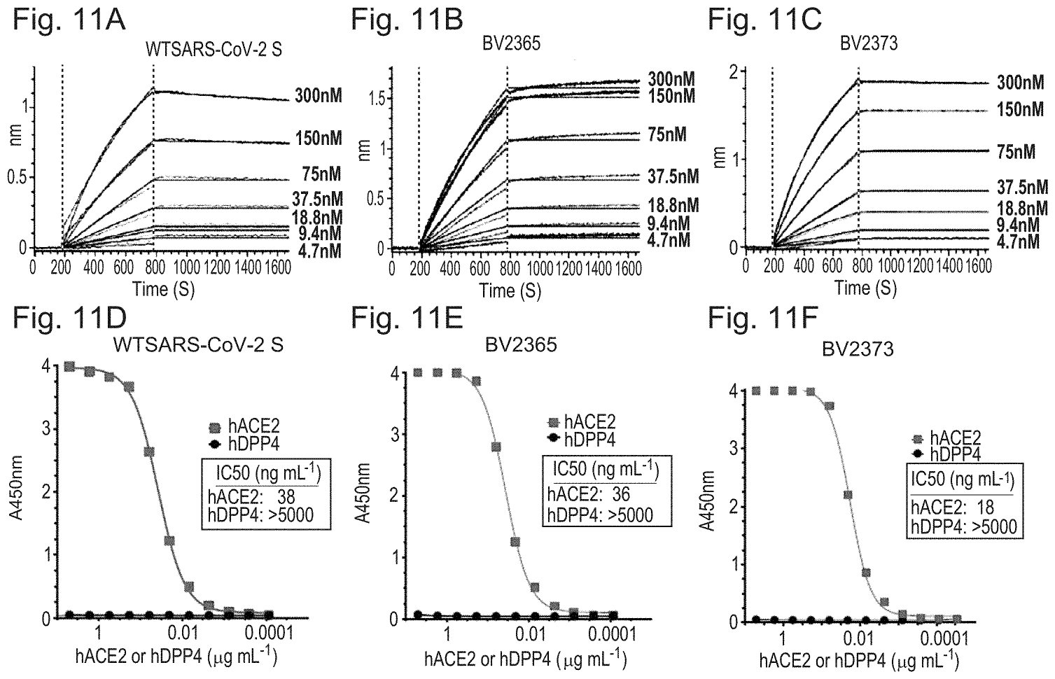

The CoV S polypeptides BV2361, BV2365, BV2369, BV2365, BV2373, BV2374 retain the ability to bind to hACE2 (FIG. 5, FIGS. 11A-C). Dissociation kinetics showed that the S-proteins remained tightly bound as evident by minimal or no dissociation over 900 seconds of observation in the absence of fluid phase S protein (FIGS. 11A-C).

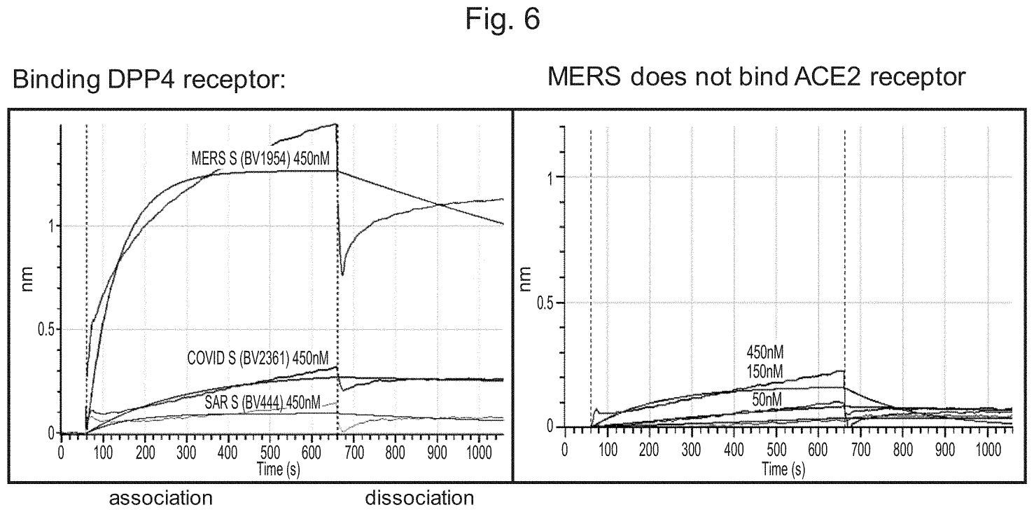

Furthermore, binding is specific. The wild-type CoV S protein, BV2361 and the CoV S polypeptides BV2365 and BV2373 do not bind the MERS-CoV receptor, dipeptidyl peptidase IV (DPP4). Additionally, the MERS S protein does not bind to human angiotensin-converting enzyme 2 precursor (hACE2) (FIG. 6 and FIGS. 11D-F).

### ELISA

The specificity of the CoV S polypeptides for hACE2 was confirmed by ELISA. Ninety-six well plates were coated with 100 μL SARS-CoV-2 spike protein (2 μg/mL) overnight at 4° C. Plates were washed with phosphate buffered saline with 0.05% Tween (PBS-T) buffer and blocked with TBS Startblock blocking buffer (ThermoFisher, Scientific). His-tagged hACE2 and hDPP4 receptors were 3-fold serially diluted (5-0.0001 μg mL-1) and added to coated wells for 2 hours at room temperature. The plates were washed with PBS-T. Optimally diluted horseradish peroxidase (HRP) conjugated anti-histidine was added and color developed by addition of and 3,3′,5,5′-tetramethylbenzidine peroxidase substrate (TMB, T0440-IL, Sigma, St. Louis, Mo., USA). Plates were read at an OD of 450 nm with a SpectraMax Plus plate reader (Molecular Devices, Sunnyvale, Calif., USA) and data analyzed with SoftMax software. EC50 values were calculated by 4-parameter fitting using GraphPad Prism 7.05 software.

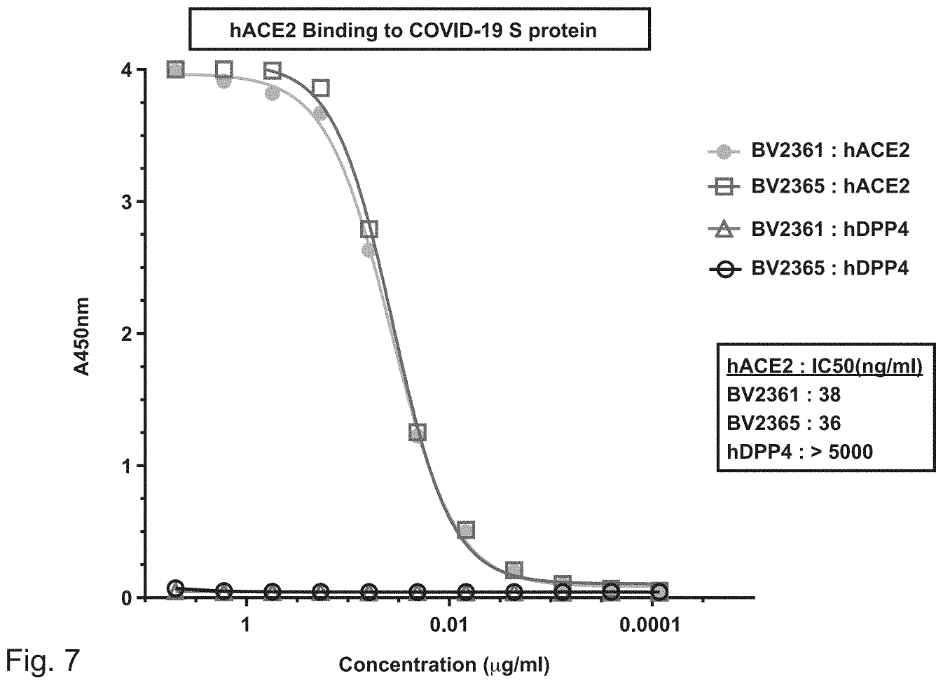

The ELISA results showed that the wild-type CoV S polypeptide (BV2361), BV2365, and BV2373 proteins specifically bound hACE2 but failed to bind the hDPP-4 receptor used by MERS-CoV (IC₅₀>5000 ng mL-1). The wild-type CoV S polypeptide and BV2365 bound to hACE2 with similar affinity (IC₅₀=36-38 ng/mL), while BV2373 attained 50% saturation of hACE2 binding at 2-fold lower concentration (IC₅₀=18 ng/mL) (FIG. 7, FIGS. 11D-F).

### Protein and Nanoparticle Production

The recombinant virus is amplified by infection of Sf9 insect cells. A culture of insect cells is infected at ˜3 MOI (Multiplicity of infection=virus ffu or pfu/cell) with baculovirus. The culture and supernatant is harvested 48-72 hrs post-infection. The crude cell harvest, approximately 30 mL, is clarified by centrifugation for 15 minutes at approximately 800×g. The resulting crude cell harvests containing the coronavirus Spike (S) protein are purified as nanoparticles as described below.

To produce nanoparticles, non-ionic surfactant TERGITOL® nonylphenol ethoxylate NP-9 is used in the membrane protein extraction protocol. Crude extraction is further purified by passing through anion exchange chromatography, lentil lectin affinity/HIC and cation exchange chromatography. The washed cells are lysed by detergent treatment and then subjected to low pH treatment which leads to precipitation of BV and Sf9 host cell DNA and protein. The neutralized low pH treatment lysate is clarified and further purified on anion exchange and affinity chromatography before a second low pH treatment is performed.

Affinity chromatography is used to remove Sf9/BV proteins, DNA and NP-9, as well as to concentrate the coronavirus Spike (S) protein. Briefly, lentil lectin is a metalloprotein containing calcium and manganese, which reversibly binds polysaccharides and glycosylated proteins containing glucose or mannose. The coronavirus Spike (S) protein-containing anion exchange flow through fraction is loaded onto the lentil lectin affinity chromatography resin (Capto Lentil Lectin, GE Healthcare). The glycosylated coronavirus Spike (S) protein is selectively bound to the resin while non-glycosylated proteins and DNA are removed in the column flow through. Weakly bound glycoproteins are removed by buffers containing high salt and low molar concentration of methyl alpha-D-mannopyranoside (MMP).

The column washes are also used to detergent exchange the NP-9 detergent with the surfactant polysorbate 80 (PS80). The coronavirus Spike (S) polypeptides are eluted in nanoparticle structure from the lentil lectin column with a high concentration of MMP. After elution, the coronavirus Spike (S) protein trimers are assembled into nanoparticles composed of coronavirus Spike (S) protein trimers and PS80 contained in a detergent core.

### Example 2

### Immunogenicity of Coronavirus Spike (S) Polypeptide Nanoparticle Vaccines in Mice

The coronavirus Spike (S) protein composition comprising a CoV S polypeptide of SEQ ID NO: 87 (also called “BV2373”) as described in Example 1 was evaluated for immunogenicity and toxicity in a murine model, using female BALB/c mice (7-9 weeks old; Harlan Laboratories Inc., Frederick, Md.). The compositions were evaluated in the presence and in the absence of a saponin adjuvant, e.g., MATRIX-M™. Compositions containing MATRIX-M™ contained 5 μg of MATRIX-M™. Vaccines containing coronavirus Spike (S) polypeptide at various doses, including 0.01 μg, 0.1 μg, 1 μg, and 10 μg, were administered intramuscularly as a single dose (also referred to as a single priming dose) (study day 14) or as two doses (also referred to as a prime/boost regimen) spaced 14-days apart (study day 0 and 14). A placebo group served as a non-immunized control. Serum was collected for analysis on study days −1, 13, 21, and 28. Vaccinated and control animals were intranasally challenged with SARS-CoV-2 42 days following one (a single dose) or two (two doses) immunizations.

### Vaccine Immunogenicity

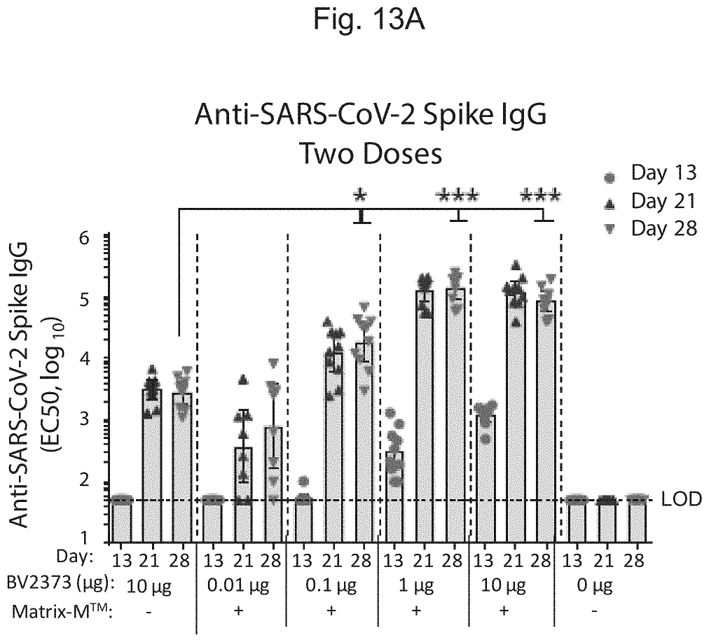

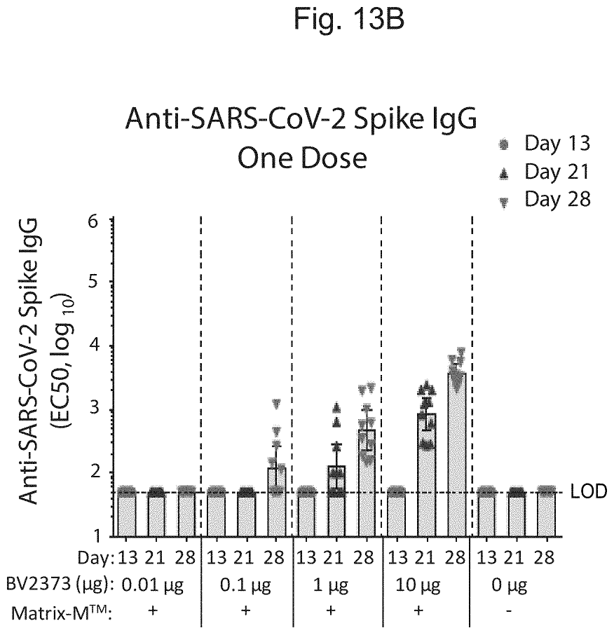

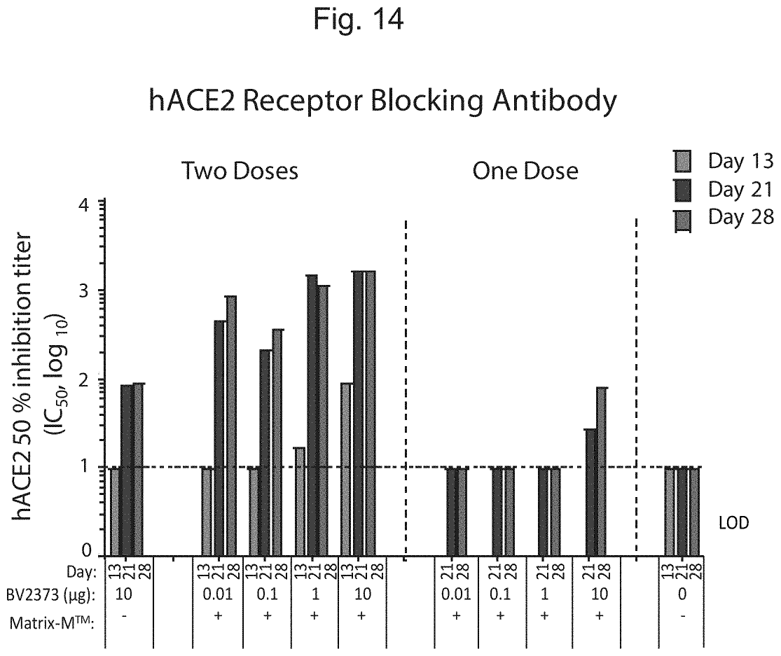

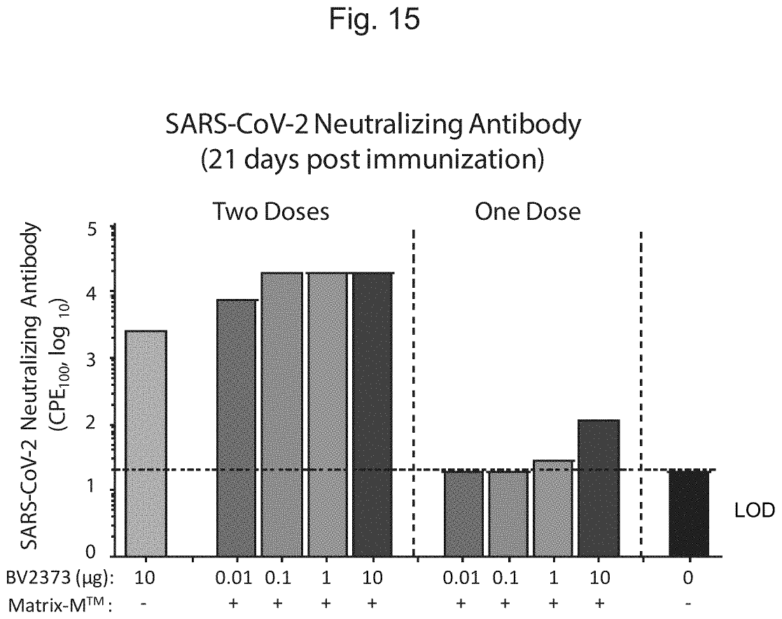

Animals immunized with a single priming dose of 0.1-10 μg BV2373 and MATRIX-M™ had elevated anti-S IgG titers that were detected 21-28 days after a single immunization (FIG. 13B). Mice immunized with a 10 μg dose of BV2373 and MATRIX-M™ produced antibodies that blocked hACE2 receptor binding to the CoV S protein and virus neutralizing antibodies that were detected 21-28 days after a single priming dose (FIG. 14 and FIG. 15). Animals immunized with the prime/boost regimen (two doses) had significantly elevated anti-S IgG titers that were detected 7-16 days following the booster immunization across all dose levels (FIG. 13A). Animals immunized with BV2373 (1 μg and 10 μg) and MATRIX-M™ had similar high anti-S IgG titers following immunization (GMT=139,000 and 84,000, respectively). Mice immunized with BV2373 (0.1 μg, 1 μg, or 10 μg) and MATRIX-M™ had significantly (p≤0.05 and p≤0.0001) higher anti-S IgG titers compared to mice immunized with 10 μg BV2373 without adjuvant (FIG. 13A). These results indicate the potential for 10- to 100-fold dose sparing provided by the MATRIX-M™ adjuvant. Furthermore, immunization with two doses of BV2373 and MATRIX-M™ elicited high titer antibodies that blocked hACE2 receptor binding to S-protein (IC50=218-1642) and neutralized the cytopathic effect (CPE) of SARS-CoV-2 on Vero E6 cells (100% blocking of CPE=7680-20,000) across all dose levels (FIG. 14 and FIG. 15).

### SARS CoV-2 Challenge

To evaluate the induction of protective immunity, immunized mice were challenged with SARS-CoV-2. Since mice do not support replication of the wild-type SARS-CoV-2 virus, on day 52 post initial vaccination, mice were intranasally infected with an adenovirus expressing hACE2 (Ad/hACE2) to render them permissive. Mice were intranasally inoculated with 1.5×10⁵ pfu of SARS-CoV-2 in 50 μL divided between nares. Challenged mice were weighed on the day of infection and daily for up to 7 days post infection. At 4- and 7-days post infection, 5 mice were sacrificed from each vaccination and control group, and lungs were harvested and prepared for pulmonary histology.

The viral titer was quantified by a plaque assay. Briefly, the harvested lungs were homogenized in PBS using 1.0 mm glass beads (Sigma Aldrich) and a Beadruptor (Omini International Inc.). Homogenates were added to Vero E6 near confluent cultures and SARS-CoV-2 virus titers determined by counting plaque forming units (pfu) using a 6-point dilution curve

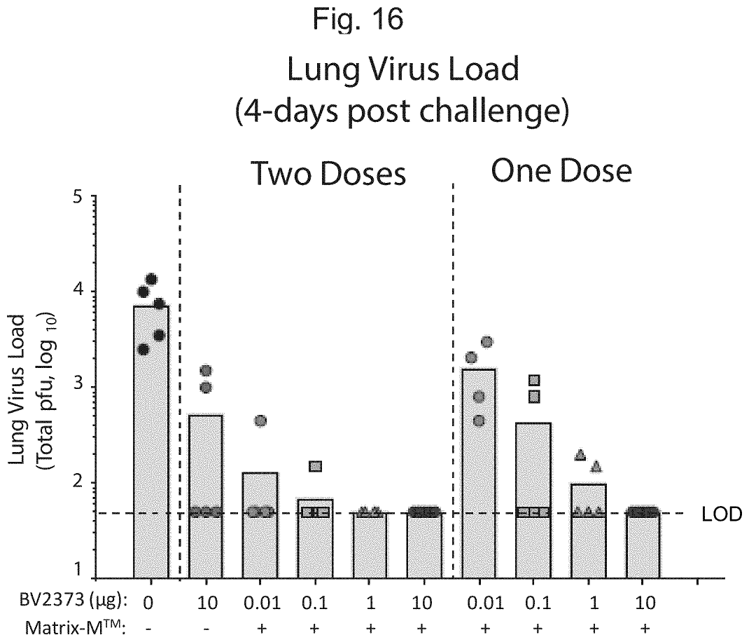

At 4 days post infection, placebo-treated mice had 10⁴ SARS-CoV-2 pfu/lung, while the mice immunized with BV2363 without MATRIX-M™ had 10³ pfu/lung (FIG. 16). The BV2373 with MATRIX-M™ prime-only groups of mice exhibited a dose dependent reduction in virus titer, with recipients of the 10 μg BV2373 dose having no detectable virus at day 4 post infection. Mice receiving 1 μg, 0.1 μg and 0.01 μg BV2373 doses all showed a marked reduction in titer compared to placebo-vaccinated mice. In the prime/boost groups, mice immunized with 10 μg, 1 μg and 0.1 μg doses had almost undetectable lung virus loads, while the 0.01 μg group displayed a reduction of 1 log reduction relative to placebo animals.

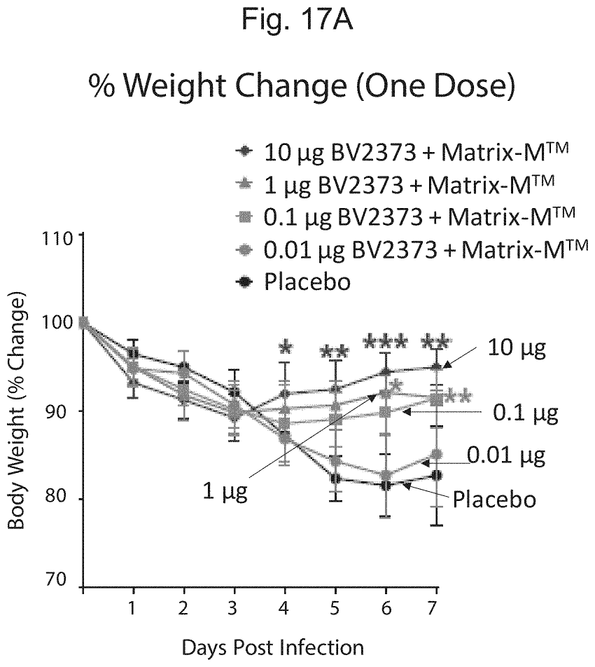

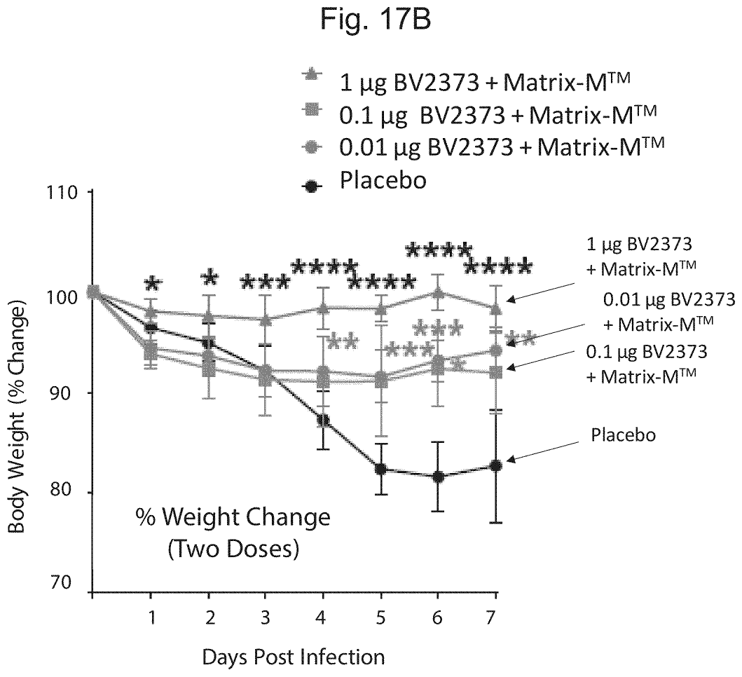

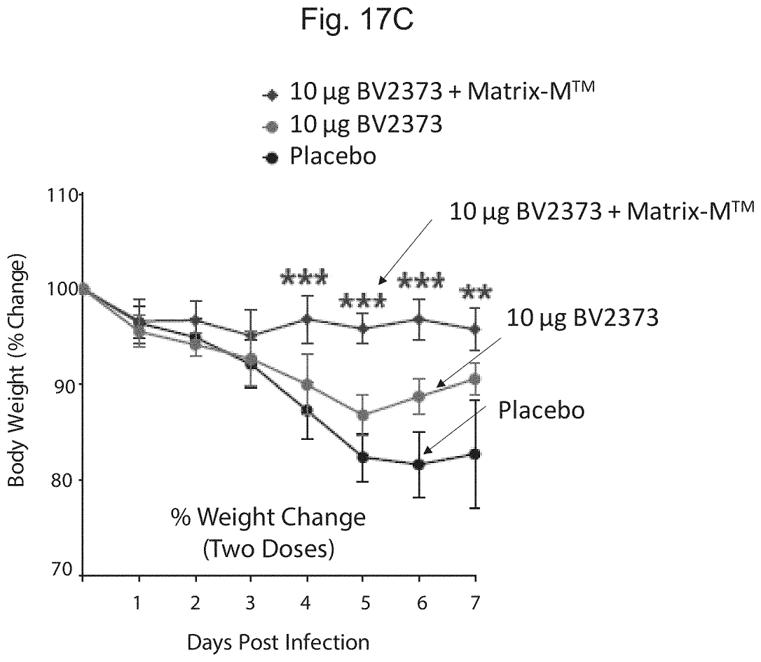

Weight loss paralleled the viral load findings. Animals receiving a single dose of BV2373 (0.1 μg, 1 μg, and 10 μg) and MATRIX-M™ showed marked protection from weight loss compared to the unvaccinated placebo animals (FIG. 17A). The mice receiving a prime and boost dose with adjuvant also demonstrated significant protection against weight loss at all dose levels (FIGS. 17B-C). The effect of the presence of adjuvant on protection against weight loss was evaluated. Mice receiving the prime/boost (two doses) plus adjuvant were significantly protected from weight loss relative to placebo, while the group immunized without adjuvant was not (FIG. 17C). These results showed that BV2373 confers protection against SAES-CoV-2 and that low doses of the vaccine associated with lower serologic responses do not exacerbate weight loss or demonstrate exaggerated illness.

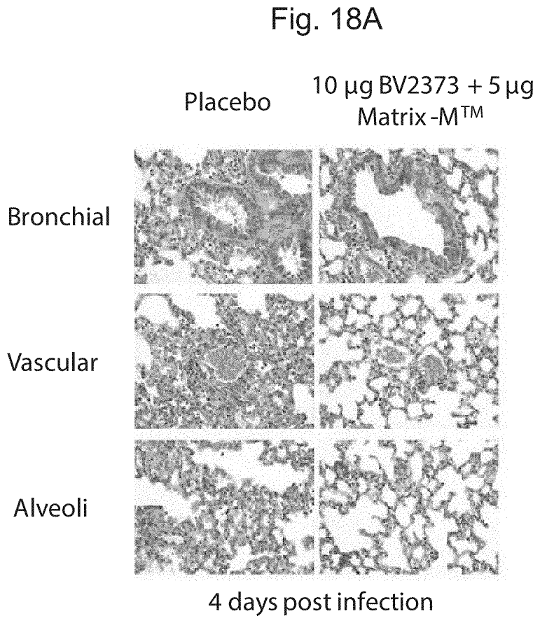

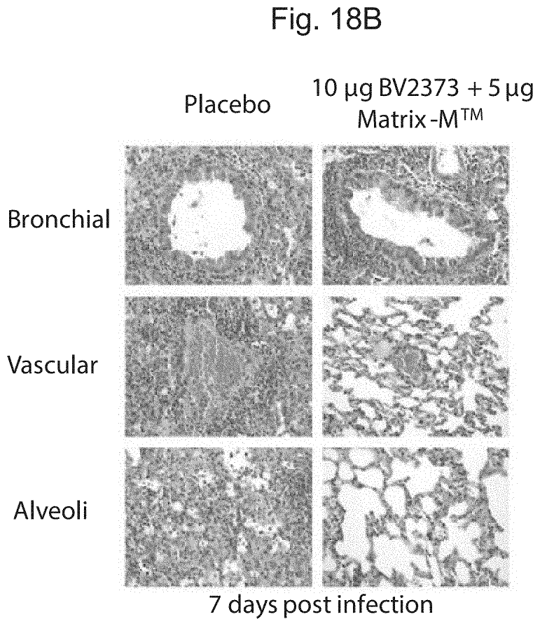

Lung histopathology was evaluated on days 4 and day 7 post infection (FIG. 18A and FIG. 18B). At day 4 post infection, placebo-immunized mice showed denudation of epithelial cells in the large airways with thickening of the alveolar septa surrounded by a mixed inflammatory cell population. Periarteriolar cuffing was observed throughout the lungs with inflammatory cells consisting primarily of neutrophils and macrophages. By day 7 post infection, the placebo-treated mice displayed peribronchiolar inflammation with increased periarteriolar cuffing. The thickened alveolar septa remained with increased diffuse interstitial inflammation throughout the alveolar septa (FIG. 18B).

The BV2373 immunized mice showed significant reduction in lung pathology at both day 4 and day 7 post infection in a dose-dependent manner. The prime only group displays reduced inflammation at the 10 μg and 1 μg dose with a reduction in inflammation surrounding the bronchi and arterioles compared to placebo mice. In the lower doses of the prime-only groups, lung inflammation resembles that of the placebo groups, correlating with weight loss and lung virus titer. The prime/boost immunized groups displayed a significant reduction in lung inflammation for all doses tested, which again correlated with lung viral titer and weight loss data. The epithelial cells in the large and small bronchi at day 4 and 7 were substantially preserved with minimal bronchiolar sloughing and signs of viral infection. The arterioles of animals immunized with 10 μg, 1 μg and 0.1 μg doses have minimal inflammation with only moderate cuffing seen with the 0.01 μg dose, similar to placebo. Alveolar inflammation was reduced in animals that received the higher doses with only the lower 0.01 μg dose associated with inflammation (FIGS. 18A-18B). These data demonstrate that BV2373 reduces lung inflammation after challenge and that even doses and regimens of BV2373 that elicit minimal or no detectable neutralizing activity are not associated with exacerbation of the inflammatory response to the virus. Furthermore, the vaccine does not cause vaccine associated enhanced respiratory disease (VAERD) in challenged mice.

### T Cell Response

The effect of the vaccine composition comprising a CoV S polypeptide of SEQ ID NO: 87 on the T cell response was evaluated. BALB/c mice (N=6 per group) were immunized intramuscularly with 10 μg BV2373 with or without 5 μg MATRIX-M™ in 2 doses spaced 21-days apart. Spleens were collected 7-days after the second immunization (study day 28). A non-vaccinated group (N=3) served as a control.

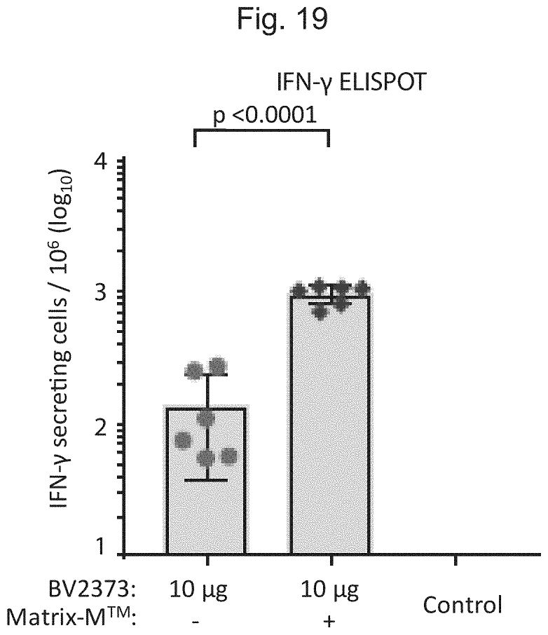

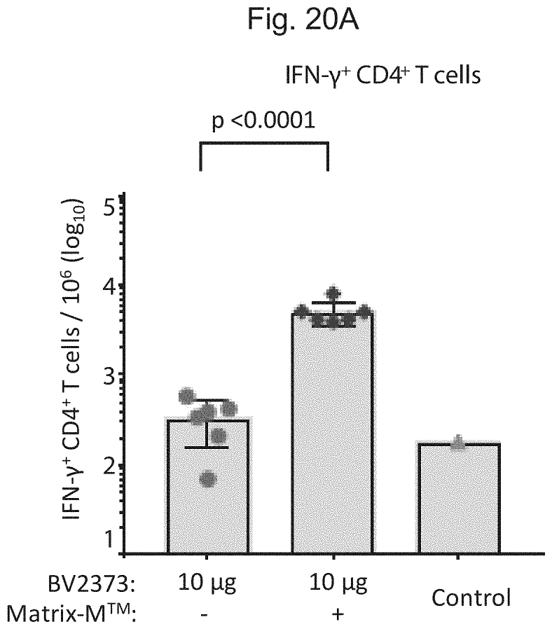

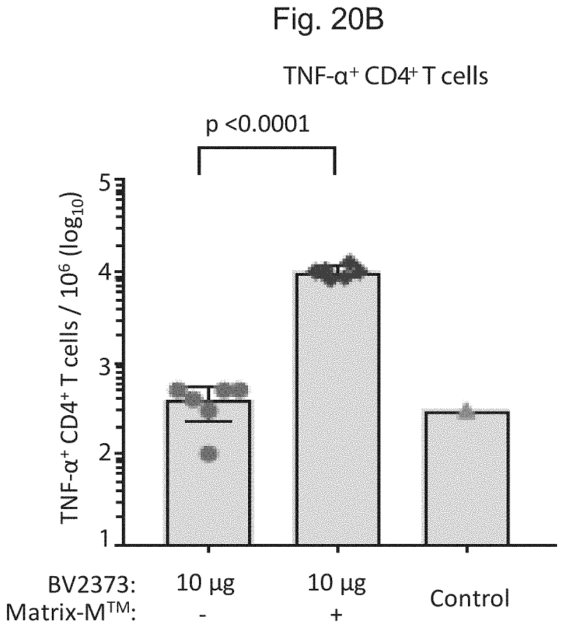

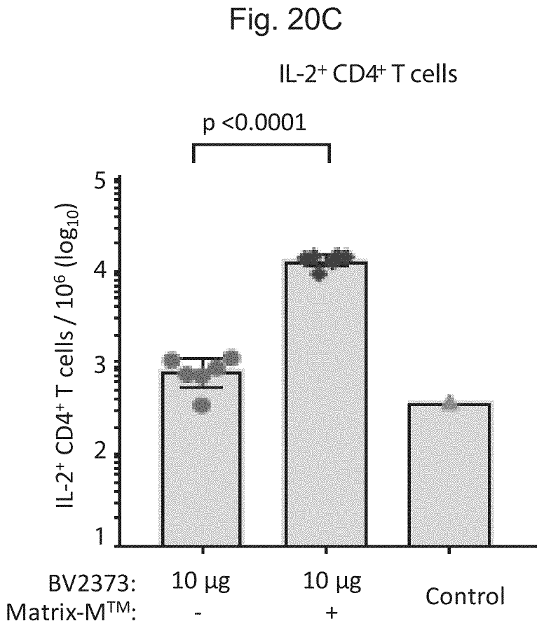

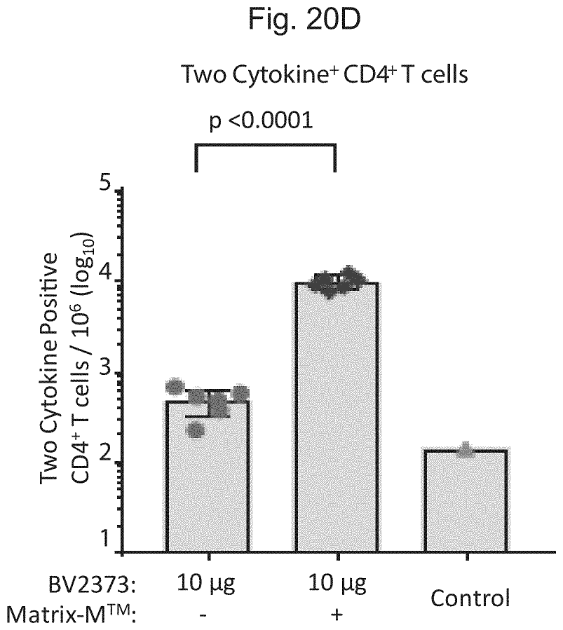

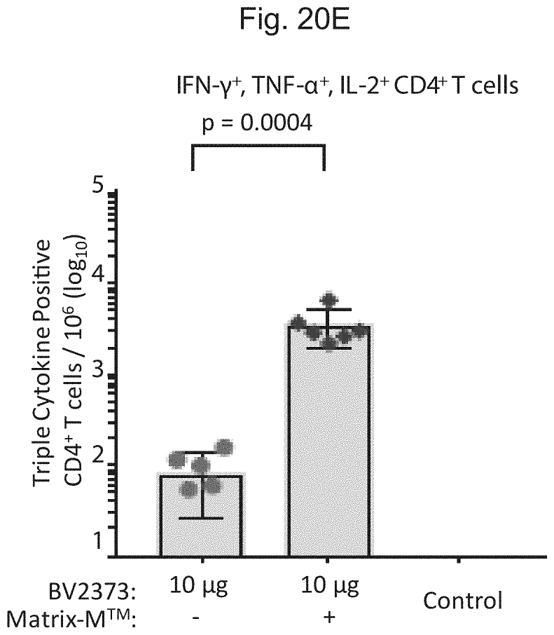

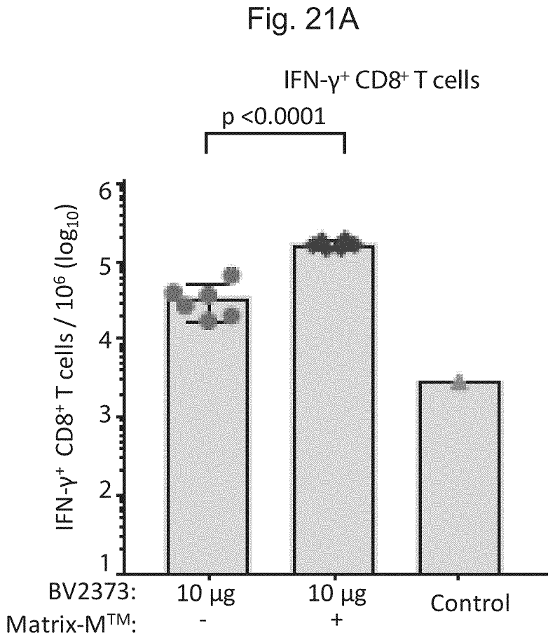

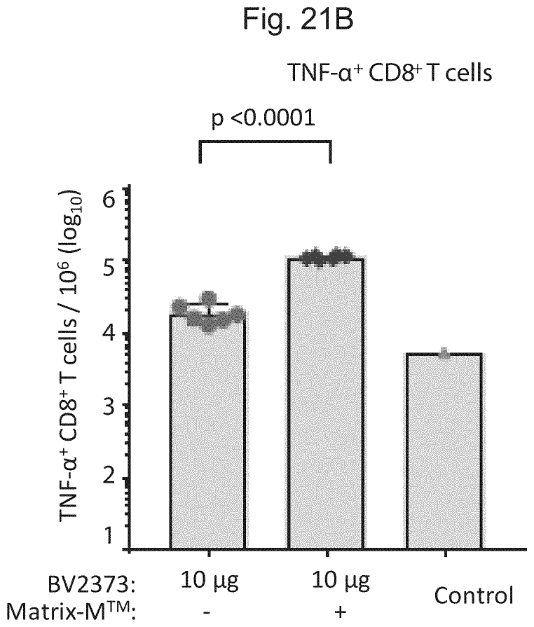

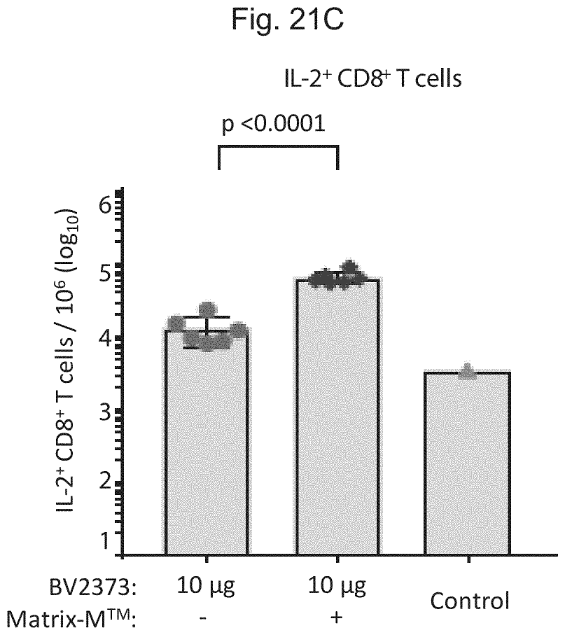

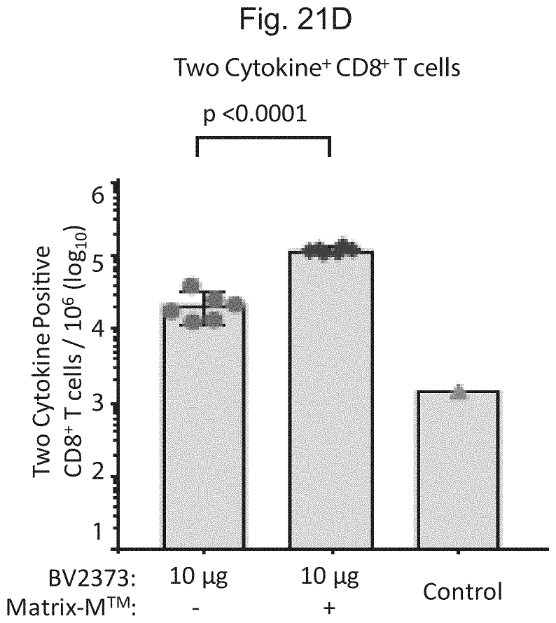

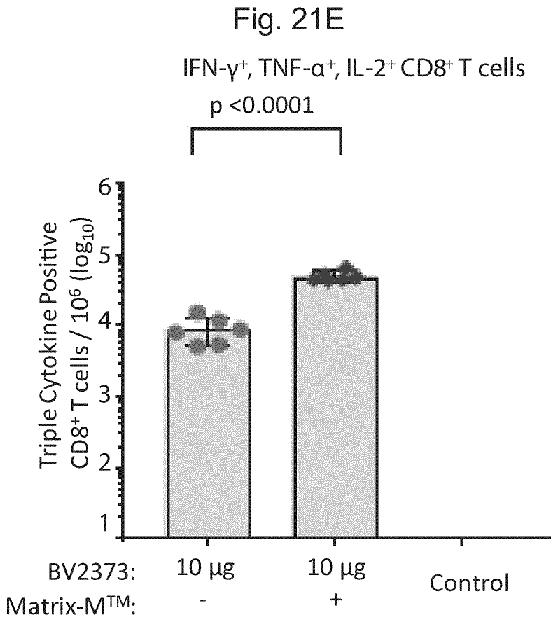

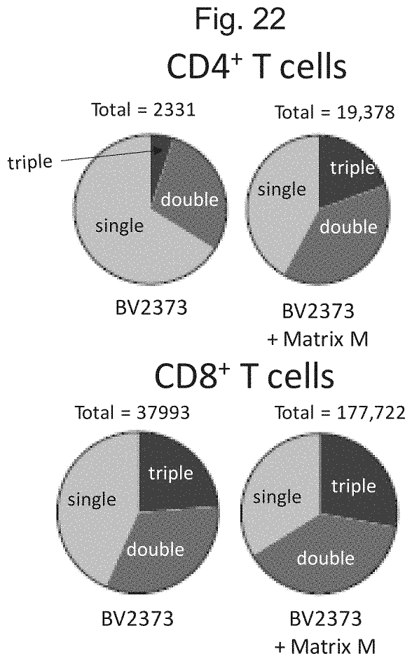

Antigen-specific T cell responses were measured by ELISPOT™ enzyme linked immunosorbent assay and intracellular cytokine staining (ICCS) from spleens collected 7-days after the second immunization (study day 28). The number of IFN-γ secreting cells after ex vivo stimulation increased 20-fold (p=0.002) in spleens of mice immunized with BV2373 and MATRIX-M™ compared to BV2373 alone as measured by the ELISPOT™ assay (FIG. 19). In order to examine CD4+ and CD8+ T cell responses separately, ICCS assays were performed in combination with surface marker staining. Data shown are gated on CD44hi CD62L− effector memory T cell population. The frequency of IFN-γ+, TNF-α+, and IL-2+ cytokine-secreting CD4+ and CD8+ T cells was significantly higher (p<0.0001) in spleens from mice immunized with BV2373 as compared to mice immunized without adjuvant (FIG. 20A-C and FIG. 21A-C). Further, the frequency of multifunctional CD4+ and CD8+ T cells, which simultaneously produce at least two or three cytokines was also significantly increased (p<0.0001) in spleens from the BV2373/MATRIX-M™ immunized mice as compared to mice immunized in the absence of adjuvant (FIGS. 20D-E and FIGS. 21D-E). Immunization with BV2373/MATRIX-M™ resulted in higher proportions of multifunctional phenotypes (e.g., T cells that secrete more than one of IFN-γ, TNF-α, and IL-2) within both CD4+ and CD8+ T cell populations. The proportions of multifunctional phenotypes detected in memory CD4+ T cells were higher than those in CD8+ T cells (FIG. 22).

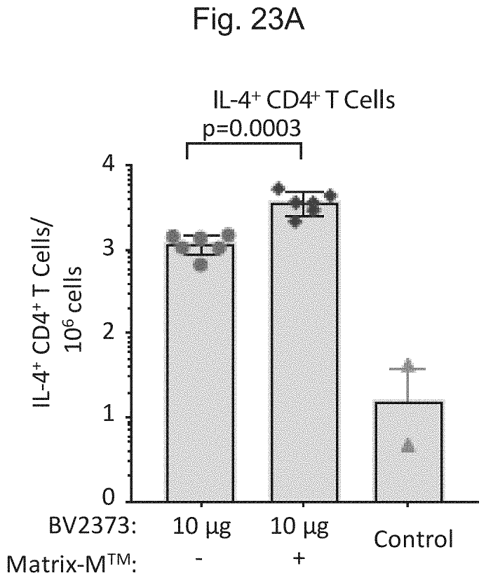

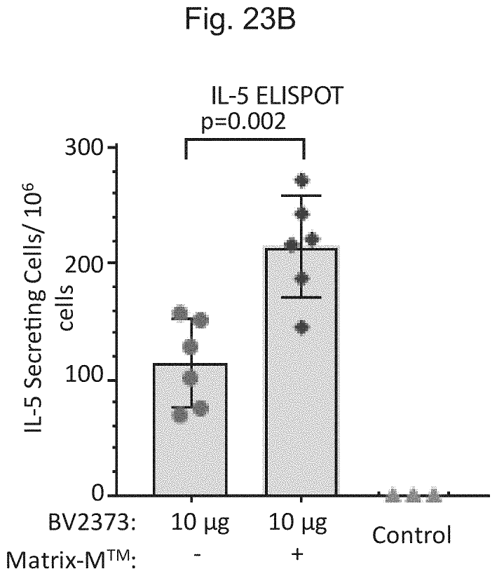

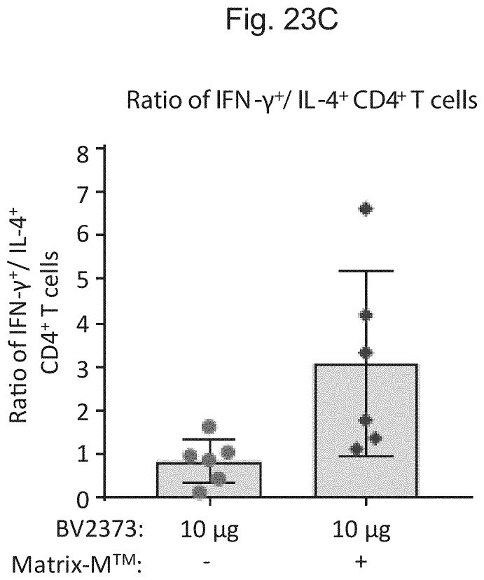

Type 2 cytokine IL-4 and IL-5 secretion from CD4+ T cells was also determined by ICCS and ELISPOT™ respectively. Immunization with BV2373/MATRIX-M™ also increased type 2 cytokine IL-4 and IL-5 secretion (2-fold) compared to immunization with BV2373 alone, but to a lesser degree than enhancement of type 1 cytokine production (e.g. IFN-γ increased 20-fold) (FIGS. 23A-C). These results indicate that administration of the MATRIX-M™ adjuvant skewed the CD4+ T cell development toward Th1 responses.

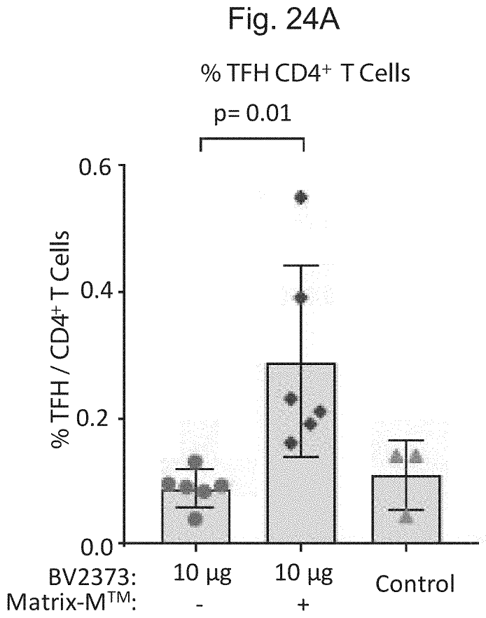

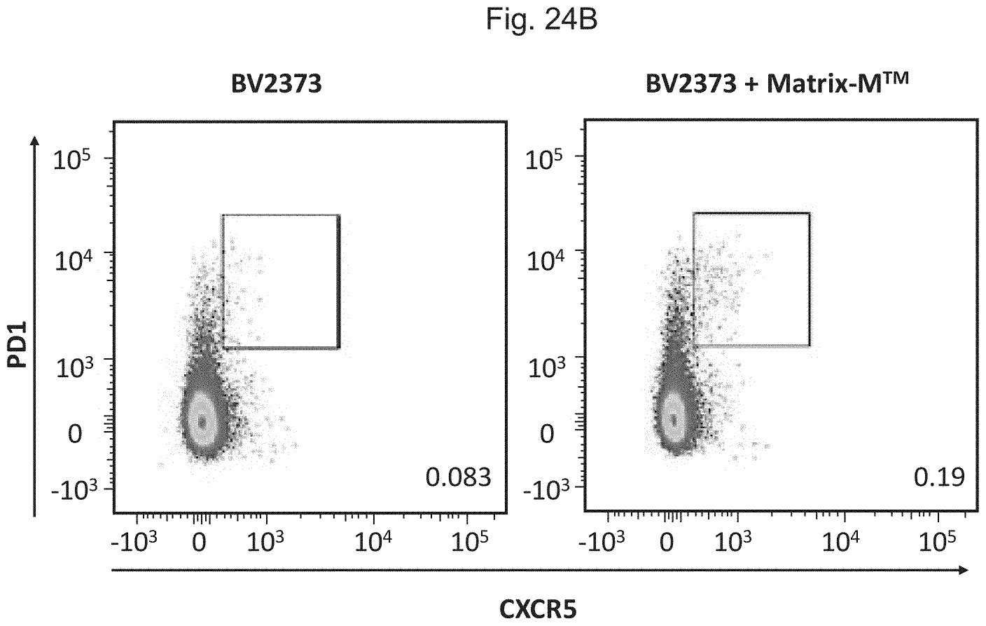

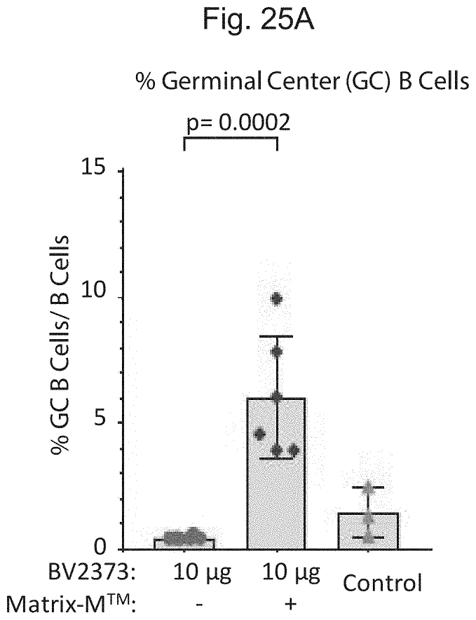

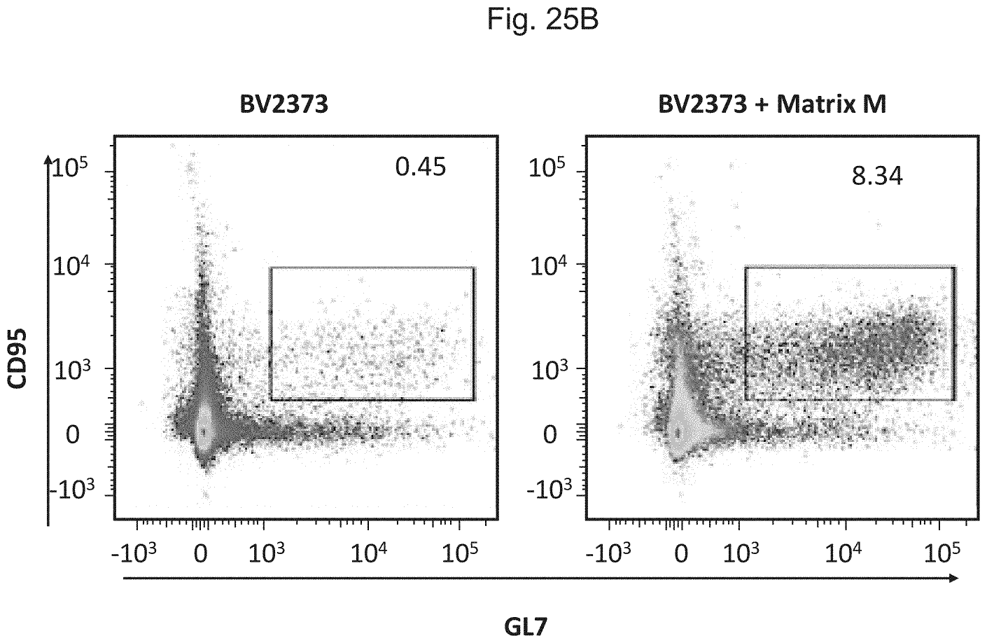

The effect of immunization on germinal center formation was assessed by measuring the frequency of CD4+ T follicular helper (TFH) cells and germinal center (GC) B cells in spleens. MATRIX-M™ administration significantly increased the frequency of TFH cells (CD4+ CXCR5+ PD-1+) was significantly increased (p=0.01), as well as the frequency of GC B cells (CD19+GL7+CD95+) (p=0.0002) in spleens (FIGS. 24A-B and FIGS. 25A-B).

### Example 3

### Immunogenicity of Coronavirus Spike (S) Polypeptide Nanoparticle Vaccines in Olive Baboons

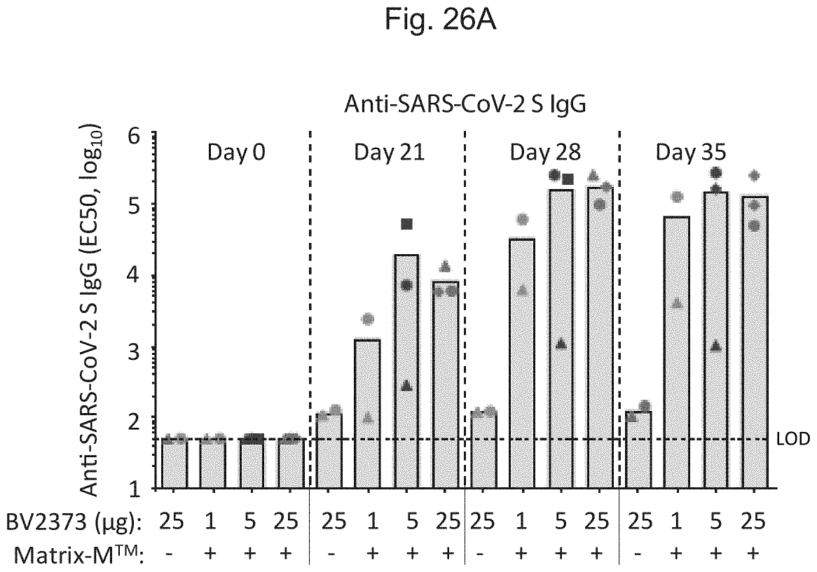

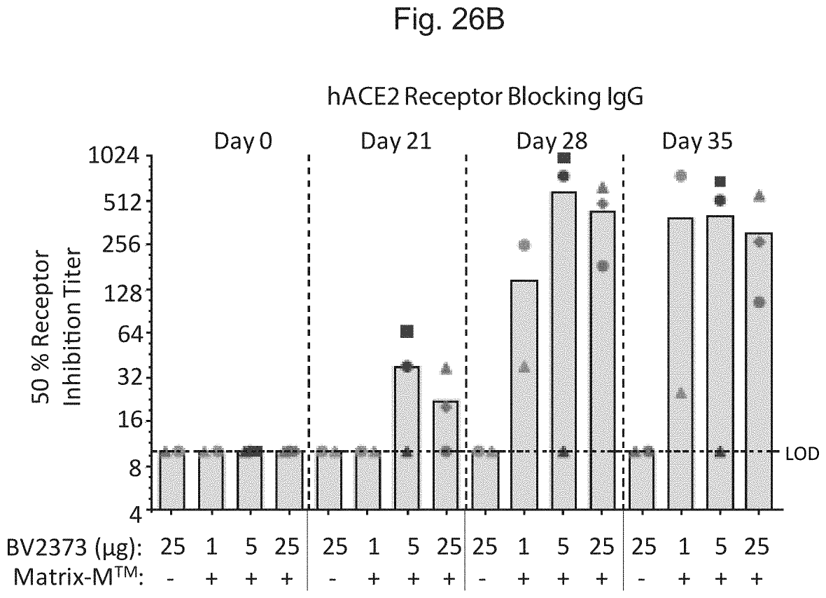

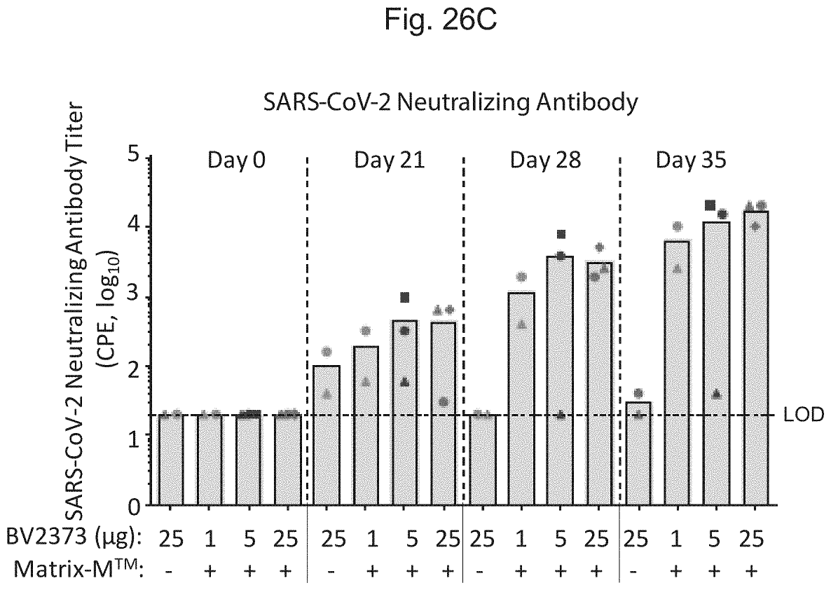

The immunogenicity of a vaccine composition comprising BV2373 in baboons was assessed. Adult olive baboons were immunized with a dose range (1 μg, 5 μg and 25 μg) of BV2373 and 50 μg MATRIX-M™ adjuvant administered by intramuscular (IM) injection in two doses spaced 21-days apart. To assess the adjuvanting activity of MATRIX-M™ in non-human primates, another group of animals was immunized with 25 μg of BV2373 without MATRIX-M™. Anti-S protein IgG titers were detected within 21-days of a single priming immunization in animals immunized with BV2373/MATRIX-M™ across all the dose levels (GMT=1249-19,000). Anti-S protein IgG titers increased over a log (GMT=33,000-174,000) within 1 to 2 weeks following a booster immunization (days 28 and 35) across all of the dose levels. (FIG. 26A).

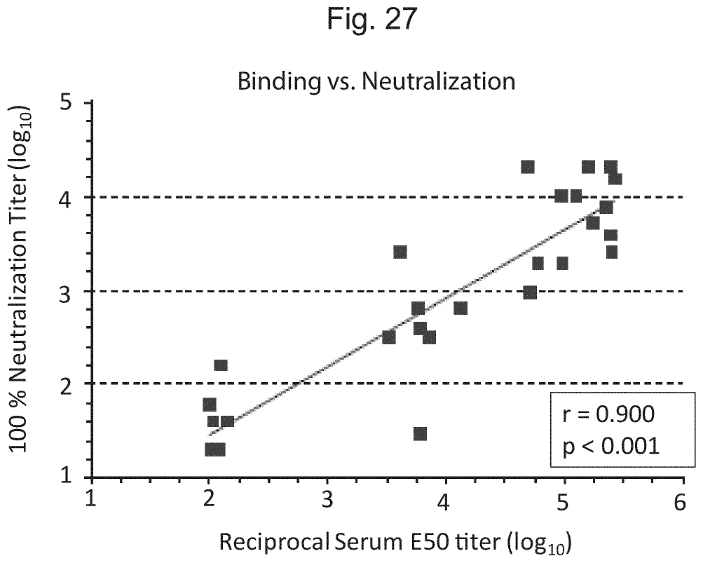

Low levels of hACE2 receptor blocking antibodies were detected in animals following a single immunization with BV2373 (5 μg or 25 μg) and MATRIX-M™ (GMT=22-37). Receptor blocking antibody titers were significantly increased within one to two weeks of the booster immunization across all groups immunized with BV2373/MATRIX-M™ (GMT=150-600) (FIG. 26B). Virus neutralizing antibodies were elevated (GMT=190-446) across all dose groups after a single immunization with BV2373/MATRIX-M™. Animals immunized with 25 μg BV2373 alone had no detectable antibodies that block S-protein binding to hACE2 (FIG. 26C). Neutralizing titers were increased 6- to 8-fold one week following the booster immunization (GMT=1160-3846). Neutralizing titers increased an additional 25- to 38-fold following the second immunization (GMT=6400-17,000) (FIG. 26C). There was a significant correlation (p<0.0001) between anti-S IgG levels and neutralizing antibody titers (FIG. 27). The immunogenicity of the adjuvanted vaccine in nonhuman primates is consistent with the results of Example 2 and further supports the role of MATRIX-M™ in promoting the generation of neutralizing antibodies and dose sparing.

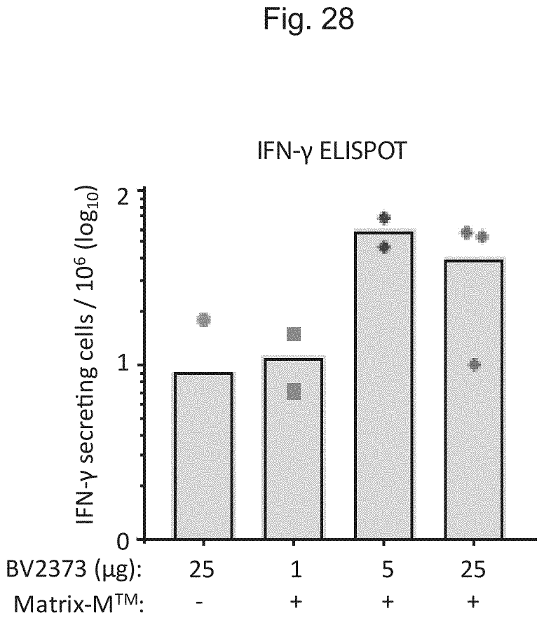

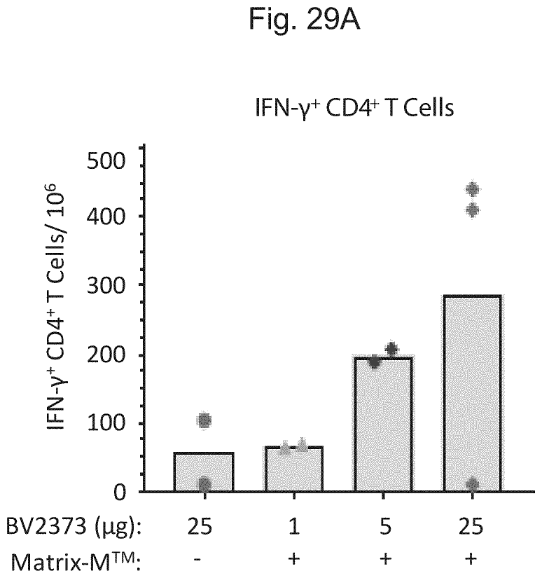

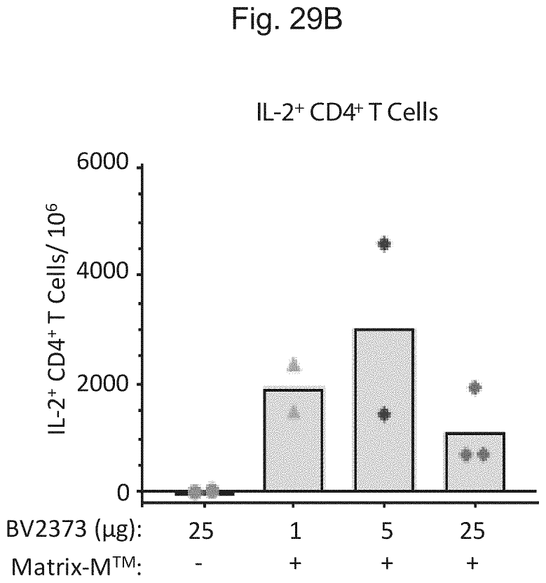

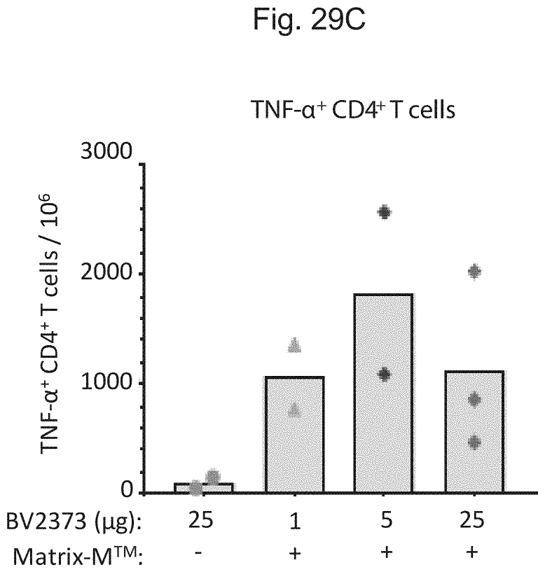

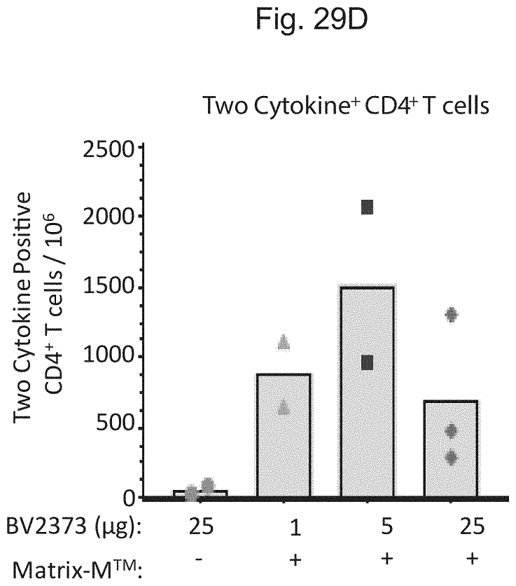

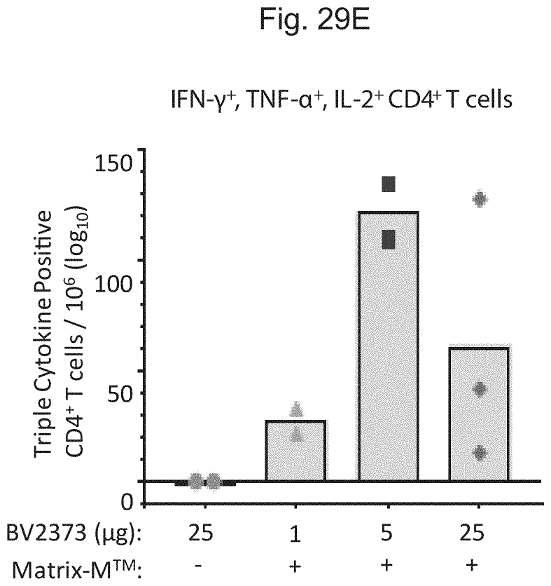

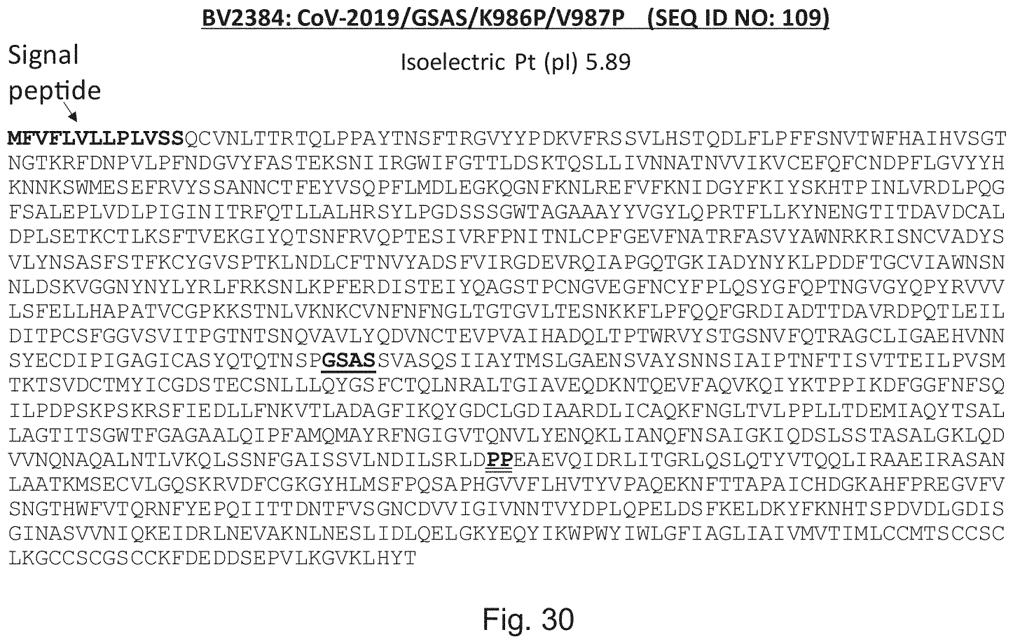

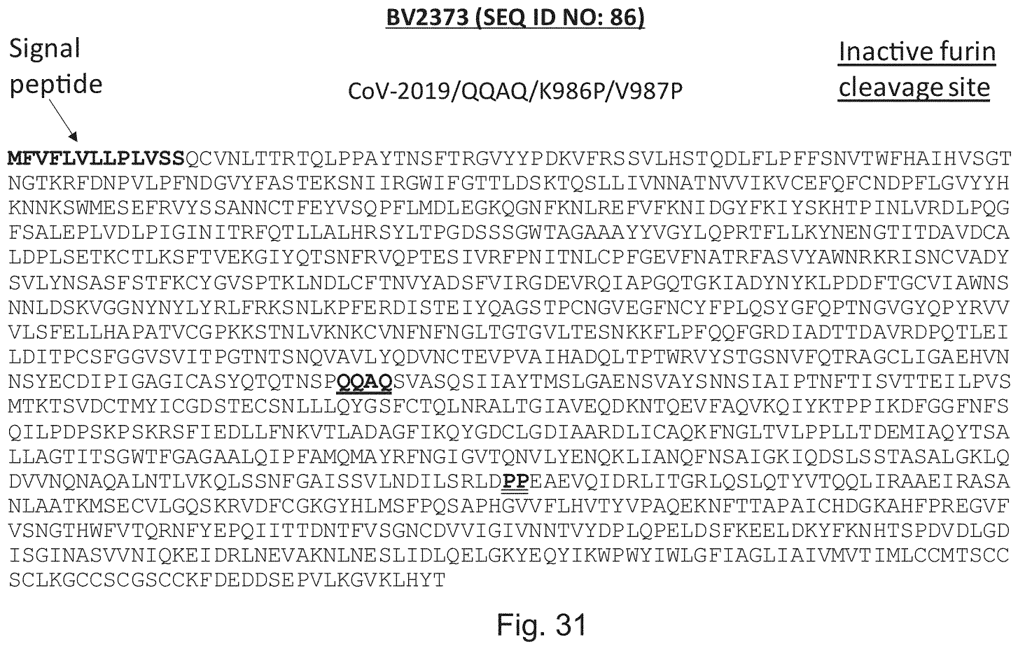

PBMCs were collected 7 days after the second immunization (day 28), and the T cell response was measured by ELISPOT assay. PBMCs from animals immunized with BV2373 (5 μg or 25 μg) and MATRIX-M™ had the highest number of IFN-γ secreting cells, which was 5-fold greater compared to animals immunized with 25 μg BV2373 alone or BV2373 (1 μg) and MATRIX-M™ (FIG. 28). By ICCS analysis, immunization with BV2373 (5 μg) and MATRIX-M™ showed the highest frequency of IFN-γ+, IL-2+, and TNF-α+ CD4+ T cells (FIGS. 29A-C). This trend was also true for multifunctional CD4+ T cells, in which at least two or three type 1 cytokines were produced simultaneously (FIGS. 29D-E).

### Example 4

### Structural Characterization of Coronavirus Spike (S) Polypeptide Nanoparticle Vaccines

Transmission electron microscopy (TEM) and two dimensional (2D) class averaging were used to determine the ultrastructure of BV2373. High magnification (67,000× and 100,000×) TEM images of negatively stained BV2373 showed particles corresponding to S-protein homotrimers.

An automated picking protocol was used to construct 2D class average images (Lander G. C. et al. J Struct Biol. 166, 95-102 (2009); Sorzano C. O. et al., J Struct Biol. 148, 194-204 (2004).). Two rounds of 2D class averaging of homotrimeric structures revealed a triangular particle appearance with a 15 nm length and 13 nm width (FIG. 10, top left). Overlaying the recently solved cryoEM structure of the SARS-CoV-2 spike protein (EMD ID: 21374) over the 2D BV2373 image showed a good fit with the crown-shaped S1 (NTD and RBD) and the S2 stem (FIG. 10, bottom left). Also apparent in the 2D images was a faint projection that protruded from the tip of the trimeric structure opposite of the NTD/RBD crown (FIG. 10, top right). 2D class averaging using a larger box size showed these faint projections form a connection between the S-trimer and an amorphous structure. (FIG. 10, bottom right).

Dynamic light scattering (DLS) show that the wild-type CoV S protein had a Z-avg particle diameter of 69.53 nm compared to a 2-fold smaller particle size of BV2365 (33.4 nm) and BV2373 (27.2 nm). The polydispersity index (PDI) indicated that BV2365 and BV2373 particles were generally uniform in size, shape, and mass (PDI=0.25-0.29) compared to the wild-type spike-protein (PDI=0.46) (Table 3).

TABLE 3
Particle Size and Thermostability of
SARS-CoV-2 Trimeric Spike Proteins

| | Differential Scanning | | Dynamic Light Scattering | |
|---|---|---|---|---|
| | Calorimetry | | (DLS) | |
| | (DSC) | | Z- avg | |
| SARS-CoV-2 S | Tₘₐₓ | ΔHcal | diameter² | |
| protein | (° C.)¹ | (kJ/mol) | (nm) | PDI³ |
| Wild-type | 58.6 | 153 | 69.53 | 0.46 |
| BV2365 | 61.3 | 466 | 33.40 | 0.25 |
| BV2373 | 60.4 | 732 | 27.21 | 0.29 |

¹Tₘₐₓ: melting temperature
²Z-avg: Z-average particle size
³PDI: polydispersity index

The thermal stability of the S-trimers was determined by differential scanning calorimetry (DSC). The thermal transition temperature of the wild-type CoV S-protein (Tₘₐₓ=58.6° C.) was similar to BV2365 and BV2373 with a Tₘₐₓ=61.3° C. and 60.4° C., respectively (Table 3). Of greater significance, was the 3-5 fold increased enthalpy of transition required to unfold the BV2365 and BV2373 variants (ΔHcal=466 and 732 kJ/mol, respectively) compared to the lower enthalpy required to unfold the WT spike protein (ΔHcal=153 kJ/mol). These results are consistent with improved thermal stability of the BV2365 and BV2373 compared to that of WT spike protein (Table 3).

The stability of the CoV Spike (S) polypeptide nanoparticle vaccines was evaluated by dynamic light scattering. Various pHs, temperatures, salt concentrations, and proteases were used to compare the stability of the CoV Spike (S) polypeptide nanoparticle vaccines to nanoparticle vaccines containing the native CoV Spike (S) polypeptide.

### Example 5

### Stability of Coronavirus Spike (S) Polypeptide Nanoparticle Vaccines

The stability of the CoV Spike (S) polypeptide nanoparticle vaccines was evaluated by dynamic light scattering. Various pHs, temperatures, salt concentrations, and proteases were used to compare the stability of the CoV Spike (S) polypeptide nanoparticle vaccines to nanoparticle vaccines containing the native CoV Spike (S) polypeptide. The stability of BV2365 without the 2-proline substitutions and BV2373 with two prolines substitution was assessed under different environmental stress conditions using the hACE2 capture ELISA. Incubation of BV2373 at pH extremes (48 hours at pH 4 and pH 9), with prolonged agitation (48 hours), and through freeze/thaw (2 cycles), and elevated temperature (48 hours at 25° C. and 37° C.) had no effect on hACE2 receptor binding (IC50=14.0-18.3 ng mL-1).

Oxidizing conditions with hydrogen peroxide reduced binding of hACE2 binding to BV2373 8-fold (IC50=120 ng mL-1) (FIG. 12A). BV2365 without the 2-proline substitutions was less stable as determined by a significant loss of hACE2 binding under multiple conditions (FIG. 12B).

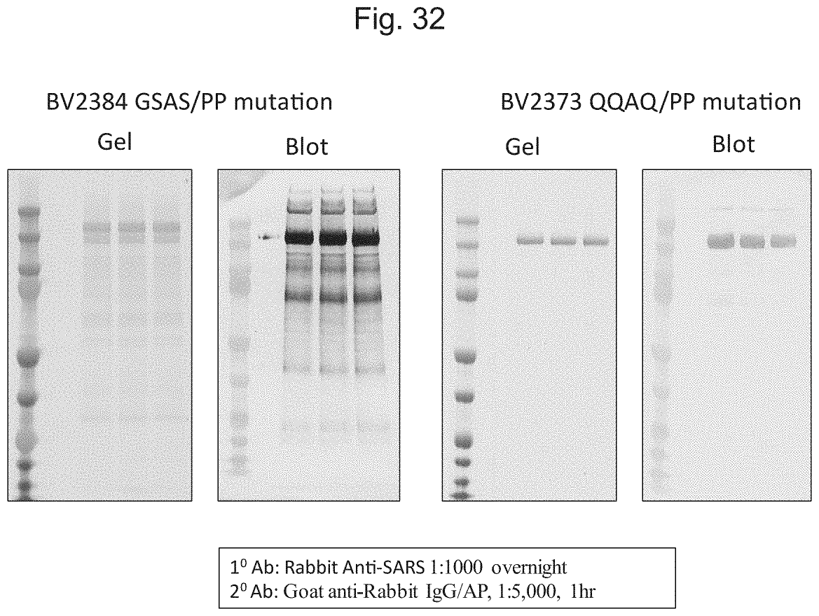

The stability of BV2384 (SEQ ID NO: 110) and BV2373 (SEQ ID NO: 87) were compared. BV2384 has a furin cleavage site sequence of GSAS (SEQ ID NO: 97), whereas BV2373 has a furin cleavage site of QQAQ (SEQ ID NO: 7). As demonstrated by SDS-PAGE and Western Blot, BV2384 showed extensive degradation in comparison to BV2373 (FIG. 32). Furthermore, scanning densitometry and recovery data demonstrate the unexpected loss of full length CoV S protein BV2384, lower purity, and recovery (FIG. 33) in comparison to BV2373 (FIG. 34).

### Example 6

### Immune Response in Cynomolgus Macaques

We assessed the immune response induced by BV2373 in a Cynomolgus macaque model of SARS-CoV-2 infection. Groups 1-6 were treated as shown in Table 4.

TABLE 4
Groups 1-6 of Cynomolgus macaque study

| | | | | Immuni- | Blood | |
|---|---|---|---|---|---|---|
| Group | BV2373 | | MATRIX-M ™ | zation | Draw | Challenge |
| (N = 4) | Dose | | Dose | (Days) | (days) | (Day) |
| 1 | Placebo | | — | 0, 21 | 0, 21, 33 | 35 |
| 2 | 2.5 | μg | 25 μg | 0, 21 | 0, 21, 33 | 35 |
| 3 | 5 | μg | 25 μg | 0 | 0, 21, 33 | 35 |
| 4 | 5 | μg | 50 μg | 0, 21 | 0, 21, 33 | 35 |
| 5 | 5 | μg | 50 μg | 0 | 0, 21, 33 | 35 |
| 6 | 25 | μg | 50 μg | 0, 21 | 0, 21, 33 | 35 |

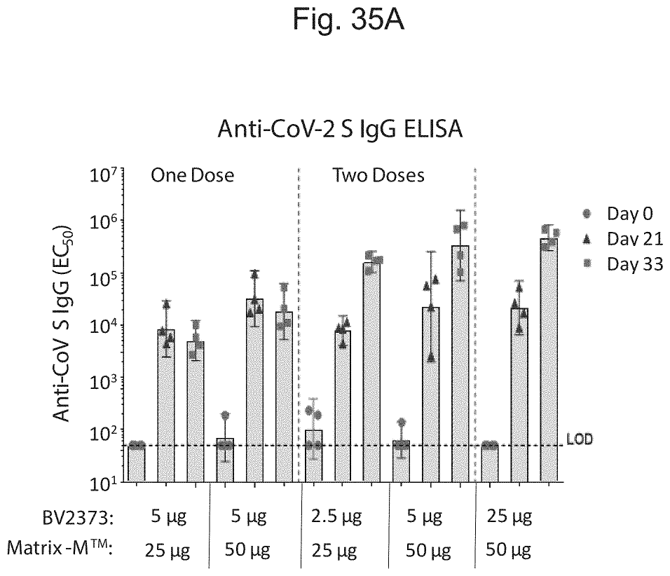

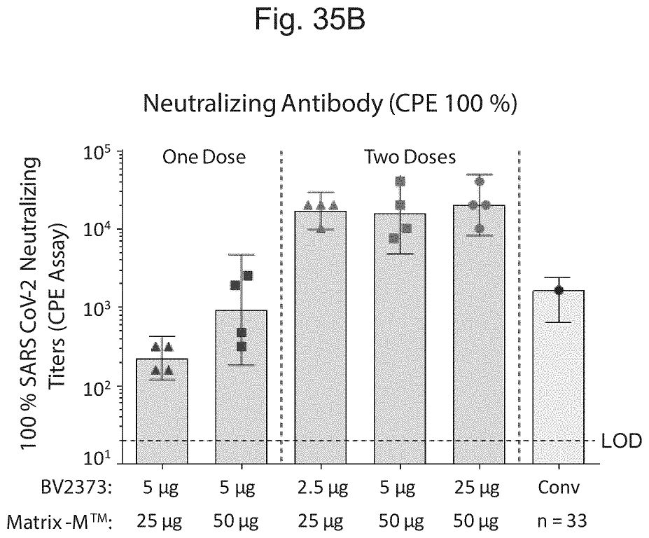

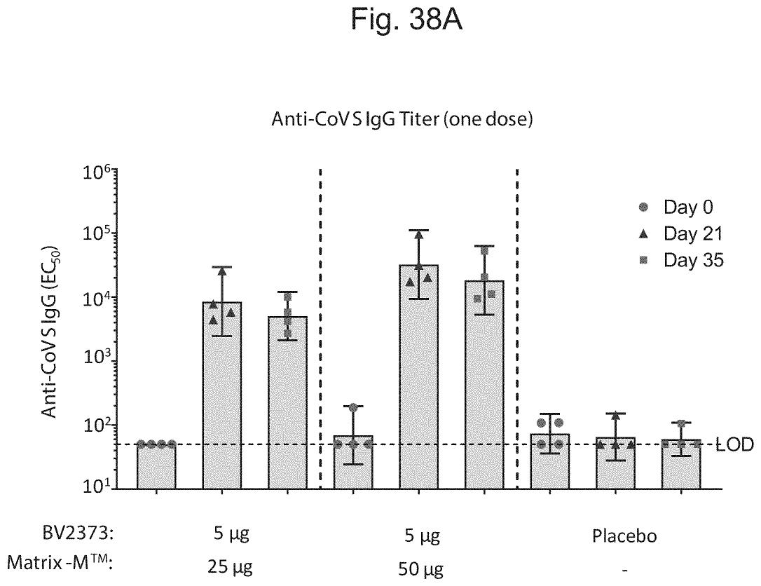

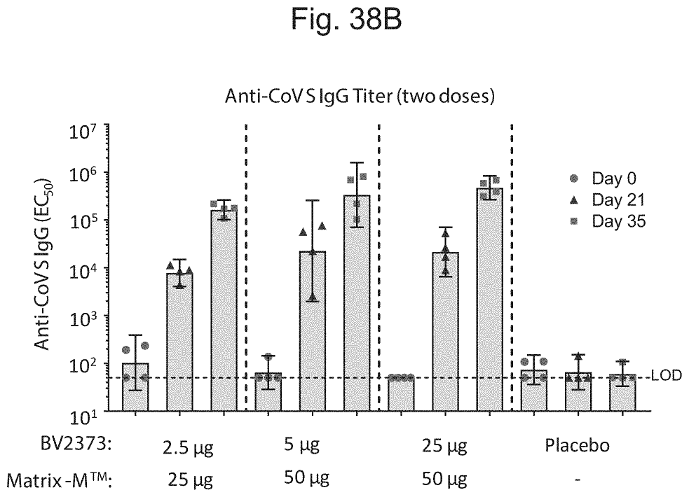

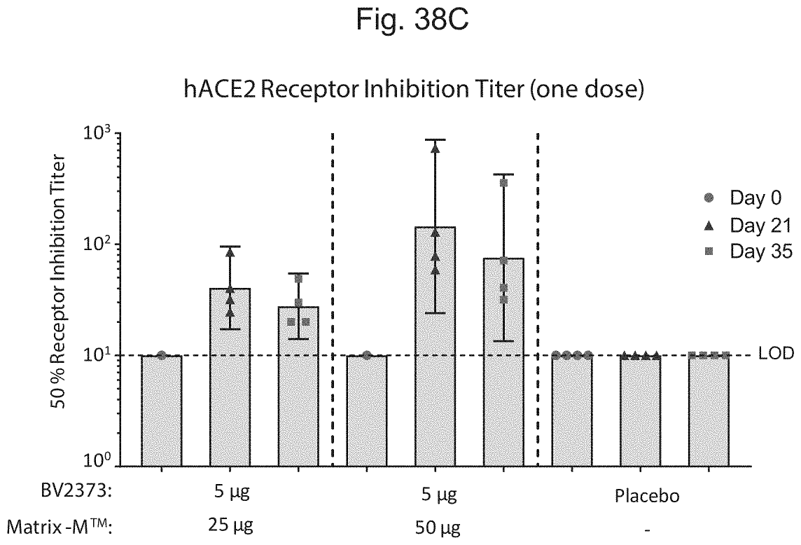

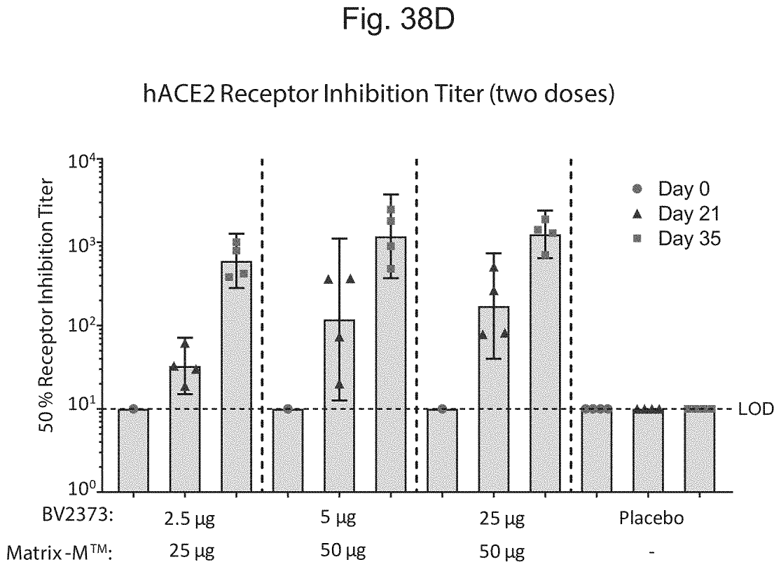

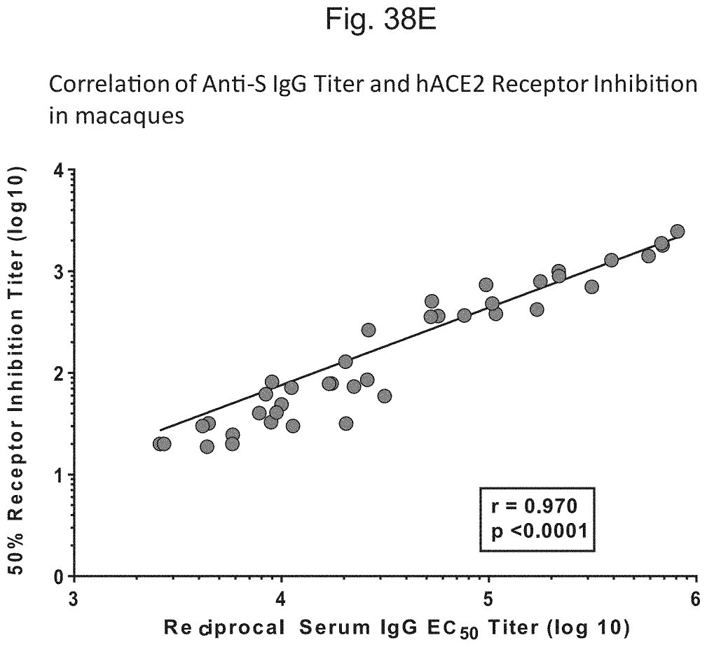

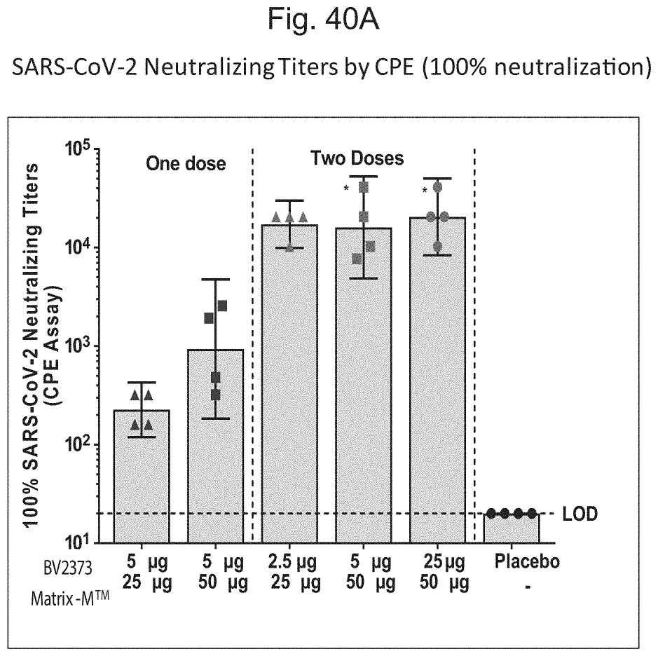

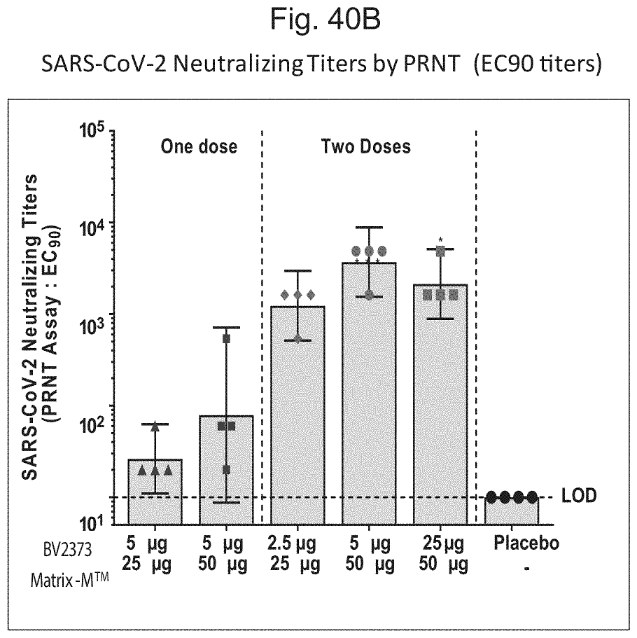

Administration of a vaccine comprising BV2373 resulted in the induction of anti-CoV-S antibodies (FIG. 35A) including neutralizing antibodies (FIG. 35B). Anti-CoV-S antibodies were induced after administration of one (FIG. 38A) or two doses (FIG. 38B) of BV2373. Administration of the vaccine comprising BV2373 also resulted in the production of antibodies that blocked binding of the CoV S protein to hACE2 (FIG. 38C and FIG. 38D). There was a significant correlation between anti-CoV S polypeptide IgG titer and hACE2 inhibition titer in Cynomolgus macaques after administration of BV2373 (FIG. 38E). The ability of BV2373 to induce the production of neutralizing antibodies was evaluated by cytopathic effect (CPE) (FIG. 40A) and plaque reduction neutralization test (PRNT) (FIG. 40B). The data revealed that vaccine formulations of Table 4 produced SARS-CoV-2 neutralizing titers, in contrast to the control.

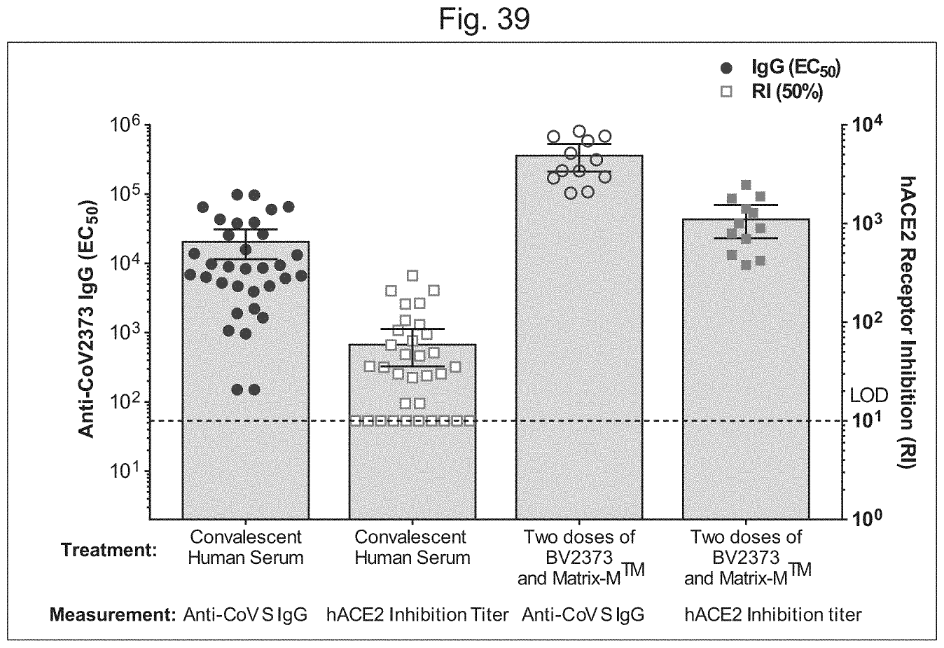

The vaccine comprising BV2373's ability to induce anti-CoV-S antibodies and antibodies that block binding of hACE2 to the CoV S protein in Cynomolgus macaques was compared to human convalescent serum. The data revealed that the BV2373 vaccine formulation induced superior anti-CoV S polypeptide and hACE2 inhibition titers as compared to human convalescent serum (FIG. 39).

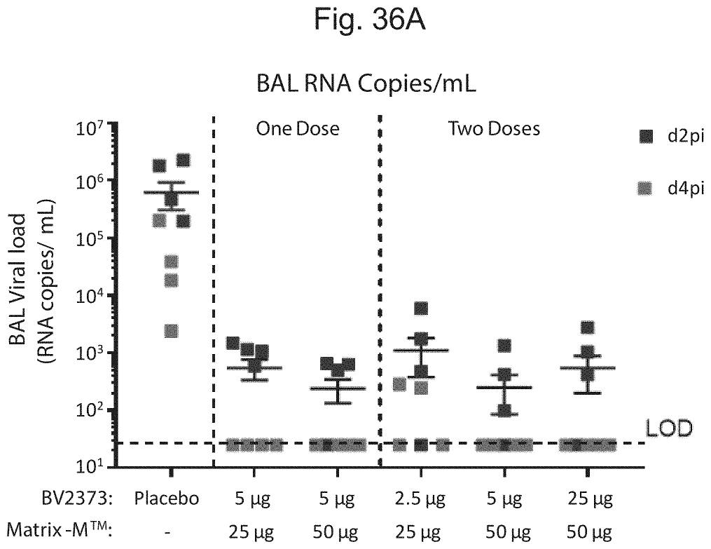

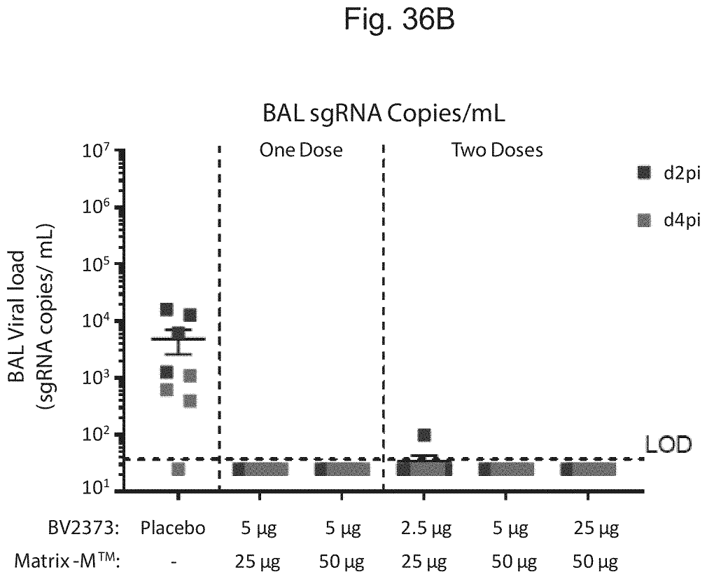

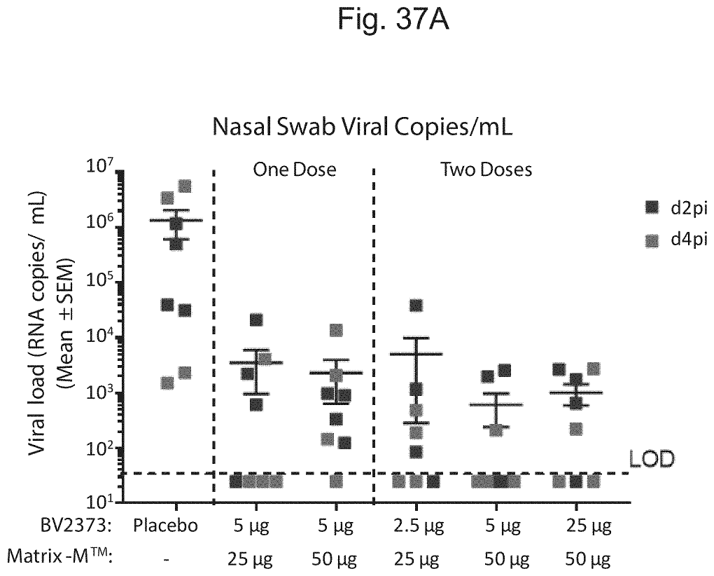

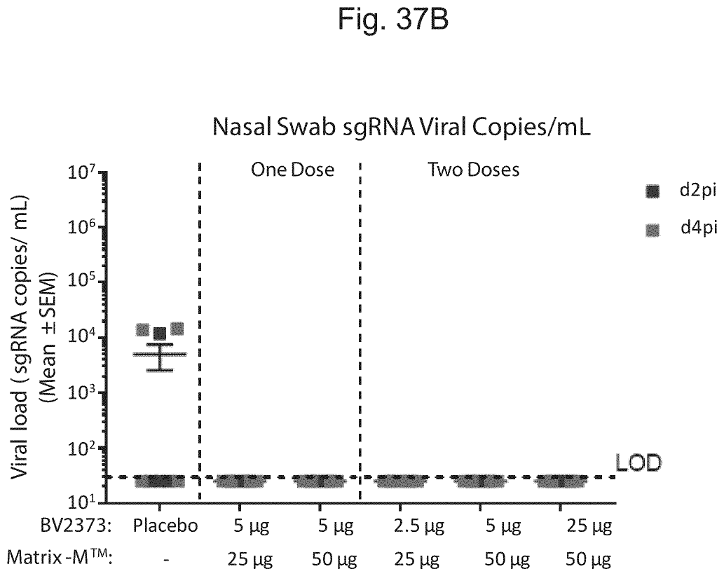

The BV2373 vaccine formulation also caused a decrease of SARS-CoV-2 viral replication (FIGS. 36A-B). Viral RNA (FIG. 36A, corresponding to total RNA present) and viral sub-genomic RNA (sgRNA) (FIG. 36B, corresponding to replicating virus) levels were assessed in bronchiolar lavage (BAL) at 2 days and 4 days post-challenge with infectious virus (d2pi and d4pi). Most subjects showed no viral RNA. At Day 2 small amounts of RNA were measured in some subjects. By Day 4, no RNA was measured except for two subjects at the lowest dose of 2.5 μg. Sub-genomic RNA was not detected at either 2 days or 4 days except for 1 subject, again at the lowest dose. Viral RNA (FIG. 37A) and viral sub-genomic (sg) RNA (FIG. 37B) were assessed by nasal swab at 2 days and 4 days post-infection (d2pi and d4pi). Most subjects showed no viral RNA. At Day 2 and Day 4 small amounts of RNA were measured in some subjects. Sub-genomic RNA was not detected at either 2 Days or 4 days. Subjects were immunized. Day 0 and in the groups with two doses Day 0 and Day 21. These data show that the vaccine decreases nose total virus RNA by 100-1000 fold and sgRNA to undetectable levels, and confirm that immune response to the vaccine will block viral replication and prevent viral spread.

### Example 7

### Evaluation of CoV S Polypeptide Nanoparticle Vaccines in Humans

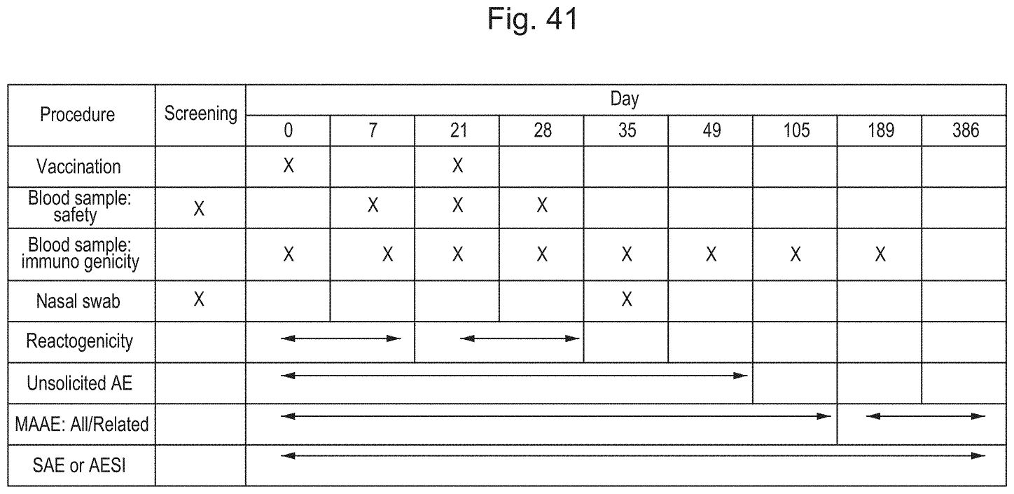

We assessed the safety and efficacy of a vaccine comprising BV2373 in a randomized, observer-blinded, placebo-controlled Phase 1 clinical trial in 131 healthy participants 18-59 years of age. Participants were immunized with two intramuscular injections, 21 days apart. Participants received BV2373 with or without MATRIX-M™ (n=106) or placebo (n=25). Groups A-E were treated as shown in Table 5. FIG. 41 shows a timeline of the evaluation of clinical endpoints.

TABLE 5
Groups A-E of Phase 1 Human Study

| | | | Day 0 | | | | Day 21 (+5 days) | | | |
|---|---|---|---|---|---|---|---|---|---|---|
| Group | Participants | | BV2373 | | MATRIX-M^{TM} | | BV2373 | | MATRIX-M^{TM} | |
| (N = 25) | Randomized | Sentinel | Dose | | Dose | | Dose | | Dose | |
| A | 25 | — | 0 | μg | 0 | μg | 0 | μg | 0 | μg |
| B | 25 | — | 25 | μg | 0 | μg | 25 | μg | 0 | μg |
| C | 25 | 3 | 5 | μg | 50 | μg | 5 | μg | 50 | μg |
| D | 25 | 3 | 25 | μg | 50 | μg | 25 | μg | 50 | μg |
| E | 25 | — | 25 | μg | 50 | μg | 0 | μg | 0 | μg |

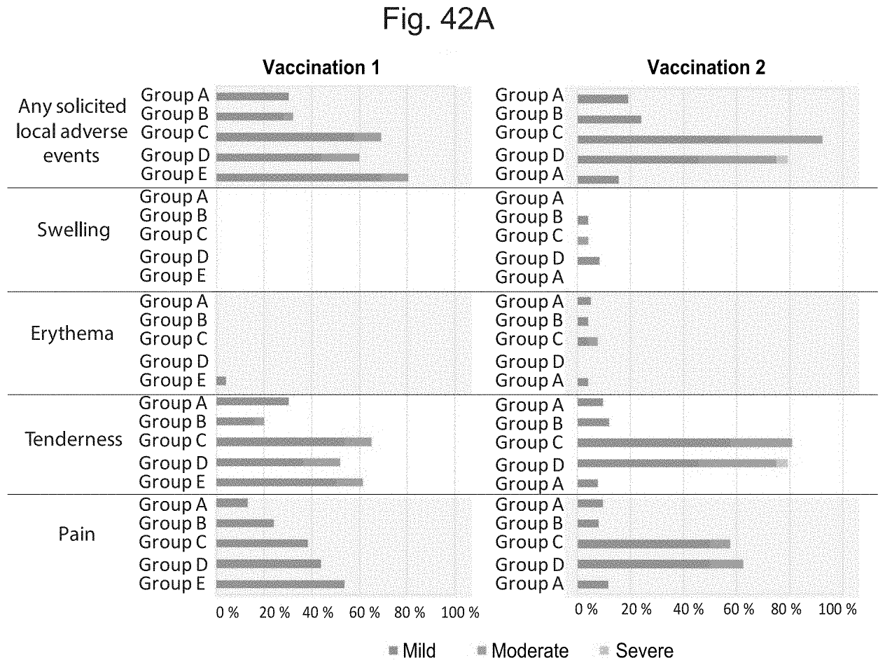

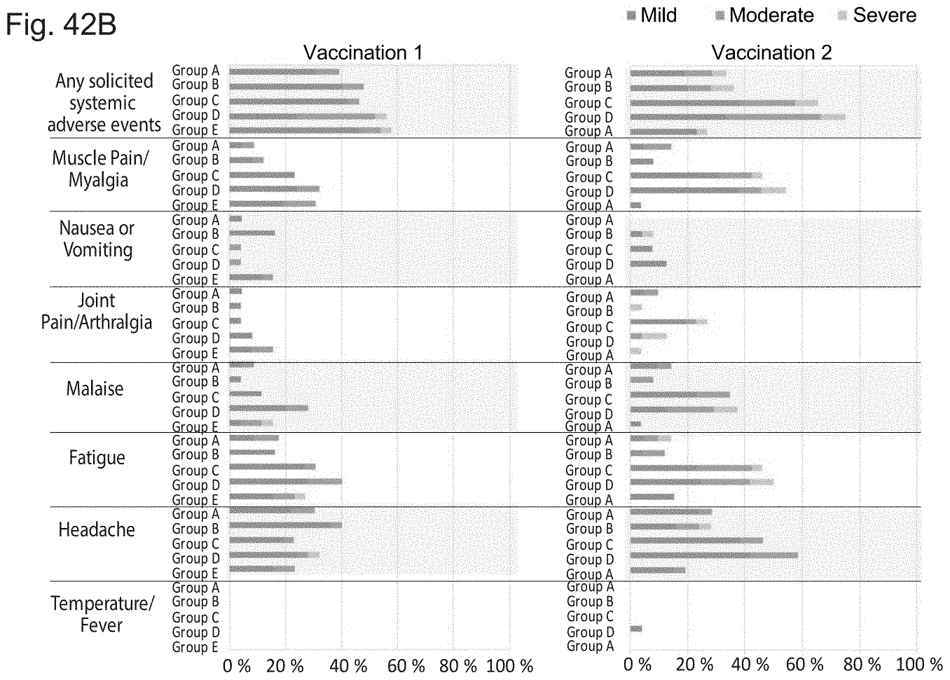

Overall reactogenicity was mild, and the vaccinations were well tolerated. Local reactogenicity was more frequent in patients treated with BV2373 and MATRIX-M™ (FIGS. 42A-B).

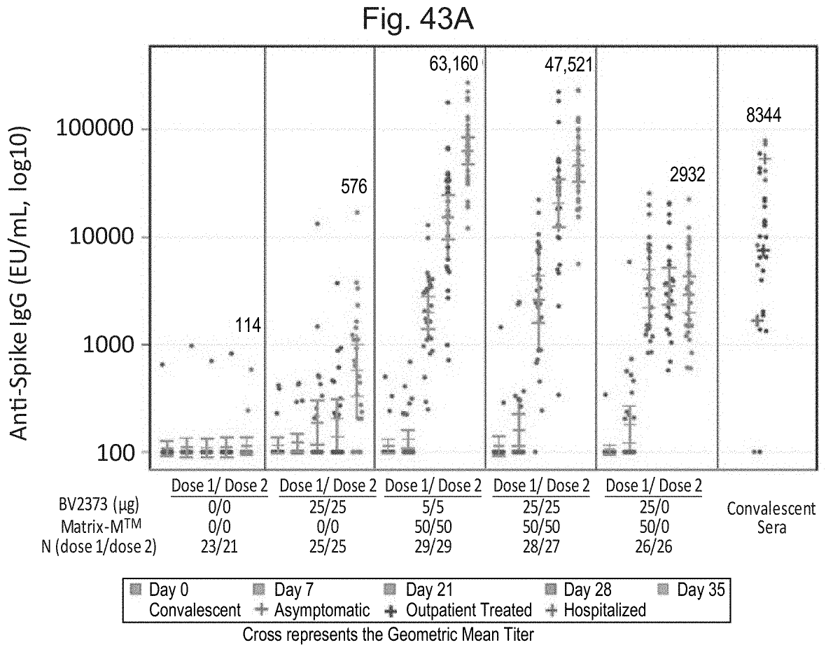

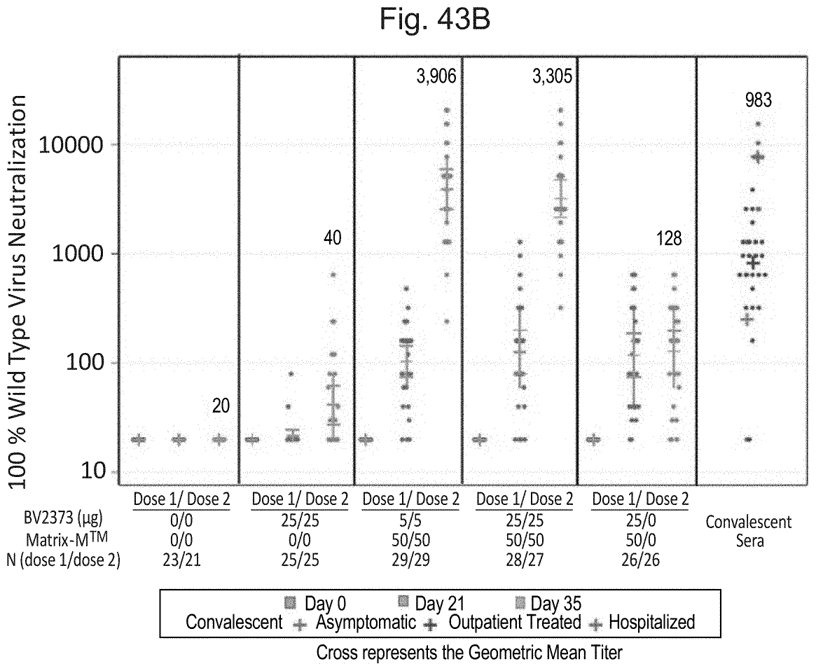

The immunogenicity of BV2373 with and without MATRIX-M™ was evaluated. 21 days after vaccination, anti-CoV-S antibodies were detected for all vaccine regimens (FIG. 43A). Geometric mean fold rises (GMFR) in vaccine regimens comprising MATRIX-M™ exceeded those induced by unadjuvanted BV2373. 7 days after a second vaccination (day 28), the anti-CoV-S titer increased an additional eight-fold over responses seen with first vaccination and within 14 days (Day 35) responses had more than doubled yet again, achieving GMFRs approximately 100-fold over those observed with BV2373 alone. A single vaccination with BV2373/MATRIX-M™ achieved similar anti-CoV-S titer levels to those in asymptomatic (exposed) COVID-19 patients. A second vaccination achieved GMEU levels that exceeded convalescent serum from outpatient-treated COVID-19 patients by six-fold, achieved levels similar to convalescent serum from patients hospitalized with COVID-19, and exceeded overall convalescent serum anti-CoV-S antibodies by nearly six-fold. The responses in the two-dose 5-μg and 25-μg BV2373/MATRIX-M™ regimens were similar. This highlights the ability of the adjuvant (MATRIX-M™) to enable dose sparing.

Neutralizing antibodies were induced in all groups treated with BV2373 (FIG. 43B). Groups treated with BV2373 and MATRIX-M™ regimens exhibited an approximately five-fold GMFR than groups treated with BV2373 alone (FIG. 43B). Second vaccinations with adjuvant had a profound effect on neutralizing antibody titers—inducing >100 fold rise over single vaccinations without adjuvant. When compared to convalescent serum, second vaccinations with BV2373/MATRIX-M™ achieved GMT levels four-fold greater than outpatient-treated COVID-19 patients, levels spanning those of patients hospitalized with COVID-19, and exceeded overall convalescent serum GMT by four fold.

Convalescent serum, obtained from COVID-19 patients with clinical symptoms requiring medical care, demonstrated proportional anti-CoV-S IgG and neutralization titers that increased with illness severity (FIGS. 43A-B).

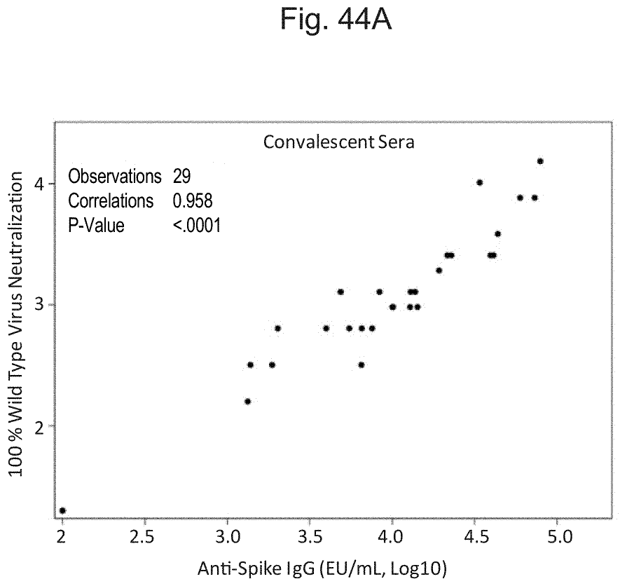

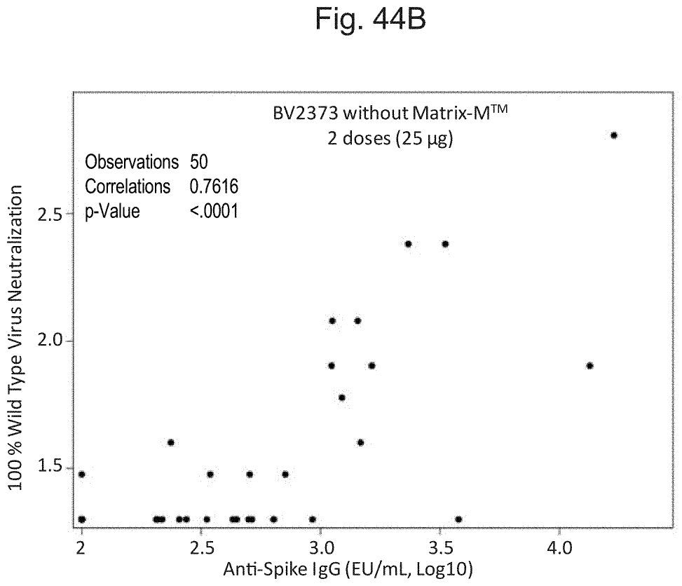

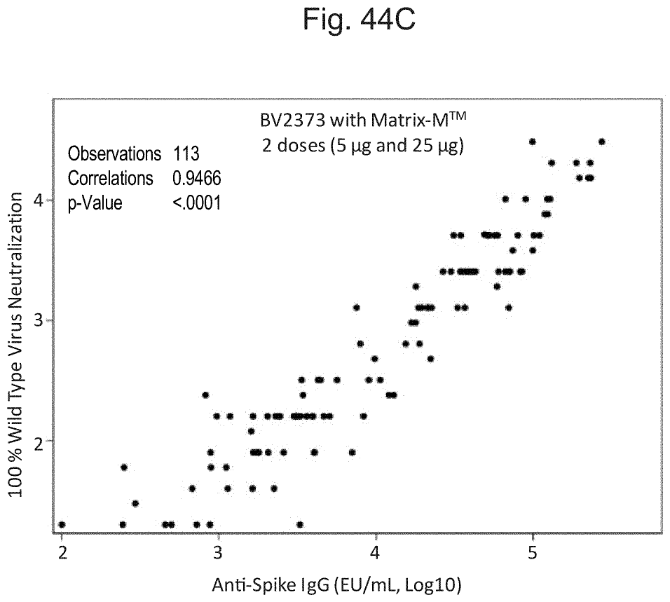

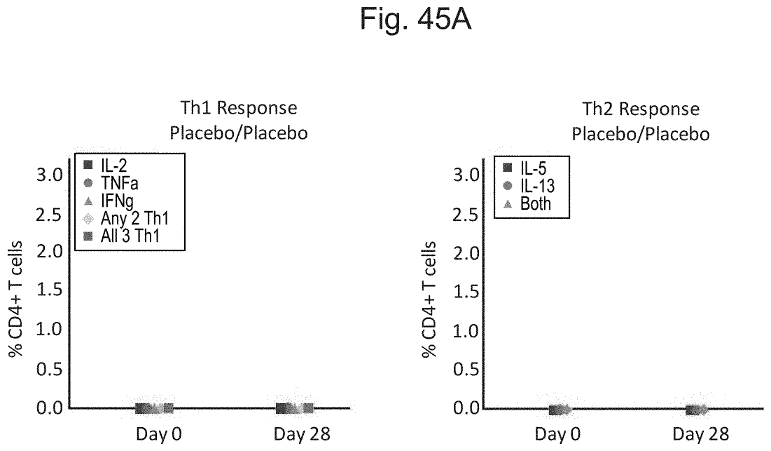

A strong correlation was observed between neutralizing antibody titers and anti-CoV-S IgG in patients treated with BV2373 and MATRIX-M™ (r=0.9466, FIG. 44C) similar to that observed in patients treated with convalescent sera (r=0.958) (FIG. 44A). This correlation was not observed in subjected administered unadjuvanted BV2373 (r=0.7616) (FIG. 44B). Both 5 μg and 25 μg BV2373/MATRIX-M™ groups (groups C-E of Table 5) demonstrated similar magnitudes of two-dose responses and every participant seroconverted using either assay measurement when a two-dose regimen was utilized.

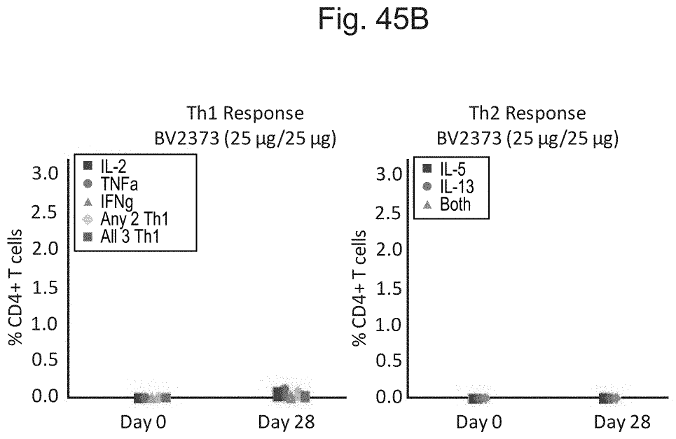

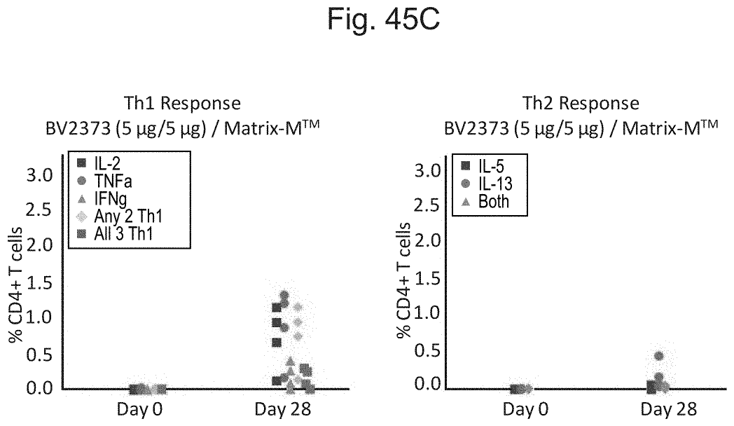

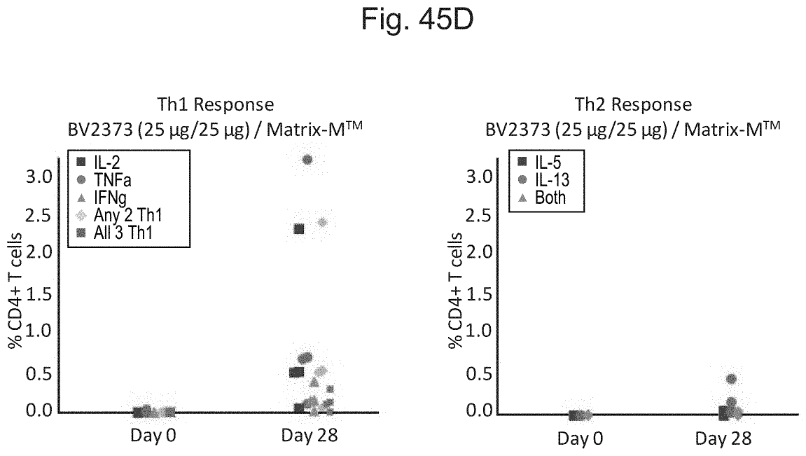

T-cell responses in 16 participants (four participants from each of Groups A through D) showed that BV2373/MATRIX-M™ regimens induced antigen-specific polyfunctional CD4+ T-cell responses in terms of IFN-γ, IL-2, and TNF-α production upon stimulation with BV2373. There was a strong bias toward production of Th1 cytokines (FIGS. 45A-D).

### Example 8

### Expression, Purification, and Evaluation of Next-Generation CoV S Polypeptide Nanoparticles

CoV S polypeptides having the amino acid sequence of SEQ ID NO: 112, SEQ ID NO: 113, SEQ ID NO: 114, or SEQ ID NO: 115 are expressed in a baculovirus expression system and recombinant plaques expressing the coronavirus Spike (S) polypeptides are picked and confirmed. CoV S polypeptides having a sequence of SEQ ID NO: 112, SEQ ID NO: 113, SEQ ID NO: 114, and SEQ ID NO: 115 are expressed using an N-terminal signal peptide having an amino acid sequence of SEQ ID NO: 5.

The CoV S polypeptide having a sequence of SEQ ID NO: 112 comprises a mutation of Asn-488 to tyrosine, mutations of Lys-973 and Val-974 to proline, and an inactivated furin cleavage site having the amino acid sequence of QQAQ (SEQ ID NO: 7).

The CoV S polypeptide having a sequence of SEQ ID NO: 113 comprises mutation of Asp-601 to glycine, mutation of Asn-488 to tyrosine, mutations of Lys-973 and Val-974 to proline, and an inactivated furin cleavage site having the amino acid sequence of QQAQ (SEQ ID NO: 7).

The CoV S polypeptide having a sequence of SEQ ID NO: 114 comprises deletion of amino acids 56, 57, and 131, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of QQAQ (SEQ ID NO: 7).

The CoV S polypeptide having a sequence of SEQ ID NO: 115 comprises mutation of Asn-488 to tyrosine, mutation of Asp-67 to alanine, mutation of Leu-229 to histidine, mutation of Asp-202 to glycine, mutation of Lys-404 to asparagine, mutation of Glu-471 to lysine, mutation of Ala-688 to valine, mutation of Asp-601 to glycine, mutations of Lys-973 and Val-974 to proline, and an inactivated furin cleavage site having the amino acid sequence of QQAQ.

CoV S polypeptide nanoparticles are generated as in Example 1. The stability and immunogenicity of CoV S polypeptides having an amino acid sequence of SEQ ID NO: 112, SEQ ID NO: 112, SEQ ID NO: 113, and SEQ ID NO: 115 is evaluated as in Examples 2-7.

### Example 9

### BV2373 and Saponin Adjuvant Induce Protective Immune Responses Against Heterogeneous SARS-CoV-2 Strains

Purpose: We conducted a phase 3, randomized, observer-blinded, placebo-controlled trial in adults 18-84 years old who received two intramuscular 5-μg doses, 21 days apart, of BV2373 and saponin adjuvant (Fraction A and Fraction C iscom matrix, also referred to as MATRIX-M™ in this example) or placebo (1:1) across 33 sites in the United Kingdom. The primary efficacy endpoint was virologically confirmed mild, moderate, or severe COVID-19 with onset 7 days after second vaccination.

A total of 15,187 participants were randomized, of whom 7569 participants received BV2373 and MATRIX-M™ and 7570 received placebo; 27.8% were 65 years or older, and 4% had baseline serological evidence of SARS-CoV-2 infection. There were 10 cases of COVID-19 among BV2373 and MATRIX-M™ recipients and 96 cases among placebo recipients, with symptom onset at least 7 days after second vaccination; BV2373 and MATRIX-M™ was 89.7% (95% confidence interval, 80.2 to 94.6) effective in preventing COVID-19. There were five cases of severe COVID-19, all of which were reported in the placebo group. A post hoc analysis revealed efficacies of 96.4% (73.8 to 99.5) and 86.3% (71.3 to 93.5) against the prototype SARS-COV-2 strain and B.1.1.7 variant, respectively. The prototype SARS-CoV-2 strain comprises a CoV S protein having the amino acid sequence of SEQ ID NO: 2. The B.1.1.7 variant comprises a CoV S protein having deletions of amino acids 56, 57, and 131 and mutations of N488Y, A557D, D601G, P668H, T703I, S969A, and D1105H, wherein the CoV S polypeptide is numbered with respect to the wild-type SARS-CoV-2 S polypeptide having the amino acid sequence of SEQ ID NO: 2. Vaccine efficacy was similar across subgroups, including participants with comorbidities and those ≥65 years old. Reactogenicity was generally mild and transient and occurred more frequently in the group administered BV2373 and MATRIX-M™. The incidence of serious adverse events was low and similar in the two groups. A two-dose regimen of BV2373 and MATRIX-M™ conferred 89.7% efficacy against a blend of prototype and B.1.1.7 variant, with a safety profile similar to that of other authorized COVID-19 vaccines.

Methods: Trial Design and Participants: We assessed the safety and efficacy of two 5-μg doses of BV2373 and MATRIX-M™ or placebo, administered intramuscularly 21 days apart. This phase 3 trial was conducted at 33 recruitment sites in the UK. Eligible participants were men and non-pregnant women 18 to 84 years old (inclusive) who were healthy or had stable chronic medical conditions, including but not limited to human immunodeficiency virus and cardiac and respiratory diseases. Health status, assessed at screening, was based on medical history, vital signs, and physical examination. Key exclusion criteria included a history of documented COVID-19, treatment with immunosuppressive therapy, or diagnosis with an immunodeficient condition.

Participants were randomly assigned in a 1:1 ratio via block randomization to receive two doses of BV2373 and MATRIX-M™ or placebo (normal saline), 21 days apart, using a centralized Interactive Response Technology system according to pre-generated randomization schedules. Randomization was stratified by site and by age≥65 years. In a 400-person sub-study, participants received a concomitant dose of seasonal influenza vaccine with the first dose. This was an observer-blinded study.

After each vaccination, participants remained under observation at the study site for at least 30 minutes to monitor for the presence of any acute reactions. Solicited local and systemic adverse events were collected via an electronic diary for 7 days after each dose in a subgroup of participants (solicited adverse event subgroup). All participants were assessed for unsolicited adverse events from the first dose through 28 days after the second dose; serious adverse events, adverse events of special interest, and medically attended adverse events were assessed from the first dose through 1 year after the second dose. Safety data are reported for all participants who received at least one dose of vaccine or placebo.

Safety and Efficacy: The primary endpoint was the efficacy of BV2373 and MATRIX-M™ against the first occurrence of virologically confirmed symptomatic mild, moderate, or severe COVID-19, with onset at least 7 days after second vaccination in participants who were seronegative at baseline. Symptomatic COVID-19 was defined according to US Food and Drug Administration (FDA) criteria.

Symptoms of suspected COVID-19 were monitored throughout the trial and collected using a COVID-19 electronic symptom diary (InFLUenza Patient-Reported Outcome [FLU-PRO©] questionnaire) for at least 10 days. At the onset of suspected symptoms of COVID-19, respiratory specimens from the nose and throat were collected daily over a 3-day period to confirm SARS-CoV-2 infection. Virological confirmation was performed using polymerase chain reaction (PCR) testing (UK DHSC laboratories) with the Thermo TaqPath™ system (Thermo Fisher Scientific, Waltham, Mass., USA).

Safety was analyzed in all participants who received at least one dose of BV2373 and MATRIX-M™ or placebo and summarized descriptively. Solicited local and systemic adverse events were also summarized by FDA toxicity grading criteria and duration after each injection. Unsolicited adverse events were coded by preferred term and system organ class using the Medical Dictionary for Regulatory Activities (MedDRA), version 23.1, and summarized by severity and relationship to study vaccine.

The trial was designed and driven by the total number of events expected to achieve statistical significance for the primary endpoint—a target of 100 mild, moderate, or severe Covid-19 cases. The target number of 100 cases for the final analysis was chosen to provide >95% power for 70% or higher vaccine efficacy. A single interim analysis of efficacy was conducted based on the accumulation of approximately 50% (50 events) of the total anticipated primary endpoints using Pocock boundary conditions. The main (hypothesis testing) event-driven analysis for the interim and final analyses of the primary objective was carried out at an overall one-sided type I error rate of 0.025 for the primary endpoint. The primary endpoint was analyzed in participants who were seronegative at baseline, received both doses of study vaccine or placebo, had no major protocol deviations affecting the primary endpoint, and had no confirmed cases of symptomatic Covid-19 within 6 days after the second injection (per-protocol efficacy population). Vaccine efficacy was defined as E (%)=(1−RR)×100, where RR=relative risk of incidence rates between the two study groups (BV2373 and MATRIX-M™ or placebo). Mean disease incidence rate was reported as incidence rate per year in 1000 people. The estimated RR and its confidence interval (CI) were derived using Poisson regression with robust error variance. Hypothesis testing of the primary endpoint was carried out against the null hypothesis: H0: vaccine efficacy≤30%. The success criterion required rejection of the null hypothesis to demonstrate a statistically significant vaccine efficacy.

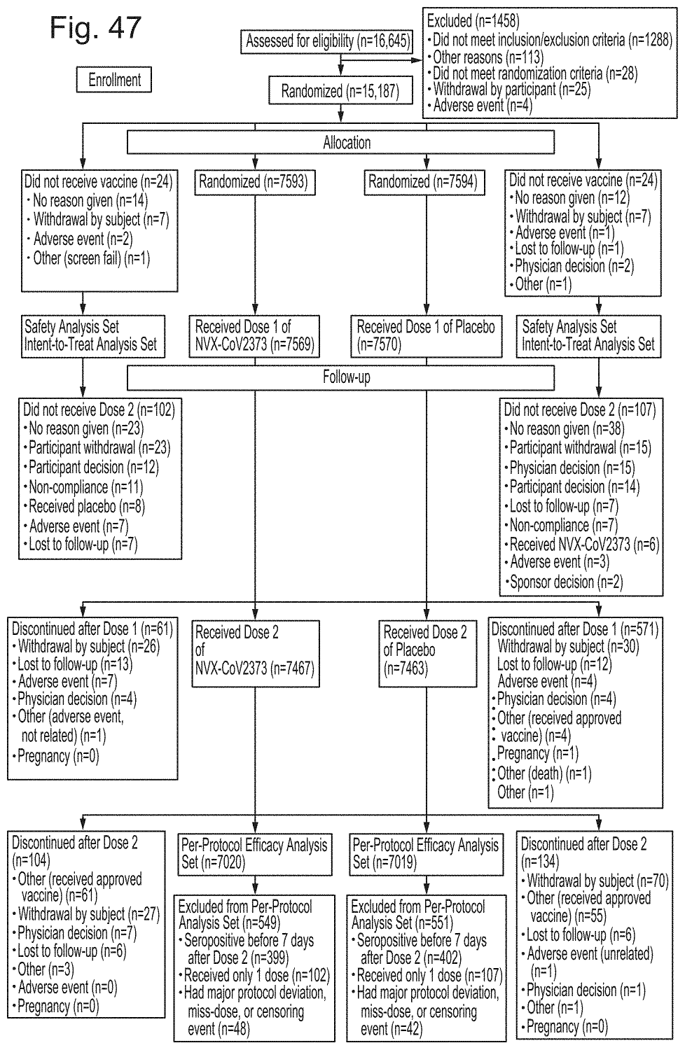

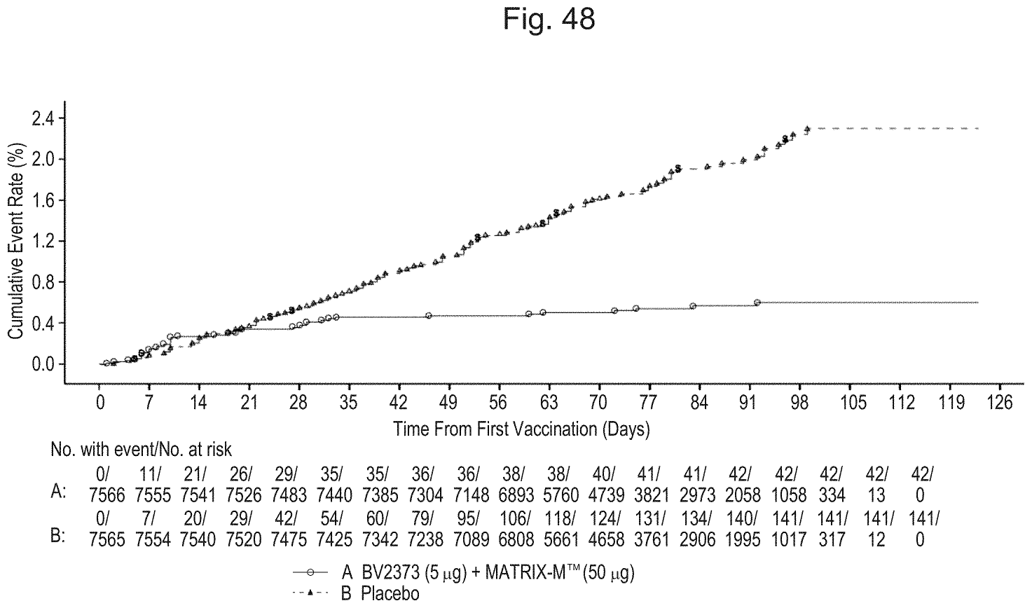

Between Sep. 28 and Nov. 28, 2020, a total of 16,645 participants were screened and 15,187 participants were randomized (FIG. 47). A total of 15,139 participants received at least one dose of BV2373 and MATRIX-M™ (7569) or placebo (7570), with 14,039 participants (7020 in the BV2373 and MATRIX-M™ group and 7019 in the placebo group) meeting the criteria for the per-protocol efficacy population. Baseline demographics were well balanced between the BV2373 and MATRIX-M™ and placebo groups in the per-protocol efficacy population, where 48.4% were female, 94.5% were White, 0.4% were Black or African American, 0.8% were Hispanic or Latino, and 44.6% had at least one comorbid condition (based on Centers for Disease Control and Prevention [CDC] definitions. The median age of these participants was 56 years, and 27.9% were ≥65 years old. Table 6 provides a summary of the baseline demographics of the participants of the clinical trial.

TABLE 6
Demographics and Baseline Characteristics
of Clinical Trial Participants

| | BV2373 and | | | | | |
|---|---|---|---|---|---|---|
| | MATRIX-M ™ | | Placebo | | Total | |
| | n = 7020 | | n = 7019 | | N = 14,039 | |
| Age, y | | | | | | |
| Median | 56.0 | | 56.0 | | 56.0 | |
| Range | 18, 84 | | 18, 84 | | 18, 84 | |
| Age group, n (%) | | | | | | |
| 18-64 y | 5067 | (72.2) | 5062 | (72.1) | 10129 | (72.1) |
| ≥65 y | 1953 | (27.8) | 1957 | (27.9) | 3910 | (27.9) |
| Sex, n (%) | | | | | | |
| Male | 3609 | (51.4) | 3629 | (51.7) | 7238 | (51.6) |
| Female | 3411 | (48.6) | 3390 | (48.3) | 6801 | (48.4) |
| Race or ethnic | | | | | | |
| group, n (%) | | | | | | |
| White | 6625 | (94.4) | 6635 | (94.5) | 13260 | (94.5) |
| Black or African | 26 | (0.4) | 26 | (0.4) | 52 | (0.4) |
| American | | | | | | |
| Asian | 201 | (2.9) | 212 | (2.9) | 413 | (2.9) |
| American Indian | 4 | (<0.1) | 0 | | 4 | (<0.1) |
| or Alaska Native | | | | | | |
| Native Hawaiian | 1 | (<0.1) | 0 | | 1 | (<0.1 |
| or other Pacific | | | | | | |
| Islander | | | | | | |
| Multiple | 70 | (1.0) | 59 | (0.8) | 136 | (0.9) |
| Not reported | 85 | (1.2) | 79 | (1.1) | 176 | (1.2) |
| Other | 4 | (<0.1) | 6 | (<0.1) | 11 | (<0.1) |
| Missing | 4 | | 2 | | 8 | |
| Hispanic or | 63 | (0.9) | 51 | (0.7) | 114 | (0.8) |
| Latinx | | | | | | |
| SARS-CoV-2 | | | | | | |
| serostatus, n (%) | | | | | | |
| Negative | 6964 | (99.2%) | 6944 | (98.9) | 13908 | (99.1) |
| Positive | 0 | | 0 | | 0 | |
| Missing | 56 | | 75 | | 131 | |
| BMI, kg/m², | | | | | | |
| n (%) | | | | | | |
| >30.0: obese | 313 | (4.5) | 323 | (4.6) | 636 | (4.5) |
| Comorbidity | | | | | | |
| status* | | | | | | |
| Yes | 3117 | (44.4) | 3143 | (44.8) | 6260 | (44.6) |
| No | 3903 | (55.6) | 3876 | (55.2) | 7779 | (55.4) |

SD, standard deviation; body mass index (BMI) is calculated as weight (kg) divided by squared height (m). Percentages are based on per-protocol efficacy analysis set within each treatment and overall. *Comorbid subjects are those identified who have at least one of the comorbid conditions reported as a medical history or have a screening BMI value greater than 30 kg/m².

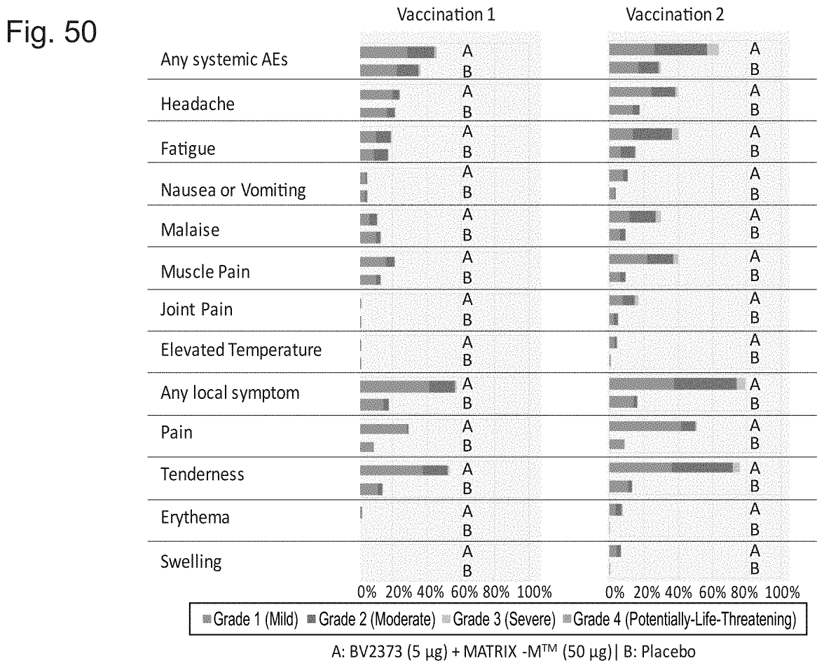

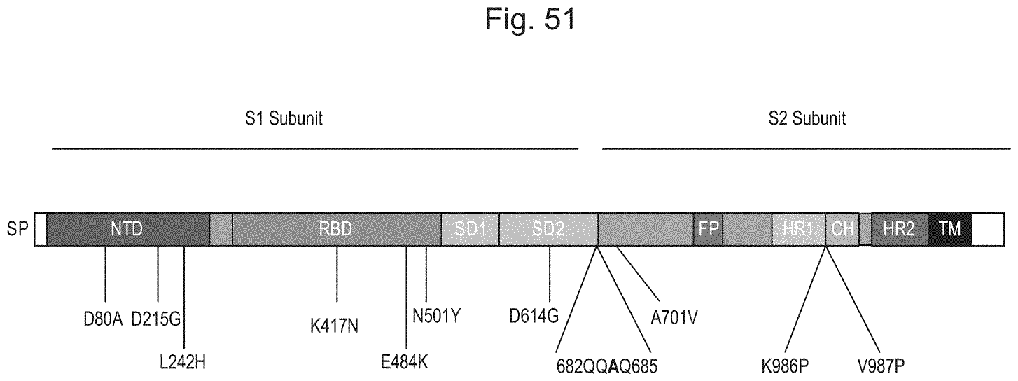

The solicited adverse event subgroup included 2714 participants. Overall, BV2373 and MATRIX-M™ recipients reported higher frequencies of solicited local adverse events than placebo recipients after both the first dose (59.4% vs. 20.9%) and the second dose (80.2% vs. 17.0%) (FIG. 50).

Among BV2373 and MATRIX-M™ recipients, the most commonly reported local adverse events were injection site tenderness and pain after both the first dose (54.9% and 30.7%) and the second dose (76.6% and 51.9%), with most events being grade 1 (mild) or 2 (moderate) in severity and of short mean duration (2.3 and 1.7 days after the first dose and 2.8 and 2.2 days after the second dose). Solicited local adverse events were reported more frequently among younger BV2373 and MATRIX-M™ recipients (18 to 64 years) than older BV2373 and MATRIX-M™ recipients (≥65 years).

Overall, BV2373 and MATRIX-M™ recipients reported higher frequencies of solicited systemic adverse events than placebo recipients after both the first dose (47.6% vs. 37.9%) and the second dose (64.6% vs. 30.8%) (FIG. 50). Among BV2373 and MATRIX-M™ recipients, the most commonly reported systemic adverse events were headache, muscle pain, and fatigue after both the first dose (24.5%, 22.3%, and 20.5%) and the second dose (40.7%, 41.1%, and 41.0%), with most events being grade 1 or 2 in severity and of short mean duration (1.6, 1.5, and 1.9 days after the first dose and 1.9, 1.8, and 1.9 days after the second dose). Grade 4 systemic adverse events were reported in two BV2373 and MATRIX-M™ participants after the first dose and in one BV2373 and MATRIX-M™ participant after the second dose. Systemic adverse events were reported more often by younger vaccine recipients than by older vaccine recipients and more often after dose 2 than dose 1. Notably, fever (temperature≥38° C.) was reported in 2.3% and 5.1% of BV2373 and MATRIX-M™ participants after the first and second doses, with grade 3 fever (39-40° C.) in 0.4% and 0.6% of participants after the first and second doses, respectively; one grade 4 fever (>40° C.) was reported after each dose of vaccine.

All 15,139 participants who received at least one dose of vaccine or placebo through the data cutoff date of the final efficacy analysis were assessed for unsolicited adverse events. The frequency of unsolicited adverse events was higher among BV2373 and MATRIX-M™ recipients than among placebo recipients (25.3% vs. 20.5%), with similar frequencies of severe adverse events (1.0% vs. 0.8%), serious adverse events (0.5% vs. 0.5%), medically attended adverse events (3.8% vs. 3.9%), adverse events leading to vaccine (0.3% vs. 0.3%) or study (0.2% vs. 0.2%) discontinuation, potential immune-mediated medical conditions (<0.1 vs. <0.1%), and adverse events of special interest relevant to COVID-19 (0.1% vs. 0.3%). One related serious adverse event was reported in an BV2373 and MATRIX-M™ recipient (myocarditis), which was considered a potentially immune-mediated condition; an independent SMC considered the event most likely a viral myocarditis. The participant recovered. There were no episodes of anaphylaxis, and no evidence of vaccine-associated enhanced disease. Two COVID-19-related deaths were reported, one in the BV2373 and MATRIX-M™ group, with onset of symptoms 7 days after receiving a single vaccine dose, and one in the placebo group.

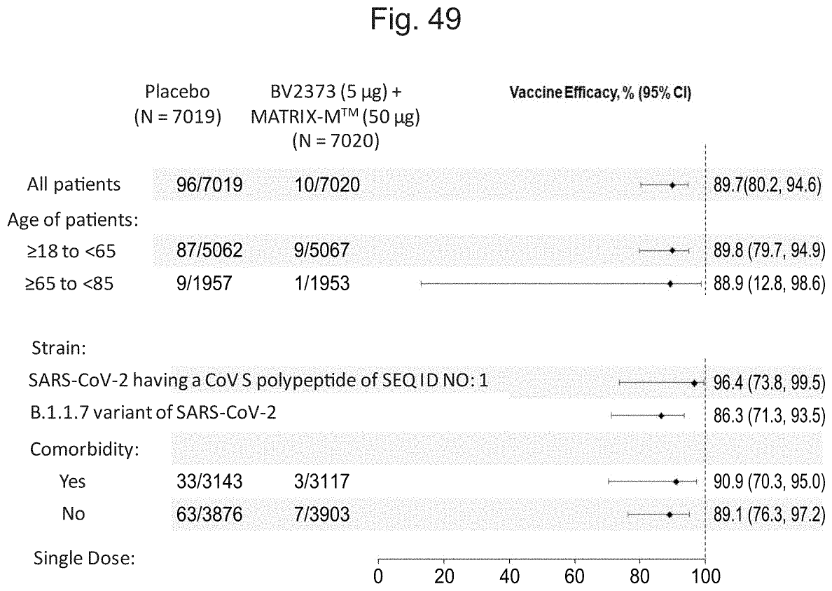

Among 14,039 participants in the per-protocol efficacy population, there were 10 cases of virologically confirmed, symptomatic mild, moderate or severe COVID-19 with onset at least 7 days after the second dose among vaccine recipients (6.53 per 1000 person-years; 95% CI: 3.32 to 12.85) and 96 cases among placebo recipients (63,43 per 1000 person-years; 95% CI: 45.19 to 89.03) for a vaccine efficacy of 89.7% (95% CI, 80.2 to 94.6; FIG. 49). Of the 10 cases ≥65 years old who had mild, moderate, or severe Covid-19, one had received BV2373 and MATRIX-M™ and nine had received placebo. Severe COVID-19 occurred in five participants, of whom none had received BV2373 and MATRIX-M™ and five had received placebo. There were no hospitalizations or deaths among per-protocol vaccine recipients. Vaccine efficacy among participants ≥65 years was 88.9% (95% CI, 12.8 to 98.6 and efficacy from 14 days after dose 1 was 83.4% (95% CI, 73.6 to 89.5) (FIG. 49). A post hoc analysis of the primary endpoint identified 29, 66, and 11 cases of Covid-19 where the isolated strain was the SARS CoV-2 prototype strain, SARS-CoV-2 B.1.1.7 variant, or unknown, respectively. Unknown samples were those where the PCR tests were performed with a non-DHSC PCR test (e.g., at a local hospital laboratory) where variant determination was not performed. Vaccine efficacy against the prototype strain was 96.4% (95% CI, 73.8 to 99.4), while efficacy against the B.1.1.7 variant was 86.3% (95% CI, 71.3 to 93.5). (FIG. 49).

Discussion: A two-dose regimen of BV2373 and MATRIX-M™, given 21 days apart, was found to be safe and 89.7% effective against symptomatic COVID-19 caused by both prototype and B.1.1.7 variants. The timing of accumulated cases in this study allowed for a post hoc assessment of vaccine efficacy against different strains, including the B.1.1.7 variant, which is now circulating widely outside of the United Kingdom and is soon expected to be the most prominent strain in United States. This variant is known to be more transmissible and to be associated with a higher case fatality rate than previous strains, emphasizing the need for an effective vaccine. This is the first vaccine to demonstrate high vaccine efficacy (86.3%) against the B.1.1.7 variant in a phase 3 trial. Although the study was not powered to assess efficacy for individual SARS-CoV-2 strains, BV2373 and saponin adjuvant demonstrated significant efficacy against all strains detected in trial participants. In particular, the 96.4% point estimate of efficacy determined against the prototype strain is similar to that reported against this strain for the BNT161b2 mRNA vaccine (95.0%) and the mRNA-1273 vaccine (94.1%) and greater than that demonstrated by the adenoviral vector vaccines.

Finally, the BV2373 and saponin adjuvant composition also showed efficacy against the B.1.351 variant.

Prevention of severe disease (including hospitalization, intensive care admission, and death) is an important objective of a vaccination program, and the two-dose regimen of BV2373 and saponin adjuvant demonstrated very high efficacy, similar to that reported for other licensed Covid-19 vaccines. In addition, BV2373 and saponin adjuvant provided levels of protection after the first dose in a range similar to that of other COVID-19 vaccines. The favorable safety profile observed during phase 1/2 studies of BV2373 and saponin adjuvant was confirmed in this phase 3 trial. Reactogenicity was generally mild or moderate, and reactions were less common and milder in older subjects and more common after the second dose. Injection site tenderness and pain, fatigue, headache, and muscle pain were the most commonly reported local and systemic adverse events and were more common with the vaccine than placebo. The incidence of serious adverse events was similar in the vaccine and placebo groups (0.5% in each) and no deaths were attributable to receipt of the vaccine.

The results of this trial provide further evidence that COVID-19 caused by prototype SARS-CoV-2 and the SARS-CoV-2 variant B.1.1.7 can be prevented by immunization, providing the first evidence for a protein-based, adjuvanted vaccine. These data confirm that BV2373 and saponin adjuvant can be stored at standard refrigerator temperatures and, moreover, can induce a broad epitope response to the spike protein antigen. This broad response provide protective efficacy against a range of heterogenous SARS-CoV-2 strains.

### Example 10

### BV2438 and Saponin Adjuvant Induce Protective Immune Responses Against Heterogeneous SARS-CoV-2 Strains

Purpose: The immunogenicity and in vivo protection of compositions containing the recombinant CoV Spike (rS) protein BV2438 (SEQ ID NO: 132), BV2373 (SEQ ID NO: 87), or both, in combination with a saponin adjuvant was evaluated. The saponin adjuvant contains two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. Fraction A and Fraction C account for 85% and 15% by weight, respectively, of the sum of the weights of fraction A of Quillaja Saponaria Molina and fraction C of Quillaja Saponaria Molina in the adjuvant.

The efficacy of BV2438 and BV2373 immunization regimens alone or in combination with the aforementioned saponin adjuvant against the SARS-CoV-2/WA1, SARS-CoV-2/B.1.1.7 and SARS-CoV-2/B.1.351 strains were evaluated. The SARS-CoV-2/WA1 strain has a CoV S polypeptide having the amino acid sequence of SEQ ID NO: 2. The SARS-CoV-2/B.1.1.7 strain has a CoV S polypeptide comprising deletions of amino acids 69, 70, and 144 and mutations of N501Y, A570D, D614G, P681H, T716I, S982A, and D1118H, wherein the CoV S polypeptide is numbered with respect to the wild-type SARS-CoV-2 S polypeptide having the amino acid sequence of SEQ ID NO: 1. The SARS-CoV-2/B.1.351 strain has a CoV S polypeptide comprising polypeptide comprising mutations of D80A, L242H, R246I, A701V, N501Y, K417N, E484K, and D614G, wherein the CoV S polypeptide is numbered with respect to the wild-type SARS-CoV-2 S polypeptide having the amino acid sequence of SEQ ID NO: 1.

Methods:

Cells and Virus: Virus and cells were processed as described previously (18). Briefly, Vero E6 cells (ATCC #CRL 1586) were cultured in DMFM (Quality Biological), supplemented with 10% (v/v) fetal bovine serum (Gibco), 1% (v/v) penicillin/streptomycin (Gemini Bio-products) and 1% (v/v) L-glutamine (2 mM final concentration, Gibco) (Vero media). Cells were maintained at 37° C. and 5% CO2. SARS-CoV-2/WA1 were provided by the CDC (BEI #NR-52281). SARS-CoV-2/B.1.17 and SARS-CoV-2/B.1.351 were generously provided by Dr. Andy Pekosz at The Johns Hopkins University obtained. Stocks for both viruses were prepared by infection of Vero E6 cells for two days when CPE was starting to be visible. Media were collected and clarified by centrifugation prior to being aliquoted for storage at −80° C. Titer of stock was determined by plaque assay using Vero E6 cells as described previously.

SARS-CoV-2 Protein Expression: SARS-CoV-2 constructs were synthetically produced from the full-length S glycoprotein gene sequence (GenBank MN908947 nucleotides 21563-25384). The full-length S-genes were codon optimized for expression in Spodoptera frugiperda (Sf9) cells and synthetically produced by GenScript (Piscataway, N.J., USA). The QuikChange Lightning site-directed mutagenesis kit (Agilent) was used to produce two spike protein variants: the furin cleavage site (682-RRAR-685) was mutated to 682-QQAQ-685 to be protease resistant and two proline substitutions at positions K986P and V987P (2P) were introduced to produce the double mutant, BV2373. To generate the recombinant Spike construct based on the B.1.351 variant, the following point mutations were also introduced: D60A, D215G, L242H, K417N, E484K, N501Y, D614G, and A701V. Full-length S-genes were cloned between the BamHI-HindIII sites in the pFastBac baculovirus transfer vector (Invitrogen, Carlsbad, Calif.) under transcriptional control of the Autographa californica polyhedron promoter. Recombinant baculovirus constructs were plaque purified and master seed stocks prepared and used to produce the working virus stocks. The baculovirus master and working stock titers were determined using rapid titer kit (Clontech, Mountain View, Calif.). Recombinant baculovirus stocks were prepared by infecting Sf9 cells with a multiplicity of infection (MOI) of ≤0.01 plaque forming units (pfu) per cell.

Expression and Purification: SARS-CoV-2 S proteins were produced in Sf9 cells as previously described. Briefly, cells were expanded in serum-free medium and infected with recombinant baculovirus. Cells were cultured at 27±2° C. and harvested at 68-72 hours post-infection by centrifugation (4000×g for 15 min). Cell pellets were suspended in 25 mM Tris HCl (pH 8.0), 50 mM NaCl and 0.5-1.0% (v/v) polyoxyethylene nonylphenol (NP-9, TERGITOL®) NP-9 with leupeptin. S-proteins were extracted from the plasma membranes with Tris buffer containing NP-9 detergent, clarified by centrifugation at 10,000×g for 30 min. S-proteins were purified by TMAE anion exchange and lentil lectin affinity chromatography. Hollow fiber tangential flow filtration was used to formulate the purified spike protein at 100-150 μg mL-1 in 25 mM sodium phosphate (pH 7.2), 300 mM NaCl, 0.02% (v/v) polysorbate 80 (PS 80). Purified S-proteins were evaluated by 4-12% gradient SDS-PAGE stained with Gel-Code Blue reagent (Pierce, Rockford, Ill.) and purity was determined by scanning densitometry using the OneDscan system (BD Biosciences, Rockville, Md.).

Differential Scanning Calorimetry: Samples (BV2426 Lot 1 Feb. 2021 and BV2373 Lot 15 Dec. 2020; rS-B.1.351BV2438 and rS-WU1BV2373, respectively) and corresponding buffers were heated from 4° C. to 120° C. at 1° C. per minute and the differential heat capacity change was measured in a NanoDSC (TA Instruments, New Castle, Del.). A separate buffer scan was performed to obtain a baseline, which was subtracted from the sample scan to produce a baseline-corrected profile. The temperature where the peak apex is located is the transition temperature (Tmax) and the area under the peak provides the enthalpy of transition (ΔHcal).

Transmission Electron Microscopy and 2D Class Averaging: Electron microscopy was perform by NanoImaging Services (San Diego, Calif.) with a FEI Tecani T12 electron microscope, operated at 120 keV equipped with a FEI Eagle 4 k×4 k CCD camera. SARS-CoV-2 S proteins were diluted to 2.5 μg mL-1 in formulation buffer. The samples (3 μL) were applied to nitrocellulose-supported 400-mesh copper grids and stained with uranyl format. Images of each grid were acquired at multiple scales to assess the overall distribution of the sample. High-magnification images were acquired at nominal magnifications of 150,000× (X nm/pixel) and 92,000× (0.16 nm/pixel). The images were acquired at a nominal defocus of −2.0 μm to −1.5 μm (110,000×) and electron doses of ˜25 e/Å².

For class averaging, particles were identified from 92,000× high magnification images, followed by alignment and classification as previously described.

Kinetics of SARS-CoV-2 S binding to hACE2 receptor by Bioluminescence Imaging (BLI): S-protein receptor binding kinetics was determined by bio-layer interferometry (BLI) using an Octet QK384 system (Pall Forté Bio, Fremont, Calif.). His-tagged human ACE2 (2 μg mL-1) was immobilized on nickel-charged Ni-NTA biosensor tips. After baseline, SARS-CoV-2 rS protein solutions were 2-fold serially diluted in kinetics buffer over a range of 300 nM to 4.7 nM, allowed to associate for 600 sec, followed by dissociation for an additional 600-900 sec. Data was analyzed with Octet software HT 10.0 by 1:1 global curve fit.

Mouse Study Designs: Female BALB/c mice (7-9 weeks old, 17-22 grams, N=20 per group) were immunized by intramuscular (IM) injection with two doses spaced 14 days apart (study day 0 and 14) of rS-WU1BV2373, rS-B.1.351BV2438 with 5 μg saponin-based Matrix-M™ adjuvant (Novavax, AB, Uppsala, SE) either alone, in combination, or as a heterologous prime/boost A placebo group was injected with vaccine formulation buffer as a negative control. Serum was collected for analysis on study days −1, 14, 21, and 32. Vaccinated and control animals were intranasally challenged with SARS-CoV-2 on study day 46.

To assess the cellular response mediated by Matrix-Msaponin adjuvant, groups of female BALB/c mice (N=8 per group) were immunized IM with the same regimens described above, with injections spaced 21 days apart. Spleens were collected 7 days after the second immunization (study day 28). A non-vaccinated group (N=5) served as a control.

Baboon Study Designs: Nine adult baboons (10-16 years of age at study initiation) were randomized into 4 groups of 2-3/group and immunized by IM injection with rS-WU1BV2373 at 1, 5, or 25 μg rS with 50 μg Matrix-Msaponin adjuvant. A separate group was immunized with 25 μg rS without adjuvant. Animals were vaccinated with 2 doses spaced 21 days apart in this primary immunization series. Immunogenicity results after the primary immunization series were previously described (15). Approximately one year later (45 weeks), all animals were boosted with one or two 3 μg doses of rS-B.1.351.BV2438 with 50 μg Matrix-Msaponin adjuvant. Sera and PBMCs were collected before and after the boost to measure antibody- and cell-mediated immune responses.

SARS-CoV-2 challenge in mice: Mice were anaesthetized by intraperitoneal injection 50 μL of a mix of xylazine (0.38 mg/mouse) and ketamine (1.3 mg/mouse) diluted in phosphate buffered saline (PBS). Mice were intranasally inoculated with either 7×104 pfu of B.1.117 or 1×105 pfu of B.1.351 strains of SARS-CoV-2 in 50 μL. Challenged mice were weighed on day of infection and daily for 4 days post infection. At days 2- and 4-days post infection, 5 mice were sacrificed from each vaccination and control group, and lungs were harvested to determine viral titer by a plaque assay and viral RNA levels by qRT-PCR.

SARS-CoV-2 Plaque Assay: SARS-CoV-2 lung titers were quantified by homogenizing harvested lungs in PBS (Quality Biological Inc.) using 1.0 mm glass beads (Sigma Aldrich) and a Beadruptor (Omini International Inc.). Homogenates were added to Vero E6 near confluent cultures and SARS-CoV-2 virus titers determined by counting plaque forming units (pfu) using a 6-point dilution curve.

Anti-SARS-CoV-2 Spike IgG by ELISA: An ELISA was used to determine anti-SARS-CoV-2 S IgG titers. Briefly, 96 well microtiter plates (ThermoFischer Scientific, Rochester, N.Y., USA) were coated with 1.0 μg mL-1 of SARS-CoV-2 spike protein. Plates were washed with PBS-T and blocked with TBS Startblock blocking buffer (ThermoFisher, Scientific). Mouse, baboon or human serum samples were serially diluted (10-2 to 10-8) and added to the blocked plates before incubation at room temperature for 2 hours. Following incubation, plates were washed with PBS-T and HRP-conjugated goat anti-mouse IgG or goat anti-human IgG (Southern Biotech, Birmingham, Ala., USA) added for 1 hour. Plates were washed with PBS-T and 3,3′,5,5′-tetramethylbenzidine peroxidase substrate (TMB, T0440-IL, Sigma, St Louis, Mo., USA) was added. Reactions were stopped with TMB stop solution (ScyTek Laboratories, Inc. Logan, Utah). Plates were read at OD 450 nm with a SpectraMax Plus plate reader (Molecular Devices, Sunnyvale, Calif., USA) and data analyzed with SoftMax software. EC50 values were calculated by 4-parameter fitting using SoftMax Pro 6.5.1 GxP software. Individual animal anti-SARS-CoV-2 S IgG titers and group geometric mean titers (GMT) and 95% confidence interval (±95% CI) were plotted GraphPad Prism 7.05 software.

hACE2 receptor blocking antibodies: Human ACE2 receptor blocking antibodies were determined by ELISA. Ninety-six well plates were coated with 1.0 μg, mL-1 SARS-CoV-2 S protein overnight at 4° C. After washing with PBS-T and blocking with StartingBlock (TBS) blocking buffer (ThermoFisher Scientific), serially diluted serum from groups of immunized mice, baboons or humans were added to coated wells and incubated for 1 hour at room temperature. After washing, 30 ng mL-1 of histidine-tagged hACE2 (Sino Biologics, Beijing, CHN) was added to wells for 1 hour at room temperature. After washing, HRP-conjugated anti-histidine IgG (Southern Biotech, Birmingham, Ala., USA) was added, followed by washing and addition of TMB substrate. Plates were read at OD 450 nm with a SpearaMax plus plate reader (Molecular Devices, Sunnyvale, Calif., USA) and data analyzed with SoftMax Pro 6.5.1 GxP software. The % Inhibition for each dilution for each sample was calculated using the following equation in the SoftMax Pro program: 100−[(MeanResults/ControlValue@PositiveControl)*100].

Serum dilution versus % Inhibition plot was generated and curve fitting was performed by 4 parameter logistic (4PL) curve fitting to data. Serum antibody titer at 50% inhibition (IC50) of hACE2 to SARS-COV-2 S protein (BV2373 or BV2438) was determined in the SoftMax Pro program.

SARS-CoV-2 Neutralization Titer by Plaque Reduction Neutralization Titer Assay (PRNT): PRNTs were processed as described previously (20). Briefly, serum samples were diluted in DMEM (Quality Biological) at an initial 1:40 dilution with 1:2 serial dilutions for a total of 11 dilutions. A no-sera control was included on every plate. SARS-CoV-2 was then added 1:1 to each dilution for a target of 50 PFU per plaque assay well and incubated at 37° C. (5.0% CO2) for 1 hr. Samples titers where then determined by plaque assay and neutralization titers determined as compared to the non-treatment control. A 4-parameter logistic curve was fit to these neutralization data in PRISM (GraphPad, San Diego, Calif.) and the dilution required to neutralize 50% of the virus (PRNT50) was calculated based on that curve fit.

Surface and intracellular cytokine staining: For surface staining, murine splenocytes were first incubated with an anti-CD16/32 antibody to block the Fc receptor. To characterize T follicular helper cells (Tfh), splenocytes were incubated with the following antibodies or dye: BV650-conjugated anti-CD3, APC-H7-conjugated anti-CD4, FITC-conjugated anti-CD8, Percp-cy5.5-conjugated anti-CXCR5, APC-conjugated anti-PD-1, Alexa Fluor 700-conjugated anti-CD19, PE-conjugated anti-CD49b (BD Biosciences, San Jose, Calif.) and the yellow LIVE/DEAD® dye (Life Technologies, NY). To stain germinal center (GC) B cells, splenocytes were labeled with FITC-conjugated anti-CD3, PerCP-Cy5.5-conjugated anti-B220, APC-conjugated anti-CD19, PE-cy7-conjugated anti-CD95, and BV421-conjugated anti-GL7 (BD Biosciences) and the yellow viability dye (LIVE/DEAD®) (Life Technologies, NY).

For intracellular cytokine staining (ICCS) of murine splenocytes, cells were cultured in a 96-well U-bottom plate at 2×106 cells per well. The cells were stimulated with rS-WU1BV2373 or rS-B.1.351BV2438 spike protein. The plate was incubated 6 h at 37° C. in the presence of BD GolgiPlug™ and BD GolgiStop™ (BD Biosciences) for the last 4 h of incubation. Cells were labeled with murine antibodies against CD3 (BV650), CD4 (APC-H7), CD8 (FITC), CD44 (Alexa Fluor 700), and CD62L (PE) (BD Pharmingen, CA) and the yellow LIVE/DEAD® dye. After fixation with Cytofix/Cytoperm (BD Biosciences), cells were incubated with PerCP-Cy5.5-conjugated anti-IFN-γ, BV421-conjugated anti-IL-2, PE-cy7-conjugated anti-TNF-α, and APC-conjugated anti-IL-4 (BD Biosciences). All stained samples were acquired using a LSR-Fortessa or a FACSymphony flow cytometer (Becton Dickinson, San Jose, Calif.) and the data were analyzed with FlowJo software version Xv10 (Tree Star Inc., Ashland, Oreg.).

For ICS of baboon PBMCs, PBMCs collected at the timepoints listed in FIG. 5A were stimulated as described above with rS-WU1BV2373 or rS-B.1.351BV2438. Cells were labeled with human/NHP antibodies BV650-conjugated anti-CD3, APC-H7-conjugated anti-CD4, FITC-conjugated anti-CD8, BV421-conjugated anti-IL-2, PerCP-Cy5.5-conjugated anti-IFN-γ, PE-cy7-conjugated anti-TNF-α, APC-conjugated anti-IL-15, BV711-conjugated anti-IL-13 (BD Biosciences), and the a yellow LIVE/DEAD® viability dye.

Enzyme Linked Immunosorbent Assay (ELISA): Murine IFN-γ and IL-5 ELISpot assays were performed following the manufacturer's procedures for mouse IFN-γ and IL-5 ELISpot kits (3321-2H and 3321-2A, Mabtech, Cincinnati, Ohio). Briefly, 4×105 splenocytes in a volume of 200 μL were stimulated with rS-WU1BV2373 or rS-B.1.351BV2438 in plates that were pre-coated with anti-IFN-γ or anti-IL-5 antibodies. Detection secondary antibodies were clone RS-6A2 IFN-γ and clone TRFK4. Each stimulation condition was carried out in triplicate. Assay plates were incubated 24-48 h at 37° C. in a 5% CO2 incubator and developed using BD ELISpot AEC substrate set (BD Biosciences, San Diego, Calif.). Spots were counted and analyzed using an ELISpot reader and ImmunoSpot software v6 (Cellular Technology, Ltd., Shaker Heights, Ohio). The number of IFN-γ- or IL-5-secreting cells was obtained by subtracting the background number in the medium controls. Data shown in the graph are the average of triplicate wells.

Similarly, baboon IFN-γ and IL-4 assays were carried out using NHP IFN-γ and Human IL-4 assay kits from Mabtech. For IFN-γ, coating antibody human IFN-γ 3420-2H and detection antibody clone 7-B6-1 were used. For IL-4, coating antibody human IL-43410-2H (clone IL4-I) and detection antibody clone IL4-II were used. Assays were performed in triplicate.

Statistical Analysis: Statistical analyses were performed with GraphPad Prism 8.0 software (La Jolla, Calif.). Serum antibody titers were plotted for individual animals and the geometric mean titer (GMT) and 95% confidence interval (95% CI) or the means±SEM as indicated in the figure. Ordinary one-way ANOVA with Tukey's multiple comparisons post-hoc test was performed on log 10-transformed data to evaluate statistical significance of differences among groups. P-values≤0.05 were considered as statistically significant.

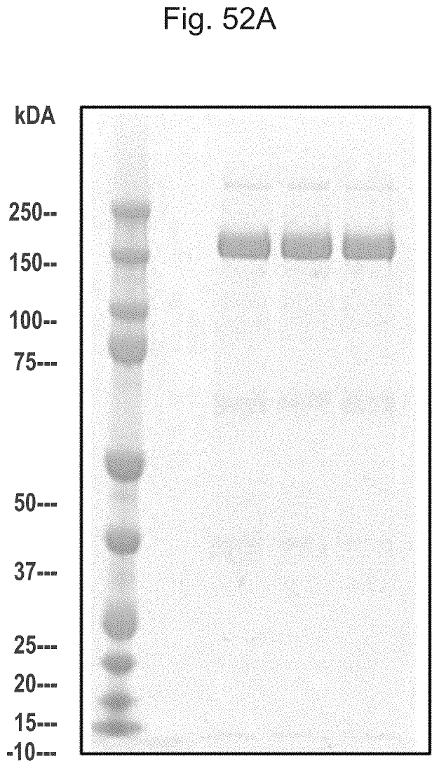

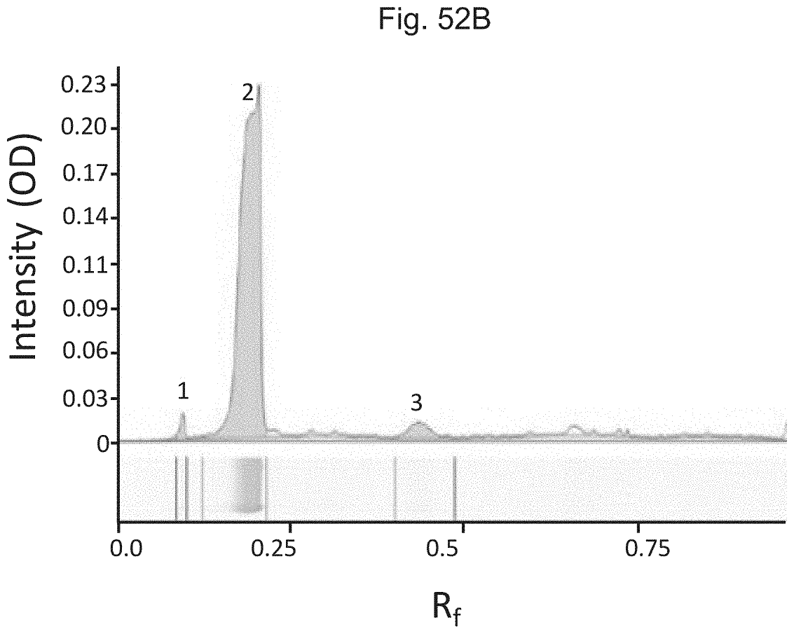

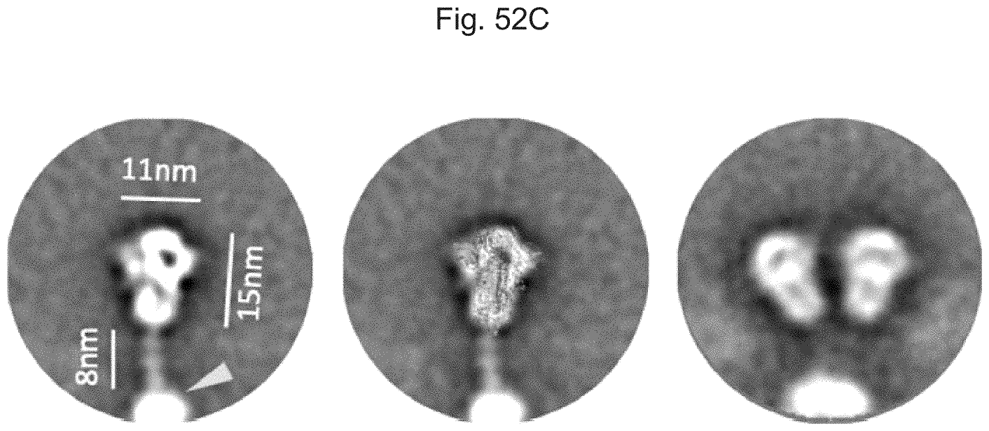

Biophysical Properties, Structure, and Function of BV2438 antigen: Purified BV2438, when reduced and subjected to SDS-PAGE, migrated with the expected molecular weight of approximately 170 kDa (FIG. 52A). The thermal stability of BV2438 was compared to that of BV2373 by differential scanning calorimetry (DSC); the main peak of the BV2438 showed a 4° C. increase in thermal transition temperature (Tₘₐₓ) and 1.3-fold higher enthalpy of transition (ΔHCal) compared to the prototype BV2373 protein, indicating increased stability of BV2438 (FIG. 52B, Table 7). Transmission electron microscopy (TEM) combined with two rounds of two-dimensional (2D) class averaging of 16,049 particles were used to confirm the ultrastructure of BV2438. High magnification (92,000× and 150,000×) TEM images revealed a lightbulb-shaped particle appearance with a 15 nm length and an 11 nm width, which was consistent with the profusion form of the SARS-CoV-2 spike trimer (PDB ID 6VXX; FIG. 52C). This is consistent with what we have previously observed for the prototype BV2373 protein.

To confirm the functional properties of the variant spike protein construct BV2438, binding of this rS protein to the hACE2 receptor was determined using bio-layer interferometry (BLT) as previously described. BV2438 was found to bind tightly and stably to hACE2, with an association constant (Ka) of 3.94×10⁴, representing a 3.6-fold greater association to hACE2 compared to the prototype protein BV2373 (Ka=1.08×10⁴). Dissociation constants of these two proteins were essentially identical (1.46×10⁻⁷ and 1.56×10⁻⁷ for BV2438 and BV2373, respectively). We additionally assessed BV2438 binding to hACE2 with an ELISA as previously described. In this assay, BV2438 attained 50% saturation of hACE2 at a slightly lower concentration (EC₅₀=8.0 ng/mL) than the prototype construct BV2373 (EC₅₀=9.4 ng/mL), confirming that BV2438 exhibited a slightly higher affinity for hACE2 compared to that of BV2373 (Table 7).

TABLE 7
Thermostability and hACE2 binding of
SARS-CoV-2 recombinant spike proteins

| | Differential | | hACE2 binding | | |
|---|---|---|---|---|---|
| | Scanning | | hACE2 Binding | | |
| | Calorimetry | | Kinetics by Bio-layer | | hACE2 |
| | (DSC) | | Interferometry | | ELISA |
| SARS-CoV-2 | Tₘₐₓ | ΔHcal | Kₐ | K_{dis} | (EC₅₀, |
| rS proteins | (° C.) | (kJ mol⁻¹) | (1/Ms) | (1/s) | ng/mL) |
| BV2438 | 67.24 | 725.1 | 3.94 × 10⁴ | 1.46 × 10⁻⁷ | 8.0 |
| BV2373 | 63.21 | 546.0 | 1.08 × 10⁴ | 1.56 × 10⁻⁷ | 9.4 |

Tₘₐₓ, melting temperature;
Kₐ, binding constant;
K_{dis}, dissociation constant;
EC₅₀, half-maximal binding.

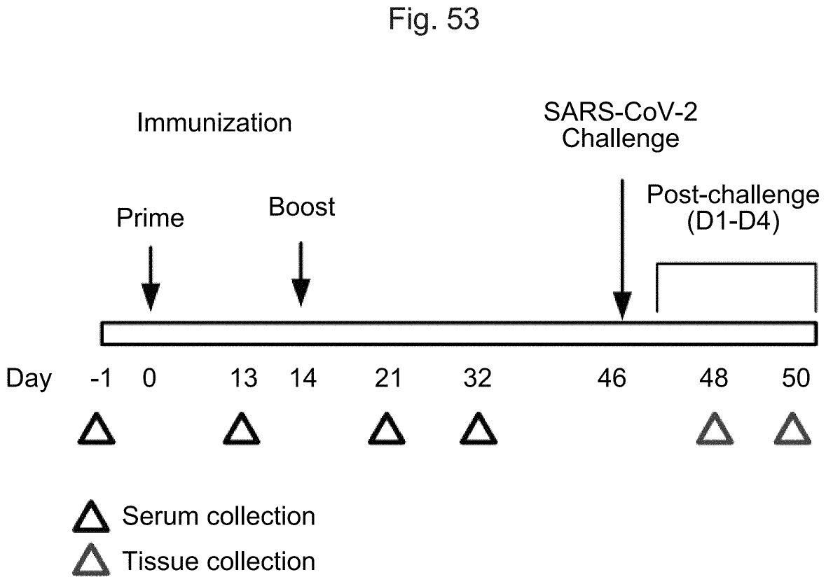

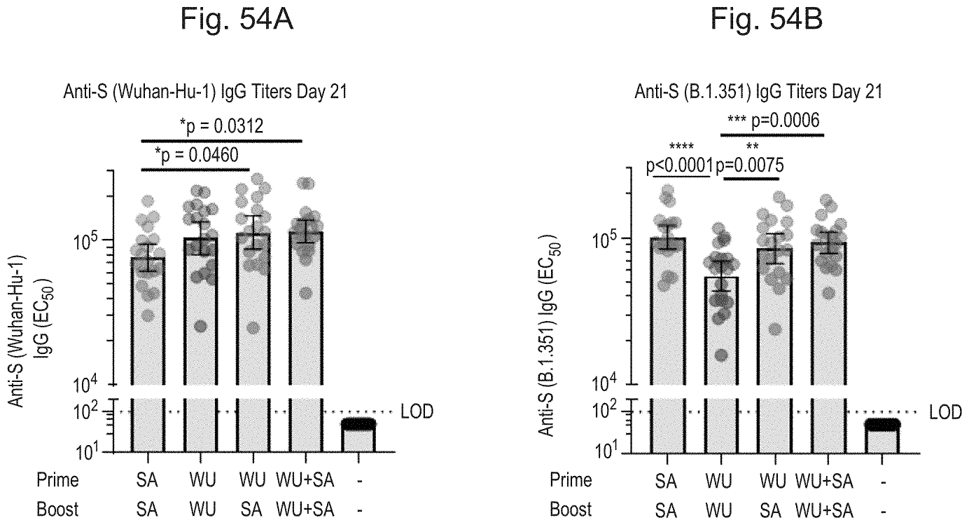

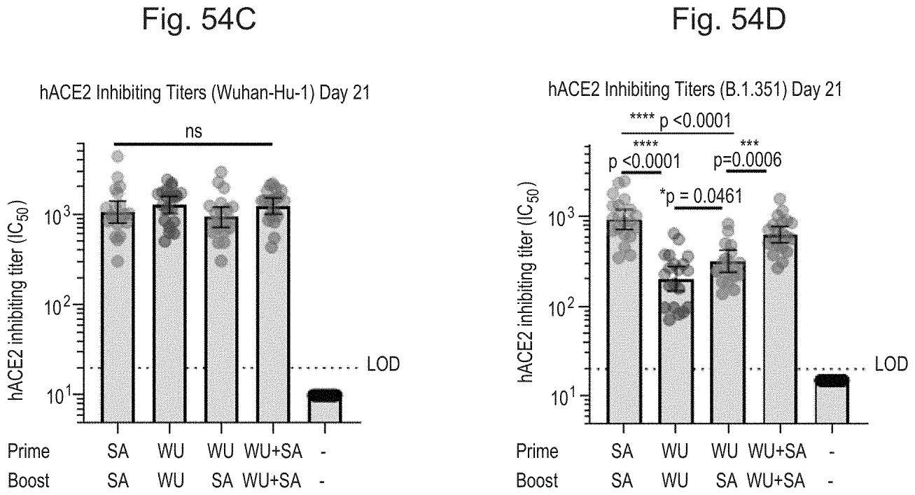

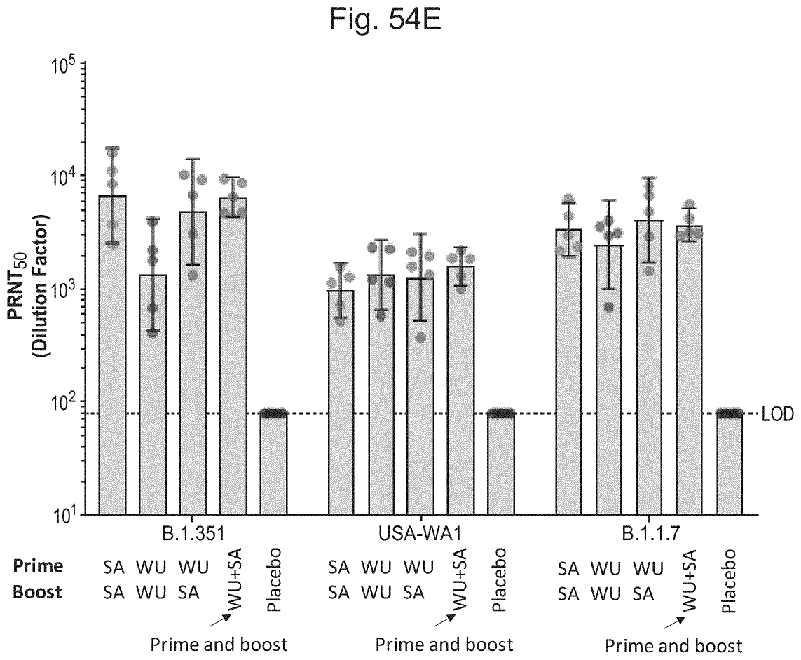

BV2438 Immunogenicity in Mice: We assessed the antibody- and cell-mediated immunogenicity of BV2438 and BV2373 formulated with saponin adjuvant. To assess antibody-mediated immunogenicity, groups of mice (n=20) were immunized with either BV2373 or BV2438 as both prime and boost, with BV2373 as the prime and BV2438 as the boost, or with both vaccines combined in a bivalent formulation for the prime and boost vaccination. A placebo group received vaccine formulation buffer as a negative control. In monovalent immunization groups, 1 μg of rS and 5 μg of saponin adjuvant was intramuscularly injected at Days 0 and 14. For bivalent immunization, 1 μg of each rS construct was administered at each immunization, for a total of 2 μg rS, with 5 μg of saponin adjuvant. The study design is shown in FIG. 53. Mice immunized with either of the 4 vaccine regimens displayed elevated antibody titers against both the B.2 Spike and B.1.351. Spike by ELISA at day 21 post vaccination. Monovalent vaccination with either BV2373 or BV2438 produced significantly lower anti-S (WU1) IgG titers than bivalent vaccination or heterologous vaccination, although neither reduced IgG titers more than 2-fold (FIG. 54A, FIG. 54B). Conversely, immunization with BV2373 alone resulted in significantly lower titers against B.1.351 Spike compared to all other immunization regimens; immunization with monovalent BV2438 or bivalent rS resulted in anti-B.1.351 spike IgG titers that were the highest among regimens tested, with no significant difference between these regimens (FIG. 54A, FIG. 54B). Animals in the placebo group exhibited undetectable anti-B.2 Spike and anti-B.1.351 spike IgG titers as expected.

The ability of serum from mice to inhibit Spike to hACE2 binding was also assessed. All immunization regimens resulted in the production of antibodies that blocked hACE2 binding to a CoV Spike polypeptide with no significant difference between any groups at Day 21 (FIG. 54C, FIG. 54D). Yet immunization with BV2373 alone resulted in significantly lower serum titers capable of disrupting binding between B.1.351 spike and hACE2; titers in the BV2373 alone immunization group were 4.6-fold lower than titers in the BV2438 alone immunization group (p<0.0001) and 3.1-fold lower than titers in the group that received bivalent rS (p<0.0001).

We next assessed neutralizing antibody titers among the different vaccination regimens. Sera collected from vaccinated animals at day 32 post vaccination were assessed using SARS-CoV-2/WA1, SARS-CoV-2/B.1.1.7 and SARS-CoV-2/B.1.351 strains in a plaque reduction neutralizing titer assay (PRNT₅₀). Sera from the monovalent BV2373 group displayed similar neutralizing antibody titers to each of the 3 virus strains. Sera from mice immunized with monovalent BV2438 produced elevated neutralizing antibody titers to the B.1.351 and the B.1.1.7 strain compared to the B.2 strain (FIG. 54E). The heterologous vaccine group produced similar elevated neutralizing antibody titers to the B.1.351 and the B.1.17 strain compared to the B.2 strain, as did the bivalent BV2373/BV2438 vaccination regimen.

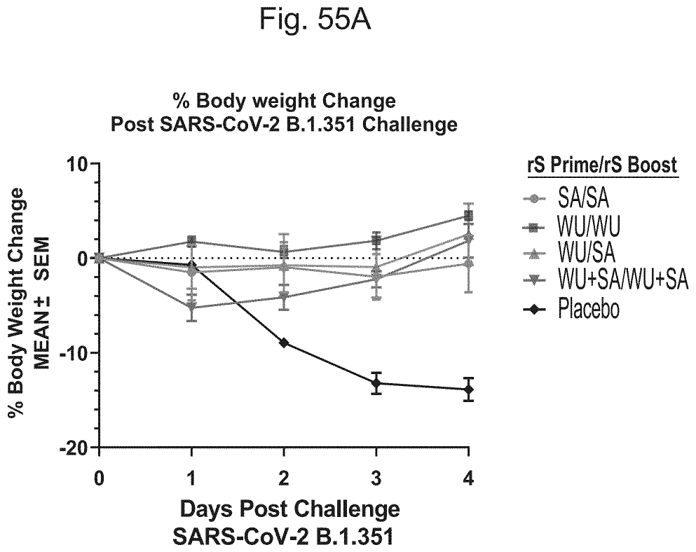

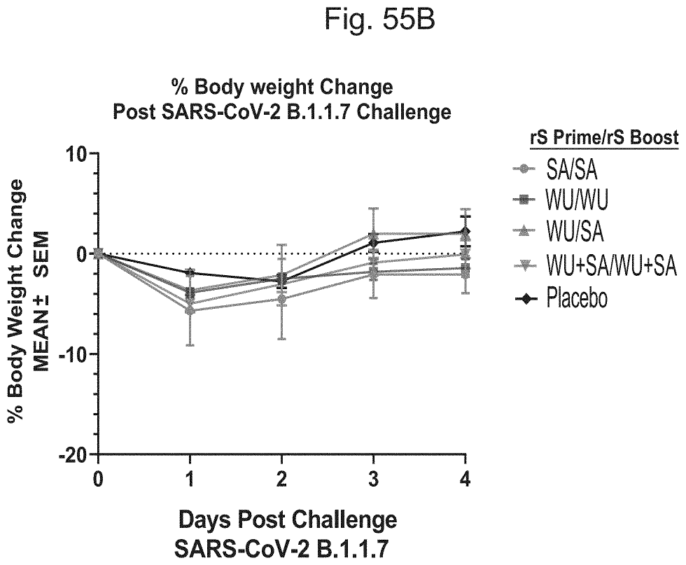

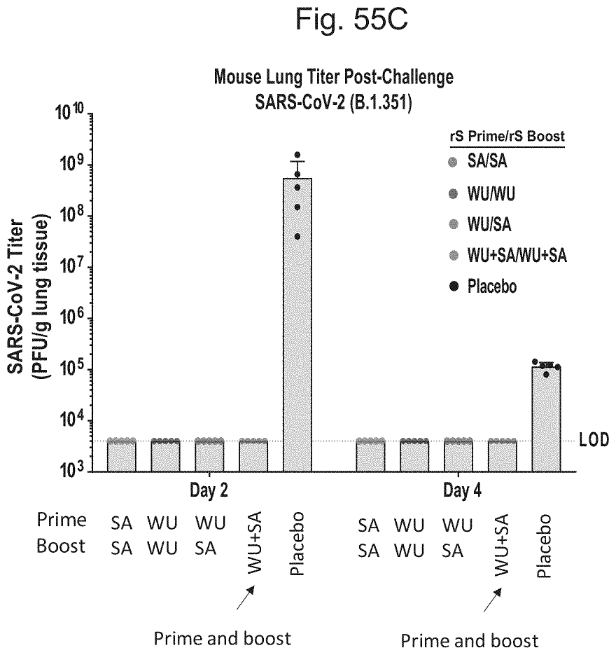

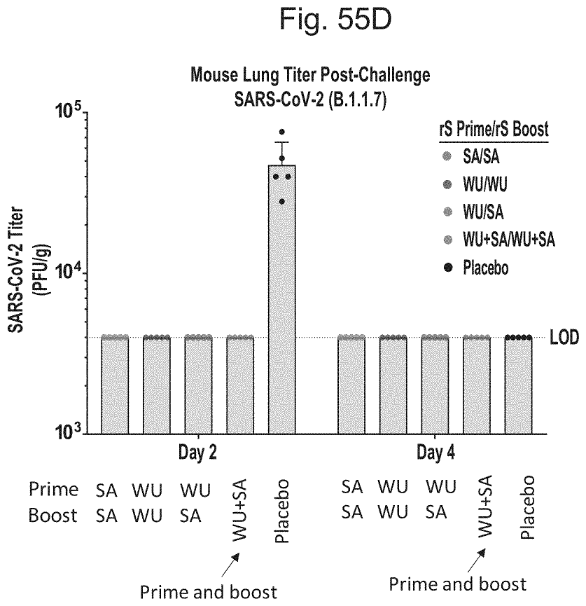

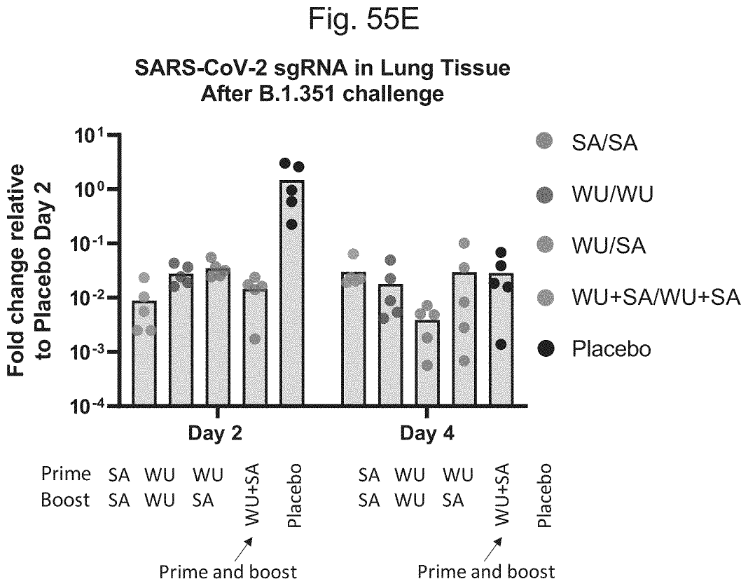

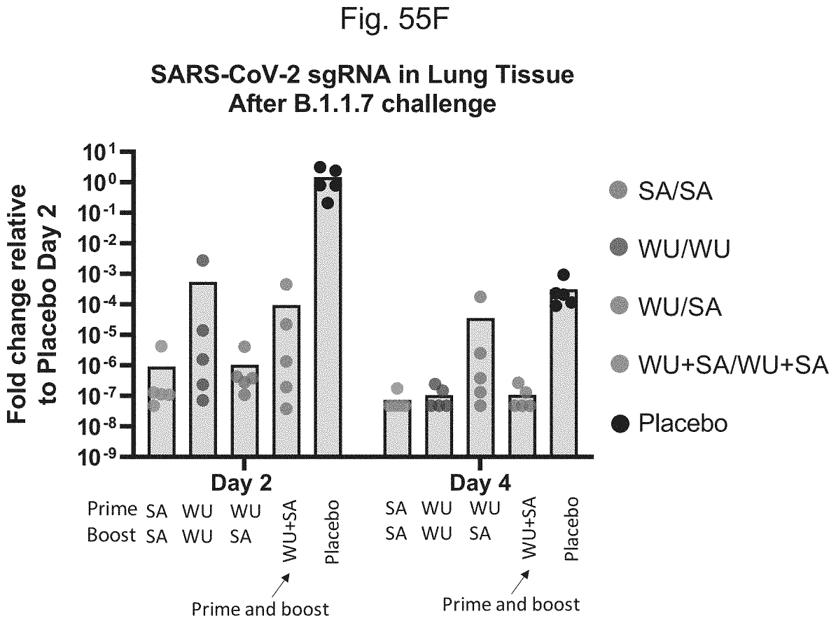

BV2438 Protection against SARS-CoV-2 in BALB/c mice: Mice vaccinated as described in FIG. 53 were evaluated for their ability to produce protective immunity against challenge with either B.1.1.7 or B.1.351. While the SARS-CoV-2/Wuhan 1 (B.2) strain does not replicate in wild type mice, the B.1.1.7 and B.1.351 strains have a 501Y mutation in the Spike ORF allowing for Spike protein to bind to mouse ACE2 and enter cells. At day 46 post vaccination, mice were intranasally inoculated with either 7×10⁴ PFU of B.1.1.7 (n=10 mice per group) or 1×10⁵ PFU of B.1.351 (n=10 mice per group). Mice were weighed daily throughout the post-challenge period, and at 2 and 4 days post infection (Study Days 48 and 50), 5 mice per group were euthanized by isoflurane inhalation. Lungs of each mouse were then assessed for viral load by plaque formation assay and viral RNA by RT-PCR. Placebo BALB/c mice infected with B.1.1.7 did not lose weight and there was no observed weight loss in any vaccinated group that was infected with this SARS-CoV-2 strain. For B.1.351 infected mice, 20% weight loss was observed in the placebo vaccination group by day 4 post infection with B.1.351 (FIG. 55A, FIG. 55B). All mice vaccinated with either regimen were protected from weight loss after infection with B.1.351, demonstrating a clinical correlate of protection in this model.

At day 2 post infection, B.1.1.7 infected mice in the placebo group exhibited 4×10⁴ pfu/g lung, which dropped to undetectable levels by day 4 post infection in the placebo vaccinated group. Upon immunization with any BV2373 or BV2438 regimen, there was no detectable live virus at day 2 or day 4 post infection, demonstrating a greater than 5-log reduction in viral load and protection from infection following vaccination (FIG. 55C, FIG. 55D). At day 2 post infection, B.1.351 infected mice in the sham vaccinated group exhibited 8×10⁸ pfu/g lung, which dropped to 2×10⁵ pfu/g lung by day 4 post infection. Upon immunization with any rS regimen, there was no detectable live virus at day 2 or day 4 post infection in the B.1.351 infected mice. This demonstrates a dramatic reduction in virus titer, with >5 log reduction in viral load by day 2 post infection from the sham vaccinated mice (FIG. 55C, FIG. 55D). Lung RNA was also assayed for subgenomic (sgRNA) SARS-CoV-2 mRNA production after challenge. Relative to levels in the respective Placebo groups, we found >99% reduction in lung sgRNA levels in immunized mice at day 2 and day 4 after infection with both strains (FIG. 55E, FIG. 55F).

These results confirm that BV2373 and BV2438 formulated with saponin adjuvant and administered as monovalent, bivalent, or heterologous regimens confer protection against both strains of SARS-CoV-2, B.1.1.7 and B.1.351, in mice. Together with the reduction in weight loss, high neutralizing antibody titers, and elimination of viral replication in the lungs of mice, we demonstrate a highly protective vaccine response by the variant Spike targeted vaccine.

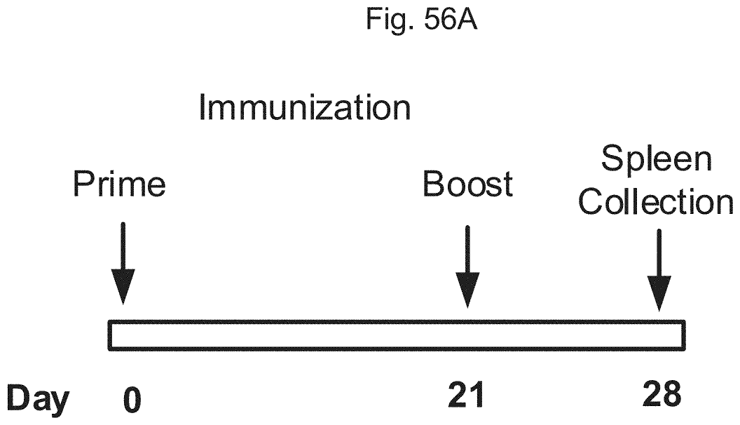

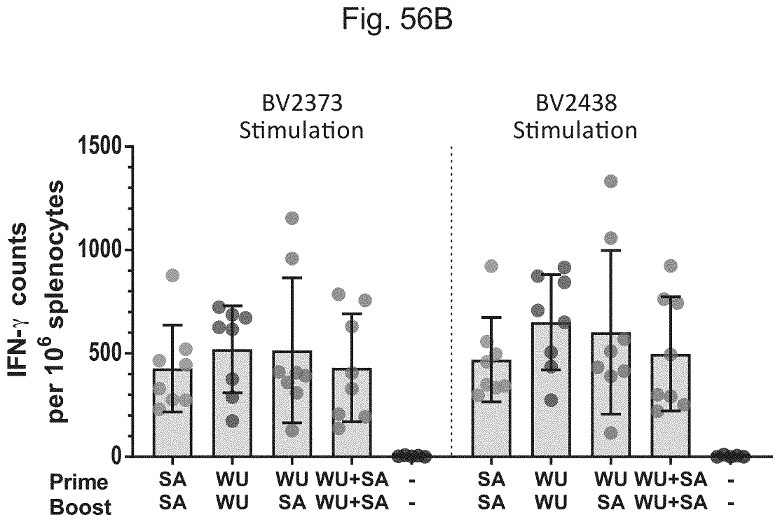

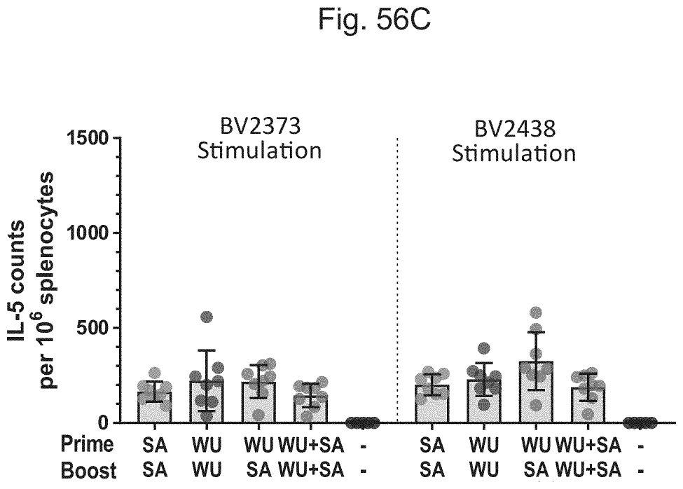

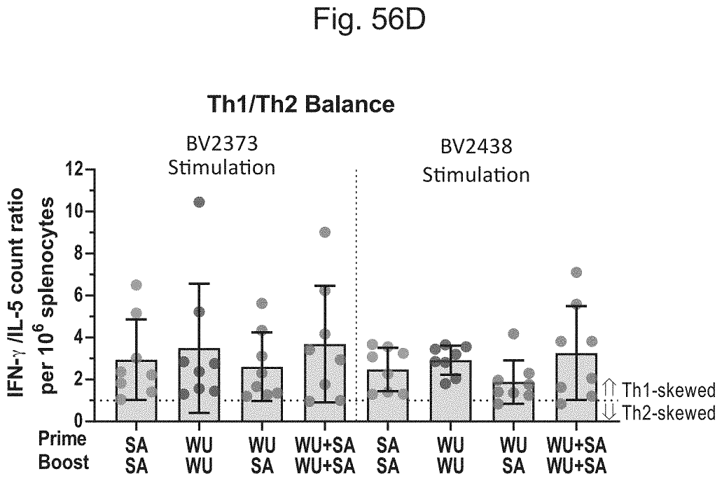

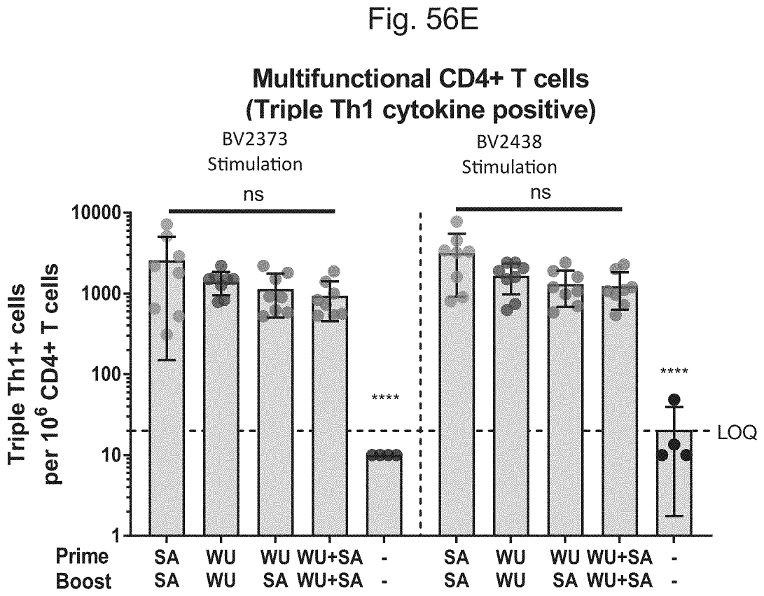

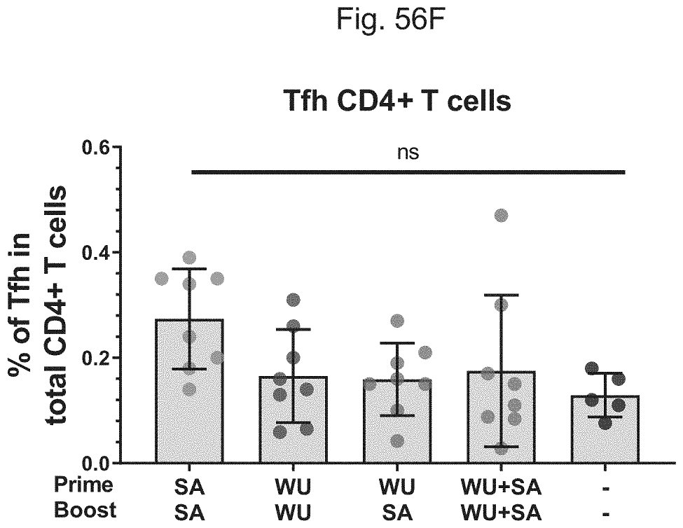

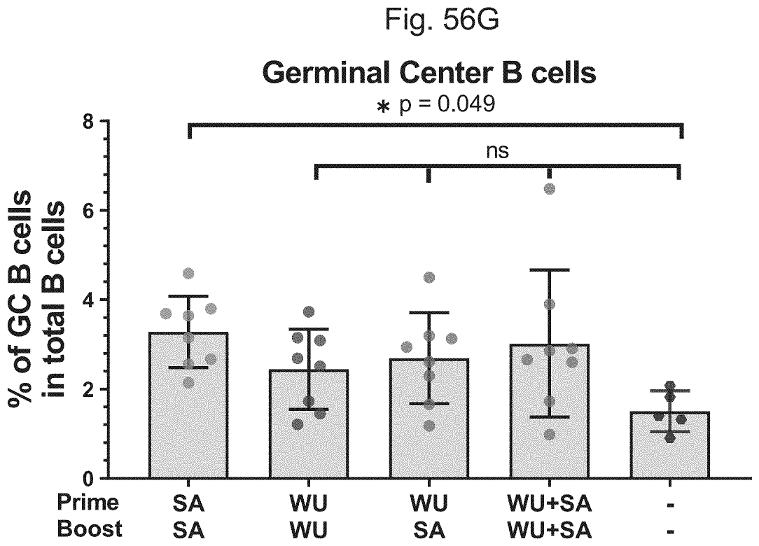

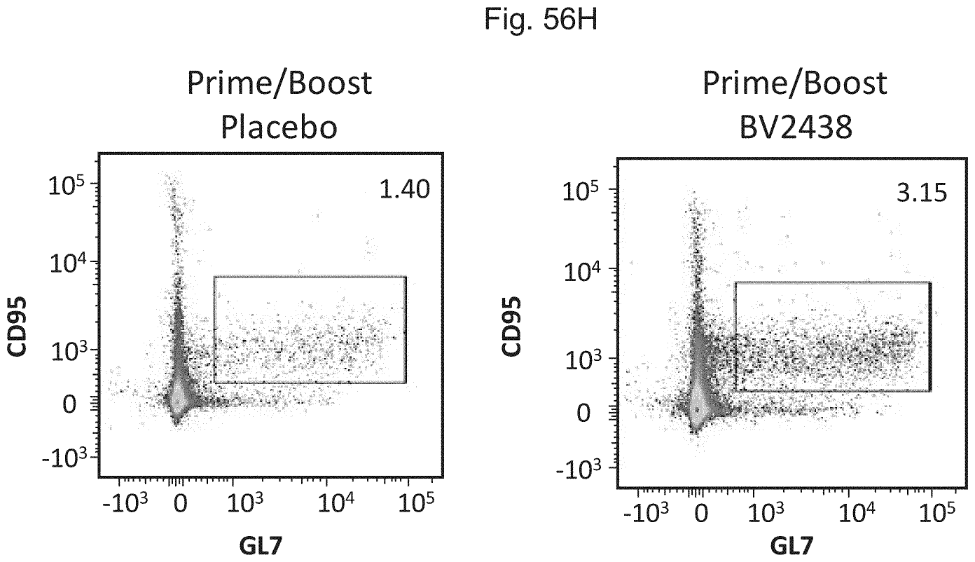

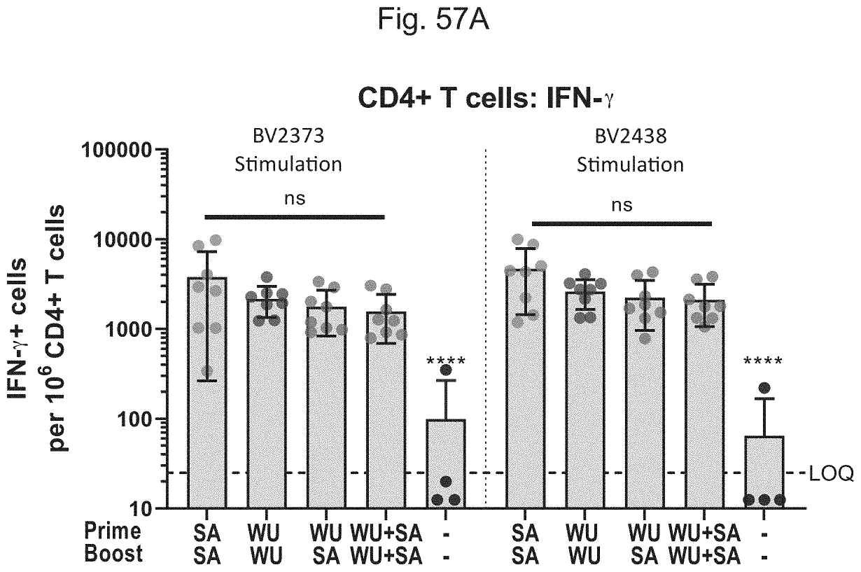

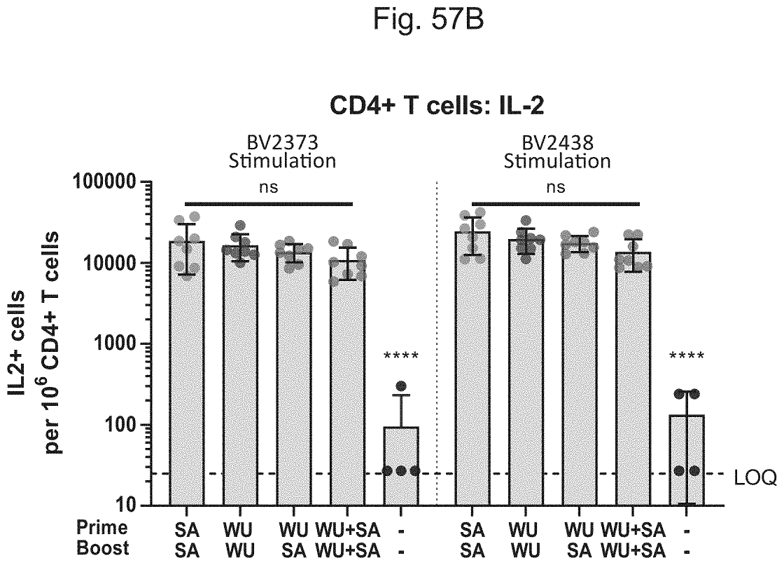

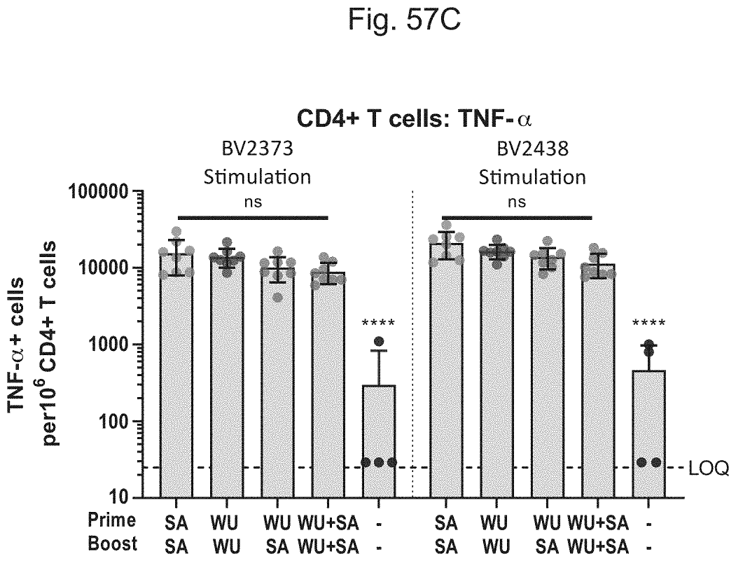

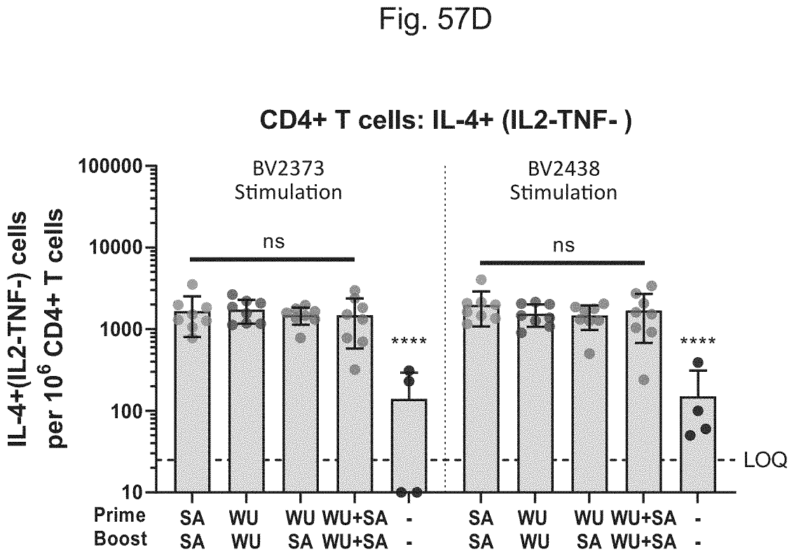

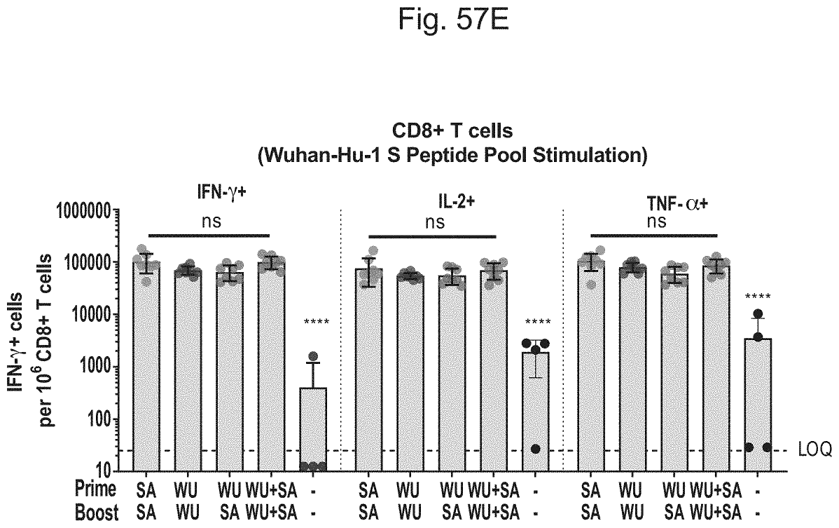

Cell-mediated immunogenicity of BV2438 in Mice: Groups of BALB/c mice (n=8/group) were immunized with the same BV2373 or BV2438 regimens mentioned above, but at a 21-day interval (FIG. 56A). A negative control group (n=4) was injected with vaccine formulation buffer. Spleens were harvested on study day 28, 7 days after the boost immunization. Splenocytes were collected and subjected to ELISpot and intracellular cytokine staining (ICS) to examine cytokine secretion upon stimulation with BV2373 or BV2438. Enzyme linked immunosorbent assay (ELISA) showed greater numbers of IFN-γ producing cells compared to the number of IL-5 producing cells upon all vaccination regimens, signifying a Th1-skewed response (FIGS. 56B-D). Upon stimulation with either rS, strong Th1 responses were observed by ICS as measured by the presence of CD4+ T cells expressing IFN-γ, IL-2, or TNF-α, and multifunctional CD4+ T cells expressing all 3 cytokines (FIG. 56E, FIGS. 57A-E). CD4+ T cells that expressed the Th2 cytokine IL-4 but were negative for IL-2 and TNF-α were also present, but at a lower proportion than that observed for Th1 cytokines (FIGS. 57A-E). No significant differences in cytokine-positive cell number were observed among vaccination groups for any cytokine tested upon stimulation with either BV2373 or BV2438.

T follicular helper cells (CSCR5+PD-1+CD4+) tended to represent a greater percentage of CD4+ T cells, though no statistically significant elevation was observed in vaccinated animals compared to placebo animals (FIG. 56F). Similarly, germinal center formation was evaluated by determining the percentage of GL7+CD95+ cells among CD19+ B cells using flow cytometry, and though a tendency toward higher percentage of germinal center B cells was observed in vaccinated groups compared to the placebo group, only animals immunized with the monovalent BV2438 regimen showed a significantly higher proportion (p=0.049 compared to placebo; FIG. 56G).

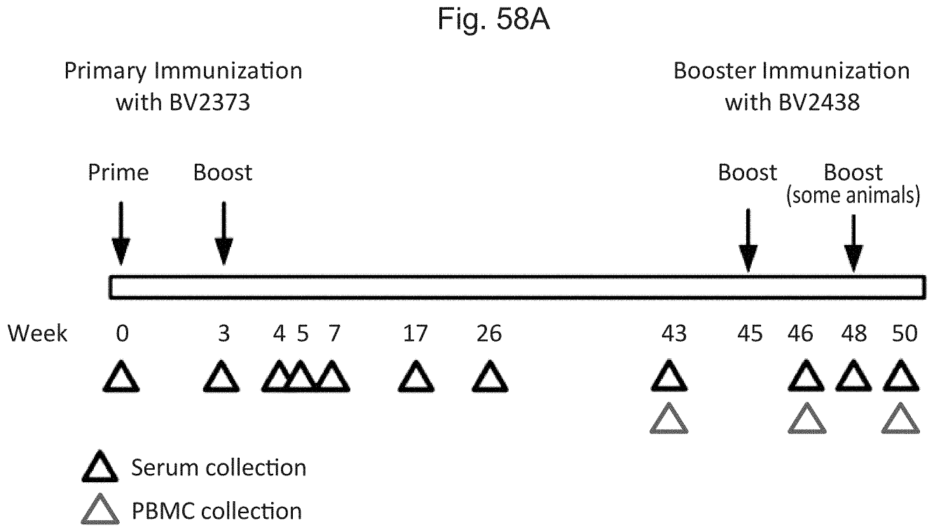

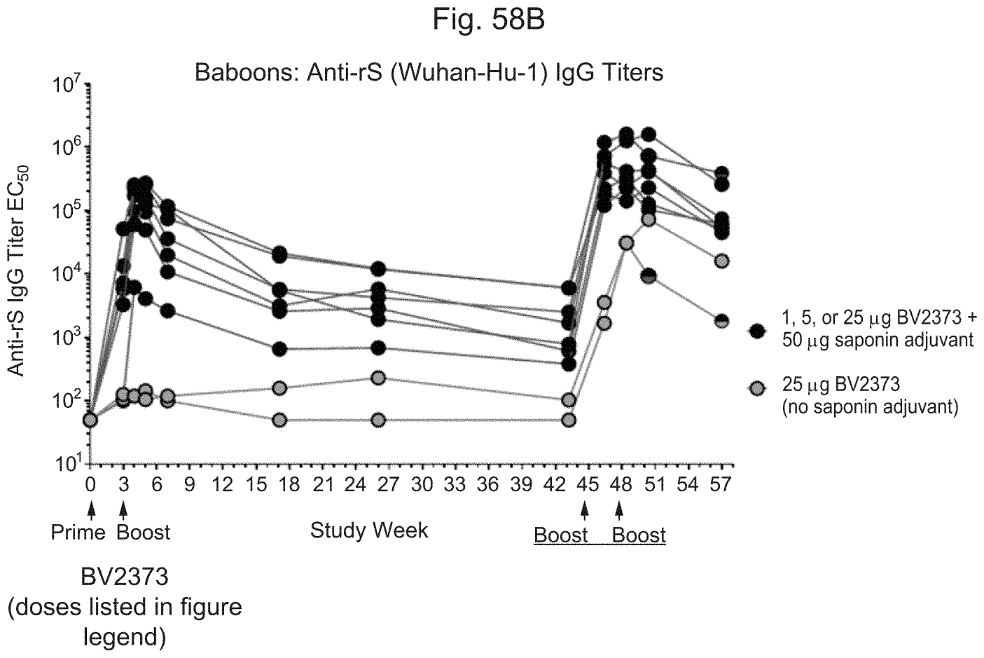

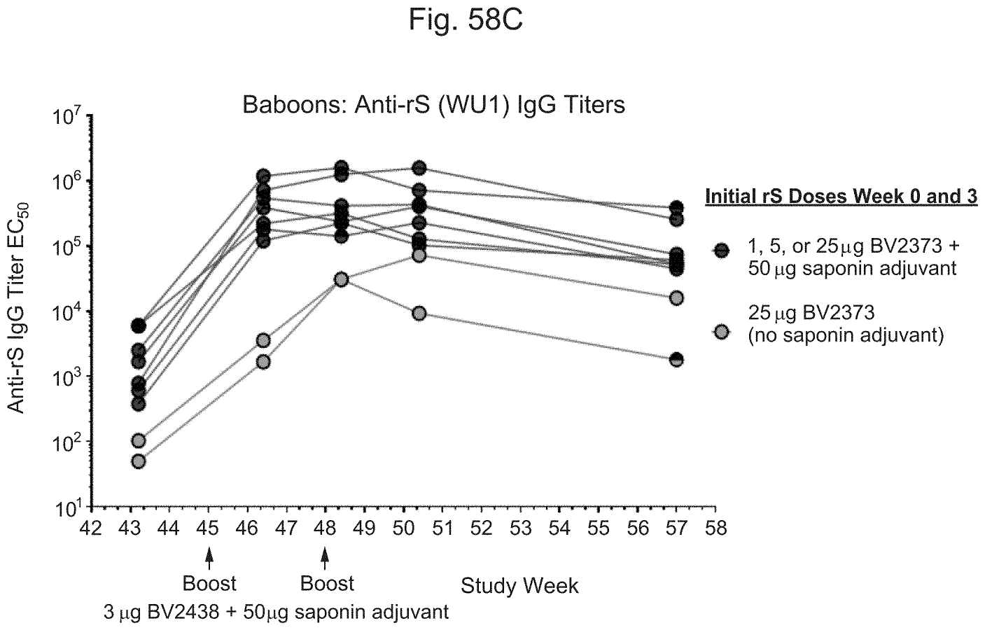

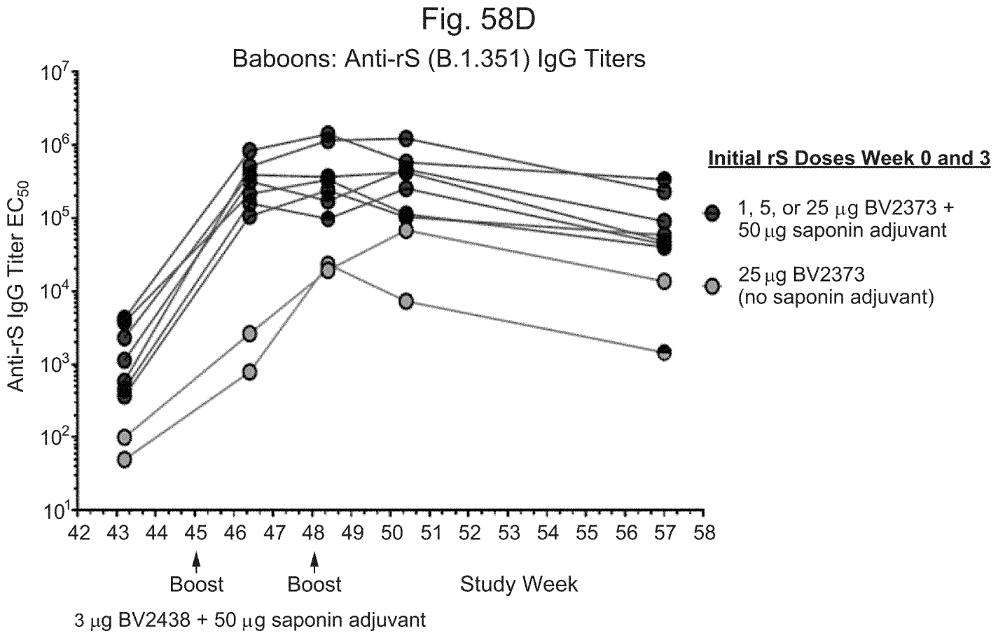

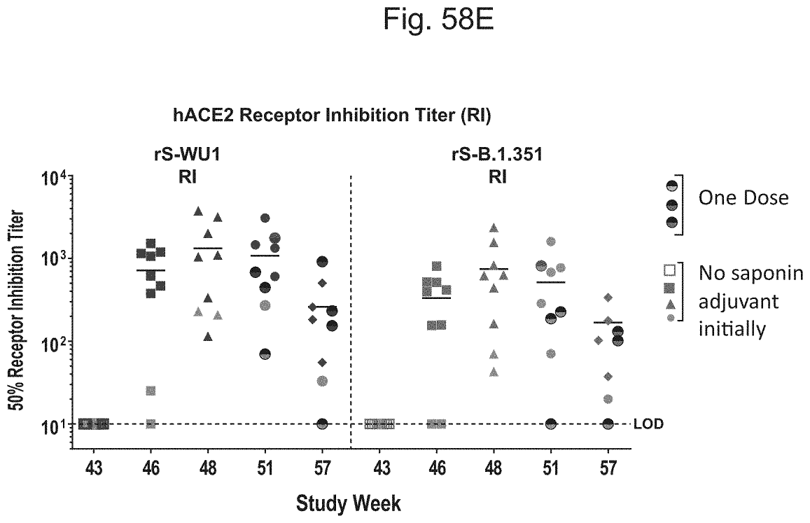

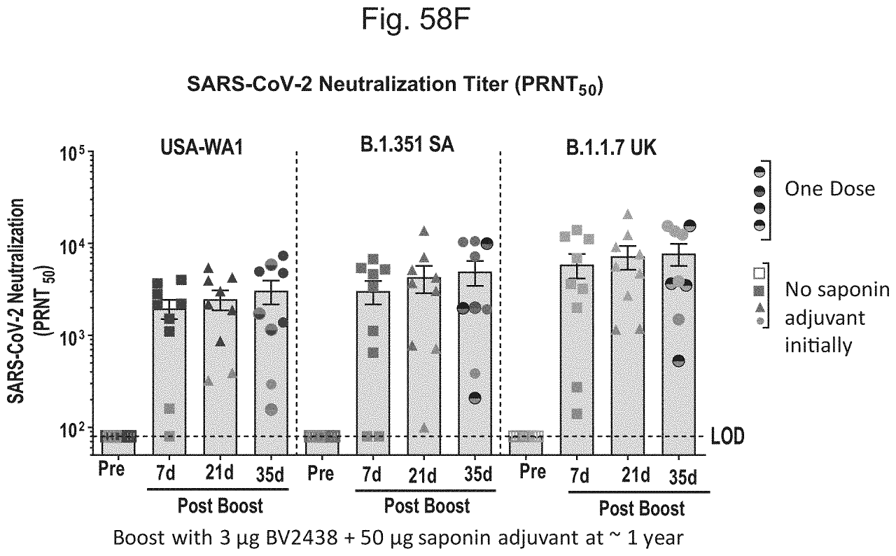

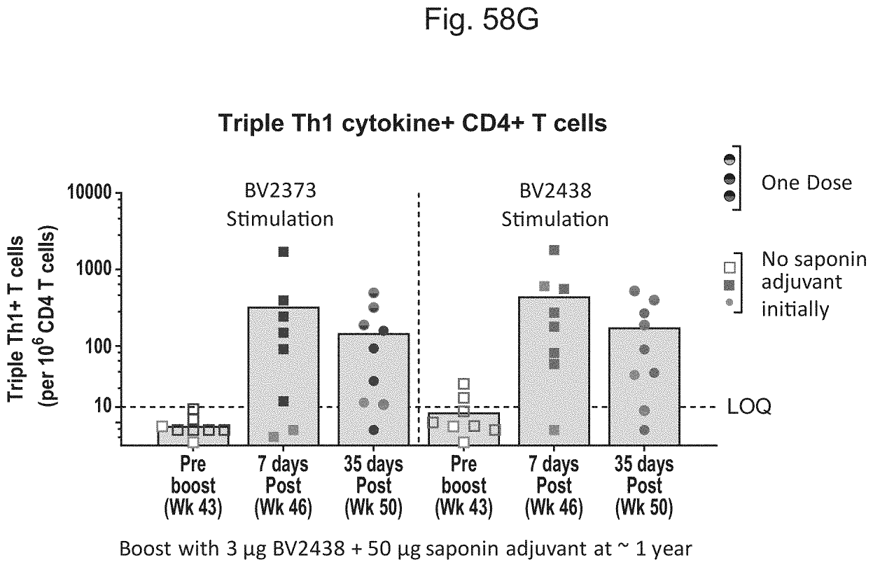

Anamnestic response induced by boosting with BV2373 one year after primary immunization with BV2373 in baboons: A small cohort of baboons (n=9 total) were subjected to a primary immunization series with BV2373 (either 1 μg, 5 μg, or 25 μg rS with 50 μg saponin adjuvant, or unadjuvanted 25 μg rS). Approximately one year later, all animals were boosted with one or two doses of 3 μg BV2438 with 50 μg saponin adjuvant to examine the resulting immune responses (FIG. 58A). Seven days after the first BV2438 boost, animals that had originally received adjuvanted BV2373 exhibited a strong anamnestic response as exhibited by levels of anti-S (WU1) IgG titers higher than that originally observed at peak immune response during the primary immunization series (FIG. 58B). This response did not seem to be further bolstered by a second booster dose of BV2438, though the small sample sizes utilized in this study prohibit a meaningful quantitative analysis. Animals that received unadjuvanted BV2373 during the primary immunization series exhibited a weaker response to boosting with BV2438, though still exhibited elevated anti-S (WU1) IgG response. The BV2438 boost elicited comparable antibody titers against BV2373 and BV2438, with animals that originally received unadjuvanted BV2373 exhibiting a weaker response (FIG. 58C, FIG. 58D).

Serum antibody titers capable of disrupting the interaction between the wild-type CoV S protein (SEQ ID NO: 2) or B.1.351 rS and hACE2 were also evaluated at before boost, and 7, 21, 35, and 89 days after the boost with 1 or 2 doses of BV2438. Similarly to what was observed for anti-S IgG titers, animals that had received adjuvanted vaccine during the primary immunization series exhibited a strong hACE2-inhibiting antibody response 7 days after the BV2438 boost, despite having undetectable titers before the boost. Titers were slightly higher for BV2373-hACE2 blocking antibodies compared to levels of BV2438-hACE2 blocking antibodies, though the small sample size prohibits a meaningful quantitative analysis. Animals that had received unadjuvanted vaccine during the primary immunization series exhibited lower hACE2 blocking titers after the BV2438 boost (FIG. 58E).

Neutralizing antibody titers were analyzed by live virus microneutralization assays by testing sera for the ability to neutralize WA1, B.1.351 and B.1.1.7. Sera collected before the BV2438 boost had undetectable neutralizing antibody levels against all these viruses. By 7 days post vaccination, high titer antibody that neutralized all 3 strains was detected and this antibody response stayed high through 35 days post vaccination. Animals immunized with unadjuvanted BV2373 in the primary series displayed significantly lower antibody levels with a much broader range of neutralization titers (FIG. 58F). Together, these data demonstrate a robust durable antibody response even 1 year after the primary vaccination series.

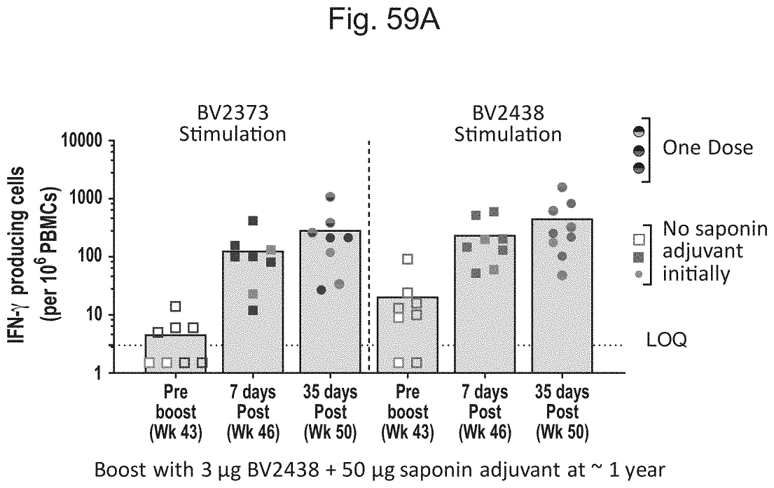

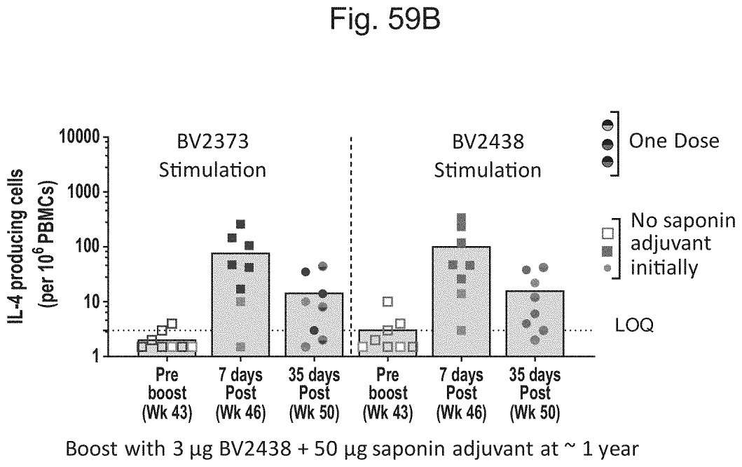

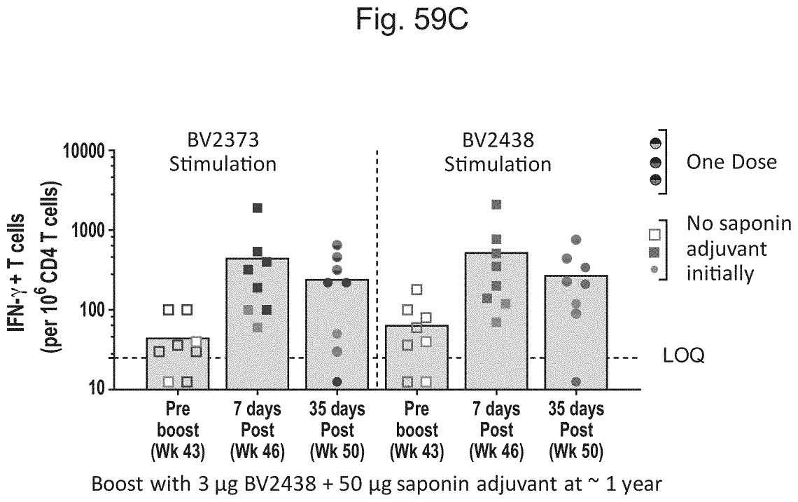

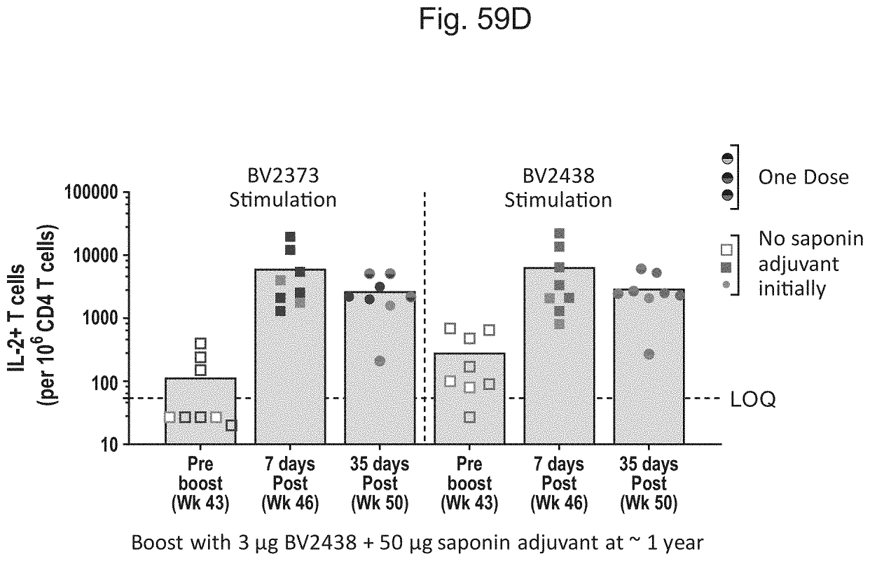

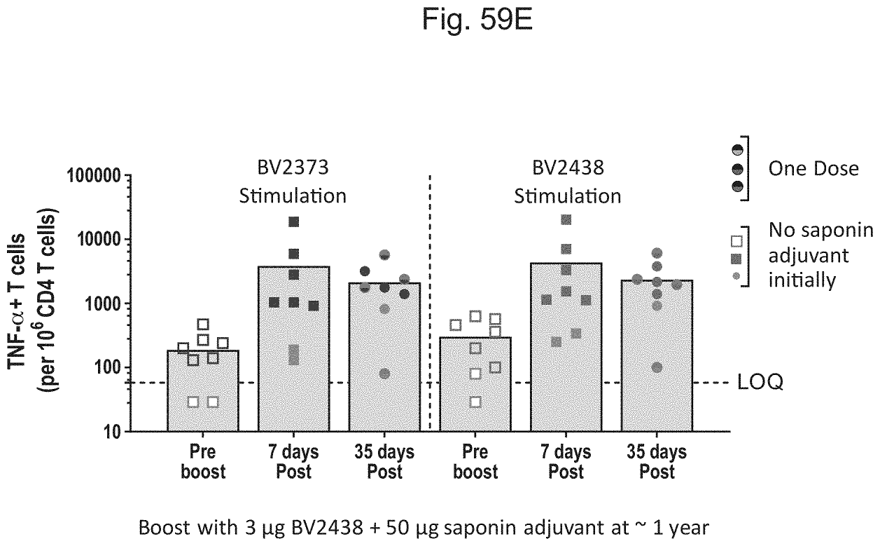

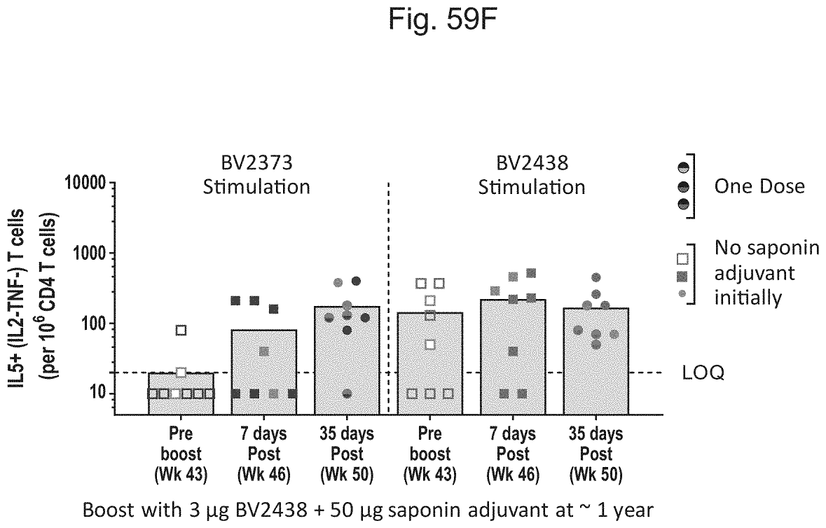

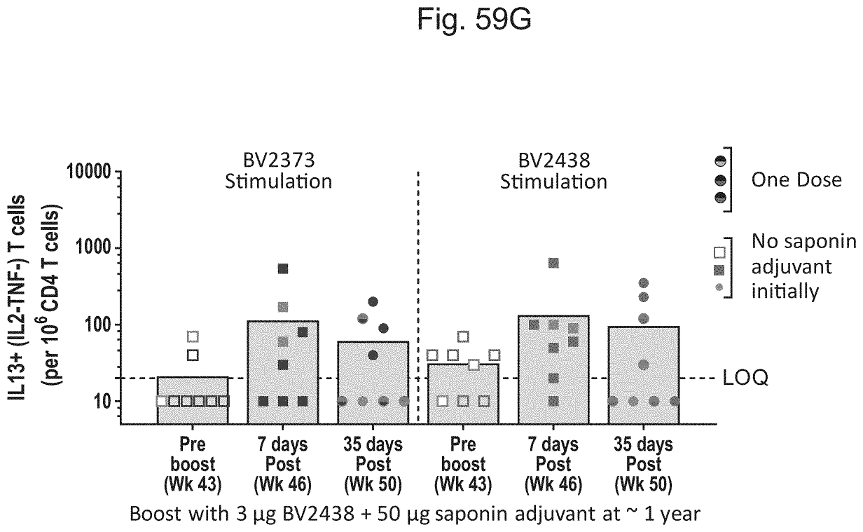

Multifunctional T cells expressing 3 Th1 cytokines were also observed 7 days after the first BV2438 booster dose in baboons, and these responses were maintained at 35 days after the first booster dose (FIG. 58G and FIGS. 59A-G).

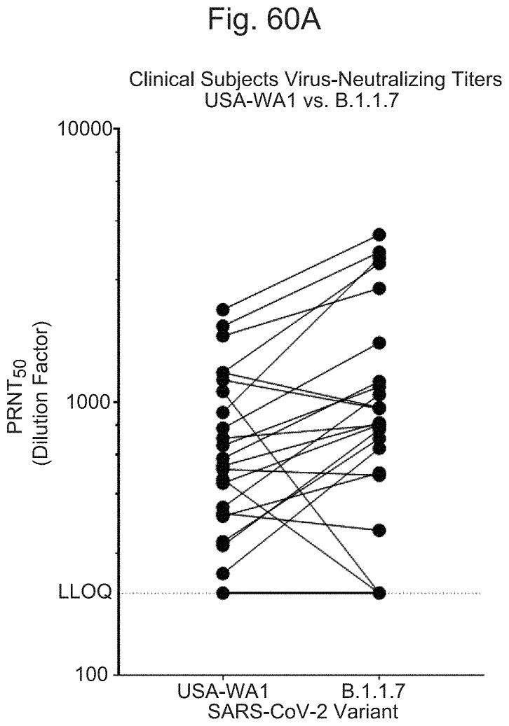

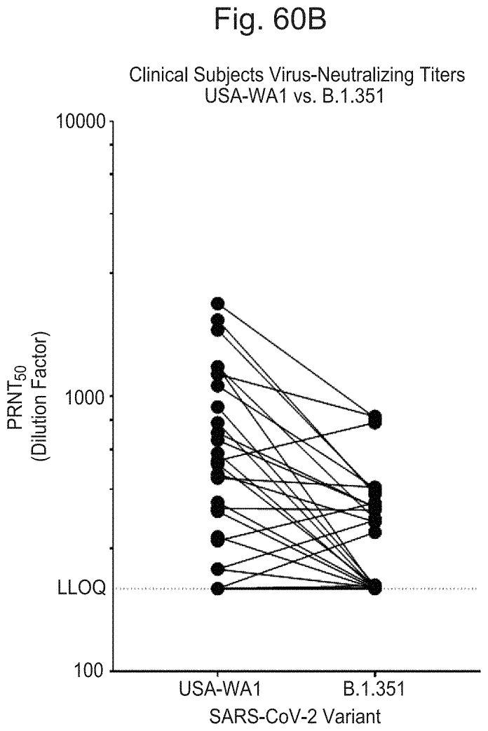

Neutralization of SARS-CoV-2 Variants by Sera from BV2373 Vaccinated Adults: A vaccine containing BV2373 and saponin adjuvant is currently in clinical trials globally, including in locations where B.1.1.7 and B.1.351 are prevalent. We assessed the capacity of sera from individuals in these trials to neutralize USA-WA1, B.1.1.7 and B.1.351. Microneutralization assays were performed with a PRNT₅₀ readout (FIG. 60A, FIG. 60B). Thirty randomly selected serum samples from clinical trial participants after their second dose of the vaccine were assayed. When comparing WA1 vs B.1.1.7, there was no change in neutralizing activity across the majority of the serum samples; only 1 sample had a statistically significant change in neutralizing antibody titers against B.1.1.7. The WA1 vs B.1.351 neutralization titers showed increased range of neutralization titers with five out of 30 samples showing reduced neutralization 1 standard deviation away from the mean in the PRNT₅₀ assay. This data demonstrates a reduced neutralization of B.1.351 in a small percentage of vaccinees receiving BV2373 and saponin adjuvant compared to B.1.1.7.

Discussion: We have shown that a full-length, stabilized prefusion SARS-CoV-2 spike glycoprotein vaccine using the B.1.351 Spike variant adjuvanted by saponin adjuvant can induce high levels of functional immunity and protects mice against both B.1.1.7 and B.1.351 SARS-CoV-2 strains. Immunizing mice or non-human primates with BV2438 induced anti-S antibodies, hACE2-receptor inhibiting antibodies, and SARS-CoV-2 neutralizing antibodies. In addition, the BV2438 vaccine induced CD4⁺ T cell responses, induced germinal center formation and provided protection against B.1.351 and B.1.1.7 challenge.

In mice, the antibodies produced after vaccination with the B.1.351 variant-directed vaccine were able to inhibit binding between hACE2 and variant spike or ancestral spike to the same degree, indicating that this variant-directed vaccine could efficiently protect “backward” against ancestral SARS-CoV-2 strains.

Analysis of human vaccine sera from our trials demonstrates a robust antibody response and minimal loss of neutralization. We observed that B.1.351 virus does not significantly reduce neutralization compared to B.1.1.7 and WA1, even though there is evidence of breakthrough infections in the WA.1 trial participants in South Africa. All breakthrough infections were B.1.351. Booster vaccinations containing a single or multiple variant rS vaccines will thus increase antibody levels as well as broaden coverage to variants as shown in this work.

### Example 11

### BV2373 and Saponin Adjuvant Induce Protective Immune Responses Against Heterogeneous SARS-CoV-2 Strains After a Single Boost Dose

Participants: Healthy male and female participants ≥18 to ≤84 years of age were recruited for enrollment in this study. Participants were eligible if they had a body mass index of 17 to 35 kg/m², were able to provide informed consent prior to enrollment, and (for female participants) agreed to remain heterosexually inactive or use approved forms of contraception. Participants with a history of severe acute respiratory syndrome (SARS) or a confirmed diagnosis of COVID-19, serious chronic medical conditions (e.g, diabetes mellitus, congestive heart failure, autoimmune conditions, malignancy), or that were currently being assessed for an undiagnosed illness which may lead to a new diagnosis, were excluded from the study. Pregnant or breastfeeding females were also excluded.

Randomization: Patients were randomly assigned to five groups. Of the five treatment groups, one was a placebo control (Group A) and two were active vaccine groups that were considered for additional vaccination with a booster (Group B and Group C). After approximately 6 months, consenting participants who had been randomized to receive a primary vaccination series of either two doses of BV2373 (5 μg) and saponin adjuvant (50 μg) on Day 0 and Day 21 (Group B) or one dose of BV2373 (5 μg) and saponin adjuvant (50 μg) on Day 0 and placebo on Day 21 (Group C) were re-randomized 1:1 to receive either a single booster dose of BV2373 and saponin adjuvant at the same dose level (Groups B2 and C2) or placebo (Groups B1 or C1) at Day 189. Group B participants are the main focus of this Example.

Purpose and Methods: We conducted a phase 2, randomized, observer-blinded, placebo-controlled trial in healthy adults aged 18 to 84 who received three intramuscular 5 μg doses of BV2373 and 50 μg saponin adjuvant (Fraction A and Fraction C iscom matrix, also referred to as MATRIX-M™ in this example) or placebo (1:1). The first and second dose were administered 21 days apart. The first and second dose are referred to as the “primary vaccination series.” The third dose (“boost” dose) was administered about 6 months following the primary vaccination series. The injection volume of all three doses was 0.5 mL. Safety and immunogenicity parameters were assessed, including assays for IgG, MN₅₀, and hACE2 inhibition against the ancestral SARS-CoV-2 strain and select variants (B.1.351 [Beta], B.1.1.7 [Alpha], B.1.617.2 [Delta]).

Participants utilized an electronic diary to record reactogenicity on the day of vaccination and for an additional 6 days thereafter. Blood samples for immunogenicity analysis were collected 28 days after receipt of the booster, with safety follow-up also being performed at this time. Measures of immune response included assays for serum immunoglobulin G (IgG) antibodies, neutralizing antibody activity (microneutralization assay at an inhibitory concentration >50% [MN₅₀]), and human angiotensin-converting enzyme 2 (hACE2) receptor binding inhibition. Serum IgG antibody levels specific for the SARS-CoV-2 rS protein antigen were detected using a qualified IgG enzyme linked immunosorbent assay (ELISA). Neutralizing antibodies specific for SARS-CoV-2 virus were measured using a qualified wild-type virus MN assay. Serum IgG and MN₅₀ assay data were collected for both the ancestral and Beta variant SARS-CoV-2 strains. A fit-for-purpose functional hACE2 inhibition assay and an anti-rS (anti-recombinant spike) IgG activity assay were both used to analyze responses against the ancestral, B.1.351 (Beta), B.1.1.7 (Alpha), and B.1.617.2 (Delta) variant strains of SARS-CoV-2.

Safety outcomes included participant-reported reactogenicity events for 7 days following the booster, as well as unsolicited adverse events occurring through 28 days post-booster. Booster reactogenicity was documented separately by solicited local and systemic adverse events. Unsolicited adverse events from booster vaccination to 28 days post-booster were recorded. Data were also collected on whether an adverse event was serious, related to vaccination, related to COVID-19, a potentially immune-mediated medical condition (PIMMC), or lead to discontinuation or an unscheduled visit to a healthcare practitioner. Participant samples for immunogenicity analyses were collected immediately prior to and 28 days after the booster.

Statistics: Analyses included safety and immunogenicity data from participants in Group B obtained during and after their primary vaccination series (Day 0, Day 21, Day 35, Day 105, and Day 189) for comparison with data collected from Group B2 28 days following their receipt of the booster dose (Day 217). Results were also analyzed by participant age group: ≥18 to ≤84 years of age, ≥18 to ≤59 years of age, and ≥60 to ≤84 years of age.

The safety analysis included all participants who received a single booster injection of BV2373 and saponin adjuvant (Group B2) or placebo (Group B1). Safety analyses were presented as numbers and percentages of participants with solicited local and systemic adverse events analyzed through 7 days after each vaccination and unsolicited adverse events through 28 days following the booster.

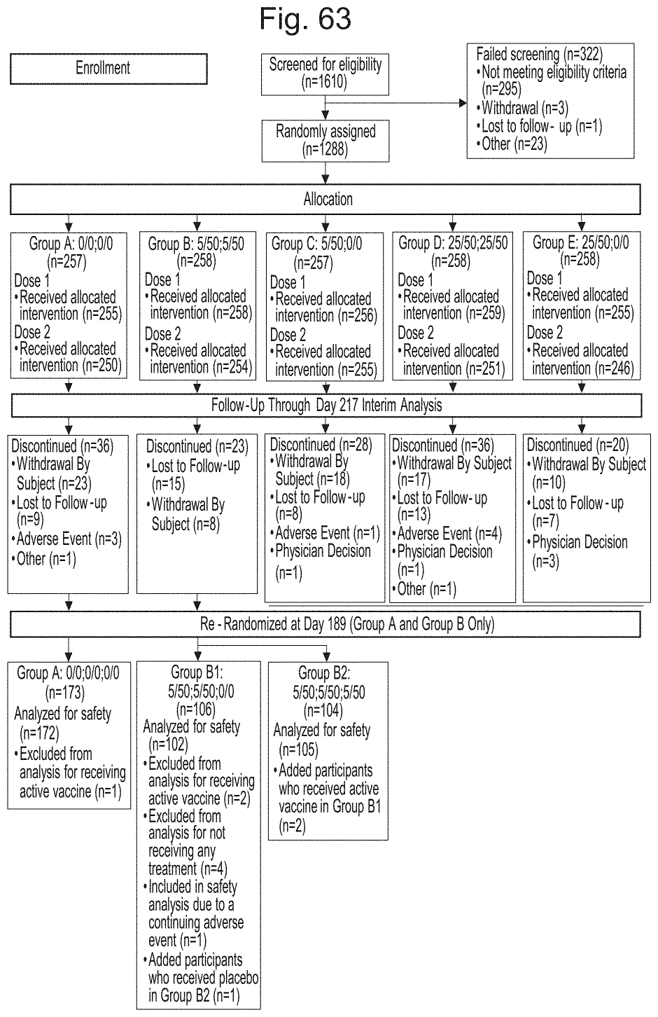

Results: A total of 1610 participants were screened. All but three participants randomized to Group B (n=2.57) received both doses of BV2373 and saponin adjuvant in their primary vaccination series and were considered for investigation of a single booster dose at the same dose level (FIG. 63). Re-randomization of Group B participants took place at Day 189, with 210 consenting participants assigned 1:1 to receive a single booster of BV2373 and saponin adjuvant in Group B2 (n=104) or placebo in Group B1 (n=106). In Group B2, all but one participant received active vaccine as a booster. All but six participants in Group B1 received placebo as a booster; of the remaining six participants, four did not receive any booster (of which, one was included in Group B1 for safety due to an ongoing adverse event) and two received active vaccine in error as a booster and were assessed for safety in Group B2. All but one participant in Group A received placebo for all three doses, with the remaining participant receiving active vaccine as a booster dose.

Demographics and baseline characteristics were generally balanced between the active (Group B2) and placebo (Group B1) booster groups (Table 8), except for a higher proportion of female participants in Group B1 (58%) than Group B2 (45%). Across Groups A, B1, and B2, the median age was approximately 57 years and 45% of participants were ≥60 to ≤84years of age. Most participants were White (87%) and not Hispanic or Latino (95%), Baseline SARS-CoV-2 serostatus was predominantly negative (98%).

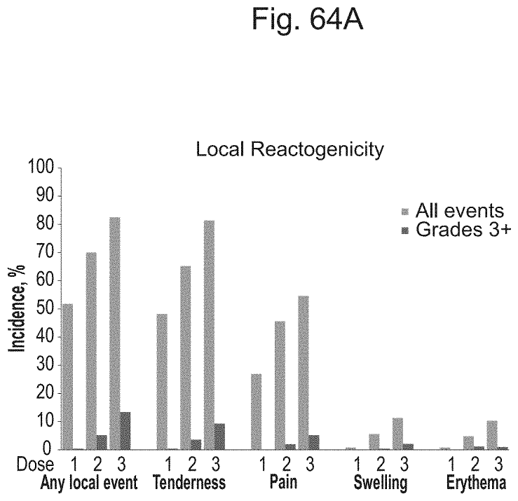

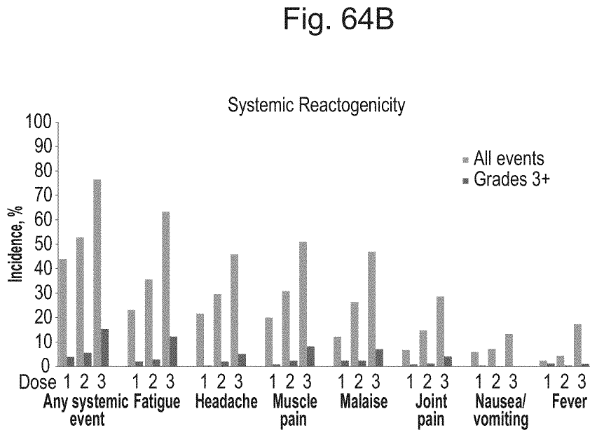

Safety reporting of solicited local and systemic reactogenicity events showed an increasing trend across all three doses of BV2373 and saponin adjuvant (FIGS. 64A-B). Following the booster, participants in Group B2 reported an incidence rate for any local reaction (tenderness, pain, swelling, and erythema) of 82.5% (13.4%≥Grade 3) compared to 70.0% (5.2%≥Grade 3) following the primary vaccination series. Grade 4 local reactions were rare, with two events (pain and tenderness) reported by one participant in Group B2 compared with no participants following the primary vaccination series. Following the booster, local reactions were short-lived with a median duration of 2.0 days for all events except erythema (2.5 days). Local reactions were also short-lived following the primary vaccination series, with median durations of 2.0 days for pain and tenderness and 1.0 day for erythema and swelling.

Systemic reactions showed a similar pattern with an incidence rate for any event (fatigue, headache, muscle pain, malaise, joint pain, nausea/vomiting, and fever) of 76.5% (15.3%≥Grade 3), compared to 52.8% (5.6%≥Grade 3), following the primary vaccination series. Grade 4 systemic reactions were rare, with three events reported by one participant in Group B2 (headache, malaise, and muscle pain) compared with no participants following the primary vaccination series. Following the booster, systemic reactions were transient in nature with a median duration of 1.0 day for all events except muscle pain which had a duration of 2.0 days. All systemic reactions were also short-lived following the primary vaccination series, with a median duration of 1.0 day for all events.

Local and systemic reactogenicity events were less frequent and less severe in older adults (≥60 to ≤84 years of age)) when compared to younger adults (≥18 to ≤59 years of age) following either the primary vaccination series or booster dose. In the younger cohort, post-booster local and systemic reactions were reported in 84.9% (18.9%≥Grade 3) and 84.9% (26.4%≥Grade 3) of participants, respectively, versus 79.5% (6.8%≥Grade 3) and 66.7% (2.2%≥Grade 3) of participants, respectively, in the older cohort.

Unsolicited adverse events were summarized across the active-boosted participants (Group B2), placebo-boosted participants (Group B1), and participants receiving three doses of placebo throughout the study (Group A). Through 28 days after the booster, participants who initially received active vaccine for their primary vaccination series (Groups B2 and B1) experienced a higher incidence of unsolicited adverse events than those who received only placebo (Group A), with 12.4%, 12.7%, and 11.0% of participants reporting such events, respectively. A similar trend was seen for unsolicited severe adverse events (5.7%, 3.9%, and 2.4%, respectively). Other types of AEs reported included medically attended. AEs (events requiring a healthcare visit; MAAEs), potential immune-mediated medical conditions (PIMMCs), events relevant to COVID 19, and serious adverse events (SAEs).

Overall, MAAEs occurred with a slightly higher frequency in active boosted participants across the three groups (30.5%, 26.1%, and 23.2% for Groups B2, B1, and A, respectively), with related events reported in few participants (1.9%, 0%, and 1.2%, respectively). Events considered PIMMCs were rare across the study, with one participant in Group B2 and Group A reporting a single event each; both events were assessed as not related to study treatment. No participant reported an as adverse event related to COVID-19.

SAEs were also infrequent across the study, occurring in 5.7%, 3.3%, and 1.6% of participants in Groups B2, B1, and A, respectively, with all events assessed as not related to study treatment.

Evaluation of SAEs for Group B2 and B1 participants did not show a relationship of with active boosting, as SAEs occurred in 0%, 4.8%, and 1.0% of participants in Group B2 and 0%, 2.0%, and 2.0% of participants in Group B1 following Dose 1, Dose 2, and the booster, respectively.

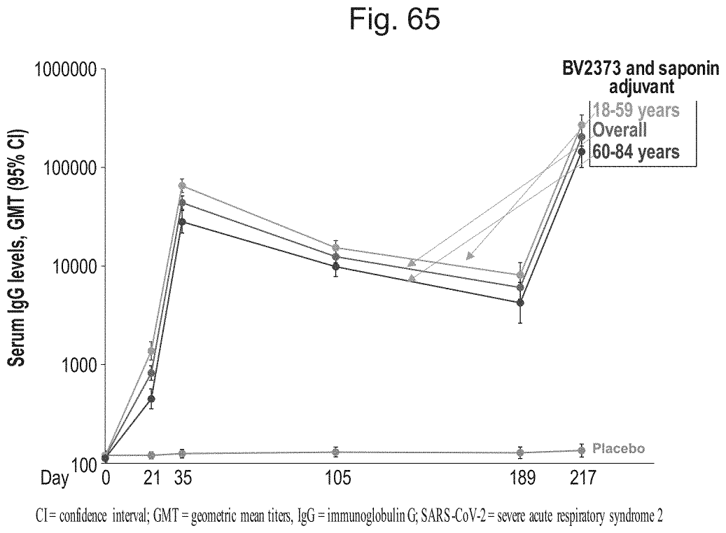

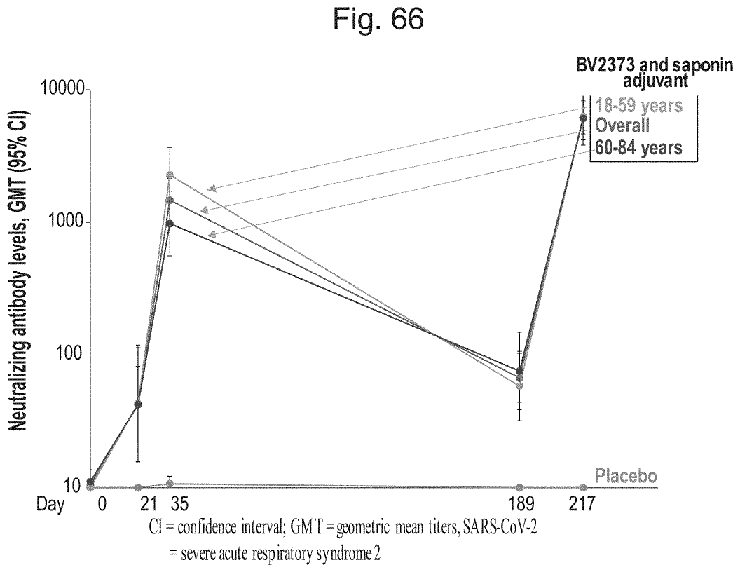

Declines in Group B IgG and MN50 geometric mean titers (GMTs) were observed following the primary vaccination series (Day 35) through Day 189 (43,905 units [EU] to 6,064 EU for IgG and 1,470 to 63 for MN50, respectively). Twenty-eight days following the booster (Day 217), IgG and MN50 titers increased robustly compared to both the pre-booster titers and to the Day 35 titers produced by the primary series (FIG. 65, FIG. 66).

For the ancestral SARS-CoV-2 strain, serum IgG GMTs increased ˜4.7-fold from 43,905 EU following the primary vaccination series (Day 35) to 204,367 EU following the booster (Day 217). Higher fold increases after boosting were seen in older adults (5.1-fold) compared to younger adults (4.1-fold). Similarly, MN50 assay GMTs specific to the ancestral SARS-CoV-2 strain increased ˜4.1-fold from 1,470 to 6,023 over the same respective time points with increases in older and younger adults of 4.0-fold and 3.8-fold, respectively.

For the Beta variant, IgG GMTs increased from 4,317 EU at Day 189 pre-booster to 175,190 EU at Day 217 reflecting a post-booster increase of ˜40.6-fold. These titers were 4-fold higher than those observed at Day 35 for the ancestral strain (GMT 175,190 EU vs 43,905 EU). Beta variant MN50 assay data showed a similar fold increase in titers from pre-booster (Day 189) to post-booster (Day 217) of ˜50.1-fold (GMT 13 vs 661), though titers were lower than those seen for the ancestral strain at Day 35 (GMT 661 vs 1,470). (Table 9, Table 10).

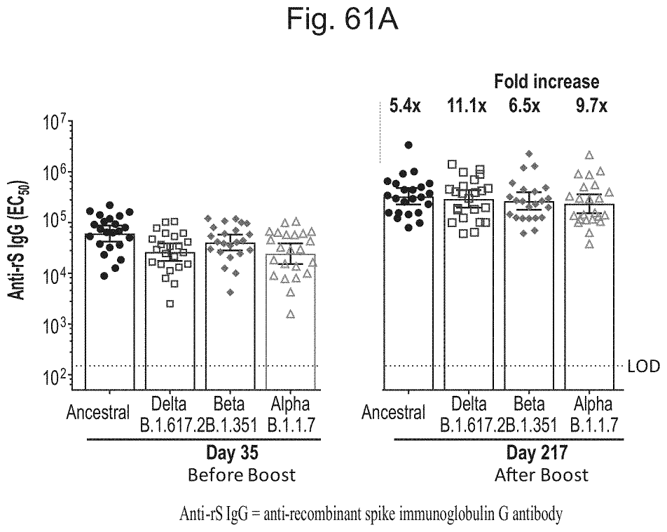

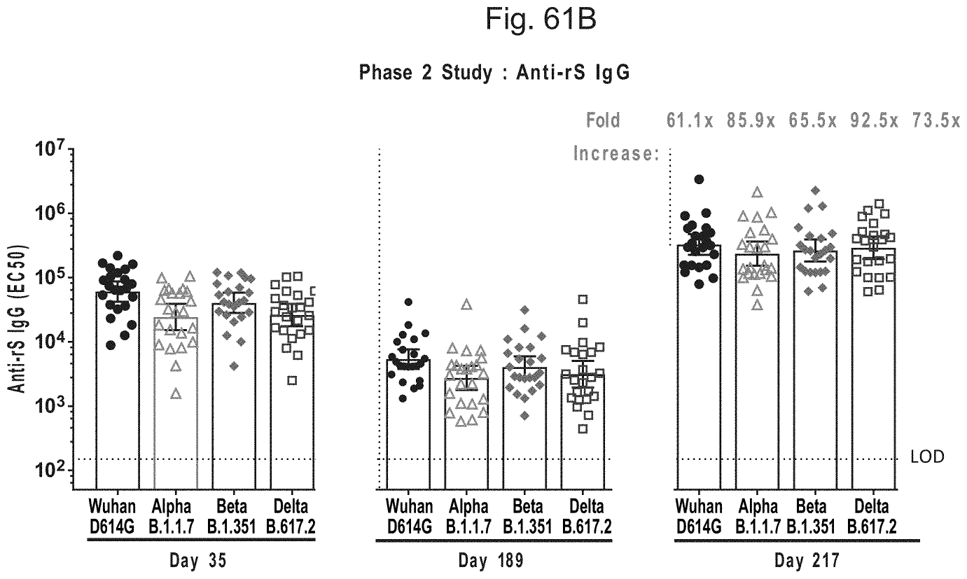

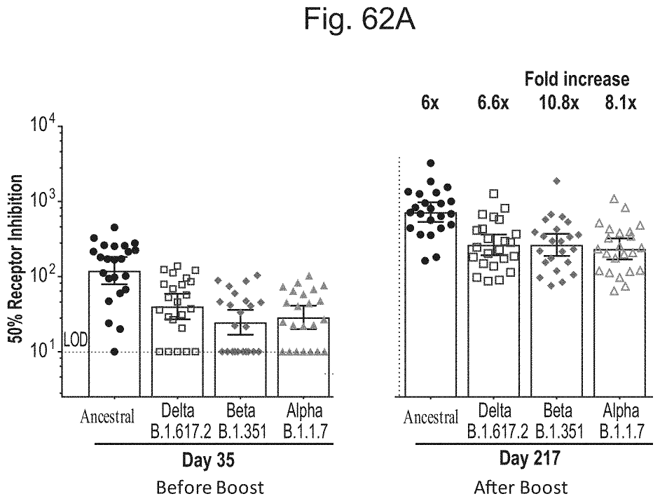

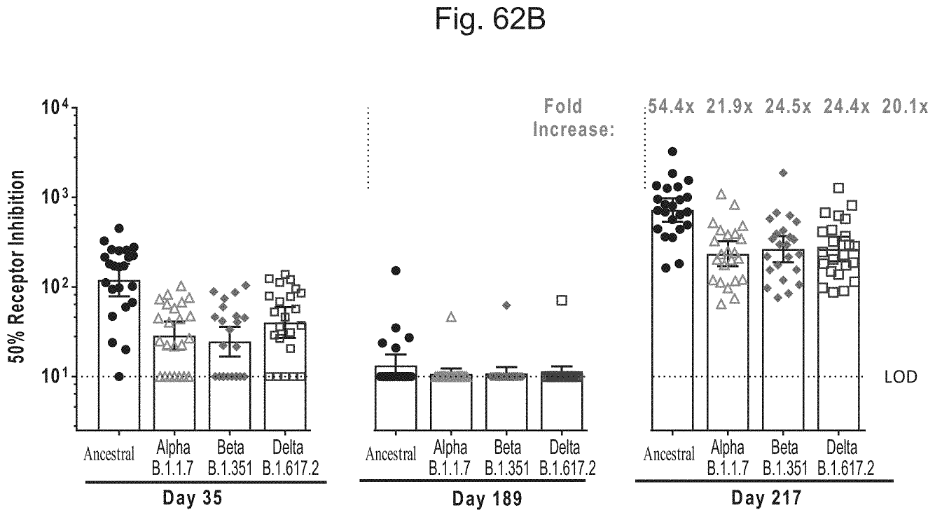

Two assays were developed to assess immune responses against additional SARS-CoV-2 variants using participant sera from Day 35 (Group B) and Day 217 (Group B2). A functional hACE2 inhibition assay was utilized to compare activity against the ancestral strain (a SARS-CoV-2 virus comprising a CoV S polypeptide with a D614G mutation compared to SEQ ID NO: 1) and the Delta, Beta, and Alpha variants of SARS-CoV-2. In respective order, 6-fold, 6.6-fold, 10.8-fold, 8.1 fold, and 19.9-fold increases in hACE2 inhibition titers were observed (Table 12A, Table 12B, FIGS. 62A-B). A second assay comparing anti-rS IgG activity across the same strains of SARS-CoV-2 found that 5.4-fold (Ancestral), 11.1-fold (Delta), 6.5-fold (Beta), 9.7-fold (Alpha) higher titers were observed after the booster (Table 11A, Table 11B, FIGS. 61A-B).

Results: Administration of a single booster dose of the vaccine approximately 6 months following the primary two-dose series resulted in an incremental increase in reactogenicity events along with significantly enhanced immunogenicity.

Prior to boosting at Day 189, anti-SARS-CoV-2 antibody titers in immunized participants were markedly lower when compared with samples taken after the primary vaccination series at Day 35 (Group B IgG and MN₅₀ GMTs lowered from 43,905 EU to 6,064 EU and 1,470 to 63, respectively). The presence of neutralizing antibodies are strongly indicative of protection against symptomatic COVID-19.

In the present study, antibody responses to the booster were assessed for the ancestral vaccine strain as well as for more recent SARS-CoV-2 variants including Alpha, Beta, and Delta. For the ancestral strain, IgG titers at Day 217 were approximately 34-fold higher than the pre-booster Day 189 titers while neutralizing antibody titers increased approximately 96-fold after the booster. Both IgG and MN titers after the booster were >4-fold higher than those seen after the primary two-dose series at Day 35, which is notable as the Day 35 titers corresponded to high levels of clinical efficacy in both a UK phase 3 study (89.7%) as well as in a USA/Mexico phase 3 study (90.4%).). When broken down by age group, higher fold increases were seen for older adults (≥60 to ≤84 years of age) compared to younger adults (≥18 to ≤59 years of age). This finding suggests that a booster dose may have added benefit in older adults as their antibody responses following the primary two-dose vaccination series were lower n those seen in younger adults.

For the Beta variant, 40- to 50-fold increases in IgG and MN antibody titers were seen following the booster and IgG titers were approximately 4-fold higher than those seen for the ancestral strain after the primary vaccination series. Unlike the observation with IgG, MN₅₀GMTs for the Beta variant were lower following the booster than those for the ancestral strain following the primary vaccination series (GMT 661 vs 1,470) in alignment with the known decreased neutralizing responses for this variant.

For the Delta variant of SARS-CoV-2, 6.6-fold increases in functional hACE2 inhibition titers were seen when comparing the post-booster Day 217 titers to the Day 35 titers. Anti-rS IgG activity compared at these same time points found 9.7-fold (Delta) higher titers associated with the booster.

The incidence of both local and systemic reactogenicity was higher following the 6-month booster dose compared to the previous doses reflecting the increased immunogenicity seen with the third dose. However, the incidence of Grade 3 or higher events remained relatively low with only fatigue (12.2%) being recorded by greater 10% of participants. In total, five Grade 4 (potentially life threatening) solicited local and systemic adverse events were reported. All five of these events (pain, tenderness, headache, malaise, and muscle pain) were reported by the same participant in the active booster group concurrently with an adverse event of drug hypersensitivity related to the vaccine. The drug hypersensitivity event was assessed as mild in severity. The participant did not seek any medical attention for this event, and all the participant's symptoms resolved over a period of 6 days.

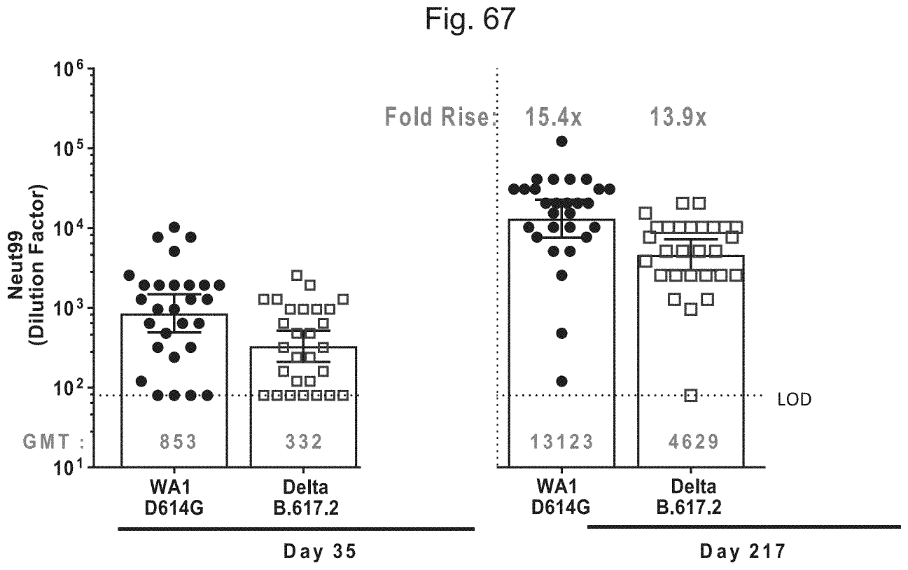

Table 13 shows the geometric mean titer for neutralization of 99% of a SARS-CoV-2 virus having a D614G mutation compared to SEQ ID NO: 1 or the SARS-CoV-2 delta variant. FIG. 67 shows the neutralizing antibody 99 (neut99) values for the immunogenic composition comprising BV2373 and saponin adjuvant of Example 11 against the SARS-CoV-2 strain containing a D614G mutation and the B.1.617.2 (delta variant).

Overall, a single booster dose of BV2373 and saponin adjuvant administered approximately 6 months after the primary series induced a substantial increase in humoral antibodies that was >4-fold higher than antibody titers associated with high levels of efficacy in two phase 3 studies while also displaying an acceptable safety profile. These findings support use of the vaccine in booster programs.

TABLE 8
Demographic and Baseline Characteristics
for Groups A, B1, and B2

| | Group A | | Group B1 | | Group B2 | |
|---|---|---|---|---|---|---|
| Parameter | N = 172 | | N = 102 | | N = 105 | |
| Age (years) | | | | | | |
| Mean (SD) | 51.9 | (17.23) | 52.0 | (16.99) | 51.7 | (17.12) |
| Median | 56.0 | | 57.5 | | 58.0 | |
| Min, Max | 18, 83 | | 19, 80 | | 19, 82 | |
| Age group (n [%]) | | | | | | |
| 18 to 59 years | 95 | (55.2) | 55 | (53.9) | 57 | (54.3) |
| 60 to 84 years | 77 | (44.8) | 47 | (46.1) | 48 | (45.7) |
| Sex (n [%]) | | | | | | |
| Male | 100 | (58.1) | 43 | (42.2) | 58 | (55.2) |
| Female | 72 | (41.9) | 59 | (57.8) | 47 | (44.8) |
| Race (n [%]) | | | | | | |
| White | 151 | (87.8) | 86 | (84.3) | 93 | (88.6) |
| Black or African | 2 | (1.2) | 3 | (2.9) | 3 | (2.9) |
| American | | | | | | |
| Asian | 15 | (8.7) | 10 | (9.8) | 7 | (6.7) |
| American Indian or | 2 | (1.2) | 1 | (1.0) | 1 | (1.0) |
| Alaska Native | | | | | | |
| Multiple | 2 | (1.2) | 1 | (1.0) | 1 | (1.0) |
| Missing | 0 | | 1 | (1.0) | 0 | |
| Ethnicity (n [%]) | | | | | | |
| Hispanic or Latino | 11 | (6.4) | 3 | (2.9) | 1 | (1.0) |
| Not Hispanic or Latino | 161 | (93.6) | 97 | (95.1) | 104 | (99.0) |
| Unknown | 0 | | 2 | (2.0) | 0 | |
| Baseline BMI (kg/m2) | | | | | | |
| Mean (SD) | 27.29 | (4.207) | 26.69 | (4.060) | 27.43 | (4.040) |
| Median | 27.40 | | 26.50 | | 27.10 | |
| Min, Max | 17.7, 35.0 | | 17.3, 34.9 | | 18.2, 34.9 | |
| Baseline SARS-CoV-2 | | | | | | |
| status (n [%]) | | | | | | |
| Negative | 169 | (98.3) | 101 | (99.0) | 102 | (97.1) |
| Positive | 2 | (1.2) | 1 | (1.0) | 3 | (2.9) |

| | | | | |
|---|---|---|---|---|
| Indeterminate | 1 | (0.6) | 0 | 0 |

BMI = body mass index;
SARS-CoV-2 = severe acute respiratory syndrome coronavirus 2;
SD = standard deviation
A = Placebo on Day 0, Day 21, and Day 189
B1 = 5 μg BV2373 + 50 μg saponin adjuvant on Day 0 and Day 21 and placebo on Day 189
B2 = 5 μg BV2373 + 50 μg saponin adjuvant on Day 0, Day 21, and Day 189
1 Participants in the Safety Analysis Set are counted according to the treatment received to accommodate for treatment errors.

TABLE 9
Serum IgG Geometric Mean Titers after Primary and Booster Vaccination for the Ancestral and Beta
Variant SARS-CoV-2 Strains by Study Day for Participants Receiving BV2373 and Saponin Adjuvant

| | Serum IgG GMT (EU [95% CI]) | | | | |
|---|---|---|---|---|---|
| | Day 35 | Day 189 | Day 217 | Day 189 | Day 217 |
| Age Group | Ancestral Strain | Ancestral Strain | Ancestral Strain | Beta variant | Beta variant |
| All Participants, | 43,905 | 6,064 | 204,367 | 4,317 | 175,190 |
| 18 to 84 years | (37,500, 51,403) | (4,625, 7,952) | (164,543, 253,828) | (3,261, 5,715) | (139,895, 219.391) |
| Participants | 65,255 | 8,102 | 270,224 | 6,310 | 226,103 |
| 18 to 59 years | (55,747, 76,385) | (6,041, 10,866) | (214,304, 340,736) | (4,642, 8,578) | (176,090, 290,321) |
| Participants | 28,137 | 4,238 | 144,440 | 2,700 | 127,601 |
| 60 to 84 years | (21,617, 36,623) | (2,631, 6,826) | (99,617, 209,431) | 1,682, 4,333) | (86,809, 187,561) |

CI = confidence interval;
ELISA = enzyme-linked immunosorbent assay;
EU = ELISA unit;
GMT = geometric mean titer

TABLE 10
Neutralizing Antibody Activity after Primary and Booster Vaccination
for the Ancestral and Beta Variant SARS-CoV-2 Strains by Study Day
for Participants Receiving BV2373 and Saponin Adjuvant

| | MN₅₀ GMT (95% CI) | | | | |
|---|---|---|---|---|---|
| | Day 35 | Day 189 | Day 217 | Day 189 | Day 217 |
| Age Group | Ancestral Strain | Ancestral Strain | Ancestral Strain | Beta variant | Beta variant |
| All Participants, | 1,470 | 63 | 6,023 | 13 | 661 |
| 18 to 84 years | (1,008, 2,145) | (49, 81) | (4,542, 7,988) | (11, 15) | (493, 886) |
| Participants | 2,281 | 80 | 8,568 | 14 | 871 |
| 18 to 59 years | (1,414, 3,678) | (56, 114) | (6,646, 11,046) | (11, 18) | (656, 1,156) |
| Participants | 981 | 47 | 3,936 | 12 | 469 |
| 60 to 84 years | (560, 1,717) | (33, 65) | (2,341, 6,620) | (10, 15) | (270, 816) |

CI = confidence interval;
GMT = geometric mean titer;
MN₅₀ = microneutralization assay at an inhibitory concentration > 50%

TABLE 11A
anti-CoV S IgG over time

| | Anti - rS BV2373 Titer | | | | | | | |
|---|---|---|---|---|---|---|---|---|
| | (EC50) | | | | | | | |
| | (SARS-CoV 2 Virus | | | | | | | |
| | comprising a Spike Protein | | | | Anti - rS BV2465 Titer | | | |
| | with a D614G mutation | | | | (EC50) | | | |
| | compared to SEQ ID NO: 1) | | | | (Delta) | | | |
| | D 0 | D 35 | D 189 | D 217 | D 0 | D 35 | D 189 | D 217 |
| GMT | 166 | 60742 | 5361 | 327758 | 156 | 26097 | 3143 | 290782 |
| Lower 95% CI | 134 | 42176 | 3782 | 225862 | 144 | 17501 | 1952 | 195349 |
| Lower 95% CI | 206 | 87481 | 7599 | 475623 | 169 | 38916 | 5059 | 432836 |
| GMFR | GMFR: 5.4 | | | | GMFR: 11.1 | | | |
| (D 35-D 217) | (CI - 3.34-8.71) | | | | (CI - 6.5-19.1) | | | |
| GMFR | GMFR: 61.2 | | | | GMFR: 92.6 | | | |
| (D 189-D 217) | (CI - 38.9-96.4) | | | | (CI - 52.8-162.4) | | | |
| | Anti - rS BV2438 Titer | | | | Anri - rS BV2425 Titer | | | |
| | (EC50) | | | | (EC50) | | | |
| | (Beta) | | | | (Alpha) | | | |
| | D 0 | D 35 | D 189 | D 217 | D 0 | D 35 | D 189 | D 217 |
| GMT | 161 | 40416 | 4066 | 264321 | 156 | 24333 | 2739 | 235145 |
| Lower 95% CI | 139 | 28091 | 2767 | 177965 | 143 | 15234 | 1777 | 152897 |
| Lower 95% CI | 188 | 58147 | 5975 | 392582 | 171 | 38865 | 4223 | 361636 |

| | | |
|---|---|---|
| GMFR | GMFR: 6.54 | GMFR: 9.7 |
| (D 35-D 217) | (CI - 3.97-10.8) | (CI - 5.56-11.9) |
| GMFR | GMFR: 65.0 | GMFR: 85.9 |
| (D 189-D 217) | (CI - 40.0-105.4) | (CI - 50.4-146.1) |

TABLE 11B
Anti- rS IgG Geometric Mean Titers after Primary and Booster Vaccination
for the Ancestral and Variant SARS-CoV-2 Strains by Study Day
for Participants Receiving BV2373 and Saponin Adjuvant

| | Ancestral | | Delta | | Beta | | Alpha | |
|---|---|---|---|---|---|---|---|---|
| Parameter | Day 35 | Day 217 | Day 35 | Day 217 | Day 35 | Day 217 | Day 35 | Day 217 |
| GMT | 60,742 | 327,758 | 26,097 | 290,782 | 40,416 | 264,321 | 24,333 | 235,145 |
| (95% CI) | (42,176, | (225,862, | (17,501, | (195,349, | (28,091, | (177,965, | (15,234, | (152,897, |
| | 87,481) | 475,623) | 38,916) | 432,836) | 58,147) | 392,582) | 38,865) | 361,636) |

| | | | | |
|---|---|---|---|---|
| GMFR | 5.4 | 11.1 | 6.54 | 9.7 |
| (95% CI) | (3.34, 8.71) | (6.5, 19.1) | (3.97, 10.8) | (5.56, 11.9) |

TABLE 12A
50% hACE2 inhibition titer over time

| | Anti - rS BV2373 RI | | | | | | | | | | | | | | | |
|---|---|---|---|---|---|---|---|---|---|---|---|---|---|---|---|---|
| | Titer (SARS-CoV 2 | | | | | | | | | | | | | | | |
| | Virus comprising a | | | | | | | | | | | | | | | |
| | Spike Protein with a | | | | | | | | | | | | | | | |
| | D614G mutation | | | | Anti - rS BV2465 RI | | | | Anti - rS BV2438 RI | | | | Anti - rS BV2425 RI | | | |
| | compared to SEQ ID | | | | Titer | | | | Titer | | | | Titer | | | |
| | NO: 1) | | | | (Delta) | | | | (Beta) | | | | (Alpha) | | | |
| | D 0 | D 35 | D 189 | D 217 | D 0 | D 35 | D 189 | D 217 | D 0 | D 35 | D 189 | D 217 | D 0 | D 35 | D 189 | D 217 |
| GMT | 10 | 119.6 | 13.3 | 723.1 | 10 | 40.0 | 10.9 | 265.3 | 10 | 24.6 | 10.8 | 265.2 | 10 | 28.7 | 10.7 | 234.4 |
| Lower 95% CI | 10 | 78.7 | 10.03 | 533.5 | 10 | 27.03 | 9.12 | 192.9 | 10 | 16.7 | 9.18 | 189.3 | 10 | 20.0 | 9.30 | 170.2 |
| Lower 95% CI | 10 | 181.9 | 17.6 | 980.0 | 10 | 59.5 | 12.99 | 364.7 | 10 | 36.04 | 12.8 | 371.5 | 10 | 41.05 | 12.3 | 322.8 |

| | | | | |
|---|---|---|---|---|
| GMFR | GMFR: 6.1 | GMFR: 6.61 | GMFR: 10.8 | GMFR: 8.1 |
| (D 35-D 217) | (CI - 3.79-9.89) | (CI - 4.34-10.09) | (CI - 7.1-16.4) | (CI - 5.56-11.9) |
| GMFR | GMFR: 54.4 | GMFR: 24.4 | GMFR: 24.5 | GMFR: 21.9 |
| (D 189-D 217) | (CI - 37.0-79.8) | (CI - 16.6-35.7) | (CI - 16.5-36.4) | (CI - 15.07-31.9) |

TABLE 12B
hACE2 Inhibition Geometric Mean Titers after Primary and Booster
Vaccination for Ancestral and Variant SARS-CoV-2 Strains by Study
Day for Participants Receiving BV2373 and Saponin Adjuvant

| | Ancestral | | Delta | | Beta | | Alpha | |
|---|---|---|---|---|---|---|---|---|
| Parameter | Day 35 | Day 217 | Day 35 | Day 217 | Day 35 | Day 217 | Day 35 | Day 217 |
| GMT | 119.6 | 723.1 | 40.0 | 265.3 | 24.6 | 265.2 | 28.7 | 234.4 |
| (95% CI) | (78.7, | (533.5, | (27.03, | (192.9, | (16.7, | (189.3, | (20.0, | (170.2, |
| | 181.9) | 980.0) | 59.5) | 364.7) | 36.04) | 371.5) | 41.05) | 322.8) |

| | | | | |
|---|---|---|---|---|
| GMFR | 6.1 | 6.61 | 10.8 | 8.1 |
| (95% CI) | (3.79, 9.89) | (4.34, 10.09) | (7.1, 16.4) | (5.56, 11.9) |

TABLE 13
Geometric Mean Titer for Neutralization of SARS-CoV-2 virus
with D614G mutation and the B.1.617-2 Delta Variant

| | SARS-CoV-2 virus with | | Delta | |
|---|---|---|---|---|
| | D614G mutation | | Neut99 Titer | |
| | Neut99 Titer | | (B.617.2) | |
| | D 35 | D 217 | D 35 | D 217 |
| GMT | 853 | 13123 | 331.6 | 4629 |
| Lower 95% CI | 490.2 | 7619 | 212 | 2961 |
| upper 95% CI | 1484 | 22603 | 518.5 | 7236 |

| | | |
|---|---|---|
| GMFR | GMFR: 15.4 | GMFR: 13.9 |
| (D 35-D 217) | (CI - 15.5-15.2) | (CI - 13.95-13.96) |

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1-66. (canceled)

67. A non-naturally occurring sudden acute respiratory syndrome coronavirus (SARS-CoV-2) spike (S) glycoprotein, wherein the SARS-CoV-2 S glycoprotein comprises:
(i) an inactivated furin cleavage site, wherein the inactivated furin cleavage site has the sequence of QQAQ (SEQ ID NO: 7);
(ii) mutation of amino acids 973 and 974 to proline;
(iii) mutation of amino acid 67 to alanine;
(iv) mutation of amino acid 202 to glycine;
(v) mutation of amino acid 229 to histidine;
(vi) mutation of amino acid 404 to asparagine;
(vii) mutation of amino acid 471 to lysine;
(viii) mutation of amino acid 488 to tyrosine;
(ix) mutation of amino acid 601 to glycine; and
(x) mutation of amino acid 688 to valine;
wherein the amino acids are numbered according to SEQ ID NO: 2.

68. The non-naturally occurring SARS-CoV-2 S glycoprotein of claim 67, wherein the glycoprotein comprises a polypeptide sequence having at least 95% identity to SEQ ID NO: 115.

69. The non-naturally occurring SARS-CoV-2 S glycoprotein of claim 67, wherein the glycoprotein comprises the polypeptide sequence of SEQ ID NO: 115.

70. An immunogenic composition comprising:
(i) the SARS-CoV-2 S glycoprotein of claim 67;
(ii) an adjuvant; and
(iii) a pharmaceutically acceptable buffer.

71. The immunogenic composition of claim 70; wherein the adjuvant is selected from alum, incomplete Freund's adjuvant; aluminum hydroxide adjuvant; a paucilamellar lipid vesicle; monophosphoryl lipid A; MF-59; or a saponin adjuvant.

72. The immunogenic composition of claim 71, wherein the adjuvant is a saponin adjuvant.

73. The immunogenic composition of claim 72, wherein the saponin adjuvant comprises at least two iscom particles, wherein:
the first iscom particle comprises fraction A of Quillaja Saponaria Molina and not fraction C of Quillaja Saponaria Molina; and
the second iscom particle comprises fraction C of Quillaja Saponaria and not fraction A of Quillaja Saponaria Molina.

74. The immunogenic composition of claim 73, wherein fraction A of Quillaja Saponaria Molina accounts for 50-96% by weight and fraction C of Quillaja Saponaria Molina accounts for the remainder, respectively, of the sum of the weights of fraction A of Quillaja Saponaria Molina and fraction C of Quillaja Saponaria Molina in the adjuvant.

75. The immunogenic composition of claim 73, wherein fraction A of Quillaja Saponaria Molina and fraction C of Quillaja Saponaria Molina account for about 85% by weight and about 15% by weight, respectively, of the sum of the weights of fraction A of Quillaja Saponaria Molina and fraction C of Quillaja Saponaria Molina in the adjuvant.

76. The immunogenic composition of claim 73, wherein fraction A of Quillaja Saponaria Molina and fraction C of Quillaja Saponaria Molina account for about 92% by weight and about 8% by weight, respectively, of the sum of the weights of fraction A of Quillaja Saponaria Molina and fraction C of Quillaja Saponaria Molina in the adjuvant.

77. The immunogenic composition of claim 70, wherein the SARS-CoV-2 S glycoprotein comprises a polypeptide sequence with at least 95% identity to SEQ ID NO: 115.

78. The immunogenic composition of claim 70, wherein the SARS-CoV-2 S glycoprotein comprises the polypeptide sequence of SEQ ID NO: 115.

79. The immunogenic composition of claim 70, comprising a second SARS-CoV-2 S glycoprotein, wherein the SARS-CoV-2 S glycoprotein comprises:
(i) an inactivated furin cleavage site, wherein the inactivated furin cleavage site has the sequence of QQAQ (SEQ ID NO: 7); and
(ii) mutation of amino acids 973 and 974 to proline;
wherein the amino acids are numbered according to SEQ ID NO: 2.

80. The immunogenic composition of claim 79, wherein the second SARS-CoV-2 S glycoprotein comprises a polypeptide sequence with at least 95% identity to SEQ ID NO: 87.

81. The immunogenic composition of claim 79, wherein the second SARS-CoV-2 S glycoprotein comprises the polypeptide sequence of SEQ ID NO: 87.

82. The immunogenic composition of claim 79; wherein the adjuvant is selected from alum, incomplete Freund's adjuvant; aluminum hydroxide adjuvant; a paucilamellar lipid vesicle; monophosphoryl lipid A; MF-59; or a saponin adjuvant.

83. The immunogenic composition of claim 82, wherein the adjuvant is a saponin adjuvant.

84. The immunogenic composition of claim 83, wherein the saponin adjuvant comprises at least two iscom particles, wherein:
the first iscom particle comprises fraction A of Quillaja Saponaria Molina and not fraction C of Quillaja Saponaria Molina; and
the second iscom particle comprises fraction C of Quillaja Saponaria Molina and not fraction A of Quillaja Saponaria Molina.

85. The immunogenic composition of claim 84, wherein fraction A of Quillaja Saponaria Molina accounts for 50-96% by weight and fraction C of Quillaja Saponaria Molina accounts for the remainder, respectively, of the sum of the weights of fraction A of Quillaja Saponaria Molina and fraction C of Quillaja Saponaria Molina in the adjuvant.

86. The immunogenic composition of claim 84, wherein fraction A of Quillaja Saponaria Molina and fraction C of Quillaja Saponaria Molina account for about 85% by weight and about 15% by weight, respectively, of the sum of the weights of fraction A of Quillaja Saponaria Molina and fraction C of Quillaja Saponaria Molina in the adjuvant.

87. The immunogenic composition of claim 84, wherein fraction A of Quillaja Saponaria Molina and fraction C of Quillaja Saponaria Molina account for about 92% by weight and about 8% by weight, respectively, of the sum of the weights of fraction A of Quillaja Saponaria Molina and fraction C of Quillaja Saponaria Molina in the adjuvant.

88. A method of stimulating an immune response against SARS-CoV-2 or a heterogeneous SARS-CoV-2 strain in a subject comprising administering the immunogenic composition of claim 67.

89. The method of claim 88, wherein from about 3 μg to about 25 μg of CoV S glycoprotein is administered.

90. The method of claim 88, wherein about 50 μg of the adjuvant is administered.

91. The method of claim 88, wherein the adjuvant of the immunogenic composition is a saponin adjuvant.

92. The method of claim 88, wherein the subject is administered a first dose of the immunogenic composition at day 0 and a boost dose at day 21.

93. A method of stimulating an immune response against SARS-CoV-2 or a heterogeneous SARS-CoV-2 strain in a subject comprising administering the immunogenic composition of claim 79.

94. The method of claim 93, wherein from about 3 μg to about 25 μg of CoV S glycoprotein is administered.

95. The method of claim 93, wherein about 50 μg of the adjuvant is administered.

96. The method of claim 93, wherein the adjuvant of the immunogenic composition is a saponin adjuvant.

97. The method of claim 93, wherein the subject is administered a first dose of the immunogenic composition at day 0 and a boost dose at day 21.
